(19) United States
(12) Patent Application Publication
(10) Pub. No.: US 2026/0008856 A1
(43) Pub. Date: Jan. 8, 2026

(54) METHODS OF TREATING DISEASE USING ANTI-IL1RAP ANTIBODIES AND ANTIBODY DRUG CONJUGATES

(71) Applicant: Bluefin Biomedicine, Inc., Beverly, MA (US)

(72) Inventors: Scott LONNING, Westford, MA (US); Ting-Lei Gu, Andover, MA (US); Christine Barbon, Groton, MA (US); Ailan Guo, Lexington, MA (US); Alain Curaudeau, Cambridge, MA (US)

(21) Appl. No.: 18/881,711

(22) PCT Filed: Jul. 6, 2023
(86) PCT No.: PCT/US2023/069728

§ 371 Date: Jan. 6, 2025

Related U.S. Application Data
(60) Provisional application No. 63/358,858, filed on Jul. 6, 2022.

(51) Int. Cl. C07K 16/28; A61K 39/00; A61K 45/06; A61P 17/00; A61P 37/06

(52) U.S. Cl. CPC C07K 16/2866; A61K 45/06; A61P 17/00; A61P 37/06; A61K 2039/505; A61K 2039/545; C07K 2317/565; C07K 2317/76; C07K 2317/77

(57) ABSTRACT

Disclosed herein are Interleukin 1 Receptor Accessory Protein (IL1RAP) antibodies, including compositions and methods of using said antibodies.

### RELATED APPLICATIONS

The instant application claims priority to U.S. Provisional Application No. 63/358,858, filed on Jul. 6, 2022, the entire contents of which are expressly incorporated by reference herein in their entirety.

### BACKGROUND

IL1RAP, also known as “Interleukin 1 Receptor Accessory Protein,” “IL-1R Accessory Protein”, “IL-1 Receptor Accessory Protein,” “Interleukin-1 Receptor 3,” “IL-1RAcP,” “C3orf13,” “IL-1R3,” “IL1R3,” “Interleukin-1 Receptor Accessory Protein Beta,” “Interleukin-1 Receptor Accessory Protein,” and “IL-1R-3,” (Wesche, H., J. Biol. Chem. 272: 7727-7731, 1997) is a necessary part of the interleukin 1 (IL-1) receptor complex which initiates signaling events that result in the activation of interleukin 1-responsive genes. In addition to IL 1-signaling, IL1RAP is critical for mediating the effects of IL-33, through the ST2/IL1RAP complex, and IL-36, through the IL 1Rrp2/IL1RAP complex (Garlanda et al, Immunity. 2013 Dec. 12; 39(6):1003-18).

Two IL-1 receptors, IL-1R type I and IL-1R type II, have been identified. Both receptors can interact with both forms of IL-1, i.e., IL-1α and IL-1β. IL-1 R1 is responsible for mediating IL-1-induced cellular activation. However, the IL-1/IL-1 R1 complex cannot signal by itself, but is dependent on association with IL1RAP (Dinarello, CA, Blood 30 1996, 87(6): 2095-147) (see, e.g., WO 2015/132602).

Alternative splicing of IL1RAP results in two transcript variants encoding two different isoforms, one membrane-bound and one soluble. The ratio of soluble to membrane-bound forms increases during acute-phase induction or stress. IL1RAP is expressed on candidate leukemic stem cells in the majority of AML patients, but not on normal hematopoietic stem cells (Agerstam, et al. PNAS USA (2015) vol. 112:34, 10786-10791).

### Interleukin-1

Interleukin 1 (IL-1) is a potent pro-inflammatory cytokine that induces synthesis of acute phase and proinflammatory proteins during infection, tissue damage, or stress, by forming a complex at the cell membrane with an interleukin 1 receptor and an accessory protein. IL-1 can be produced by a variety of cell types, including mononuclear phagocytes, in response to infection and inflammation. The IL-1 family consists of seven agonists, including IL-1α and IL-1β, and three naturally occurring receptor antagonists, including the IL-1 receptor antagonist (IL-1Ra) (Dinarello, CA, Blood 1996, 87(6): 2095-147).

IL-1 is capable of activating several cell types including leukocytes and endothelial cells. IL-1 induces and amplifies immunological responses by promoting the production and expression of adhesion molecules, cytokines, chemokines and other inflammatory mediators such as prostaglandin E2 and nitric oxide (NO). As a consequence, local inflammation is amplified and sustained. In addition, the IL-1 induced production of inflammatory mediators results in fever, headache, hypotension and weight loss. Furthermore, IL-1 is a hematopoietic growth factor and has been shown to reduce the nadir of leukocytes and platelets in patients during bone marrow transplantation. IL-1 has also been shown to promote angiogenesis by inducing the production of vascular endothelial growth factor, thereby promoting pannus formation and blood supply in rheumatic joints. Finally, IL-1 has been shown to promote the bone and cartilage degradation in rheumatic diseases.

IL-1 is implicated in a wide range of diseases and conditions ranging from gout to cancer (for reviews, see Dinarello et al., 2012, Nature Reviews 11:633-652 and Dinarello, 2014, Mol. Med. 20 (suppl. 1):S43-S58. A number of therapies for blocking IL-1 activity are approved and in development. Targeting IL-1 began in 1993 with the introduction of anakinra (Kineret™; Amgen), a recombinant form of the naturally occurring IL-1 receptor antagonist (IL-1 Ra), which blocks the activity of both IL-1α and IL-1β. This therapeutic has since been used to demonstrate a role for IL-1 in numerous diseases. Neutralizing IL-1 with antibodies or soluble receptors has also proved to be effective, and the soluble decoy receptor rilonacept (Arcalyst™; Regeneron) and the anti-IL-1β (neutralizing monoclonal antibody canakinumab (IIaris™; Novartis) have now been approved. Other therapeutic approaches, including IL-1α neutralisation, a therapeutic vaccine targeting IL-1β and a chimeric IL-1 Ra, are in clinical trials. In addition, orally active small-molecule inhibitors of IL-1 production, such as caspase 1 inhibitors, have been developed.

However, there remains a need in the art for therapeutic agents that can be used for therapeutic purposes in the treatment of inflammatory, autoimmune, and atopic diseases, including atopic dermatitis and asthma, as well as suppurative inflammatory diseases such as hidradenitis suppurativa.

### SUMMARY

Provided herein are anti-IL1RAP antibodies, antigen-binding portions thereof, and antibody drug conjugates (ADCs) (e.g., as set forth in U.S. Pat. No. 11,248,054 which is incorporated by reference in its entirety for all purposes) for use in treating a variety of diseases, disorders, and conditions, including inflammatory or autoimmune diseases such as, for example, rheumatoid arthritis, psoriasis, psoriatic arthritis, ankylosing spondylitis, Crohn's disease, ulcerative colitis, atopic dermatitis, asthma, and the like.

In another aspect of the invention, the present disclosure provides methods for treating inflammatory or autoimmune diseases, disorders, or conditions, comprising administering a therapeutically effective amount of an antibody or antigen binding portion thereof, as described herein to a subject in need thereof.

In some embodiments, the inflammatory or autoimmune disease is rheumatoid arthritis, psoriasis, psoriatic arthritis, ankylosing spondylitis, Crohn's disease, ulcerative colitis, atopic dermatitis, or asthma. In some embodiments, the inflammatory or autoimmune disease is an atopic disease. In some embodiments, the inflammatory or autoimmune disease is characterized by neutrophil or eosinophil dysfunction. In some embodiments, the treatment decreases levels of TARK/CCL17 at the site of atopy or in serum. In some embodiments, the inflammatory or autoimmune disease is atopic dermatitis.

In some embodiments, the present disclosure provides a method for inhibiting or decreasing inflammation or autoimmune response in a subject having inflammation or autoimmunity, said method comprising administering an effective amount of the antibody or antigen binding portion thereof, as described herein, to the subject having the inflammation or autoimmunity, such that the inflammation or autoimmunity is inhibited or decreased.

In some embodiments, the methods of treating inflammatory or autoimmune diseases, disorders, or conditions, or of inhibiting or decreasing inflammation or autoimmune response include a step of identifying a mammal in need of such treatment according to abnormal serum levels of IL-1α, IL-1β, IL-33, IL-36α, IL-36β, and IL-36γ, or of CXCL1, CXCL8, TARC, LCN2, or combinations thereof.

In some embodiments, the antibody or antigen binding portion thereof is characterized by its inhibition of IL-8 release by intestinal epithelial cells, intestinal myofibroblasts, or dermal fibroblasts stimulated with IL-1α, IL-1β, IL-36α, IL-36β, and IL-36γ.

In some embodiments, the antibody or antigen binding portion thereof is administered in combination with at least one other agent or therapy. In some embodiments the at least one other agent or therapy is a therapeutic antibody, small molecule drug, siRNA, mRNA, or any other modality known to those skilled in the art. In some embodiments, the antibody or antigen binding portion thereof is administered before, simultaneously with, or after the at least one other agent or therapy. In some embodiments, the at least one other agent or therapy agent inhibits the TH2-associated immune response, the TH1-associated immune response, pruritis, IL-4 signaling, IL-13 signaling, IL-22 signaling, IL-33 signaling, IL-17 signaling, IL-36 signaling, IL-18 signaling, IL-23 signaling, OX40 signaling, IL-5 signaling, T cell migration, IRAK4 signaling, complement signaling, PDE4 signaling, or combinations thereof.

Accordingly, provided herein are the following items:

1. A method of treating an inflammatory or autoimmune condition in a mammal in need thereof, comprising administering a therapeutically effective amount of an antibody against IL1RAP.
2. The method of item 1, wherein the inflammatory or autoimmune condition is an atopic condition, and wherein the antibody decreases levels of TARC/CCL17, PARC, periostin, IL-22, eotaxin-1, eotaxin-3, or combinations thereof at the site of atopy or in serum.
3. The method of item 1, wherein the inflammatory or autoimmune condition is characterized or caused by increased neutrophil or eosinophil cell counts; neutrophil or eosinophil dysfunction; or increased TARC/CCL17 levels.
4. The method of item 3, wherein the inflammatory or autoimmune condition is characterized or caused by increased neutrophil cell counts or neutrophil dysfunction.
5. The method of item 4, wherein the inflammatory or autoimmune condition characterized or caused by increased neutrophil cell counts or neutrophil dysfunction is selected from the group consisting of: hidradenitis suppurativa, generalized pustular psoriasis (GPP), COPD, idiopathic fibrosis, neutrophilic asthma, Neutrophil dermatose, Pyoderma Gangrenosum Schnitzler syndrome, Behçet's disease, Sweet's syndrome, rheumatoid arthritis, systemic lupus erythematosus (SLE), inflammatory bowel disease (Crohn's disease, ulcerative colitis), psoriasis, vasculitis, Alzheimer Disease, COVID-19, and Gout.
6. The method of item 3, wherein the inflammatory or autoimmune condition is further characterized by increased eosinophil cell counts or eosinophil dysfunction.
7. The method of item 6, wherein the inflammatory or autoimmune condition characterized or caused by increased eosinophil cell counts or eosinophil dysfunction is selected from the group consisting of: Atopic dermatitis (eczema), Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, and Eosinophilic Myocarditis.
8. The method of item 3, wherein the inflammatory or autoimmune condition is further characterized by increased TARC/CCL17 levels.
9. The method of item 8, wherein the inflammatory or autoimmune condition characterized by increased TARC/CCL17 levels is selected from the group consisting of: Atopic Dermatitis, COPD, bronchial asthma, allergic rhinitis, eosinophilic pneumonia, Hypersensitivity Pneumonitis, Lichen Planus, Sarcoidosis, Urticaria, Mastocytosis, eosinophilic-associated disorders.
10. The method of item 1, wherein the inflammatory or autoimmune condition is selected from the group consisting of sepsis, acute respiratory distress syndrome, COVID-19, myocardial infarction, cystic fibrosis, irritable bowel disease, ulcerative colitis, Crohn's disease, atopic dermatitis, psoriasis, multiple sclerosis, asthma, neutrophilic asthma, Alzheimer's disease, stroke, diabetic kidney disease, diabetes, diabetic retinopathy, Chronic obstructive pulmonary disease (COPD), idiopathic pulmonary fibrosis, non-alcoholic fatty liver disease, rheumatoid arthritis, psoriatic arthritis, ankylosing spondylitis, axial spondyloarthritis, systemic lupus erythematosus (SLE), vasculitis, Gout. Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria, and Mastocytosis.
11. The method of item 1, further comprising a step of identifying the mammal in need thereof according to abnormal serum levels of IL-1α, IL-1β, IL-33, IL-36α, IL-36β, IL-36γ, or combinations thereof.
12. The method of item 1, further comprising a step of identifying the mammal in need thereof according to abnormal serum levels of CXCL1, CXCL8, LCN-2, TARC, or combinations thereof.
13. The method of item 11, wherein the serum levels of IL-1α, IL-1β, IL-33, IL-36α, IL-36β, IL-36γ, or combinations thereof are elevated.
14. The method of item 12, wherein the serum levels of CXCL1, CXCL8, LCN-2, TARC, or combinations thereof are elevated.
15. The method of item 1, wherein the antibody is characterized by its inhibition of IL-8 release by intestinal epithelial cells, intestinal myofibroblasts, or dermal fibroblasts stimulated with IL-1α, IL-1β, IL-36α, IL-36β, and/or IL-36γ, or combinations thereof.
16. The method of item 2, wherein the atopic condition is selected from the group consisting of atopic dermatitis, asthma, COPD, allergic rhinitis, allergic conjunctivitis, food allergy, drug allergy, and angioedema.
17. The method of item 2, wherein the atopic condition is atopic dermatitis and the antibody decreases levels of TARC/CCL17, PARC, periostin, IL-22, eotaxin-1, eotaxin-3, or combinations thereof in the skin or serum.
18. The method of item 2, wherein the atopic condition is asthma.
19. The method of item 2, wherein the atopic condition is COPD.
20. The method of item 1, wherein the antibody is administered in combination with at least one other therapeutic agent.
21. The method of item 20, wherein the at least one other therapeutic agent is a therapeutic antibody, corticosteroid, small molecule, siRNA, mRNA, or combination thereof.
22. The method of item 20, wherein the at least one other therapeutic agent inhibits IL-4 signaling.
23. The method of item 22, wherein the at least one other therapeutic agent is an IL-4Ra inhibitor or antagonist, a Pan-JAK inhibitor or antagonist, or combinations thereof.
24. The method of item 22, wherein the at least one other therapeutic agent is dupilumab, CBP-201, AK120, cerdulatinib, CEE321, jaktinib, delgocitinib, filgotinib, tofacitinib, dexamethasone, triamcinolone, prednisone, or combinations thereof.
25. The method of item 22, wherein the inflammatory or autoimmune condition is rheumatoid arthritis, psoriasis, psoriatic arthritis, ankylosing spondylitis, axial spondyloarthritis, Polyarticular Course Juvenile Idiopathic Arthritis, Crohn's disease, ulcerative colitis, atopic dermatitis, COPD, or asthma.
26. The method of item 20, wherein the at least one other therapeutic agent inhibits IL-13 signaling.
27. The method of item 26, wherein the at least one other therapeutic agent is an IL-13 inhibitor or antagonist, a JAK1 inhibitor or antagonist, TYK2 inhibitor or antagonist, or combinations thereof.
28. The method of item 26, wherein the at least one other therapeutic agent is upadacitinib, abrocitinib, tralokinumab, lebrikizumab, eblasakimab, baricitinib, ruxolitinib, filgotinib, PF-06651600, dexamethasone, triamcinolone, prednisone, or combinations thereof.
29. The method of item 26, wherein the inflammatory or autoimmune condition is rheumatoid arthritis, Systemic lupus erythematosus, vitiligo, psoriasis, psoriatic arthritis, ankylosing spondylitis, axial spondyloarthritis, Crohn's disease, ulcerative colitis, atopic dermatitis, COPD or asthma.
30. The method of item 20, wherein the at least one other therapeutic agent inhibits IL-22 signaling.
31. The method of item 30, wherein the at least one other therapeutic agent is an IL-22 inhibitor or antagonist, an IL-22R1 inhibitor or antagonist, a JAK1 inhibitor or antagonist, a JAK2 inhibitor or antagonist, a TYK2 inhibitor or antagonist, or combinations thereof.
32. The method of item 30, wherein the at least one other therapeutic agent is fezakinumab, LEO 138559, brepocitinib, ATI-1777, deucravacitinib, TAK-279, upadacitinib and abrocitinib, or combinations thereof.
33. The method of item 30, wherein the inflammatory or autoimmune condition is rheumatoid arthritis, Systemic lupus erythematosus, vitiligo, psoriasis, psoriatic arthritis, ankylosing spondylitis, axial spondyloarthritis, Crohn's disease, ulcerative colitis, atopic dermatitis, COPD, or asthma.
34. The method of item 20, wherein the at least one other therapeutic agent inhibits IL-33 signaling.
35. The method of item 34, wherein the at least one other therapeutic agent is an IL-33 or IL-33R inhibitor or antagonist.
36. The method of item 34, wherein the at least one other therapeutic agent is etokimab, itepekimab, astegolimab, PF-06817024, tozorakimab, CNTO 7160, or combinations thereof.
37. The method of item 34, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, chronic obstructive pulmonary disease (COPD), asthma, allergic contact dermatitis, irritant contact dermatitis, rosacea, psoriasis vulgaris, pustular psoriasis, mastocytosis, systemic lupus erythematosus, systemic sclerosis, chronic spontaneous urticaria, autoimmune blistering diseases, Behcet's disease, or vitiligo.
38. The method of item 20, wherein the at least one other therapeutic agent inhibits IL-17 signaling.
39. The method of item 38, wherein the at least one other therapeutic agent is an IL-17A, IL-17C, IL-17F, IL17A/F, or an IL-17RA inhibitor or antagonist; or combinations thereof.
40. The method of item 38, wherein the at least one other therapeutic agent is secukinumab, ixekizumab, brodalumab, bimekizumab, izokibep, sonelokimab, or combinations thereof.
41. The method of item 38, wherein the inflammatory or autoimmune condition is rheumatoid arthritis, psoriasis, psoriatic arthritis, ankylosing spondylitis, axial spondyloarthritis, enthesitis-related arthritis, Crohn's disease, ulcerative colitis, atopic dermatitis, hidradenitis suppurativa, COPD, or asthma.
42. The method of item 20, wherein the at least one other therapeutic agent inhibits IL-36 signaling.
43. The method of item 42, wherein the at least one other therapeutic agent is an IL-36 inhibitor or antagonist or an IL-36R inhibitor or antagonist.
44. The method of item 42, wherein the at least one other therapeutic agent is spesolimab, imsidolimab, REGN6490, or combinations thereof.
45. The method of item 42, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, asthma, neutrophilic asthma, neutrophilic lung inflammation, COVID-19, Chronic obstructive pulmonary disease (COPD), idiopathic pulmonary fibrosis, non-alcoholic fatty liver disease, neutrophilic dermatoses, hidradenitis suppurativa, generalized pustular psoriasis, palmoplantar pustular psoriasis, deficiency of IL-36 receptor antagonist (DITRA), psoriasis vulgaris, CARD14-mediated psoriasis, acute generalized exanthematous pustulosis, pyoderma gangrenosum, Sweet's syndrome, systemic lupus erythematosus, systemic sclerosis, autoimmune blistering diseases, acne, allergic contact dermatitis, or folliculitis and eosinophilic pustular folliculitis.
46. The method of item 20, wherein the at least one other therapeutic agent inhibits IL-18 signaling.
47. The method of item 46, wherein the at least one other therapeutic agent is an IL-18 inhibitor or antagonist.
48. The method of item 46, wherein the at least one other therapeutic agent is tadekinig alfa.
49. The method of item 46, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, asthma, adult-onset Still disease, cutaneous lupus erythematosus, chronic spontaneous urticaria, contact dermatitis, alopecia areata, cutaneous drug eruptions, graft-versus-host disease, cryopyrin-associated periodic syndromes, granulomatosis with polyangiitis, systemic sclerosis, hidradenitis suppurativa, pyogenic arthritis, pyoderma gangrenosum and acne (PAPA), familial Mediterranean fever, rosacea, synovitis, acne, pustulosis, hyperostosis, osteitis (SAPHO), bullous pemphigoid, pemphigus vulgaris, Behcet's disease, or Schnitzler syndrome.
50. The method of item 20, wherein the at least one other therapeutic agent inhibits IL-23 signaling.
51. The method of item 50, wherein the at least one other therapeutic agent is an IL-23 inhibitor or antagonist, an IL-12/23 p40 subunit inhibitor or antagonist, an IL-23 p19 subunit inhibitor or antagonist, or combinations thereof.
52. The method of item 50, wherein the at least one other therapeutic agent is risankizumab, ustekinumab, guselkumab, tildrakizumab, mirikizumab, brazikumab, or combinations thereof.
53. The method of item 50, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.
54. The method of item 20, wherein the at least one other therapeutic agent inhibits OX40 signaling.
55. The method of item 54 wherein the at least one other therapeutic agent is an OX40 or OX40L inhibitor or antagonist.
56. The method of item 54, wherein the at least one other therapeutic agent is rocatinlimab, GBR 830, amlitelimab, or combinations thereof.
57. The method of item 54, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.
58. The method of item 20, wherein the at least one other therapeutic agent inhibits IL-5 signaling.
59. The method of item 58 wherein the at least one other therapeutic agent is an IL-5Ra inhibitor or antagonist, a JAK1 inhibitor or antagonist, or combinations thereof.
60. The method of item 58, wherein the at least one other therapeutic agent is benralizumab, upadacitinib, abrocitinib, SHR0302, filgotinib, PF-06651600, dexamethasone, triamcinolone, prednisone, or combinations thereof.
61. The method of item 58, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.
62. The method of item 20, wherein the at least one other therapeutic agent inhibits T cell migration.
63. The method of item 62 wherein the at least one other therapeutic agent is an S1PR1 inhibitor or antagonist, an S1PR4 inhibitor or antagonist, an S1PR5 inhibitor or antagonist, a CCR4 inhibitor or antagonist, or combinations thereof.
64. The method of item 62, wherein the at least one other therapeutic agent is etrasimod, ozanimod, SCD-044, LC51-0255, BMS-986166, RPT193, or combinations thereof.
65. The method of item 62, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.
66. The method of item 20, wherein the at least one other therapeutic agent inhibits pruritis.
67. The method of item 66 wherein the at least one other therapeutic agent is an IL-1α inhibitor or antagonist, an OSMRβ inhibitor or antagonist, an NK1R inhibitor or antagonist, a P2X3 inhibitor or antagonist, an IL-31 inhibitor or antagonist, or combinations thereof.
68. The method of item 66, wherein the at least one other therapeutic agent is bermekimab, vixarelimab, serlopitant, tradipitant, BLU-5937, nemolizumab, or combinations thereof.
69. The method of item 66, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.
70. The method of item 20, wherein the at least one other therapeutic agent inhibits TH1-associated immune response.
71. The method of item 70 wherein the at least one other therapeutic agent is an IL-1α inhibitor or antagonist, IL-1β inhibitor or antagonist, IL-1R1 inhibitor or antagonist, IL-36R inhibitor or antagonist, a TNFα inhibitor or antagonist, or combinations thereof.
72. The method of item 70, wherein the at least one other therapeutic agent is bermekimab, anakinra, canakinumab, gevokizumab, rilonacept, MEDI8968, spesolimab, imsidolimab, REGN6490, adalimumab, infliximab, etanercept, certolizumab, golimumab, or combinations thereof.
73. The method of item 70, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, or monogenic autoinflammatory skin disorders.
74. The method of item 20, wherein the at least one other therapeutic agent inhibits TH2-associated immune responses.
75. The method of item 74 wherein the at least one other therapeutic agent is an IL-4 inhibitor or antagonist, Type I IL-4 receptor inhibitor or antagonist, Type II IL-4 receptor inhibitor or antagonist, IL-13 inhibitor or antagonist, Type I IL-13 receptor inhibitor or antagonist, Type II IL-13 receptor inhibitor or antagonist, and IL-5 inhibitor or antagonist, Type I IL-5 receptor inhibitor or antagonist, Type II IL-5 receptor inhibitor or antagonist, IL-9 inhibitor or antagonist, Type I IL-9 receptor inhibitor or antagonist, IL-10 inhibitor or antagonist, homodimeric IL-10 receptor inhibitor or antagonist, heterodimeric IL-10 receptor inhibitor or antagonist or combinations thereof.
76. The method of item 74, wherein the at least one other therapeutic agent is dupilumab, omalizumab, mepolizumab, benralizumab, tralokinumab, lebrikizumab, or combinations thereof.
77. The method of item 74, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, or monogenic autoinflammatory skin disorders Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria.
78. The method of item 20, wherein the at least one other therapeutic agent inhibits TH17-associated immune responses.
79. The method of item 78 wherein the at least one other therapeutic agent is an IL-17 inhibitor or antagonist, Type I IL-17 receptor inhibitor or antagonist, Type II IL-17 receptor inhibitor or antagonist, IL-23 inhibitor or antagonist, IL-23 receptor inhibitor or antagonist, or combinations thereof.
80. The method of item 78, wherein the at least one other therapeutic agent is secukinumab, ixekizumab, brodalumab, bimekizumab, izokibep, sonelokimab, risankizumab, ustekinumab, guselkumab, tildrakizumab, mirikizumab, brazikumab or combinations thereof.
81. The method of item 78, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria, or monogenic autoinflammatory skin disorders.
82. The method of item 20, wherein the at least one other therapeutic agent inhibits IRAK4 signaling.
83. The method of item 82 wherein the at least one other therapeutic agent is an IRAK4 inhibitor, degrader, or antagonist.
84. The method of item 82, wherein the at least one other therapeutic agent is PF-06650833, CA-4948, KT-474, or combinations thereof.
85. The method of item 82, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, hidradenitis suppurativa, COPD, or asthma.
86. The method of item 20, wherein the at least one other therapeutic agent inhibits complement signaling.
87. The method of item 86 wherein the at least one other therapeutic agent is a C5a inhibitor or antagonist, a C5aR inhibitor or antagonist, a TSLP inhibitor or antagonist, a CD80/CD86 inhibitor or antagonist, an IL-6 inhibitor or antagonist, a CD20 inhibitor or antagonist, an integrin α4 inhibitor or antagonist, an AhR agonist, or combinations thereof.
88. The method of item 86, wherein the at least one other therapeutic agent is vilobelimab, FX002, INF904, tezepelumab, abatacept, tocilizumab, sarilumab, rituximab, vedolizumab, tapinarof, tadekinig alfa, or combinations thereof.
89. The method of item 86, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, hidradenitis suppurativa, COVID-19, COPD, or asthma.
90. The method of item 20, wherein the at least one other therapeutic agent inhibits PDE4 signaling.
91. The method of item 90, wherein the at least one other therapeutic agent is a PDE4 inhibitor or antagonist.
92. The method of item 90, wherein the at least one other therapeutic agent is apremilast, crisaborole, difamilast, roflumilast, or combinations thereof.
93. The method of item 90, wherein the inflammatory or autoimmune condition is rheumatoid arthritis, psoriasis, psoriatic arthritis, ankylosing spondylitis, ankylosing spondylitis, axial spondyloarthritis, Crohn's disease, ulcerative colitis, atopic dermatitis, hidradenitis suppurativa, COPD or asthma Crohn's disease, ulcerative colitis, atopic dermatitis, or asthma.
94. The method of item 20, wherein the at least one other therapeutic agent is administered before, simultaneously with, or after the at least one other therapeutic agent.
95. The method of items 1, 17 and 20, wherein the antibody is administered subcutaneously or intravenously at a dose selected from: 1-80 mg/kg, 1-60 mg/kg, 1-50 mg/kg, 1-40 mg/kg, 1-30 mg/kg, 1-25 mg/kg, or 1-20 mg/kg.
98. The method of items 1, 17 and 20, wherein the antibody is administered as a subcutaneous injection or as a bolus intravenously, less frequently than once per week, twice per week, more than twice per week, or in a continuous infusion.
101. The method of item 1, 17 or 20, wherein the antibody comprises:
BFB759 (i.e., 37E10_15B5) corresponding to a heavy chain variable region comprising a CDR3 domain comprising the amino acid sequence of SEQ ID NO: 67, a CDR2 domain comprising the amino acid sequence of SEQ ID NO: 66, and a CDR1 domain comprising the amino acid sequence of SEQ ID NO: 65; and a light chain variable region comprising a CDR3 domain comprising the amino acid sequence of SEQ ID NO: 70, a CDR2 domain comprising the amino acid sequence of SEQ ID NO: 62, and a CDR1 domain comprising the amino acid sequence of SEQ ID NO: 69;
a heavy chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10; and a light chain variable region comprising a CDR3 domain comprising the amino acid sequence of SEQ ID NO: 16, a CDR2 domain comprising the amino acid sequence of SEQ ID NO: 15, and a CDR1 domain comprising the amino acid sequence of SEQ ID NO: 14;
a heavy chain variable region comprising a CDR3 domain comprising the amino acid sequence of SEQ ID NO: 51, a CDR2 domain comprising the amino acid sequence of SEQ ID NO: 50, and a CDR1 domain comprising the amino acid sequence of SEQ ID NO: 49; and a light chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53;
a heavy chain variable region comprising a CDR3 domain comprising the amino acid sequence of SEQ ID NO: 59, a CDR2 domain comprising the amino acid sequence of SEQ ID NO: 58, and a CDR1 domain comprising the amino acid sequence of SEQ ID NO: 57; and a light chain variable region comprising a CDR3 domain comprising the amino acid sequence of SEQ ID NO: 63, a CDR2 domain comprising the amino acid sequence of SEQ ID NO: 62, and a CDR1 domain comprising the amino acid sequence of SEQ ID NO: 61; or
a heavy chain variable region comprising a CDR3 domain comprising the amino acid sequence of SEQ ID NO: 176, a CDR2 domain comprising the amino acid sequence of SEQ ID NO: 175, and a CDR1 domain comprising the amino acid sequence of SEQ ID NO: 174; and a light chain variable region comprising a CDR3 domain comprising the amino acid sequence of SEQ ID NO: 179, a CDR2 domain comprising the amino acid sequence of SEQ ID NO: 178, and a CDR1 domain comprising the amino acid sequence of SEQ ID NO: 79.
   102. The method of item 1, 17 or 20, wherein the antibody comprises: BFB759 (i.e., 37E10_15B5) corresponding to a heavy chain variable region comprising the amino acid sequence of SEQ ID NO: 64 and a light chain variable region comprising the amino acid sequence of SEQ ID NO: 68;
a heavy chain variable region comprising the amino acid sequence of SEQ ID NO: 9 and a light chain variable region comprising the amino acid sequence of SEQ ID NO: 82;
a heavy chain variable region comprising the amino acid sequence of SEQ ID NO: 48 and a light chain variable region comprising the amino acid sequence of SEQ ID NO: 52;
a heavy chain variable region comprising the amino acid sequence of SEQ ID NO: 56 and a light chain variable region comprising the amino acid sequence of SEQ ID NO: 60; or
a heavy chain variable region comprising the amino acid sequence of SEQ ID NO: 173 and a light chain variable region comprising the amino acid sequence of SEQ ID NO: 177.

### BRIEF DESCRIPTION OF THE DRAWINGS

FIG. 1 shows surface expression of IL1RAP in human acute myeloid leukemia cell lines EOL1, Monomac 6, OCI/AML1, and KG-1, as well as T cell leukemia cell line, Karpas 299, as determined by flow cytometry analysis.

FIG. 2 shows specific binding of IL1RAP antibody 44E5_15C5 to IL1RAP positive cell lines EOL1, and Karpas 299. DMS79 cell line, which is IL1RAP negative, shows lack of binding by IL1RAP antibody 44E5_15C5.

FIG. 3 is a diagram representing the arrangement of competing bins of antibodies.

FIGS. 4A, 4B, 4C and 4D show binding of anti-IL1RAP antibodies to IL1RAP orthologs. Anti-IL1RAP antibodies were evaluated for cell surface binding to 293 cells expressing human (FIG. 4A), Macaca fascicularis (FIG. 4B), rat IL1RAP (FIG. 4C), and mouse IL1RAP (FIG. 4D) by flow cytometry. 21H5 was the mouse antibody against human IL1RAP. CBlgG1 (anti-hen egg lysozyme antibody, CrownBio™), was included as a negative control.

FIG. 5 shows internalization of IL1RAP antibody 44E5_15C5 into EOL1 cells. Live EOL1 cells were incubated with IL1RAP antibody 44E5_15C5 for 0.5 hours at 37° C. After cytospin, cells were then fixed, permeabilized and co-stained with LAMP1 antibody.

FIGS. 6A, 6B, and 6C show blockage of IL1α/β signaling by anti-IL1RAP antibodies. IL1RAP antibodies 37E10_15B5, 44E5_15C5, 16H2_17D2, and 36A10_21B6 displayed potent inhibition of IL1R1 signaling in a dose dependent manner (FIG. 8A). Antibodies 37E10_15B5 and 44E5_15C5 block IL-1β and IL-1α signaling with subnanomolar EC₅₀ (FIGS. 6B and 6C).

FIGS. 7A and 7B show blockage of IL-33 signaling by anti-IL1RAP antibodies. HEK-Blue IL-33 cells (Invivogen, CA) were harvested and plated in technical duplicates at a density of 50,000 cells per well in a 96-well plate. Antibodies, or a corresponding human IgG1 control antibody, was added to the wells in a serial dilution starting at 10 μg/ml (FIG. 7A), or a concentration of 1 and 10 μg/ml (FIG. 7B). After incubating cells with antibodies for 30 minutes, IL-33 was added to a final concentration of 0.5 ng/ml, and the plate was incubated overnight. 24 hours later, substrate was added to the supernatants, and samples were analyzed for absorbance at 620 nm. CBIgG1 (anti-hen egg lysozyme antibody, CrownBio™) was included as a negative control.

FIG. 8 shows the experimental design for the efficacy study of an anti-IL1RAP antibody in a mouse model of atopic dermatitis.

FIG. 9 shows the reduction of pruritis by an anti-IL1RAP antibody of the present invention in a mouse model of atopic dermatitis.

FIGS. 10A, 10B, and 10C show the prevention or reduction of inhibition of weight gain (FIG. 10A), prevention or reduction of skin thickening (FIG. 10B), and prevention or reduction of spleen enlargement (FIG. 10C) by an anti-IL1RAP antibody of the present invention in a mouse model of atopic dermatitis.

FIG. 11 shows that an anti-IL1RAP antibody of the present invention reduced the levels of the inflammatory/atopic mediators eotaxin, lipocalin-2, TARC/CCL17, TSLP, and IL-6 in the skin in a mouse model of atopic dermatitis.

FIG. 12 shows that an anti-IL1RAP antibody of the present invention reduced the levels of neutrophils, macrophages, and eosinophils (i.e., inflammatory cells) in the skin in a mouse model of atopic dermatitis.

FIG. 13 shows that FB759 inhibits HDM induced TARC level in PBMCs, and that Dupilumab does not or minimally inhibits TARC in the same assay. In the data shown in FIG. 13, hIgG4 (human IgG4 isotype Ab), BFB759, and Dupilumab were used at 30 ug/ml, and HDM was used at 10 ug/ml. Data shown are mean±s.d.

FIG. 14 shows a summary of inhibition of HDM induced TARC level in PBMCs from different donors by BFB759. In the data shown in FIG. 14, HDM was used at 10 ug/ml, and BFB759 from 1-30 ug/ml, PBMCs were from 12 different donors. Data shown are mean s.d.

FIG. 15 shows BFB759 and Dupilumab can inhibit TARC level in PBMC treated by HDM and IL-4. In the data shown in FIG. 15, HDM was used at 10 ug/ml, IL-4 was used at 750 pg/ml, antibodies were at three different concentrations as shown in the graph. Data shown are mean±s.d.

FIG. 16 shows BFB759 and Tralokinumab can inhibit TARC level in PBMC treated by HDM and IL-13. In the data shown in FIG. 16, HDM was used at 10 ug/ml, IL-13 was used at 0.5 nM, BFB759 was used at 1 ug/ml, tralokinumab was titrated from 10 ug/ml to 0.16 ug/ml (4 fold dilution, 4 series). Data shown are mean±s.d.

FIGS. 17A and 17B show that a combination of BFB759 and JAK inhibitors or Dexamethasone can inhibit TARC in PBMC treated by HDM. In the data shown in FIGS. 17A and 17B, HDM was used at 10 ug/ml, BFB759 was used at 1 ug/ml, JAKi and Dexamethasone were tested at three different concentrations (0.1 nM, 10 nM, and 1 uM) as shown in the graph. Data shown are mean±s.d.

FIGS. 18A and 18B show that a combination of BFB759 and Upadacitinib inhibit TARC in PBMC treated by HDM. In the data shown in FIGS. 18A and 18B, M was used at 10 ug/ml, BFB759 and hIgG4 (human IgG4 isotype Ab) were used at 1 ug/ml, JAKi Upadacitinib was tested at 5 fold serial titration from 1 uM to 0.01 nM. Data shown are mean±s.d.

FIGS. 19A and 19B show that a combination of BFB759 and Adalimumab inhibit IL-6 secretion in human whole blood cultures stimulated with HKCA. In the data shown in FIG. 19A, IL-6 Levels from whole blood cultures is measured by ELISA. In the data shown in FIG. 19B, the percentage of IL-6 measured in cultures treated as indicated compared to the levels detected with heat-killed Candida albicans (HKCA) and no intervening treatment.

FIGS. 20A and 20B show that a combination of BFB759 with Secukinumab inhibit IL-6 and IL-8 release in NHDF cells stimulated with cytokine combo.

FIGS. 21A and 21B show that a combination of BFB759 with Adalimumab inhibit IL-6 and IL-8 release in NHDF cells stimulated with cytokine combo.

### DETAILED DESCRIPTION

Various aspects of the disclosure relate to anti-IL1RAP antibodies, e.g., BFB 759 and the like, and antibody fragments and pharmaceutical compositions thereof, described herein to bind to and inhibit human IL1RAP on IL1RAP expressing cells, to inhibit IL-1, e.g., IL-1β and/or IL-1α, IL-33, IL-36 (IL-36α, IL-36β, IL-36γ) signaling, in vivo, and/or to treat inflammatory or autoimmune diseases, disorders, or conditions, e.g., rheumatoid arthritis, psoriasis, psoriatic arthritis, ankylosing spondylitis, Crohn's disease, ulcerative colitis, atopic dermatitis, asthma, and the like.

In particular embodiments, the anti-IL1RAP antibodies, e.g., BFB 759, or antigen binding portions thereof of the invention are administered in combination with one or more inhibitors or antagonists of TH2-associated immune response, TH1-associated immune response, TH17-associated immune response pruritis, IL-4 signaling, IL-13 signaling, IL-22 signaling, IL-33 signaling, IL-17 signaling, IL-36 signaling, IL-18 signaling, IL-23 signaling, OX40 signaling, IL-5 signaling, T cell migration, IRAK4 signaling, complement signaling, PDE4 signaling, or combinations thereof for treatment of inflammatory or autoimmune diseases or disorders such as rheumatoid arthritis, psoriasis, psoriatic arthritis, ankylosing spondylitis, Crohn's disease, ulcerative colitis, atopic dermatitis, or asthma.

### I. Definitions

In order that the invention may be more readily understood, certain terms are first defined. In addition, it should be noted that whenever a value or range of values of a parameter are recited, it is intended that values and ranges intermediate to the recited values are also intended to be part of this invention.

The terms “Interleukin 1 Receptor Accessory Protein antibody” or “anti-IL1RAP antibody”, used interchangeably herein, refer to an antibody that specifically binds to IL1RAP, e.g., human IL1RAP. An antibody “which binds” an antigen of interest, i.e., IL1RAP, is one capable of binding that antigen with sufficient affinity such that the antibody is useful in targeting a cell expressing the antigen. In a preferred embodiment, the antibody specifically binds to human IL1RAP (hIL1RAP). Examples of anti-IL1RAP antibodies are disclosed in the Examples, below; and are set forth in Table 5. Unless otherwise indicated, the term “anti-IL1RAP antibody” is meant to refer to an antibody which binds to wild type IL1RAP, a variant, or an isoform of IL1RAP.

As set forth below, in particular embodiment, the human anti-IL1RAP antibody for use in the invention methods herein is referred to as BFB759 (i.e., 37E10_15B5) corresponding to a heavy chain variable region comprising a CDR3 domain comprising the amino acid sequence of SEQ ID NO: 67, a CDR2 domain comprising the amino acid sequence of SEQ ID NO: 66, and a CDR1 domain comprising the amino acid sequence of SEQ ID NO: 65; and a light chain variable region comprising a CDR3 domain comprising the amino acid sequence of SEQ ID NO: 70, a CDR2 domain comprising the amino acid sequence of SEQ ID NO: 62, and a CDR1 domain comprising the amino acid sequence of SEQ ID NO: 69. In another particular embodiment, the human anti-IL1RAP antibody BFB759 (i.e., 37E10_15B5) corresponds to a heavy chain variable region comprising the amino acid sequence of SEQ ID NO: 64 and a light chain variable region comprising the amino acid sequence of SEQ ID NO: 68.

Several different isoforms of IL1RAP have been identified. An exemplary amino acid sequence of wild type human IL1RAP, which contains 570 amino acids, is provided below as SEQ ID NO: 260. The extracellular domain (ECD) of IL1RAP comprises amino acids 21-367 of SEQ ID NO:260.

```
                10 20 30 40 50
MTLLWCVVSL YFYGILQSDA SERCDDWGLD TMRQIQVFED EPARIKCPLF
                60 70 80 90 100
EHFLKFNYST AHSAGLTLIW YWTRQDRDLE EPINFRLPEN RISKEKDVLW
              110 120 130 140 150
FRPTLLNDTG NYTCMLRNTT YCSKVAFPLE VVQKDSCFNS PMKLPVHKLY
              160 170 180 190 200
IEYGIQRITC PNVDGYFPSS VKPTITWYMG CYKIQNFNNV IPEGMNLSFL
              210 220 230 240 250
IALISNNGNY TCVVTYPENG RTFHLTRTLT VKVVGSPKNA VPPVIHSPND
              260 270 280 290 300
HVVYEKEPGE ELLIPCTVYF SFLMDSRNEV WWTIDGKKPD DITIDVTINE
              310 320 330 340 350
SISHSRTEDE TRTQILSIKK VTSEDLKRSY VCHARSAKGE VAKAAKVKQK
              360 370 380 390 400
VPAPRYTVEL ACGFGATVLL VVILIVVYHV YWLEMVLFYR AHFGTDETIL
              410 420 430 440 450
DGKEYDIYVS YARNAEEEEF VLLTLRGVLE NEFGYKLCIF DRDSLPGGIV
              460 470 480 490 500
TDETLSFIQK SRRLLVVLSP NYVLQGTQAL LELKAGLENM ASRGNINVIL
              510 520 530 540 550
VQYKAVKETK VKELKRAKTV LTVIKWKGEK SKYPQGRFWK QLQVAMPVKK
              560 570
SPRRSSSDEQ GLSYSSLKNV
```

The terms “specific binding” or “specifically binding”, as used herein, in reference to the interaction of a IL1RAP antibody or antigen binding portion thereof with a second chemical species, mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

In one embodiment, the phrase “specifically binds to hIL1RAP” or “specific binding to hIL1RAP”, as used herein, refers to the ability of an anti-IL1RAP antibody to interact with IL1RAP (human or cynomolgus monkey IL1RAP) with a dissociation constant (K_{D}) of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less. In another embodiment, the phrase “specifically binds to hIL1RAP” or “specific binding to hIL1RAP”, as used herein, refers to the ability of an anti-IL1RAP antibody to interact with hIL1RAP with a dissociation constant (K_{D}) of between about 1 pM (0.001 nM) to 2,000 nM, between about 500 pM (0.5 nM) to 1,000 nM, between about 500 pM (0.5 nM) to 500 nM, between about 1 nM) to 200 nM, between about 1 nM to 100 nM, between about 1 nM to 50 nM, between about 1 nM to 20 nM, or between about 1 nM to 5 nM. In one embodiment, K_{D} is determined by surface plasmon resonance or Bio-Layer Interferometry, or by any other method known in the art. Bio-Layer Interferometry refers to an optical phenomenon that allows for the analysis of real-time biospecific interactions by measuring the interference patterns of reflected white light, for example using the Octet™ system (ForteBio, Pall Corp. Fremont, CA). For further description of the Octet™ system, see Li, B et al. (2011) J. Pharm. Biomed. Anal. 54(2):286-294 and Abdiche, Y. N., et al. (2009) Anal. Biochem. 386(2):172-180, the contents of which are incorporated herein by reference.

The term “antibody” broadly refers to an immunoglobulin (Ig) molecule, generally comprised of four polypeptide chains, two heavy (H) chains and two light (L) chains, or any functional fragment, mutant, variant, or derivative thereof, that retains the essential target binding features of an Ig molecule. Such mutant, variant, or derivative antibody formats are known in the art. Non-limiting embodiments of which are discussed below.

The terms TH1 and TH-1, and TH2 and TH-2, and TH17 and TH-17, are used interchangeably herein. The terms “mediated” and “associated” are used interchangeably herein.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. Immunoglobulin molecules can be of any type (e.g., IgG, IgE, IgM, IgD, IgA and IgY) and class (e.g., IgG1, IgG2, IgG3, IgG4, IgA1 and IgA2) or subclass.

The term “antigen binding portion” of an antibody (or simply “antibody portion”), as used herein, refers to one or more fragments of an antibody that retain the ability to specifically bind to an antigen (e.g., hIL1RAP). It has been shown that the antigen binding function of an antibody can be performed by fragments of a full-length antibody. Such antibody embodiments may also be bispecific, dual specific, or multi-specific formats; specifically binding to two or more different antigens. Examples of binding fragments encompassed within the term “antigen binding portion” of an antibody include (i) a Fab fragment, a monovalent fragment consisting of the VL, VH, CL and CH1 domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546, Winter et al., PCT publication WO 90/05144 A1 herein incorporated by reference), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen binding portion” of an antibody. In certain embodiments, scFv molecules may be incorporated into a fusion protein. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, P., et al. (1993) Proc. Natl. Acad. Sci. USA 90:6444-6448; Poljak, R. J., et al. (1994) Structure 2:1121-1123). Such antibody binding portions are known in the art (Kontermann and Dubel eds., Antibody Engineering (2001) Springer-Verlag. New York. 790 pp. (ISBN 3-540-41354-5).

The term “antibody construct” as used herein refers to a polypeptide comprising one or more the antigen binding portions disclosed herein linked to a linker polypeptide or an immunoglobulin constant domain. Linker polypeptides comprise two or more amino acid residues joined by peptide bonds and are used to link one or more antigen binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Antibody portions, such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein.

An “isolated antibody”, as used herein, is intended to refer to an antibody that is substantially free of other antibodies having different antigenic specificities (e.g., an isolated antibody that specifically binds IL1RAP is substantially free of antibodies that specifically bind antigens other than IL1RAP). An isolated antibody that specifically binds IL1RAP may, however, have cross-reactivity to other antigens, such as IL1RAP molecules from other species. Moreover, an isolated antibody may be substantially free of other cellular material and/or chemicals.

The term “humanized antibody” refers to antibodies which comprise heavy and light chain variable region sequences from a nonhuman species (e.g., a mouse) but in which at least a portion of the VH and/or VL sequence has been altered to be more “human-like”, i.e., more similar to human germline variable sequences. In particular, the term “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, preferably at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In other embodiments, a humanized antibody only contains a humanized heavy chain. In specific embodiments, a humanized antibody only contains a humanized variable domain of a light chain and/or humanized heavy chain.

The humanized antibody can be selected from any class of immunoglobulins, including IgM, IgG, IgD, IgA and IgE, and any isotype, including without limitation IgG1, IgG2, IgG3 and IgG4. The humanized antibody may comprise sequences from more than one class or isotype, and particular constant domains may be selected to optimize desired effector functions using techniques well-known in the art.

The terms “Kabat numbering,” “Kabat definitions,” and “Kabat labeling” are used interchangeably herein. These terms, which are recognized in the art, refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad, Sci. 190:382-391 and, Kabat, E. A., et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

As used herein, the term “CDR” refers to the complementarity determining region within antibody variable sequences. There are three CDRs in each of the variable regions of the heavy chain (HC) and the light chain (LC), which are designated CDR1, CDR2 and CDR3 (or specifically HC CDR1, HC CDR2, HC CDR3, LC CDR1, LC CDR2, and LC CDR3), for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Chothia and coworkers (Chothia & Lesk, J. Mol. Biol. 196:901-917 (1987) and Chothia et al., Nature 342:877-883 (1989)) found that certain sub-portions within Kabat CDRs adopt nearly identical peptide backbone conformations, despite having great diversity at the level of amino acid sequence. These sub-portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262(5):732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (See e.g., Winnaker, From Genes to Clones (Verlagsgesellschaft, Weinheim, Germany 1987). In a family of immunoglobulins, each position in the consensus sequence is occupied by the amino acid occurring most frequently at that position in the family. If two amino acids occur equally frequently, either can be included in the consensus sequence.

“Percent (%) amino acid sequence identity” with respect to a peptide or polypeptide sequence is defined as the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. In one embodiment, the disclosure includes an amino acid sequence having at least 80%, at least 85%, at least 90%, at least 95%, at least 96%, at least 97%, at least 98%, or at least 99% identity to an amino acid sequence set forth in any one of SEQ ID NOs: 1 to 190.

In one embodiment, the anti-IL1RAP antibody, e.g., BFB759 or the like, or antigen-binding portion thereof, is capable of reducing inflammation. In one embodiment, the anti-IL1RAP antibody, e.g., BFB759 or the like, or antigen-binding portion thereof, is capable of reducing autoimmunity. In one embodiment, the anti-IL1RAP antibody, e.g., BFB759 or the like, or antigen-binding portion thereof, is capable of reducing TH-1 mediated immune responses. In one embodiment, the anti-IL1RAP antibody, e.g., BFB759 or the like, or antigen-binding portion thereof, is capable of reducing TH2-mediated immune responses. In one embodiment, the anti-IL1RAP antibody, e.g., BFB759 or the like, or antigen-binding portion thereof, is capable of reducing TH17-mediated immune responses. In one embodiment, the at least one therapeutic agent used in combination with the anti-IL1RAP antibody, e.g., BFB759 or the like, inhibits both TH-1- and TH-2 mediated immune responses. In one embodiment, the anti-IL1RAP antibody, e.g., BFB759 or the like, or antigen-binding portion thereof, is capable of reducing levels of TARC/CCL17, PARC/CCL18, CCL22/MDC, IgE, periostin, IL-22, IL-13, IL-18, IL-19, CCL27/CTACK, S100A7/12, E-selectin, MMP-12, LDH, neotaxin-1, eotaxin-3/CCL26, or combinations thereof. In one embodiment, the anti-IL1RAP antibody, e.g., BFB759 or the like, or antigen-binding portion thereof, is capable of increasing or decreasing levels of IL-1α, IL-1β, IL-33, IL-36α, IL-36β, IL-36γ, or combinations thereof, or of CXCL1, CXCL8, TARC, LCN2, or combinations thereof towards restoration of normal levels.

The term “multivalent antibody” is used herein to denote an antibody comprising two or more antigen binding sites. In certain embodiments, the multivalent antibody may be engineered to have the three or more antigen binding sites, and is generally not a naturally occurring antibody.

The term “multispecific antibody” refers to an antibody capable of binding two or more unrelated antigens.

The term “dual variable domain” or “DVD,” as used interchangeably herein, are antigen binding proteins that comprise two or more antigen binding sites and are tetravalent or multivalent binding proteins. Such DVDs may be monospecific, i.e., capable of binding one antigen or multispecific, i.e. capable of binding two or more antigens. DVD binding proteins comprising two heavy chain DVD polypeptides and two light chain DVD polypeptides are referred to a DVD Ig. Each half of a DVD Ig comprises a heavy chain DVD polypeptide, and a light chain DVD polypeptide, and two antigen binding sites. Each binding site comprises a heavy chain variable domain and a light chain variable domain with a total of 6 CDRs involved in antigen binding per antigen binding site. In one embodiment, the CDRs described herein are used in an anti-IL1RAP DVD.

The term “activity” includes activities such as the binding specificity/affinity of an antibody for an antigen, for example, an anti-hIL1RAP antibody or ADC that binds to a IL1RAP antigen. In one embodiment, an anti-IL1RAP antibody or anti-IL1RAP ADC activity includes, but it not limited to, binding to IL1RAP in vitro; binding to IL1RAP on cells expressing IL1RAP in vivo; modulating (e.g., inhibiting) IL-1, e.g., IL-1β and/or IL-1α, signaling; reducing inflammation; reducing autoimmunity; reducing TH-1 mediated immune responses; reducing TH2-mediated immune responses; reducing levels of TARC/CCL17, PARC/CCL18, CCL22/MDC, IgE, periostin, IL-22, IL-13, IL-18, IL-19, CCL27/CTACK, S100A7/12, E-selectin, MMP-12, LDH, neotaxin-1, eotaxin-3/CCL26, or combinations thereof; increasing or decreasing levels of IL-1α, IL-1β, IL-33, IL-36α, IL-36β, IL-36γ, or combinations thereof, or of CXCL1, CXCL8, TARC, LCN2, or combinations thereof towards restoration of normal levels; reducing rheumatoid arthritis, psoriasis, psoriatic arthritis, ankylosing spondylitis, Crohn's disease, ulcerative colitis, atopic dermatitis, or asthma; reducing allergic rhinitis, allergic conjunctivitis, food allergy, drug allergy, or angioedema; or reducing sepsis, acute respiratory distress syndrome, myocardial infarction, cystic fibrosis, irritable bowel disease, multiple sclerosis, neutrophilic asthma, Alzheimer's disease, stroke, diabetic kidney disease, diabetes, diabetic retinopathy, Chronic obstructive pulmonary disease (COPD), or idiopathic pulmonary fibrosis or non-alcoholic fatty liver disease.

The term “epitope” refers to a region of an antigen that is bound by an antibody or antibody fragment. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. In certain embodiments, an antibody is said to specifically bind an antigen when it preferentially recognizes its target antigen in a complex mixture of proteins and/or macromolecules.

The term “kₒₙ” or “kₐ”, as used herein, is intended to refer to the on rate constant for association of an antibody to the antigen to form the antibody/antigen complex.

The term “k_{off}” or “k_{d}”, as used herein, is intended to refer to the off rate constant for dissociation of an antibody from the antibody/antigen complex.

The term “K_{D}”, as used herein, is intended to refer to the equilibrium dissociation constant of a particular antibody-antigen interaction. K_{D} is calculated by kₐ/k_{d}. In one embodiment, the antibodies of the invention have a K_{D} of about 2,000 nM or less, about 1,000 nM or less, about 500 nM or less, about 200 nM or less, about 100 nM or less, about 75 nM or less, about 25 nM or less, about 21 nM or less, about 12 nM or less, about 11 nM or less, about 10 nM or less, about 9 nM or less, about 8 nM or less, about 7 nM or less, about 6 nM or less, about 5 nM or less, about 4 nM or less, about 3 nM or less, about 2 nM or less, about 1 nM or less, about 0.5 nM or less, about 0.3 nM or less, about 0.1 nM or less, about 0.01 nM or less, or about 0.001 nM or less.

The term “competitive binding”, as used herein, refers to a situation in which a first antibody competes with a second antibody, for a binding site on a third molecule, e.g., an antigen. In one embodiment, competitive binding between two antibodies is determined using FACS analysis.

The term “competitive binding assay” is an assay used to determine whether two or more antibodies bind to the same epitope. In one embodiment, a competitive binding assay is a competition fluorescent activated cell sorting (FACS) assay which is used to determine whether two or more antibodies bind to the same epitope by determining whether the fluorescent signal of a labeled antibody is reduced due to the introduction of a non-labeled antibody, where competition for the same epitope will lower the level of fluorescence.

The term “labeled antibody” as used herein, refers to an antibody, or an antigen binding portion thereof, with a label incorporated that provides for the identification of the binding protein, e.g., an antibody. Preferably, the label is a detectable marker, e.g., incorporation of a radiolabeled amino acid or attachment to a polypeptide of biotinyl moieties that can be detected by marked avidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm); fluorescent labels (e.g., FITC, rhodamine, lanthanide phosphors), enzymatic labels (e.g., horseradish peroxidase, luciferase, alkaline phosphatase); chemiluminescent markers; biotinyl groups; predetermined polypeptide epitopes recognized by a secondary reporter (e.g., leucine zipper pair sequences, binding sites for secondary antibodies, metal binding domains, epitope tags); and magnetic agents, such as gadolinium chelates.

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8. Non-limiting examples of drugs that may be included in the ADCs are mitotic inhibitors, antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

The terms “V-set domain containing T cell activation inhibitor 1 antibody drug conjugate,” “anti-IL1RAP antibody drug conjugate,” or “anti-IL1RAP ADC”, used interchangeably herein, refer to an ADC comprising an antibody that specifically binds to IL1RAP, whereby the antibody is conjugated to one or more chemical agent(s) or payloads. In one embodiment, the chemical agent is linked to the antibody via a linker.

The term “drug-to-antibody ratio” or “DAR” refers to the number of drugs, e.g., IGN, auristatin, or maytansinoid, attached to the antibody of the ADC. The DAR of an ADC can range from 1 to 8, although higher loads, e.g., 10, are also possible depending on the number of linkage site on an antibody. The term DAR may be used in reference to the number of drugs loaded onto an individual antibody, or, alternatively, may be used in reference to the average or mean DAR of a group of ADCs.

The term “IL1RAP associated disorder,” as used herein, includes any disorder or disease (including proliferative disorders, e.g., cancer) that is marked, diagnosed, detected or identified by a phenotypic or genotypic aberration of IL1RAP genetic components or expression during the course or etiology of the disease or disorder. In this regard a IL1RAP phenotypic aberration or determinant may, for example, comprise increased or decreased levels of IL1RAP protein expression on one cell population, e.g., a cancer cell population, as compared to another cell population, e.g., a normal cell population, or increased or decreased IL1RAP protein expression on certain definable cell populations, or increased or decreased IL1RAP protein expression at an inappropriate phase or stage of a cell lifecycle. It will be appreciated that similar expression patterns of genotypic determinants (e.g., mRNA transcription levels) of IL1RAP may also be used to classify or detect IL1RAP associated disorders.

The term “inflammatory disease,” as used herein, is meant to refer to or describe diseases and disorders characterized by inflammation, which is an immune reaction characterized by capillary dilatation, leukocytic infiltration, redness, heat, pain, and/or up-regulation of inflammatory cytokines including but not limited to interleukin-1 (IL-1), IL-2, IL-6, IL-12, and IL-18, tumor necrosis factor alpha (TNF-α), interferon gamma (IFNγ), and granulocyte-macrophage colony stimulating factor (GM-CSF).

In some embodiments, the inflammatory disease is characterized by elevated serum levels of IL-1α, IL-1β, IL-33, IL-36α, IL-36β, IL-36γ, or combinations thereof, or by elevated serum levels of CXCL1, CXCL8, TARC, LCN2, or combinations thereof.

The term “autoimmune disease” or “autoimmunity,” as used herein, is meant to refer to or describe diseases, disorders, or conditions characterized by an autoimmune response, where the immune system attacks the body's own healthy cells, tissues, and organs.

The term “dysfunction” and grammatical variations thereon, such as in the context of neutrophil or eosinophil cells dysfunction, refers to cells that are not behaving, working, or operating normally or properly.

The term “increased cell counts,” or grammatical variations thereof as used herein, such as with increased neutrophil or eosinophil cell counts, refers to abnormal cell counts above a non-diseased basal (normal) level, such that the increased cell counts are well-known in the art to be indicative of disease, such an inflammatory or autoimmune disease.

The term “abnormal serum levels,” or grammatical variations thereof as used herein, such as with increased levels of IL-1α, IL-1β, IL-33, IL-36α, IL-36β, IL-36γ, or combinations thereof, refers to abnormal cytokine levels above a non-diseased basal (normal) level, such that the increased cytokine levels are well-known in the art to be indicative of disease, such an inflammatory or autoimmune disease.

In some embodiments, the autoimmune disease or autoimmunity is characterized by the presence of autoantibodies against autoantigens in the body's own healthy cells, tissues, and organs.

In some embodiments, the autoimmune disease or autoimmunity is characterized by elevated serum levels of IL-1α, IL-1β, IL-33, IL-36α, IL-36β, IL-36γ, or combinations thereof, or by elevated serum levels of CXCL1, CXCL8, TARC, LCN2, or combinations thereof.

In some embodiments, the inflammatory or autoimmune disease is sepsis, acute respiratory distress syndrome, myocardial infarction, cystic fibrosis, irritable bowel disease, ulcerative colitis, Crohn's disease, atopic dermatitis, psoriasis, multiple sclerosis, neutrophilic asthma, Alzheimer's disease, stroke, diabetic kidney disease, diabetes, diabetic retinopathy, hidradenitis suppurativa, rheumatoid arthritis, psoriatic arthritis, ankylosing spondylitis, asthma, allergic rhinitis, allergic conjunctivitis, food allergy, drug allergy, and angioedema, allergic contact dermatitis, irritant contact dermatitis, rosacea, psoriasis vulgaris, pustular psoriasis, mastocytosis, systemic lupus erythematosus, systemic sclerosis, chronic spontaneous urticaria, autoimmune blistering diseases, Behcet's disease, vitiligo, deficiency of IL-36 receptor antagonist (DITRA), CARD14-mediated psoriasis, acute generalized exanthematous pustulosis, pyoderma gangrenosum, Sweet's syndrome, acne, folliculitis and eosinophilic pustular folliculitis, adult-onset Still disease, cutaneous lupus erythematosus, contact dermatitis, alopecia areata, cutaneous drug eruptions, graft-versus-host disease, cryopyrin-associated periodic syndromes, granulomatosis with polyangiitis, pyogenic arthritis, pyoderma gangrenosum and acne (PAPA), familial Mediterranean fever, rosacea, synovitis, pustulosis, hyperostosis, osteitis (SAPHO), bullous pemphigoid, pemphigus vulgaris, Schnitzler syndrome, SAPHO, acne vulgaris, urticarial vasculitis, cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), Chronic obstructive pulmonary disease (COPD), idiopathic pulmonary fibrosis, non-alcoholic fatty liver disease, neutrophilic dermatoses, or monogenic autoinflammatory skin disorders.

The term “atopy” or “atopic disease,” as used herein, is meant to refer to or describe diseases, disorders, or conditions characterized by the tendency to produce an exaggerated immunoglobulin E (IgE) immune response to diverse antigens/allergens, including otherwise harmless substances.

In some embodiments, the atopic disease is atopic dermatitis, asthma, allergic rhinitis, allergic conjunctivitis, food allergy, drug allergy, or angioedema. In an embodiment, the atopic disease is atopic dermatitis. The clinical phenotypes that characterize atopic dermatitis are skin barrier dysfunction and immune dysregulation. Filaggrin gene mutation leads to skin barrier dysfunction and transepidermal water loss, resulting in atopic dermatitis. Atopic dermatitis is characterized by increased serum IgE levels as well as Th2 immune responses with increased levels of IL-4, IL-5, IL-10, and IL-13. This leads to increased allergen exposure, which is picked up by Langerhans cells to lymph node and stimulates naïve CD+ T cells (Th0) to differentiate into Th2 cells. The associated cytokines produced, such as IL-4 and IL-13, are known to stimulate the production of IgE, whereas IL-5 is one of the most important cytokines for generation of eosinophils. IgE mediates mast cell activation, which can induce the expression of proinflammatory cytokines by keratinocytes as well as migration of dendritic cells. Impaired barrier function could stimulate signaling cascades, engaging an epidermal homeostatic response, as well as a type 2 inflammatory reaction.

In one embodiment, the antibodies of the invention are administered to a patient having an inflammatory or autoimmune disease, or inflammation or autoimmunity. In one embodiment, the antibodies of the invention are administered to a patient having an atopic disorder or disease. In one embodiment, the antibodies of the invention are administered to a patient having atopic dermatitis. In one embodiment, administration of antibodies of the invention decreases inflammation or autoimmunity. In one embodiment, administration of antibodies of the invention reduces atopic dermatitis.

Methods for detecting expression of IL1RAP are known in the art.

The terms “overexpress,” “overexpression,” or “overexpressed” interchangeably refer to a gene that is transcribed or translated at a detectably greater level in comparison to a normal cell or a cell under normal conditions. Overexpression therefore refers to both overexpression of protein and RNA (due to increased transcription, post transcriptional processing, translation, post translational processing, altered stability, and altered protein degradation), as well as local overexpression due to altered protein traffic patterns (increased nuclear localization), and augmented functional activity, e.g., as in an increased enzyme hydrolysis of substrate. Thus, overexpression refers to either protein or RNA levels. Overexpression can also be by 5%, 10%, 20%, 30%, 40%, 50%, 60%, 70%, 80%, 90% or more in comparison to a normal cell or comparison cell.

The term “administering” as used herein is meant to refer to the delivery of a substance (e.g., an anti-IL1RAP antibody) to achieve a therapeutic objective (e.g., the treatment of an inflammatory or autoimmune disease or disorder, or the reduction of inflammation or autoimmunity, or the reduction of an atopic disease). Modes of administration may be parenteral, enteral and topical. Parenteral administration is usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The term “combination therapy”, as used herein, refers to the administration of two or more therapeutic substances, e.g., an anti-IL1RAP antibody and at least one additional therapeutic agent. The additional therapeutic agent may be administered concomitant with, prior to, or following the administration of the anti-IL1RAP antibody. In embodiments, the anti-IL1RAP antibodies or ADCs of the invention are administered in combination with one or more inhibitors of TH2-associated immune response, TH1-associated immune response, pruritis, IL-4 signaling, IL-13 signaling, IL-22 signaling, IL-33 signaling, IL-17 signaling, IL-36 signaling, IL-18 signaling, IL-23 signaling, OX40 signaling, IL-5 signaling, T cell migration, IRAK4 signaling, complement signaling, PDE4 signaling, or combinations thereof for treatment of inflammatory or autoimmune diseases including rheumatoid arthritis, psoriasis, psoriatic arthritis, ankylosing spondylitis, Crohn's disease, ulcerative colitis, atopic dermatitis, and asthma.

As used herein, the term “effective amount” or “therapeutically effective amount” refers to the amount of a drug, e.g., an antibody, which is sufficient to reduce or ameliorate the severity and/or duration of a disorder, e.g., an inflammatory or autoimmune disease such as rheumatoid arthritis, psoriasis, psoriatic arthritis, ankylosing spondylitis, Crohn's disease, ulcerative colitis, atopic dermatitis, or asthma, or one or more symptoms thereof, prevent the advancement of a disorder, cause regression of a disorder, prevent the recurrence, development, onset or progression of one or more symptoms associated with a disorder, detect a disorder, or enhance or improve the prophylactic or therapeutic effect(s) of another therapy (e.g., prophylactic or therapeutic agent). The effective amount of an antibody may, for example, decrease inflammation (e.g., decrease levels of inflammatory markers), decrease autoimmunity (e.g., decrease autoantibody levels or decrease the immune response against the body's own healthy cells and tissues), reduce the number or size of inflammatory, autoimmune, and/or atopic lesions, and/or relieve to some extent one or more of the symptoms associated with the inflammatory, autoimmune, or atopic disease.

Various aspects of the invention are described in further detail in the following subsections.

### II. Anti-IL1RAP Antibodies

One aspect disclosed herein provides humanized anti-IL1RAP antibodies, or antigen binding portions thereof. Another aspect disclosed herein provides human anti-IL1RAP antibodies, or antigen binding portions thereof. In one embodiment, the antibodies disclosed herein bind human IL1RAP. In another embodiment, the antibodies disclosed herein bind cynomolgus monkey IL1RAP. In another embodiment, the antibodies disclosed herein bind human IL1RAP expressed on cells.

In one embodiment, anti-IL1RAP antibodies are disclosed which have the ability to bind to IL1RAP, as described in the Examples below. Collectively, the novel antibodies are referred to herein as “IL1RAP antibodies.” The anti-IL1RAP antibodies or antigen binding fragments thereof, are able to inhibit or decrease inflammatory, autoimmune, and/or atopic responses in vivo. In various embodiments, anti-IL1RAP antibodies or antigen binding fragments thereof are capable of modulating a biological function of IL1RAP. In other embodiments of the foregoing aspects, the anti-IL1RAP antibodies or antigen binding fragments thereof bind IL1RAP on cells expressing IL1RAP. Thus, the disclosure includes anti-IL1RAP antibodies or antigen binding fragments thereof that are effective at inhibiting or decreasing inflammation, autoimmunity, and/or atopy. Without wishing to be bound by any particular theory, in one embodiment, the anti-IL1RAP antibodies, antigen-binding portions thereof, and ADCs are capable of inhibiting multiple IL1RAP activities including, but not limited to, IL-1β signaling through IL1RAP; IL-1α, IL-1β, and IL-38 signaling through the IL-1R; IL-33 signaling through the IL-33R, and IL-36α, IL-36β, and IL-36γ signaling through the IL-36R.

In addition, the present inventors have further shown that the anti-IL1RAP antibody, e.g., BFB759, is effective in a mouse model of atopic dermatitis (see Example 8). Accordingly, the anti-IL1RAP antibodies and antigen-binding portions thereof can be used for the treatment of atopic dermatitis in a subject, or of contact dermatitis, allergic dermatitis, or allergic contact dermatitis.

Antibodies having combinations of any of the aforementioned characteristics are contemplated as aspects of the disclosure.

While the term “antibody” is used throughout, it should be noted that antibody fragments (i.e., antigen-binding portions of an anti-IL1RAP antibody) are also included in the disclosure and may be included in the embodiments (methods and compositions) described throughout. In certain embodiments, an anti-IL1RAP antibody binding portion is a Fab, a Fab′, a F(ab′)2, a Fv, a disulfide linked Fv, an scFv, a single domain antibody, or a diabody.

Example 2 describes the generation of fully human IL1RAP antibodies against the extracellular domain of human IL1RAP that are contemplated for use in the therapeutic methods set forth herein. The heavy and light chain variable region amino acid sequences for these human anti-IL1RAP antibodies are set forth in Table 5. The heavy and light chain variable region nucleotide sequences for these human antibodies are set forth in Table 6.

Thus, in particular embodiments for use in the methods provided herein, the disclosure provides human anti-IL1RAP antibodies, or antigen binding portions thereof, comprising a heavy chain variable region comprising an amino acid sequence selected from the group consisting of SEQ ID NOs: 1, 9, 17, 25, 32, 40, 48, 56, 64, 71, 74, 83, 90, 96, 100, 106, 109, 116, 118, 121, 123, 125, 127, 130, 136, 140, 144, 151, 158, 163, 170, 173, 180, and 185; and a light chain variable region comprising an amino acid sequence selected from the group consisting of 5, 13, 21, 29, 36, 44, 52, 60, 68, 73, 78, 82, 87, 93, 98, 103, 108, 113, 114, 120, 122, 124, 126, 128, 134, 137, 143, 147, 154, 160, 167, 172, 177, 184, and 189.

In particular embodiments for use in the methods provided herein, the disclosure includes a human anti-IL1RAP antibody, or antigen binding portion thereof, comprising an HC CDR set (CDR1, CDR2, and CDR3) selected from those set forth in Table 5; and an LC CDR set (CDR1, CDR2, and CDR3) selected from those set forth in Table 5.

In particular embodiments, the human anti-IL1RAP antibody for use in the invention methods herein, is selected from: 37E10_15B5, 44E5_15C5, 16H2_17D2, and/or 36A10_21B6 (see Table 5).

In particular embodiments, the human anti-IL1RAP antibody for use in the invention methods herein, is selected from an antibody comprising:
BFB759 (i.e., 37E10_15B5) corresponding to a heavy chain variable region comprising a CDR3 domain comprising the amino acid sequence of SEQ ID NO: 67, a CDR2 domain comprising the amino acid sequence of SEQ ID NO: 66, and a CDR1 domain comprising the amino acid sequence of SEQ ID NO: 65; and a light chain variable region comprising a CDR3 domain comprising the amino acid sequence of SEQ ID NO: 70, a CDR2 domain comprising the amino acid sequence of SEQ ID NO: 62, and a CDR1 domain comprising the amino acid sequence of SEQ ID NO: 69;
a heavy chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10; and a light chain variable region comprising a CDR3 domain comprising the amino acid sequence of SEQ ID NO: 16, a CDR2 domain comprising the amino acid sequence of SEQ ID NO: 15, and a CDR1 domain comprising the amino acid sequence of SEQ ID NO: 14;
a heavy chain variable region comprising a CDR3 domain comprising the amino acid sequence of SEQ ID NO: 51, a CDR2 domain comprising the amino acid sequence of SEQ ID NO: 50, and a CDR1 domain comprising the amino acid sequence of SEQ ID NO: 49; and a light chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53;
a heavy chain variable region comprising a CDR3 domain comprising the amino acid sequence of SEQ ID NO: 59, a CDR2 domain comprising the amino acid sequence of SEQ ID NO: 58, and a CDR1 domain comprising the amino acid sequence of SEQ ID NO: 57; and a light chain variable region comprising a CDR3 domain comprising the amino acid sequence of SEQ ID NO: 63, a CDR2 domain comprising the amino acid sequence of SEQ ID NO: 62, and a CDR1 domain comprising the amino acid sequence of SEQ ID NO: 61; and/or
a heavy chain variable region comprising a CDR3 domain comprising the amino acid sequence of SEQ ID NO: 176, a CDR2 domain comprising the amino acid sequence of SEQ ID NO: 175, and a CDR1 domain comprising the amino acid sequence of SEQ ID NO: 174; and a light chain variable region comprising a CDR3 domain comprising the amino acid sequence of SEQ ID NO: 179, a CDR2 domain comprising the amino acid sequence of SEQ ID NO: 178, and a CDR1 domain comprising the amino acid sequence of SEQ ID NO: 79.

In other embodiments, the human anti-IL1RAP antibody for use in the invention methods herein, is selected from an antibody comprising:
BFB759 (i.e., 37E10_15B5) corresponding to a heavy chain variable region comprising the amino acid sequence of SEQ ID NO: 64 and a light chain variable region comprising the amino acid sequence of SEQ ID NO: 68;
a heavy chain variable region comprising the amino acid sequence of SEQ ID NO: 9 and a light chain variable region comprising the amino acid sequence of SEQ ID NO: 82;
a heavy chain variable region comprising the amino acid sequence of SEQ ID NO: 48 and a light chain variable region comprising the amino acid sequence of SEQ ID NO: 52;
a heavy chain variable region comprising the amino acid sequence of SEQ ID NO: 56 and a light chain variable region comprising the amino acid sequence of SEQ ID NO: 60; or
a heavy chain variable region comprising the amino acid sequence of SEQ ID NO: 173 and a light chain variable region comprising the amino acid sequence of SEQ ID NO: 177.

In a particular embodiment, the human anti-IL1RAP antibody for use in the invention methods herein is BFB759 (i.e., 37E10_15B5) corresponding to a heavy chain variable region comprising a CDR3 domain comprising the amino acid sequence of SEQ ID NO: 67, a CDR2 domain comprising the amino acid sequence of SEQ ID NO: 66, and a CDR1 domain comprising the amino acid sequence of SEQ ID NO: 65; and a light chain variable region comprising a CDR3 domain comprising the amino acid sequence of SEQ ID NO: 70, a CDR2 domain comprising the amino acid sequence of SEQ ID NO: 62, and a CDR1 domain comprising the amino acid sequence of SEQ ID NO: 69. In another particular embodiment, BFB759 (i.e., 37E10_15B5) corresponds to a heavy chain variable region comprising the amino acid sequence of SEQ ID NO: 64 and a light chain variable region comprising the amino acid sequence of SEQ ID NO: 68.

Replacements of amino acid residues in the Fc portion to alter antibody effector function have been described (Winter, et al. U.S. Pat. Nos. 5,648,260 and 5,624,821, incorporated by reference herein). The Fc portion of an antibody mediates several important effector functions e.g. cytokine induction, ADCC, phagocytosis, complement dependent cytotoxicity (CDC) and half-life/clearance rate of antibody and antigen-antibody complexes. In some cases these effector functions are desirable for therapeutic antibody but in other cases might be unnecessary or even deleterious, depending on the therapeutic objectives. Certain human IgG isotypes, particularly IgG1 and IgG3, mediate ADCC and CDC via binding to FcγRs and complement C1q, respectively. Neonatal Fc receptors (FcRn) are the critical components determining the circulating half-life of antibodies. In still another embodiment at least one amino acid residue is replaced in the constant region of the antibody, for example the Fc region of the antibody, such that effector functions of the antibody are altered.

One embodiment includes a labeled anti-IL1RAP antibody, or antibody portion thereof, where the antibody is derivatized or linked to one or more functional molecule(s) (e.g., another peptide or protein). For example, a labeled antibody can be derived by functionally linking an antibody or antibody portion of the disclosure (by chemical coupling, genetic fusion, noncovalent association or otherwise) to one or more other molecular entities, such as another antibody (e.g., a bispecific antibody or a diabody), a detectable agent, a pharmaceutical agent, a protein or peptide that can mediate the association of the antibody or antibody portion with another molecule (such as a streptavidin core region or a polyhistidine tag), and/or a therapeutic agent selected from the group consisting of a mitotic inhibitor, an immunomodulating agent, a vector for gene therapy, an alkylating agent, an antiangiogenic agent, an antimetabolite, a boron-containing agent, a chemoprotective agent, a hormone, an antihormone agent, a corticosteroid, a photoactive therapeutic agent, an oligonucleotide, a radionuclide agent, a topoisomerase inhibitor, a tyrosine kinase inhibitor, a radiosensitizer, and a combination thereof.

Another embodiment of the disclosure provides a glycosylated binding protein wherein the anti-IL1RAP antibody or antigen binding portion thereof comprises one or more carbohydrate residues. Nascent in vivo protein production may undergo further processing, known as post-translational modification. In particular, sugar (glycosyl) residues may be added enzymatically, a process known as glycosylation. The resulting proteins bearing covalently linked oligosaccharide side chains are known as glycosylated proteins or glycoproteins. Antibodies are glycoproteins with one or more carbohydrate residues in the Fc domain, as well as the variable domain. Carbohydrate residues in the Fc domain have important effect on the effector function of the Fc domain, with minimal effect on antigen binding or half-life of the antibody (R. Jefferis, Biotechnol. Prog. 21 (2005), pp. 11-16). In contrast, glycosylation of the variable domain may have an effect on the antigen binding activity of the antibody. Glycosylation in the variable domain may have a negative effect on antibody binding affinity, likely due to steric hindrance (Co, M. S., et al., Mol. Immunol. (1993) 30:1361-1367), or result in increased affinity for the antigen (Wallick, S. C., et al., Exp. Med. (1988) 168:1099-1109; Wright, A., et al., EMBO J. (1991) 10:2717-2723).

One aspect of the disclosure is directed to generating glycosylation site mutants in which the O- or N-linked glycosylation site of the binding protein has been mutated. One skilled in the art can generate such mutants using standard well-known technologies. Glycosylation site mutants that retain the biological activity, but have increased or decreased binding activity, are another object of the disclosure.

Expressing glycosylated proteins different from that of a host cell may be achieved by genetically modifying the host cell to express heterologous glycosylation enzymes. Using recombinant techniques, a practitioner may generate antibodies or antigen binding portions thereof exhibiting human protein glycosylation. For example, yeast strains have been genetically modified to express non-naturally occurring glycosylation enzymes such that glycosylated proteins (glycoproteins) produced in these yeast strains exhibit protein glycosylation identical to that of animal cells, especially human cells (U.S. patent Publication Nos. 20040018590 and 20020137134 and PCT publication WO2005100584 A2).

Antibodies may be produced by any of a number of techniques. For example, expression from host cells, wherein expression vector(s) encoding the heavy and light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express antibodies in either prokaryotic or eukaryotic host cells, expression of antibodies in eukaryotic cells is preferable, and most preferable in mammalian host cells, because such eukaryotic cells (and in particular mammalian cells) are more likely than prokaryotic cells to assemble and secrete a properly folded and immunologically active antibody.

Preferred mammalian host cells for expressing the recombinant antibodies disclosed herein include Chinese Hamster Ovary (CHO cells) (including dhfr− CHO cells, described in Urlaub and Chasm, (1980) Proc. Natl. Acad. Sci. USA 77:4216-4220, used with a DHFR selectable marker, e.g., as described in R. J. Kaufman and P. A. Sharp (1982) Mol. Biol. 159:601-621), NS0 myeloma cells, COS cells and SP2 cells. When recombinant expression vectors encoding antibody genes are introduced into mammalian host cells, the antibodies are produced by culturing the host cells for a period of time sufficient to allow for expression of the antibody in the host cells or, more preferably, secretion of the antibody into the culture medium in which the host cells are grown. Antibodies can be recovered from the culture medium using standard protein purification methods.

Host cells can also be used to produce functional antibody fragments, such as Fab fragments or scFv molecules. It will be understood that variations on the above procedure are within the scope of the disclosure. For example, it may be desirable to transfect a host cell with DNA encoding functional fragments of either the light chain and/or the heavy chain of an antibody. Recombinant DNA technology may also be used to remove some, or all, of the DNA encoding either or both of the light and heavy chains that is not necessary for binding to the antigens of interest. The molecules expressed from such truncated DNA molecules are also encompassed by the antibodies of the disclosure. In addition, bifunctional antibodies may be produced in which one heavy and one light chain are an antibody of the disclosure and the other heavy and light chain are specific for an antigen other than the antigens of interest by crosslinking an antibody of the disclosure to a second antibody by standard chemical crosslinking methods.

In a preferred system for recombinant expression of an antibody, or antigen binding portion thereof, a recombinant expression vector encoding both the antibody heavy chain and the antibody light chain is introduced into CHO cells comprising a glutamine synthase expression system, commercially available from Lonza (hereafter GS-CHO) (Bebbington, C. R. et al. (1992), Biotechnology, 10, pages 169-175). In another system for recombinant expression of an antibody, or antigen binding portion thereof, a recombinant expression vector encoding both the antibody heavy chain and the antibody light chain is introduced into dhfr-CHO cells by calcium phosphate-mediated transfection. Within the recombinant expression vector, the antibody heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. Still further the disclosure provides a method of synthesizing a recombinant antibody by culturing a host cell in a suitable culture medium until a recombinant antibody is synthesized. Recombinant antibodies may be produced using nucleic acid molecules corresponding to the amino acid sequences disclosed herein. In one embodiment, the nucleic acid molecules set forth in SEQ ID NOs: 191-259 are used in the production of a recombinant antibody. The method can further comprise isolating the recombinant antibody from the culture medium.

### III. Anti-IL1RAP Antibody Drug Conjugates (ADCs)

Anti-IL1RAP antibodies described herein may be conjugated to a drug moiety to form an anti-IL1RAP Antibody Drug Conjugate (ADC). Antibody-drug conjugates (ADCs) may increase the therapeutic efficacy of antibodies in treating disease due to the ability of the ADC to selectively deliver one or more drug moiety(s) to target tissues or cells. Thus, in certain embodiments, the disclosure provides anti-IL1RAP ADCs for therapeutic use.

Anti-IL1RAP ADCs comprise an anti-IL1RAP antibody, i.e., an antibody that specifically binds to IL1RAP, linked to one or more drug moieties. The specificity of the ADC is defined by the specificity of the antibody, i.e., anti-IL1RAP.

Examples of linkers that may be used to conjugate the antibody and the one or more drug(s) in the anti-IL1RAP ADCs are provided below. The terms “drug,” “agent,” and “drug moiety” are used interchangeably herein. The terms “linked” and “conjugated” are also used interchangeably herein and indicate that the antibody and moiety are covalently linked.

In some embodiments, the ADC has the following formula (formula I):

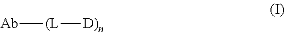

wherein Ab an anti-IL1RAP antibody described herein, and (L-D) is a Linker-Drug moiety. The Linker-Drug moiety is made of L- which is a Linker, and -D, which is a drug moiety having, for example, cytostatic, cytotoxic, or otherwise therapeutic activity against a target cell, e.g., a cell expressing IL1RAP; and n is an integer from 1 to 20. In some embodiments, n ranges from 1 to 8, 1 to 7, 1 to 6, 1 to 5, 1 to 4, 1 to 3, 1 to 2, or is 1. The DAR of an ADC is equivalent to the “n” referred to in Formula I.

Additional details regarding drugs (D of Formula I) and linkers (L of Formula I) that may be used in the ADCs, as well as alternative ADC structures, are described US U.S. Pat. No. 11,248,054, which is incorporated herein by reference in its entirety for all purposes.

The conjugation of the drug to the antibody via a linker can be accomplished by any technique known in the art. A number of different reactions are available for covalent attachment of drugs and linkers to antibodies. This may be accomplished by reaction of the amino acid residues of the antibody, including the amine groups of lysine, the free carboxylic acid groups of glutamic and aspartic acid, the sulfhydryl groups of cysteine and the various moieties of the aromatic amino acids. One of the most commonly used non-specific methods of covalent attachment is the carbodiimide reaction to link a carboxy (or amino) group of a compound to amino (or carboxy) groups of the antibody. Additionally, bifunctional agents such as dialdehydes or imidoesters have been used to link the amino group of a compound to amino groups of an antibody. Also available for attachment of drugs to antibodies is the Schiff base reaction. This method involves the periodate oxidation of a drug that contains glycol or hydroxy groups, thus forming an aldehyde which is then reacted with the binding agent. Attachment occurs via formation of a Schiff base with amino groups of the antibody. Isothiocyanates can also be used as coupling agents for covalently attaching drugs to antibodies. Other techniques are known to the skilled artisan and within the scope of the disclosure.

In certain embodiments, an intermediate, which is the precursor of the linker, is reacted with the drug under appropriate conditions. In certain embodiments, reactive groups are used on the drug or the intermediate. The product of the reaction between the drug and the intermediate, or the derivatized drug, is subsequently reacted with the anti-IL1RAP antibody under appropriate conditions. The synthesis and structure of exemplary linkers, stretcher units, amino acid units, self-immolative spacer units are described in U.S. Patent Application Publication Nos. 20030083263, 20050238649 and 20050009751, each if which is incorporated herein by reference.

Stability of the ADC may be measured by standard analytical techniques such as mass spectroscopy, HPLC, and the separation/analysis technique LC/MS.

### IV. Methods of Using Anti-IL1RAP Antibodies

The anti-IL1RAP antibodies, e.g., BFB759 and the like, and antibody portions (and ADCs) are capable of neutralizing human IL1RAP activity both in vivo and in vitro. Accordingly, such antibodies and antibody portions can be used to inhibit hIL1RAP activity, e.g., in a cell culture containing hIL1RAP, in human subjects or in other mammalian subjects having IL1RAP with which an antibody disclosed herein cross-reacts. In one embodiment, the disclosure provides a method for inhibiting hIL1RAP activity comprising contacting hIL1RAP with an antibody, e.g., BFB759 and the like, or antibody portion such that hIL1RAP activity is inhibited. For example, in a cell culture containing, or suspected of containing hIL1RAP, an anti-IL1RAP antibody, e.g., BFB759 and the like, or antibody portion can be added to the culture medium to inhibit hIL1RAP activity in the culture.

In another embodiment, disclosed herein is a method for reducing hIL1RAP activity in a subject in need thereof, wherein said subject is suffering from an inflammatory, autoimmune, or atopic disease or disorder such as rheumatoid arthritis, psoriasis, psoriatic arthritis, ankylosing spondylitis, Crohn's disease, ulcerative colitis, atopic dermatitis, and asthma, or a disorder in which IL1RAP activity is detrimental. The disclosure provides methods for reducing IL1RAP activity in a subject suffering from such a disease or disorder, which method comprises administering to the subject an anti-IL1RAP antibody or antibody portion of the disclosure such that IL1RAP activity in the subject is reduced.

Preferably, the IL1RAP is human IL1RAP, and the subject is a human subject. Alternatively, the subject can be a mammal expressing an IL1RAP to which antibodies of the disclosure are capable of binding. Still further the subject can be a mammal into which IL 1RAP has been introduced (e.g., by administration of IL1RAP or by expression of a IL1RAP transgene). Anti-IL1RAP antibodies of the disclosure can be administered to a human subject for therapeutic purposes. Moreover, anti-IL1RAP antibodies of the disclosure can be administered to a non-human mammal expressing a IL1RAP with which the antibody is capable of binding for veterinary purposes or as an animal model of human disease. Regarding the latter, such animal models may be useful for evaluating the therapeutic efficacy of antibodies and ADCs of the disclosure (e.g., testing of efficacy, dosages and time courses of administration).

As used herein, the term “a disorder in which IL1RAP activity is detrimental” is intended to include diseases and other disorders in which the presence of IL1RAP in a subject suffering from the disorder has been shown to be or is suspected of being either responsible for the pathophysiology of the disorder or a factor that contributes to a worsening of the disorder. Accordingly, a disorder in which IL1RAP activity is detrimental is a disorder in which reduction of IL1RAP activity is expected to alleviate the symptoms and/or progression of the disorder. Such disorders may be evidenced, for example, by an increase in the concentration of IL1RAP in a biological cell, fluid or tissue of a subject suffering from the disorder (e.g., an increase in the concentration of IL1RAP in a tumor, serum, plasma, synovial fluid, etc. of the subject), which can be detected, for example, using an anti-IL1RAP antibody as described above.

In accordance with the present invention, non-limiting examples of diseases and disorders that can be treated with an anti-IL1RAP antibody, e.g., BFB759 or the like, or antigen binding fragments thereof, include those inflammatory, autoimmune, and atopic diseases and disorders discussed below. For example, suitable diseases and disorders include, but are not limited to, sepsis, acute respiratory distress syndrome, myocardial infarction, cystic fibrosis, irritable bowel disease, ulcerative colitis, Crohn's disease, atopic dermatitis, psoriasis, multiple sclerosis, neutrophilic asthma, Alzheimer's disease, stroke, diabetic kidney disease, diabetes, diabetic retinopathy, hidradenitis suppurativa, rheumatoid arthritis, psoriatic arthritis, ankylosing spondylitis, asthma, allergic rhinitis, allergic conjunctivitis, food allergy, drug allergy, angioedema, allergic contact dermatitis, irritant contact dermatitis, rosacea, psoriasis vulgaris, pustular psoriasis, mastocytosis, systemic lupus erythematosus, systemic sclerosis, chronic spontaneous urticaria, autoimmune blistering diseases, Behcet's disease, vitiligo, generalized pustular psoriasis, palmoplantar pustular psoriasis, CARD14-mediated psoriasis, pyoderma gangrenosum, Sweet's syndrome, acne, folliculitis and eosinophilic pustular folliculitis, adult-onset Still disease, cutaneous lupus erythematosus, contact dermatitis, alopecia areata, cutaneous drug eruptions, graft-versus-host disease, cryopyrin-associated periodic syndromes, granulomatosis with polyangiitis, pyogenic arthritis, pyoderma gangrenosum and acne (PAPA), familial Mediterranean fever, synovitis, pustulosis, hyperostosis, osteitis (SAPHO), bullous pemphigoid, pemphigus vulgaris, Schnitzler syndrome, SAPHO, acne vulgaris, urticarial vasculitis, cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, acute generalized exanthematous pustulosis (AGEP), CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), Chronic obstructive pulmonary disease (COPD), idiopathic pulmonary fibrosis, non-alcoholic fatty liver disease, neutrophilic dermatoses, and monogenic autoinflammatory skin disorders.

In a particular embodiment, the anti-IL1RAP antibody is BFB759, and is used to treat atopic dermatitis. In another embodiment, the anti-IL1RAP antibody is BFB759, and is used to treat irritable bowel syndrome, Crohn's diseases, or ulcerative colitis. In another embodiment, the anti-IL1RAP antibody is BFB759, and is used to treat asthma.

In one embodiment, the anti-IL1RAP antibodies, e.g., BFB759 or the like, disclosed herein are used to treat inflammation, autoimmunity, or atopy. In another embodiment, the anti-IL1RAP antibodies, e.g., BFB759 or the like, and ADCs disclosed herein are used to treat inflammatory, autoimmune, or atopic diseases. Diseases and disorders described herein may be treated by anti-IL1RAP antibodies or ADCs, as well as pharmaceutical compositions comprising such anti-IL1RAP antibodies or ADCs.

In certain embodiments, the antibodies and ADCs disclosed herein are administered to a subject in need thereof in order to treat inflammatory, autoimmune, or atopic diseases, disorders, or conditions that exhibit or are likely to exhibit elevated levels of IL1RAP.

In certain embodiments, the disclosure includes a method for treating (e.g., curing, suppressing, ameliorating, delaying or preventing the onset of, or preventing recurrence or relapse of) an inflammatory or autoimmune disease, disorder, or condition in a mammal in need thereof, comprising administering a therapeutically effective amount of an anti-IL1RAP antibody, e.g., BFB759 or the like, against IL1RAP described herein. In further embodiments, the inflammatory or autoimmune condition is an atopic condition such as atopic dermatitis, and anti-IL1RAP antibody, e.g., BFB759 or the like, decreases levels of TARC/CCL17 (a marker) at the site of atopy or in serum. In still further embodiments, the inflammatory or autoimmune condition is characterized or caused by neutrophil or eosinophil dysfunction. In still further embodiments, the inflammatory or autoimmune condition is further characterized by suppurative inflammation. In still further embodiments, the antibody is characterized by its inhibition of IL-8 release by intestinal epithelial cells, intestinal myofibroblasts, or dermal fibroblasts stimulated with IL-1α, IL-1β, IL-36α, IL-36β, and IL-36γ. In further embodiments, the antibody is administered topically, intradermally, subcutaneously or intravenously at a dose of 1-20 mg/kg. In further embodiments, the antibody is administered as a bolus, less than once per week, twice per week, more than twice per week, or in a continuous infusion.

In further embodiments, the treatment method further comprises a step of identifying the mammal in need thereof according to abnormal serum levels of IL-1α, IL-1β, IL-33, IL-36α, IL-36β, IL-36γ, or combinations thereof, or abnormal serum levels of CXCL1, CXCL8, TARC, LCN2, or combinations thereof. In further embodiments, the treatment method results in the return of these markers to or toward normal levels.

In further embodiments, the treatment method results in a reduction of inflammation, autoimmunity, or atopy determined by reduced levels of inflammation, autoimmunity, or atopy markers known in the art. In further embodiments, the treatment method results in a reduction or resolution of one or more symptoms of the inflammatory, autoimmune, or atopic disease, disorder, or condition.

In some embodiments, the anti-IL1RAP antibody or fragment thereof used in the methods of the invention is a human or humanized anti-IL1RAP antibody or fragment thereof. In one embodiment, an antibody, or antigen binding portion thereof, of the invention comprises an isotype lacking effector function (e.g., human IgG4).

The anti-IL1RAP antibody, e.g., BFB759 or the like, or ADCs, or antigen binding portions thereof, can be used alone or in combination to treat such diseases. It should be understood that the anti-IL1RAP antibody, e.g., BFB759 or the like, or antigen binding portion thereof can be used alone or in combination with at least one additional agent, e.g., a therapeutic agent, said additional agent being selected by the skilled artisan for its intended purpose. For example, the additional agent can be a therapeutic agent art-recognized as being useful to treat the disease or condition being treated by the antibody. The additional agent also can be an agent that imparts a beneficial attribute to the therapeutic composition, e.g., an agent which affects the viscosity of the composition. The additional agent can be a therapeutic antibody, small molecule, siRNA, mRNA, or any other modality known to those skilled in the art.

It should further be understood that the combinations which are to be included within this disclosure are those combinations useful for their intended purpose. The agents set forth below are illustrative for purposes and not intended to be limited. The combinations, which are part of this disclosure, can be the antibodies of the disclosure and at least one additional agent selected from the lists below. The combination can also include more than one additional agent, e.g., two or three additional agents if the combination is such that the formed composition can perform its intended function.

The combination therapy can include one or more IL1RAP antagonists, such as an anti-IL1RAP antibody, e.g., BFB759 or the like, or fragments thereof, formulated with, and/or co-administered with, one or more additional therapeutic agents, e.g., one or more inhibitor of the TH2-associated immune response, the TH1-associated immune response, TH17-associated immune response, pruritis, IL-4 signaling, IL-13 signaling, IL-22 signaling, IL-33 signaling, IL-17 signaling, IL-36 signaling, IL-18 signaling, IL-23 signaling, OX40 signaling, IL-5 signaling, T cell migration, IRAK4 signaling, complement signaling, PDE4 signaling, or combinations thereof.

Provided herein are methods for treating inflammatory, autoimmune, and atopic diseases, disorders, and conditions in a patient comprising administering to the patient an anti-IL1RAP antibody, e.g., BFB759 or the like, or fragment thereof, or an ADC of the invention in combination with at least one additional therapeutic agent, wherein the combination therapy exhibits synergy, e.g., therapeutic synergy, in the subject. As used herein, “synergy” or “therapeutic synergy” refers to a phenomenon where treatment of patients with a combination of therapeutic agents manifests a therapeutically superior outcome to the outcome achieved by each individual constituent of the combination used at its optimum dose (Corbett, T. H. et al., Cancer Treatment Reports, 66:1187 (1982)). For example, a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components.

In particular embodiments, the anti-IL1RAP antibodies, e.g., BFB759 or the like, can be administered alone or with at least one other therapeutic agent which acts in conjunction with or synergistically with the antibody to treat the inflammatory, autoimmune, or atopic disease, disorder, or condition.

In some embodiments, the at least one therapeutic agent used in combination with the anti-IL1RAP antibody, e.g., BFB759 or the like, inhibits the TH2-associated immune response. In some embodiments, the at least one therapeutic agent used in combination with the anti-IL1RAP antibody, e.g., BFB759 or the like, inhibits the TH1-associated immune response. In some embodiments, the at least one therapeutic agent used in combination with the anti-IL1RAP antibody, e.g., BFB759 or the like, inhibits the TH17-associated immune response. In some embodiments, the at least one therapeutic agent used in combination with the anti-IL1RAP antibody, e.g., BFB759 or the like, inhibits both TH1- and TH2-associated immune responses. In some embodiments, the at least one therapeutic agent used in combination with the anti-IL1RAP antibody, e.g., BFB759 or the like, inhibits pruritis.

In some embodiments, the anti-IL1RAP antibody, e.g., BFB759 or the like, is administered in combination with at least one therapeutic agent that inhibits IL-4 signaling. In some embodiments, the therapeutic agent used in combination with the anti-IL1RAP antibody, e.g., BFB759 or the like, is an IL-4Ra inhibitor or antagonist, a Pan-JAK inhibitor or antagonist, or combinations thereof. In some embodiments, the therapeutic agent used in combination with the anti-IL1RAP antibody, e.g., BFB759 or the like, is dupilumab, CBP-201, AK120, cerdulatinib, CEE321, jaktinib, delgocitinib, filgotinib, PF-06651600, tofacitinib, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits IL-13 signaling. In some embodiments, the therapeutic agent used in combination with the anti-IL1RAP antibody, e.g., BFB759 or the like, is an IL-13 inhibitor or antagonist, a JAK1/JAK2 inhibitor or antagonist, or combinations thereof. In some embodiments, the therapeutic agent used in combination with the anti-IL1RAP antibody, e.g., BFB759 or the like, is tralokinumab, lebrikizumab, ASLAN004, baricitinib, ruxolitinib, filgotinib, PF-06651600, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits IL-22 signaling. In some embodiments, the therapeutic agent used in combination with the anti-IL1RAP antibody, e.g., BFB759 or the like, is an IL-22 inhibitor or antagonist, an IL-22R inhibitor or antagonist, a JAK1/TYK2 inhibitor or antagonist, a JAK1/JAK3 inhibitor or antagonist, or combinations thereof. In some embodiments, the therapeutic agent used in combination with the anti-IL1RAP antibody, e.g., BFB759 or the like, is fezakinumab, LEO 138559, brepocitinib, ATI-1777, deucravacitinib, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits IL-33 signaling. In some embodiments, the therapeutic agent used in combination with the anti-IL1RAP antibody, e.g., BFB759 or the like, is an IL-33 or IL-33R inhibitor or antagonist. In some embodiments, the therapeutic agent used in combination with the anti-IL1RAP antibody, e.g., BFB759 or the like, is etokimab, REGN3500, astegolimab, PF-06817024, MEDI3506, CNTO 7160, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis. In some embodiments, such combinations are administered in methods of treating psoriasis. In some embodiments, such combinations are administered in methods of treating allergic contact dermatitis, irritant contact dermatitis, rosacea, psoriasis vulgaris, pustular psoriasis, mastocytosis, systemic lupus erythematosus, systemic sclerosis, chronic spontaneous urticaria, autoimmune blistering diseases, Behcet's disease, or vitiligo.

In some embodiments, the anti-IL1RAP antibody, e.g., BFB759 or the like, is administered in combination with at least one therapeutic agent that inhibits IL-17 signaling. In some embodiments, the therapeutic agent used in combination with the anti-IL1RAP antibody, e.g., BFB759 or the like, is an IL-17A inhibitor or antagonist or an IL-17RA inhibitor or antagonist. In some embodiments, the therapeutic agent used in combination with the anti-IL1RAP antibody, e.g., BFB759 or the like, is secukinumab, ixekizumab, brodalumab, bimekizumab, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease.

In some embodiments, the anti-IL1RAP antibody, e.g., BFB759 or the like, is administered in combination with at least one therapeutic agent that inhibits IL-36 signaling. In some embodiments, the therapeutic agent used in combination with the anti-IL1RAP antibody, e.g., BFB759 or the like, is an IL-36 inhibitor or antagonist or an IL-36R inhibitor or antagonist. In some embodiments, the therapeutic agent used in combination with the anti-IL1RAP antibody, e.g., BFB759 or the like, is spesolimab, imsidolimab, REGN6490, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis. In some embodiments, such combinations are administered in methods of treating neutrophilic dermatoses or hidradenitis suppurativa. In some embodiments, such combinations are administered in methods of treating generalized pustular psoriasis, palmoplantar pustular psoriasis, deficiency of IL-36 receptor antagonist (DITRA), psoriasis vulgaris, CARD14-mediated psoriasis, acute generalized exanthematous pustulosis, hidradenitis suppurativa, pyoderma gangrenosum, Sweet's syndrome, systemic lupus erythematosus, systemic sclerosis, autoimmune blistering diseases, acne, atopic dermatitis, allergic contact dermatitis, or folliculitis and eosinophilic pustular folliculitis.

In some embodiments, the anti-IL1RAP antibody, e.g., BFB759 or the like, is administered in combination with at least one therapeutic agent that inhibits IL-18 signaling. In some embodiments, the therapeutic agent used in combination with the anti-IL1RAP antibody, e.g., BFB759 or the like, is an IL-18 inhibitor or antagonist. In some embodiments, the therapeutic agent used in combination with the anti-IL1RAP antibody, e.g., BFB759 or the like, is tadekinig alfa. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis. In some embodiments, such combinations are administered in methods of treating adult-onset Still disease, lupus erythematosus, or psoriasis. In some embodiments, such combinations are administered in methods of treating adult-onset Still disease, cutaneous lupus erythematosus, psoriasis, atopic dermatitis, chronic spontaneous urticaria, contact dermatitis, alopecia areata, cutaneous drug eruptions, graft-versus-host disease, cryopyrin-associated periodic syndromes, granulomatosis with polyangiitis, systemic sclerosis, hidradenitis suppurativa, pyogenic arthritis, pyoderma gangrenosum and acne (PAPA), familial Mediterranean fever, rosacea, synovitis, acne, pustulosis, hyperostosis, osteitis (SAPHO), bullous pemphigoid, pemphigus vulgaris, Behcet's disease, or Schnitzler syndrome.

In some embodiments, the anti-IL1RAP antibody, e.g., BFB759 or the like, is administered in combination with at least one therapeutic agent that inhibits IL-23 signaling. In some embodiments, the therapeutic agent used in combination with the anti-IL1RAP antibody, e.g., BFB759 or the like, is an IL-23 inhibitor or antagonist, an IL-12/23 p40 subunit inhibitor or antagonist, an IL-23 p19 subunit inhibitor or antagonist, or combinations thereof. In some embodiments, the therapeutic agent used in combination with the anti-IL1RAP antibody, e.g., BFB759 or the like, is risankizumab, ustekinumab, guselkumab, tildrakizumab, mirikizumab, brazikumab, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits OX40 signaling. In some embodiments, the therapeutic agent used in combination with the anti-IL1RAP antibody, e.g., BFB759 or the like, is an OX40 or OX40L inhibitor or antagonist. In some embodiments, the therapeutic agent used in combination with the anti-IL1RAP antibody, e.g., BFB759 or the like, is KHK4083, GBR 830, KY1005, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits IL-5 signaling. In some embodiments, the therapeutic agent used in combination with the anti-IL1RAP antibody, e.g., BFB759 or the like, is an IL-5Ra inhibitor or antagonist, a JAK1 inhibitor or antagonist, or combinations thereof. In some embodiments, the therapeutic agent used in combination with the anti-IL1RAP antibody, e.g., BFB759 or the like, is benralizumab, upadacitinib, abrocitinib, SHR0302, filgotinib, PF-06651600, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits T cell migration. In some embodiments, the therapeutic agent used in combination with the anti-IL1RAP antibody, e.g., BFB759 or the like, is an S1PR1 inhibitor or antagonist, an S1PR4 inhibitor or antagonist, an S1PR5 inhibitor or antagonist, a CCR4 inhibitor or antagonist, or combinations thereof. In some embodiments, the therapeutic agent used in combination with the anti-IL1RAP antibody, e.g., BFB759 or the like, is etrasimod, ozanimod, SCD-044, LC51-0255, BMS-986166, RPT193, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits pruritis. In some embodiments, the therapeutic agent used in combination with the anti-IL1RAP antibody, e.g., BFB759 or the like, is an IL-1α inhibitor or antagonist, an OSMRβ inhibitor or antagonist, an NK1R inhibitor or antagonist, a P2X3 inhibitor or antagonist, an IL-31 inhibitor or antagonist, or combinations thereof. In some embodiments, the therapeutic agent used in combination with the anti-IL1RAP antibody, e.g., BFB759 or the like, is bermekimab, vixarelimab, serlopitant, tradipitant, BLU-5937, nemolizumab, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits the TH-1 associated immune response. In some embodiments, the therapeutic molecule used in combination with the anti-IL1RAP antibody, e.g., BFB759 or the like, is an IL-1α inhibitor or antagonist, IL-1β inhibitor or antagonist, IL-1R1 inhibitor or antagonist, IL-36R inhibitor or antagonist, a TNFα inhibitor or antagonist, or combinations thereof. In some embodiments, the therapeutic agent used in combination with the anti-IL1RAP antibody, e.g., BFB759 or the like, is bermekimab, anakinra, canakinumab, gevokizumab, rilonacept, MEDI8968, spesolimab, imsidolimab, REGN6490, adalimumab, infliximab, etanercept, certolizumab, golimumab, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis. In some embodiments, such combinations are administered in methods of treating adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), atopic dermatitis, allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), or NLRC4-related macrophage activation syndrome (NLRC4-MAS). In some embodiments, such combinations are administered in methods of treating neutrophilic dermatoses, hidradenitis suppurativa, monogenic autoinflammatory skin disorders, or psoriasis.

In some embodiments, the anti-IL1RAP antibody, e.g., BFB759 or the like, is administered in combination with at least one therapeutic molecule that inhibits IRAK4 signaling. In some embodiments, the therapeutic agent used in combination with the anti-IL1RAP antibody, e.g., BFB759 or the like, is an IRAK4 inhibitor or antagonist. In some embodiments, the therapeutic agent used in combination with the anti-IL1RAP antibody, e.g., BFB759 or the like, is PF-06650833, CA-4948, KT-474, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits complement signaling. In some embodiments, the therapeutic agent used in combination with the anti-IL1RAP antibody, e.g., BFB759 or the like, is a C5a inhibitor or antagonist, a C5aR inhibitor or antagonist, a TSLP inhibitor or antagonist, a CD80/CD86 inhibitor or antagonist, an IL-6 inhibitor or antagonist, a CD20 inhibitor or antagonist, an integrin α4 inhibitor or antagonist, an AhR agonist, or combinations thereof. In some embodiments, the therapeutic agent used in combination with the anti-IL1RAP antibody, e.g., BFB759 or the like, is vilobelimab, FX002, INF904, tezepelumab, abatacept, tocilizumab, sarilumab, rituximab, vedolizumab, tapinarof, tadekinig alfa, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments, the anti-IL1RAP antibody, e.g., BFB759 or the like, is administered in combination with at least one therapeutic agent that inhibits PDE4 signaling. In some embodiments, the therapeutic agent used in combination with the anti-IL1RAP antibody, e.g., BFB759 or the like, is a PDE4 inhibitor or antagonist. In some embodiments, the therapeutic agent used in combination with the anti-IL1RAP antibody, e.g., BFB759 or the like, is apremilast, crisaborole, difamilast, roflumilast, or combinations thereof. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

The anti-IL1RAP antibody, e.g., BFB759 or the like, may be administered in combination with multiple therapeutic agents with differing mechanisms of action, for example with a therapeutic agent that inhibits or antagonizes IL-33 and a therapeutic agent that inhibits or antagonizes IL-36. Any of the molecules or classes of molecules described above may be used in such combinations. In some embodiments, such combinations are administered in methods of treating rheumatoid arthritis, psoriasis, psoriatic arthritis, ankylosing spondylitis, Crohn's disease, ulcerative colitis, atopic dermatitis, or asthma. In some embodiments, such combinations are administered in methods of treating atopic disease. In some embodiments, such combinations are administered in methods of treating atopic dermatitis.

In some embodiments related to Examples 8 and 9 herein, provided is a method of treating atopic dermitis or asthma in a patient in need thereof, said method comprising administering a therapeutically effective amount of BFB759 in combination with Dupilumab. Also provided is a method of treating a disease responsive to decreasing serum TARC levels in a patient in need thereof, said method comprising administering a therapeutically effective amount of BFB759 in combination with Dupilumab. Also provided is a method for decreasing serum TARC levels in a patient in need thereof, said method comprising administering a therapeutically effective amount of BFB759 in combination with Dupilumab. In some embodiments, diseases contemplated for treatment herein with the BFB759 and Dupilumab combination, include among others: Atopic Dermatitis, COPD, bronchial asthma, allergic rhinitis, eosinophilic pneumonia, Hypersensitivity Pneumonitis, Lichen Planus, Sarcoidosis, Urticaria, Mastocytosis, and/or eosinophilic-associated disorders.

In some embodiments related to Example 10 herein, provided is a method of treating atopic dermitis or asthma in a patient in need thereof, said method comprising administering a therapeutically effective amount of BFB759 in combination with tralokinumab. Also provided is a method of treating a disease responsive to decreasing serum TARC levels in a patient in need thereof, said method comprising administering a therapeutically effective amount of BFB759 in combination with tralokinumab. Also provided is a method for decreasing serum TARC levels in a patient in need thereof, said method comprising administering a therapeutically effective amount of BFB759 in combination with tralokinumab. In some embodiments, diseases contemplated for treatment herein with the BFB759 and tralokinumab combination, include among others: Atopic Dermatitis, COPD, bronchial asthma, allergic rhinitis, eosinophilic pneumonia, Hypersensitivity Pneumonitis, Lichen Planus, Sarcoidosis, Urticaria, Mastocytosis, eosinophilic-associated disorders.

In some embodiments related to Example 11 herein, provided is a method of treating atopic dermitis or asthma in a patient in need thereof, said method comprising administering a therapeutically effective amount of BFB759 in combination with one or more of: Abrocitinib, Upadacitinib, dexamethasone, triamcinolone and/or prednisone. Also provided is a method of treating a disease responsive to decreasing serum TARC levels in a patient in need thereof, said method comprising administering a therapeutically effective amount of BFB759 in combination with one or more of: Abrocitinib, Upadacitinib, dexamethasone, triamcinolone and/or prednisone. Also provided is a method for decreasing serum TARC levels in a patient in need thereof, said method comprising administering a therapeutically effective amount of BFB759 in combination with one or more of Abrocitinib, Upadacitinib, dexamethasone, triamcinolone and/or prednisone. In some embodiments, diseases contemplated for treatment herein with the BFB759 and Abrocitinib, Upadacitinib, dexamethasone, triamcinolone and/or prednisone combination, include among others: Atopic Dermatitis, COPD, bronchial asthma, allergic rhinitis, eosinophilic pneumonia, Hypersensitivity Pneumonitis, Lichen Planus, Sarcoidosis, Urticaria, Mastocytosis, eosinophilic-associated disorders.

In some embodiments related to Examples 12 and 14 herein, provided is a method of treating atopic dermitis or asthma in a patient in need thereof, said method comprising administering BFB759 in combination with Adalimumab. Also provided is a method of treating a disease responsive to decreasing serum IL-6 levels in a patient in need thereof, said method comprising administering a therapeutically effective amount of BFB759 in combination with Adalimumab. Also provided is a method for decreasing serum IL-6 levels in a patient in need thereof, said method comprising administering a therapeutically effective amount of BFB759 in combination with Adalimumab. Also provided is a method of treating a disease responsive to decreasing serum IL-6 and IL-8 levels in a patient in need thereof, said method comprising administering a therapeutically effective amount of BFB759 in combination with Adalimumab. Also provided is a method for decreasing serum IL-6 and IL-8 levels in a patient in need thereof, said method comprising administering a therapeutically effective amount of BFB759 in combination with Adalimumab. In some embodiments, diseases contemplated for treatment herein with the BFB759 and Adalimumab combination, include among others: rheumatoid arthritis, systemic juvenile idiopathic arthritis (sJIA), Castleman disease, giant cell arteritis, Takayasu arteritis and cytokine release syndrome, adult-onset Still's disease, Ankylosing spondylitis, Crohn disease, Ulcerative colitis, Hidradenitis suppurativa, Plaque psoriasis, Psoriatic arthritis Uveitis, Neutrophilic dermatosis—pyoderma gangrenosum, Behcet disease, Granulomatosis with polyangiitis (also known as Wegener granulomatosis), Sarcoidosis, Pemphigus, Multicentric reticulohistiocytosis, Alopecia areata, inflammatory bowel disease, and/or palmoplantar pustulosis (PPP).

In some embodiments related to Example 13 herein, provided is a method of treating atopic dermitis or asthma in a patient in need thereof, said method comprising administering BFB759 in combination with Secukinumab. Also provided is a method of treating a disease responsive to decreasing serum IL-6 and IL-8 levels in a patient in need thereof, said method comprising administering a therapeutically effective amount of BFB759 in combination with Secukinumab. Also provided is a method for decreasing serum IL-6 and IL-8 levels in a patient in need thereof, said method comprising administering a therapeutically effective amount of BFB759 in combination with Secukinumab. In some embodiments, diseases contemplated for treatment herein with the BFB759 and Adalimumab combination, include among others: rheumatoid arthritis, systemic juvenile idiopathic arthritis (sJIA), Castleman disease, giant cell arteritis, Takayasu arteritis and cytokine release syndrome, adult-onset Still's disease, Ankylosing spondylitis, Crohn disease, Ulcerative colitis, Hidradenitis suppurativa, Plaque psoriasis, Psoriatic arthritis Uveitis, Neutrophilic dermatosis—pyoderma gangrenosum, Behcet disease, Granulomatosis with polyangiitis (also known as Wegener granulomatosis), Sarcoidosis, Pemphigus, Multicentric reticulohistiocytosis, Alopecia areata, inflammatory bowel disease, and/or palmoplantar pustulosis (PPP).

In other embodiments of the invention combination methods provided herein utilizing a human anti-hIL1RAP antibody in combination with other therapeutic agents, other human anti-hIL1RAP antibodies known in the art are also contemplated for use herein.

The pharmaceutical compositions may include a “therapeutically effective amount” or a “prophylactically effective amount” of an antibody or antibody portion. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. In other embodiments, the anti-IL1RAP antibody, e.g., BFB759 or the like, and/or therapeutic agents are administered as a subcutaneous injection or as a bolus intravenously, less frequently than once per week, twice per week, more than twice per week, or in a continuous infusion. It is contemplated herein to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

In certain embodiments, the effective amount of a pharmaceutical composition comprising an anti-IL1RAP antibody, e.g., BFB759 or the like, with or without at least one additional therapeutic agent, to be employed therapeutically will depend, for example, upon the therapeutic context and objectives. One skilled in the art will appreciate that the appropriate dosage levels for treatment, according to certain embodiments, will thus vary depending, in part, upon the molecule delivered, the indication for which an anti-IL1RAP antibody, e.g., BFB759 or the like, with or without at least one additional therapeutic agent, is being used, the route of administration, and the size (body weight, body surface or organ size) and/or condition (the age and general health) of the patient. In certain embodiments, the clinician can titer the dosage and modify the route of administration to obtain the optimal therapeutic effect. In certain embodiments, a typical dosage can range from about 0.1 μg/kg to up to about 100 mg/kg or more, depending on the factors mentioned above. In certain embodiments, the dosage can range from 0.1 pg/kg up to about 100 mg/kg; or 1 μg/kg up to about 100 mg/kg; or 5 μg/kg up to about 100 mg/kg. Another exemplary, non-limiting range for a therapeutically or prophylactically effective amount of an antibody or antibody portion is 0.1-20 mg/kg, more preferably 1-10 mg/kg. It is to be noted that dosage values may vary with the type and severity of the disease, disorder, or condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

Also contemplated herein, is the administration of fixed doses and fixed dosing regiments for use, for example with subcutaneous administration, and the like. Exemplary fixed dosed contemplated for use herein can be selected from: 10 mg, 20 mg, 30 mg, 40 mg, 50 mg, 75 mg, 100 mg, 150 mg, 200 mg, 250 mg, 300 mg, 400 mg, 500 mg, 600 mg, 700 mg, 800 mg, 900 mg, 1000 mg, 1100 mg, 1200 mg, 1300 mg, 1400 mg, 1500 mg, 1600 mg, 1700 mg, 1800 mg, 1900 mg, 2000 mg, and the like. These fixed doses can be used for either the initial loading dose or for subsequent maintenance doses. Accordingly, exemplary dosing regimens contemplated for use herein can include, among others: a 1200 mg loading dose and 600 mg maintenance dose; a 1200 mg loading dose and 300 mg maintenance dose; a 900 mg loading dose and 600 mg maintenance dose; a 900 mg loading dose and 300 mg maintenance dose, a 800 mg loading dose and 400 mg maintenance dose; a 800 mg loading dose and 300 mg maintenance dose; a 800 mg loading dose and 200 mg maintenance dose a 700 mg loading dose and 300 mg maintenance dose; a 600 mg loading dose and 300 mg maintenance dose; a 600 mg loading dose and 250 mg maintenance dose; a 600 mg loading dose and 200 mg maintenance dose; a 600 mg loading dose and 150 mg maintenance dose; a 600 mg loading dose and 100 mg maintenance dose; a 300 mg loading dose and 150 mg maintenance dose; a 300 mg loading dose and 100 mg maintenance dose, and the like.

In another aspect, this application provides a method for detecting the presence of IL1RAP in a sample in vitro (e.g., a biological sample, such as serum, plasma, tissue, biopsy). The subject method can be used to diagnose a disease, disorder, or condition. The method includes: (i) contacting the sample or a control sample with the anti-IL1RAP antibody or fragment thereof as described herein; and (ii) detecting formation of a complex between the anti-IL1RAP antibody or fragment thereof, and the sample or the control sample, wherein a statistically significant change in the formation of the complex in the sample relative to the control sample is indicative of the presence of IL1RAP in the sample.

Given their ability to bind to human IL1RAP, the anti-human IL1RAP antibodies, or portions thereof, (as well as ADCs thereof) can be used to detect human IL1RAP (e.g., in a biological sample, such as serum or plasma), using a conventional immunoassay, such as an enzyme linked immunosorbent assays (ELISA), an radioimmunoassay (RIA) or tissue immunohistochemistry. In one aspect, the disclosure provides a method for detecting human IL1RAP in a biological sample comprising contacting a biological sample with an antibody, or antibody portion, and detecting either the antibody (or antibody portion) bound to human IL1RAP or unbound antibody (or antibody portion), to thereby detect human IL1RAP in the biological sample. The antibody is directly or indirectly labeled with a detectable substance to facilitate detection of the bound or unbound antibody. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm.

Alternative to labeling the antibody, human IL1RAP can be assayed in biological fluids by a competition immunoassay utilizing rhIL1RAP standards labeled with a detectable substance and an unlabeled anti-human IL1RAP antibody. In this assay, the biological sample, the labeled rhIL1RAP standards and the anti-human IL1RAP antibody are combined and the amount of labeled rhIL1RAP standard bound to the unlabeled antibody is determined. The amount of human IL1RAP in the biological sample is inversely proportional to the amount of labeled rhIL1RAP standard bound to the anti-IL1RAP antibody. Similarly, human IL1RAP can also be assayed in biological fluids by a competition immunoassay utilizing rhIL1RAP standards labeled with a detectable substance and an unlabeled anti-human IL1RAP antibody.

In yet another aspect, this application provides a method for detecting the presence of IL1RAP in vivo (e.g., in vivo imaging in a subject). The subject method can be used to diagnose a disorder, e.g., a IL1RAP-associated disorder. The method includes: (i) administering the anti-IL1RAP antibody or fragment thereof as described herein to a subject or a control subject under conditions that allow binding of the antibody or fragment to IL1RAP; and (ii) detecting formation of a complex between the antibody or fragment and IL1RAP, wherein a statistically significant change in the formation of the complex in the subject relative to the control subject is indicative of the presence of IL1RAP.

### V. Pharmaceutical Compositions

The disclosure also provides pharmaceutical compositions comprising an antibody, or antigen binding portion thereof, or ADC and a pharmaceutically acceptable carrier. The pharmaceutical compositions comprising antibodies or ADCs are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies. In another embodiment, the pharmaceutical composition comprises one or more antibodies or ADCs and one or more prophylactic or therapeutic agents other than antibodies or ADCs for treating an inflammatory, autoimmune, or atopic disease, disorder, or condition, or a disease, disorder, or condition in which IL1RAP activity is detrimental. Preferably, the prophylactic or therapeutic agents known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody-portions or ADCs can be incorporated into pharmaceutical compositions suitable for administration to a subject. Typically, the pharmaceutical composition comprises an antibody or antibody portion and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible; and those set forth in U.S. Pat. Nos. 8,829,165 and 8,859,741, which are incorporated by reference in their entirety for all purposes. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion or ADC. Bioavailability of the antibody or antibody portion can be increased by including excipients such as hyaluronidase in the carrier or composition. Viscosity of the composition, carrier, antibody, or antibody portion can be increased by including excipients as caffeine.

Various delivery systems are known and can be used to administer one or more antibodies or ADCs or the combination of one or more antibodies and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem. 262:4429-4432 (1987)), construction of a nucleic acid as part of a retroviral or other vector, etc. Methods of administering a prophylactic or therapeutic agent include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. No. 6,019,968 and WO 99/66903, each of which is incorporated herein by reference their entireties. In one embodiment, an antibody, combination therapy, or a composition is administered using Alkermes AIR® pulmonary drug delivery technology (Alkermes, Inc., Cambridge, Mass.). In a specific embodiment, prophylactic or therapeutic agents are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

In a specific embodiment, it may be desirable to administer the prophylactic or therapeutic agents locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies antagonists is administered locally to the affected area to a subject to prevent, treat, manage, and/or ameliorate a disorder or a symptom thereof. In another embodiment, an effective amount of one or more antibodies is administered locally to the affected area in combination with an effective amount of one or more therapies (e.g., one or more prophylactic or therapeutic agents) other than an antibody of a subject to prevent, treat, manage, and/or ameliorate a disorder or one or more symptoms thereof.

A pharmaceutical composition is formulated to be compatible with its intended route of administration. Examples of routes of administration include, but are not limited to, parenteral, e.g., intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic such as lignocaine to ease pain at the site of the injection.

If the method of the disclosure comprises oral administration, compositions can be formulated orally in the form of tablets, capsules, cachets, gel caps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method may comprise administration of a composition formulated for parenteral administration by injection (e.g., by bolus injection or continuous infusion). Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the disclosure also provides that one or more of the prophylactic or therapeutic agents, or pharmaceutical compositions is packaged in a hermetically sealed container such as an ampoule or sachette indicating the quantity of the agent. In one embodiment, one or more of the prophylactic or therapeutic agents, or pharmaceutical compositions is supplied as a dry sterilized lyophilized powder or water free concentrate in a hermetically sealed container and can be reconstituted (e.g., with water or saline) to the appropriate concentration for administration to a subject. The antibodies and antibody portions or ADCs can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

It will be readily apparent to those skilled in the art that other suitable modifications and adaptations of the methods of the invention described herein are obvious and may be made using suitable equivalents without departing from the scope of the invention or the embodiments disclosed herein. Having now described the invention in detail, the same will be more clearly understood by reference to the following examples, which are included for purposes of illustration only and are not intended to be limiting.

### EXAMPLES

### Example 1. IL1RAP is Expressed in Leukemia Cell Lines

The following experiments were performed to determine IL1RAP protein expression in leukemia cell lines.

### Methods

### Tissue Culture and Cell Lines

Human leukemia cell lines EOL1, Monomac 6, OCI/AML1, KG-1, and Karpas 299 were obtained from DSMZ. Cells were maintained in RPMI-1640 medium (Invitrogen) with 10% fetal bovine serum (FBS) (Sigma).

### Flow Cytometry

Staining for flow cytometry was performed in 1× cold PBS with 0.5% BSA. Primary antibodies (1 ug/ml) were incubated with live cells on ice for 30 minutes, after a brief wash, cells were incubated with Alexa Fluro® 488-conjugated anti-mouse IgG secondary antibody @1:1000 (#4408, Cell Signaling Technology). Acquisition of the data was performed on a MACSQuant® Flow Cytometers (Miltenyi Biotec) and analyzed with FlowJo® software.

### Results

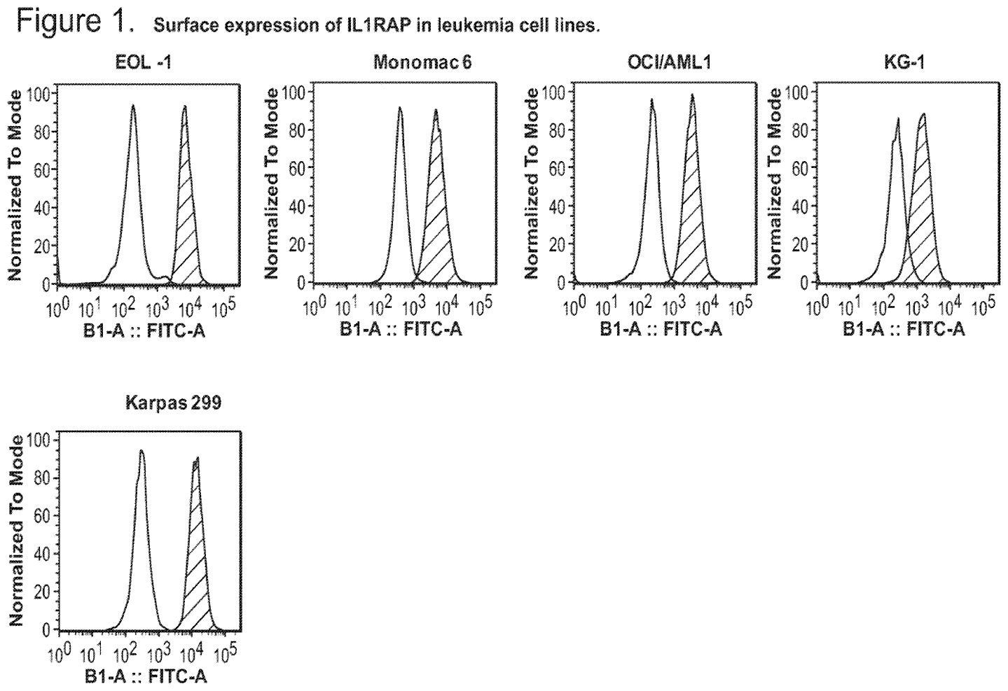

A panel of leukemia cell lines was examined for their surface expression of IL1RAP using a mouse monoclonal anti-IL1RAP antibody (MAB676, R&D System). Flow cytometry analysis was performed for acute myeloid leukemia cell lines EOL1, Monomac 6, OCI/AML1, and KG-1, as well as a T cell leukemia cell line, Karpas 299. Abundant surface expression of IL1RAP was observed in all cell lines tested (see FIG. 1).

### Example 2. Generation of Human Monoclonal Antibodies Against the IL1RAP Extracellular Domain

The following experiments were performed to generate fully human antibodies against the extracellular domain of IL1RAP (SEQ ID NO:260) (IL1RAP-ECD).

### Methods

### Immunizations in Humanized Mice

Monoclonal antibodies were obtained by immunizing with either recombinant human IL1RAP-ECD or 293T cells expressing full-length human IL1RAP tagged with MYC-DDK at the C-terminus. The transgenic mice were engineered with the capacity to produce human immunoglobulins at the variable region. The mice received 5 rounds of either recombinant protein or cells by intraperitoneal injection (IP) and allowed to rest for one month. Then, mice were boosted 4 and 2 days prior to fusion of the spleen with rabbit splenocytes expressing full length IL1RAP or recombinant protein of the extracellular domain (ECD) of IL1RAP. Human IL1RAP-ECD recombinant protein was expressed by EBNA293 cells and purified. For each hybridoma, each variable domain was cloned by RT-PCR into an expression vector that provided the appropriate constant regions. Four plasmid isolates of each cloning were subjected to Sanger Sequencing. After analysis, unique recombinant heavy chains were paired with unique recombinant light chains. These plasmid pairs were transfected into CHO cells in 24-well plates. Eight to twelve days later conditioned medium from each pairing was screened by FLOW™ or Octet™ for binding to IL1RAP.

### Recombinant IL1RAP Cloning

Human, rat, mouse IL1RAP cDNA were purchased from Origene (RC211970, RR213032, MR223729, Rockville, MD). The encoded protein aligns 100% with GenBank IL1RAP_HUMAN. Macaca fascicularis IL1RAP cDNA was synthesized from Gen9 (CST-35853, Cambridge, MA).

Ectodomains of human, Macaca fascicularis, rat and mouse were cloned by PCR. The synthetic genes were based on GenBank sequences (see Table 1).

All DNA sequences were cloned into appropriate CMV-based expression vectors with non-native signal peptides and C-terminal histidine tags for purification.

TABLE 1
Source of IL1RAP protein sequences

| Species | GenBank Protein Reference |
|---|---|
| Human | NP_002173.1 |
| Macaca fascicularis | G7NYP7 |
| Rattus norvegicus | NP_037100.1 |
| Mus musculus | NP_032390.1 |

The retroviral MSCV construct was used to express full-length human IL1RAP protein on the surface of HEK-293T, CHO, and rabbit splenocytes (see Table 2). Mouse, rat and Macaca fascicularis IL1RAP were also expressed on the surface of HEK-293T.

TABLE 2
IL1RAP cell-surface expression vectors

| Plasmid name | Species | Sequence feature | Comment |
|---|---|---|---|
| CST-30815 | Human | Met1-Val570 | Full construct |
| | | (Plus Myc-DDK) | |
| CST-35853 | Macaca | Met1-Val570 | Full construct |
| | Fascicularis | (Plus Myc-DDK) | |
| RR213032 | Rattus | Met1-Val570 | Full construct |
| | norvegicus | (Plus Myc-DDK) | |
| MR223729 | Mus musculus | Met1-Val570 | Full construct |
| | | (Plus Myc-DDK) | |

A series of plasmid constructs designed to secrete a soluble IL1RAP ectodomain were constructed from the full-length plasmids. Each of the constructs in Table 3 was cloned as a fusion protein with an N-terminal maltose binding protein (MBP) and a C-terminal tag of eight histidines (8×His). Both human and mouse versions of the constructs were generated.

TABLE 3
Secreted, soluble recombinant IL1RAP vectors

| Plasmid | | | |
|---|---|---|---|
| name | Species | Sequence feature | Comment |
| BBP1554 | Human | MBP-IL1RAP(S21-T367)- | Full |
| | | 8xHis | ectodomain |
| BBP2182 | Mus musculus | MBP-IL1RAP(S21-T367)- | Full |
| | | 8xHis | ectodomain |

Cloning VH and VL Sequences from Hybridomas

For determination of CDR sequences, total RNA was isolated from hybridoma cells using an RNeasy® kit (Qiagen, Hilden, Germany). First and second-strand cDNA synthesis was performed using a OneTaq® One-Step RT-PCR kit (New England BioLabs, Ipswich, MA). Several primer sets were used from Table 4 of U.S. Pat. No. 11,248,054; SEQ ID NOs:260-285 of U.S. Pat. No. 11,248,054; which is incorporated herein by reference in its entirety for all purposes. PCR products were separated by agarose electrophoresis and fragments were excised and purified by a QIAquick® gel extraction kit (Qiagen, Hilden, Germany). Fragments were cloned directly into expression vectors with BspQI (New England BioLabs, Ipswich, MA) by Golden Gate cloning techniques. Four colonies from each reaction were scaled up for miniprep-scale plasmid purification by SequeMid® DNA Purification Kit (Aline Biosciences, Woburn, MA).

### Transient Expression System of Medium Scale Antibody Production or Recombinant Proteins

The IL1RAP recombinant proteins and anti-IL1RAP antibodies were expressed in Chinese hamster ovary (CHO) cells in a 1 shake flask (working volume of 100-mL) using recommended transfection and media components of the ExpiCHO™ system (Invitrogen, Carlsbad, CA). Cell culture supernatants were harvested 14 days post-transfection, centrifuged, and filtered (0.22 um).

### Antibody and Protein Purification

Conditioned medium from CHO cell cultures was clarified, filtered, and purified by loading onto an ÄKTA Pure™ system with a SmLMabSelect SuRe® column (GE Healthcare). Antibodies were eluted with 100 mM glycine, pH 3.5 and neutralized with 1M Tris-Cl, pH 8.5, and dialyzed against PBS.

Recombinant target proteins were purified from conditioned medium by Ni-NTA chromatography. His-tagged proteins were eluted and dialyzed against PBS.

### Recombinant Antibody Analyses

Concentration: Concentration of recombinant antibodies was determined on a Fortebio Octet Red™ (Pall ForteBio, Fremont, CA) instrument using Protein A tips and a human IgG1 antibody for the standard curve.

Purity testing by SDS-PAGE: Purity testing was performed by sodium dodecyl sulfate polyacrylamide gel electrophoresis (SDS-PAGE) of reduced and non-reduced samples. Samples (10 ug) were mixed with loading buffer (+/−β-mercaptoethanol), heated, and electrophoresed on a 4-20% gel (Invitrogen, Carlsbad, CA). Bands were visualized by Coomassie InstantBlue™ (Expedeon, San Diego, CA) staining.

Purity testing by Endotoxin: Endotoxin concentrations were measured by the Limulus amoebocyte lysate (LAL) kinetic turbidometric method using the Endosafe-PTS™ system (Charles River Laboratories, Wilmington, MA).

Purity testing by HPLC-SEC: Samples were screened for aggregation or other forms of antibody on a 1260 Infinity System™ (Agilent, Santa Clara, CA) with a TSKgel UltraSW Aggregate Guard™ column and HPLC column (Tosoh Bioscience). Samples and standards were detected by absorbance at 280 nm. Comparison against the standard curve provided the molar mass of sample components.

Affinity: The affinity of antibodies to various recombinant IL1RAP protein was determined on an Octet Red™ instrument. After loading reagents into a 96-well plate, the Octet Red™ with Protein A-conjugated biosensors was programmed as follows: 30 seconds for baseline #1; 120 seconds to immobilize the antibody; 30 seconds for baseline #2; 300 seconds for association of antibody to recombinant IL1RAP; and 300-600 seconds for dissociation of recombinant IL1RAP from the antibody.

Binding Competition binning: Binding competition among different antibodies was determined using a real-time, interferometry assay on an Octet Red™ instrument with Protein A-conjugated biosensors. To assess whether two antibodies competed for binding to a recombinant IL1RAP protein, the assay was performed as follows. Protein A biosensors were first submerged into wells containing 10 ug/mL of individual monoclonal antibodies for 5 minutes. Following the capture step, the biosensors were dipped briefly (15 seconds) into buffer and then any unoccupied sites on the biosensor were saturated by submerging them for 5 minutes into wells containing 100 ug/mL of an irrelevant monoclonal antibody. The Octet™ biosensors were then dipped briefly (15 seconds) in buffer before immersion for 1 minute into wells containing recombinant IL1RAP. The biosensors were dipped briefly (15 seconds) in buffer before immersion for 1 minute into wells containing a second recombinant antibody.

For the control case where the second antibody was the same as the first, there was no increase in signal, because there was no additional binding to the recombinant target.

For the control case where buffer was used instead of the first antibody, no recombinant target bound the non-quenching antibody on the biosensor and no second antibody bound the biosensor.

For cases where a boost in signal was seen with the second antibody, the two antibodies were determined not to compete.

For cases where no boost in signal was seen with the second antibody, the two antibodies were determined to compete for binding.

### Immunofluorescence (IF) Based High Content Screening (HCS)

High content immunofluorescence was used to identify wells that contain immunoglobulin that preferentially bound cells expressing IL1RAP. Briefly, CHO cells and CHO-hIL1RAP cells seeded 24 hours before the assay were incubated for 60 minutes at 37° C. with hybridoma supernatant diluted 2-fold in DMEM+10% fetal bovine serum (FBS). After incubation, cells were fixed in 4% formaldehyde, washed with PBS, permeabilized with 0.3% Triton-X-100, and labeled with anti-rat Alexa 488 (at hybridoma stage) or anti-human Alexa 488 secondary antibodies (with recombinant IL1RAP antibodies) for 1 hour at room temperature. Unbound secondary antibody was removed with PBS washes, and cells were stained with DNA dye (propidium iodide and Hoechst 33342).

Potential hits were initially identified via low-resolution, high throughput screening using a TTP Labtech Acumen eX3™ (TTP Labtech, Cambridge, MA), quantifying the fluorescence differential for each sample on both positive and negative cell lines. Those hits were subsequently verified and the subcellular localization of each sample was characterized using a Thermo ArrayScan VTi™ (Thermo Fisher Scientific, Waltham, MA) to obtain high-resolution images of both cell lines.

### Flow Cytometry

Staining for flow cytometry was performed in 1× cold PBS with 0.5% BSA. Primary antibodies (1 ug/ml) were incubated with live cells on ice for 30 minutes, after a brief wash, cells were incubated with Alexa Fluro® 488-conjugated anti-human IgG secondary antibody @1:1000 (709-546-149, Jackson ImmunoResearch). Acquisition of the data was performed on a MACSQuant® Flow Cytometers (Miltenyi Biotec) and analyzed with FlowJo software.

### Correction of Sequence Liabilities

There are a number of amino acid sequences that are predictors of poor performance in clinical-scale production and stability. These include, for example, non-consensus cysteine residues (Cys), non-consensus N-linked glycosylation sites (Asn-xxx-Ser/Thr), acid-sensitive sequences (Asp-Pro). Some of the antibodies derived from hybridoma cloning contain one or more of these sequence liabilities yet otherwise have properties with desirable biological effects.

Some antibody sequences with these sequence liabilities were engineered to eliminate the sequence liability with the intent of retaining or improving the binding properties. Antibodies with a nonconsensus Cys residue have been mutated by replacing the Cys with a germline sequence (if the Cys is in a framework), a Ser residue, or an Ala residue. Constructs of this type were generated and tested for function after expression in CHO cells.

In one case, antibody 5D12_18A4, with a dissociation constant (KD) value of 19 nM, contains a non-consensus Cys sequence in the VH sequence. This heavy chain sequence was engineered to contain a Cys108Tyr mutation. The new heavy chain plasmid was paired with the original light chain plasmid and transfected into CHO cells. The antibody was screened for expression and affinity for human IL1RAP. The 5D12-C108Y antibody expresses at a comparable level to the 5D12_18A4 parent and has a KD value of 13 nM.

In a second case, antibody 10C8_15A1, with a dissociation constant (KD) value of 30 nM, contains a non-consensus Cys sequence in the VH sequence. This heavy chain sequence was engineered to contain a Cys43Ala mutation. The new heavy chain plasmid was paired with the original light chain plasmid and transfected into CHO cells. The antibody was screened for expression and affinity for human IL1RAP. The 10C8_C43A antibody expresses at a comparable level to the 10C8_15A1 parent and has a KD value of 13 nM.

Some antibody sequences with a non-consensus N-linked glycosylation site have been modified at either the Asn site or the Ser/Thr site. Where possible, the Asn or Ser/Thr codons can be mutated back to the germline sequence. In addition, replacing the Asn with Gln or similar amino acid and the Ser or Thr with a similar or smaller amino acid offer a reasonable chance of success.

In one case, antibody 32C12_21A4, with a dissociation constant (KD) value of 1 nM, contains a non-consensus N-linked glycosylation site in CDR1 of the VL sequence. This light chain was engineered to contain an Asn26Ser mutation. The new light chain plasmid was paired with the original heavy chain sequence and transfected into CHO cells. The antibody was screened for expression and affinity for human IL1RAP. The 32C12-N26S antibody expresses 40% higher than the 32C12 parent. The 32C12-N26S antibody has a KD value of 19 nM.

Complete amino acid sequences of the heavy and light chains from 36 antibodies are set forth in Table 5, below.

TABLE 5

| SEQ ID | | Protein | |
|---|---|---|---|
| NO: | Clone Name | Domain | Sequence |
| 1 | 5G8_18A1 | VH | QVELQESGPGLVKPSDTLSLTCTVTGGSISTYYWSWIRQPPGKGLEWI |
| | | | GYIFYTGTTNYNPSLKSRVTISVDASKNQFSLKLNSVTAADTAVYYCA |
| | | | RDGSLDYWGQGALVTVSS |
| 2 | 5G8_18A1 | CDR-H1 | GGSISTYYWS |
| 3 | 5G8_18A1 | CDR-H2 | YIFYTGTTNYNPSLKS |
| 4 | 5G8_18A1 | CDR-H3 | DGSLDY |
| 5 | 5G8_18A1 | VL | DIQMTQSPSSLSASVGDRVTITCRASQSISNYLNWYQQKPGKAPKLLIF |
| | | | VASLLQSGVPSRFSGSGSGTDFTLTISSLQPEDFATYYCQQSYSTPFTF |
| | | | GPGTKLEIK |
| 6 | 5G8_18A1 | CDR-L1 | RASQSISNYLN |
| 7 | 5G8_18A1 | CDR-L2 | VASLLQS |
| 8 | 5G8_18A1 | CDR-L3 | QQSYSTPFT |
| 9 | 10C8_15A1 | VH | QVQLVESGGGVVQPGRSLRISCAASGFTFRSYGMHWVRQAPGKGLE |
| | | | WVAIIWHDESYKYYVDSVKGRFTISRDNSKNTLYLQMNSLRAEDTA |
| | | | VYYCARGDYYGSGSYYDAFDIWGQGTMVTVSS |
| 10 | 10C8_15A1 | CDR-H1 | GFTFRSYGMH |
| 11 | 10C8_15A1 | CDR-H2 | IIWHDESYKYYVDSVKG |
| 12 | 10C8_15A1 | CDR-H3 | GDYYGSGSYYDAFDI |
| 13 | 10C8_15A1 | VL | DIQMTQSPSSLSASVGDRVIITCRASQGISNYLAWFQQKPGKCPNLLIY |
| | | | AASTLRSGVPSRFSGSGSGTDFTLTISSLQPEDVATYYCQKYNSAPYTF |
| | | | GQGTKVEIK |
| 14 | 10C8_15A1 | CDR-L1 | RASQGISNYLA |
| 15 | 10C8_15A1 | CDR-L2 | AASTLRS |
| 16 | 10C8_15A1 | CDR-L3 | QKYNSAPYT |
| 17 | 12F3_17C2 | VH | QVQLVESGGGVVQPGRSLRLSCAASGFTFSHYGMHWVRQAPGKGLE |
| | | | WVAVIWYDGSKKYYVDSVKGRFTISRDNSKNTLYLQMNSLRGEDTA |
| | | | VYYCARDGGAVADNWIDSWGQGTLVTVSS |
| 18 | 12F3_17C2 | CDR-H1 | GFTFSHYGMH |
| 19 | 12F3_17C2 | CDR-H2 | IWYDGSKKYYVDSVKG |
| 20 | 12F3_17C2 | CDR-H3 | DGGAVADNWIDS |
| 21 | 12F3_17C2 | VL | DIQMTQSPSSLSASVGDRVTITCRASQSIRSYLNWYQQKPGKAPKLLIF |
| | | | AASSLQSGVPSRFSGSGSGTDFTLTVSSLQPEDFATYSCQQSYSTPYTF |
| | | | GQGTKVEIK |
| 22 | 12F3_17C2 | CDR-L1 | RASQSIRSYLN |
| 23 | 12F3_17C2 | CDR-L2 | AASSLQS |
| 24 | 12F3_17C2 | CDR-L3 | QQSYSTPYT |
| 25 | 16H2_17D2 | VH | QVQLVESGGGVVQPGGSLRLSCAASGFTFSSHGMHWVRQAPGKGLE |
| | | | WVAVIWYDGSNKFYTDSVQGRFTISRDNSKNTLNLQMNSLRAEDTA |
| | | | VYYCAREGLRAGYYFDFWGQGTLVTVSS |
| 26 | 16H2_17D2 | CDR-H1 | GFTFSSHGMH |
| 27 | 16H2_17D2 | CDR-H2 | VIWYDGSNKFYTDSVQG |
| 28 | 16H2_17D2 | CDR-H3 | EGLRAGYYFDF |
| 29 | 16H2_17D2 | VL | DIQMTQSPSSLSASVGDRVTITCRASQRISFYSNWYQQKPGKAPKLLI |
| | | | YAASSLQSGVPSRFSGSGSGTDFTLTISSLQPEDFATYFCQQSHSTPLTF |
| | | | GGGTKLEIK |
| 30 | 16H2_17D2 | CDR-L1 | RASQRISFYSN |
| 23 | 16H2_17D2 | CDR-L2 | AASSLQS |
| 31 | 16H2_17D2 | CDR-L3 | QQSHSTPLT |
| 32 | 32C12_21A4 | VH | QVQLVESGGGVVQPGRSLRLSCAASGFTFRNYGIHWVRQAPGKGLE |
| | | | WVAVIWYDGSNKFYADSVKGRFTISRDNSKNTLYLQMNSLRVEDTA |
| | | | VYYCAREEGIAVAPFDYWGQGTLVTVSS |
| 33 | 32C12_21A4 | CDR-H1 | GFTFRNYGIH |
| 34 | 32C12_21A4 | CDR-H2 | VIWYDGSNKFYADSVKG |
| 35 | 32C12_21A4 | CDR-H3 | EEGIAVAPFDY |
| 36 | 32C12_21A4 | VL | DIQMTQSPSSLSASVGDRVAITCRANQSIASYLNWYQQKPGKAPKLLI |
| | | | YGASSLQNGVPSRFSGSGSRTDFTLTISSLQPEDFATYYCQQSYSTEITF |
| | | | GQGTRLEIK |
| 37 | 32C12_21A4 | CDR-L1 | RANQSIASYLN |
| 38 | 32C12_21A4 | CDR-L2 | GASSLQN |
| 39 | 32C12_21A4 | CDR-L3 | QQSYSTEIT |
| 40 | 34C11_21B2 | VH | EVQVVESGGGLVQPGGSLRLSCAASGFTFRSYWMSWVRQAPGKGLE |
| | | | WVANIKQDGSERHYVDSVKGRFTISRDNAKTSLYLQMSSLRAEDTAV |
| | | | YYCAREGYFGSGYFDYWGQGTLVTVSS |
| 41 | 34C11_21B2 | CDR-H1 | GFTFRSYWMS |
| 42 | 34C11_21B2 | CDR-H2 | NIKQDGSERHYVDSVKG |
| 43 | 34C11_21B2 | CDR-H3 | EGYFGSGYFDY |
| 44 | 34C11_21B2 | VL | DIQMTQSPSTLSASVGDRVIITCRASQSISRGLAWYQQKPGKAPKLLIY |
| | | | KASNLESGVPSRFSGSGSGTEFTLTISSLQPDDFAAYYCQQYSYYSHTF |
| | | | GQGTKLEIK |
| 45 | 34C11_21B2 | CDR-L1 | RASQSISRGLA |
| 46 | 34C11_21B2 | CDR-L2 | KASNLES |
| 47 | 34C11_21B2 | CDR-L3 | QQYSYYSHT |
| 48 | 36A10_21B6 | VH | QGQVVESGGGVVQPGRSLRLSCAASGFTFSSYTLHWVRQAPGKGLE |
| | | | WVAVIWYDGSNKYYVDSVKGRFTISRDNSKNTLYLQMNSLRAEDTA |
| | | | VYYCAREYGSGSYYNVYYGMDVWGQGTTVTLSS |
| 49 | 36A10_21B6 | CDR-H1 | GFTFSSYTLH |
| 50 | 36A10_21B6 | CDR-H2 | VIWYDGSNKYYVDSVKG |
| 51 | 36A10_21B6 | CDR-H3 | EYGSGSYYNVYYGMDV |
| 52 | 36A10_21B6 | VL | DIVMTQSPLSLPVTPGEPASISCRSSQSLLHSNGYNYLDWYLQKPGQS |
| | | | PHLLIYLASNRASGVPDRFSGSGSGTDFTLKISRVEAEDVGFYYCMQT |
| | | | LQIPLTFGGGTKVEIK |
| 53 | 36A10_21B6 | CDR-L1 | RSSQSLLHSNGYNYLD |
| 54 | 36A10_21B6 | CDR-L2 | LASNRAS |
| 55 | 36A10_21B6 | CDR-L3 | MQTLQIPLT |
| 56 | 39G1_21C4 | VH | QVQLQESGPGLVKPSETLSLTCAVSGYSASGVYYWGWIRQPPGKGLE |
| | | | WIGNIYHSGSTYYNPSLERRVSISLDTSKNHFSLRLNFVTAADTAVYY |
| | | | CARDRFDAFDIWGQGTMVTVSS |
| 57 | 39G1_21C4 | CDR-H1 | GYSASGVYYWG |
| 58 | 39G1_21C4 | CDR-H2 | NIYHSGSTYYNPSLER |
| 59 | 39G1_21C4 | CDR-H3 | DRFDAFDI |
| 60 | 39G1_21C4 | VL | EIVMTQSPATLSVSPGERATLSCRASQSINNILAWYQQKPGQAPRLLIY |
| | | | GASTRATGIPARFSGSGSGTEFTLTISSLQSEDFAVYYCQQYNNWPLTF |
| | | | GGGTKVEIK |
| 61 | 39G1_21C4 | CDR-L1 | RASQSINNILA |
| 62 | 39G1_21C4 | CDR-L2 | GASTRAT |
| 63 | 39G1_21C4 | CDR-L3 | QQYNNWPLT |
| 64 | 37E10_15B5 | VH | QVQLVESGGGVVQPGRSLRLSCAASGFTFRSHGMHWVRQAPGKGLE |
| | | | WVAVIWYDGSSEYYADSVKGRFTISRDNSKNTLYLQMNSLRAEDTA |
| | | | VYYCARENVYGSGWFFDYWGQGTLVTVSS |
| 65 | 37E10_15B5 | CDR-H1 | GFTFRSHGMH |
| 66 | 37E10_15B5 | CDR-H2 | VIWYDGSSEYYADSVKG |
| 67 | 37E10_15B5 | CDR-H3 | ENVYGSGWFFDY |
| 68 | 37E10_15B5 | VL | EIVMTQSPATLSVSPGERASLSCRASQSVGSNLAWYQQRPGQAPRLLI |
| | | | FGASTRATGIPARFSGSRSGTEFTLTISSLQSEDFALYYCQQYNNWPP |
| | | | WTFGQGTKLEIK |
| 69 | 37E10_15B5 | CDR-L1 | RASQSVGSNLA |
| 62 | 37E10_15B5 | CDR-L2 | GASTRAT |
| 70 | 37E10_15B5 | CDR-L3 | QQYNNWPPWT |
| 71 | 44E5_15C5 | VH | QVQLVESGGGVVQPGRSLRLSCAASGFTFRSHGMHWVRQAPGKGLE |
| | | | WVAVIWYDGSSDYYADSVKGRFTISRDNSKNTLFLQMNSLSAEDTA |
| | | | VYYCARENVYGSGWFFDYWGQGTLVTVSS |
| 65 | 44E5_15C5 | CDR-H1 | GFTFRSHGMH |
| 72 | 44E5_15C5 | CDR-H2 | VIWYDGSSDYYADSVKG |
| 67 | 44E5_15C5 | CDR-H3 | ENVYGSGWFFDY |
| 73 | 44E5_15C5 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYNNWPP |
| | | | WTFGQGTKLEIK |
| 69 | 44E5_15C5 | CDR-L1 | RASQSVGSNLA |
| 62 | 44E5_15C5 | CDR-L2 | GASTRAT |
| 70 | 44E5_15C5 | CDR-L3 | QQYNNWPPWT |
| 74 | 38E10_21C3 | VH | EVQLLESGGGLVQPGGSLRLSCAASGFTFSNYAMTWVRQAPGKGLE |
| | | | WVSSISISRAGTYYADSVKGRFTISRDNSKNTLNLQMNSLRAEDTAEY |
| | | | YCAREYYYGMDVWGQGTTVTVSS |
| 75 | 38E10_21C3 | CDR-H1 | GFTFSNYAMT |
| 76 | 38E10_21C3 | CDR-H2 | SISISRAGTYYADSVKG |
| 77 | 38E10_21C3 | CDR-H3 | EYYYGMDV |
| 78 | 38E10_21C3 | VL | EIVLTQSPATLSLSPGERATLSCRASQSVSSYLAWYQQKPGQTPRLLIY |
| | | | DASNRATGIPARFSGSGSGTDFTLTISSLETEDFAVYYCQQLINWPLTF |
| | | | GGGTKLEIK |
| 79 | 38E10_21C3 | CDR-L1 | RASQSVSSYLA |
| 80 | 38E10_21C3 | CDR-L2 | DASNRAT |
| 81 | 38E10_21C3 | CDR-L3 | QQLINWPLT |
| 9 | 10C8_C43A | VH | QVQLVESGGGVVQPGRSLRISCAASGFTFRSYGMHWVRQAPGKGLE |
| | | | WVAIIWHDESYKYYVDSVKGRFTISRDNSKNTLYLQMNSLRAEDTA |
| | | | VYYCARGDYYGSGSYYDAFDIWGQGTMVTVSS |
| 10 | 10C8_C43A | CDR-H1 | GFTFRSYGMH |
| 11 | 10C8_C43A | CDR-H2 | IIWHDESYKYYVDSVKG |
| 12 | 10C8_C43A | CDR-H3 | GDYYGSGSYYDAFDI |
| 82 | 10C8_C43A | VL | DIQMTQSPSSLSASVGDRVIITCRASQGISNYLAWFQQKPGKAPNLLIY |
| | | | AASTLRSGVPSRFSGSGSGTDFTLTISSLQPEDVATYYCQKYNSAPYTF |
| | | | GQGTKVEIK |
| 14 | 10C8_C43A | CDR-L1 | RASQGISNYLA |
| 15 | 10C8_C43A | CDR-L2 | AASTLRS |
| 16 | 10C8_C43A | CDR-L3 | QKYNSAPYT |
| 83 | 16H2_17D1 | VH | EVQLLESGADLVQPGGSLRLSCAASGFTFSSYAMSWVRQAPGKGLE |
| | | | WVSTIRISGGTTYYADSVKGRFTISRDNSKNTLYLQMNSLRAEDTAV |
| | | | YYCVRDYYNGMDVWGQGTTVTVSS |
| 84 | 16H2_17D1 | CDR-H1 | GFTFSSYAMS |
| 85 | 16H2_17D1 | CDR-H2 | TIRISGGTTYYADSVKG |
| 86 | 16H2_17D1 | CDR-H3 | DYYNGMDV |
| 87 | 16H2_17D1 | VL | EIVLTQSPATLSLSPGERATLSCRASQRVSSYLAWYQQKPGQAPRLLI |
| | | | YDASNRATGIPARFSGSGSGTDFTLTISSLEPEDFAVYYCQQRSYWPP |
| | | | AFGQGTKLEIK |
| 88 | 16H2_17D1 | CDR-L1 | RASQRVSSYLA |
| 80 | 16H2_17D1 | CDR-L2 | DASNRAT |
| 89 | 16H2_17D1 | CDR-L3 | QQRSYWPPA |
| 90 | 24G3_17C5 | VH | EVQLLESGGGLVQRGGSLRLSCAASGFTFSSYAMSWVRQAPGKGLE |
| | | | WVSSISGSGDSTNYADSVKGRFTISRDNSKNTLHLQMNSLRAEDTAV |
| | | | YYCVRDYYYGMDVWGHGTTVTVSS |
| 84 | 24G3_17C5 | CDR-H1 | GFTFSSYAMS |
| 91 | 24G3_17C5 | CDR-H2 | SISGSGDSTNYADSVKG |
| 92 | 24G3_17C5 | CDR-H3 | DYYYGMDV |
| 93 | 24G3_17C5 | VL | EIVLTQSPATLSLSPGERATLSCRASQSVNSYLAWYQQKPGQAPRLLI |
| | | | YDASNRATGIPARFSGSGSGTDFTLTISSLEPEDFAVYYCQQRSYWPIT |
| | | | FGQGTRLEIK |
| 94 | 24G3_17C5 | CDR-L1 | RASQSVNSYLA |
| 80 | 24G3_17C5 | CDR-L2 | DASNRAT |
| 95 | 24G3_17C5 | CDR-L3 | QQRSYWPIT |
| 96 | 26C5_15B4 | VH | EAQLLESGGGLVQPGGSLRLSCAASGFTFSSYAMSWVRQAPGKGLE |
| | | | WVSTISGSGGSTHYADSVKGRFTISRDNSKNTLYLQMNSLRAEDTAV |
| | | | YYCVRDYYYGMDVWGQGTTVTVSS |
| 84 | 26C5_15B4 | CDR-H1 | GFTFSSYAMS |
| 97 | 26C5_15B4 | CDR-H2 | TISGSGGSTHYADSVKG |
| 92 | 26C5_15B4 | CDR-H3 | DYYYGMDV |
| 98 | 26C5_15B4 | VL | EIVLTQSPATLSLSPGERATLSCRASQSVSSYLAWYQQKPGQAPRLLIY |
| | | | DASNRATGIPARFSGSGSGTDFTLTISSLEPEDFAVYYCQQRSYWPPTF |
| | | | GQGTKLEIK |
| 79 | 26C5_15B4 | CDR-L1 | RASQSVSSYLA |
| 80 | 26C5_15B4 | CDR-L2 | DASNRAT |
| 99 | 26C5_15B4 | CDR-L3 | QQRSYWPPT |
| 100 | 17E9_15B1 | VH | QVQLVESGGGVVQPGRSLRLSCAASGFTFSSYGMHWVRQAPGKGLE |
| | | | WVAVIWYDGSKKYYVDSVQGRFTISRDNSKNTLYLQMNSLRGEDTA |
| | | | VYYCARDGGAVADNWIDSWGQGTLVTVSS |
| 101 | 17E9_15B1 | CDR-H1 | GFTFSSYGMH |
| 102 | 17E9_15B1 | CDR-H2 | VIWYDGSKKYYVDSVQG |
| 20 | 17E9_15B1 | CDR-H3 | DGGAVADNWIDS |
| 103 | 17E9_15B1 | VL | DIQMTQSPSSLSASVGDRVTLTCRASQNIRSYLNWYQQKPGKAPKLLI |
| | | | FAASSLPSGVPSRFSGSGSVTDFTLTVSSLQPEDFATYSCQQSYSTPYT |
| | | | FGQGTKLEIK |
| 104 | 17E9_15B1 | CDR-L1 | RASQNIRSYLN |
| 105 | 17E9_15B1 | CDR-L2 | AASSLPS |
| 24 | 17E9_15B1 | CDR-L3 | QQSYSTPYT |
| 106 | 10C8-15A4 | VH | EEQLLESGADLAQPGGSLRLSCAASGFTFSSYAMSWVRQAPGKGLE |
| | | | WVSTIRISGDTTYYADSVKGRFTISRDNSKNTLFLQMNSLRAEDTAVY |
| | | | YCVRDYYNGMDVWGHGTTVTVSS |
| 84 | 10C8-15A4 | CDR-H1 | GFTFSSYAMS |
| 107 | 10C8-15A4 | CDR-H2 | TIRISGDTTYYADSVKG |
| 86 | 10C8-15A4 | CDR-H3 | DYYNGMDV |
| 108 | 10C8-15A4 | VL | EIVLTQSPATLSLSPGERATLSCRASQRVSSYLAWYQQKPGQAPRLLI |
| | | | YDASNRATGIPARFSGSGSGTDFTLTISSLEPEDFAVYYCQQRSYWPP |
| | | | AFGQGTKLEIK |
| 88 | 10C8-15A4 | CDR-L1 | RASQRVSSYLA |
| 80 | 10C8-15A4 | CDR-L2 | DASNRAT |
| 89 | 10C8-15A4 | CDR-L3 | QQRSYWPPA |
| 109 | 32A2_21A3 | VH | QVQLVQSGAEVKKPGASVKVSCKASGYTFTGYYMHWVRQAPGQGL |
| | | | EWMGWINPNSGGTNYAQKFQGRVTMTRDTSISTAYMELSRLRSDDT |
| | | | AVYYCAREAVAGTSDAFDIWGQGTMVTVSS |
| 110 | 32A2_21A3 | CDR-H1 | GYTFTGYYMH |
| 111 | 32A2_21A3 | CDR-H2 | WINPNSGGTNYAQKFQG |
| 112 | 32A2_21A3 | CDR-H3 | EAVAGTSDAFDI |
| 113 | 32A2_21A3 | VL | DIQMTQSPSSLSASVGDRVTITCRANQSIASYLNWYQQKPGKVPKLLI |
| | | | YGASSLQNGVPSRFSGSGSRTDFTLTISSLQPEDFATYYCQQSYSTEITF |
| | | | GQGTRLEIK |
| 37 | 32A2_21A3 | CDR-L1 | RANQSIASYLN |
| 38 | 32A2_21A3 | CDR-L2 | GASSLQN |
| 39 | 32A2_21A3 | CDR-L3 | QQSYSTEIT |
| 32 | 32C12-N26S | VH | QVQLVESGGGVVQPGRSLRLSCAASGFTFRNYGIHWVRQAPGKGLE |
| | | | WVAVIWYDGSNKFYADSVKGRFTISRDNSKNTLYLQMNSLRVEDTA |
| | | | VYYCAREEGIAVAPFDYWGQGTLVTVSS |
| 33 | 32C12-N26S | CDR-H1 | GFTFRNYGIH |
| 34 | 32C12-N26S | CDR-H2 | VIWYDGSNKFYADSVKG |
| 35 | 32C12-N26S | CDR-H3 | EEGIAVAPFDY |
| 114 | 32C12-N26S | VL | DIQMTQSPSSLSASVGDRVAITCRASQSIASYLNWYQQKPGKAPKLLI |
| | | | YGASSLQNGVPSRFSGSGSRTDFTLTISSLQPEDFATYYCQQSYSTEITF |
| | | | GQGTRLEIK |
| 115 | 32C12-N26S | CDR-L1 | RASQSIASYLN |
| 38 | 32C12-N26S | CDR-L2 | GASSLQN |
| 39 | 32C12-N26S | CDR-L3 | QQSYSTEIT |
| 116 | 41G4_15B6 | VH | QVQLVESGGGVVQPGRSLRLSCAASGFTFRNYGMHWVRQAPGKGLE |
| | | | WVAVIWYDGSNKFYADSVKGRFTISRDNSKNTLYLQMNSLRVEDTA |
| | | | VYYCAREEGIAVAPFDYWGQGTLVTVSS |
| 117 | 41G4_15B6 | CDR-H1 | GFTFRNYGMH |
| 34 | 41G4_15B6 | CDR-H2 | VIWYDGSNKFYADSVKG |
| 35 | 41G4_15B6 | CDR-H3 | EEGIAVAPFDY |
| 36 | 41G4_15B6 | VL | DIQMTQSPSSLSASVGDRVAITCRANQSIASYLNWYQQKPGKAPKLLI |
| | | | YGASSLQNGVPSRFSGSGSRTDFTLTISSLQPEDFATYYCQQSYSTEITF |
| | | | GQGTRLEIK |
| 37 | 41G4_15B6 | CDR-L1 | RANQSIASYLN |
| 38 | 41G4_15B6 | CDR-L2 | GASSLQN |
| 39 | 41G4_15B6 | CDR-L3 | QQSYSTEIT |
| 118 | 31F9_21A1 | VH | QVQLVESGGGMVQPGRSLRLSCTASGFTFSSHGMHWVRQAPGKGLE |
| | | | WVAVIWFDGSNEYYVDSVKGRFTISRDNSKNTLYLQMNSLRAEDTA |
| | | | VYYCARENVYGSGWFFDYWGQGTLVTVSS |
| 26 | 31F9_21A1 | CDR-H1 | GFTFSSHGMH |
| 119 | 31F9_21A1 | CDR-H2 | VIWFDGSNEYYVDSVKG |
| 67 | 31F9_21A1 | CDR-H3 | ENVYGSGWFFDY |
| 120 | 31F9_21A1 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYNNWPP |
| | | | WTFGQGTKVEIK |
| 69 | 31F9_21A1 | CDR-L1 | RASQSVGSNLA |
| 62 | 31F9_21A1 | CDR-L2 | GASTRAT |
| 70 | 31F9_21A1 | CDR-L3 | QQYNNWPPWT |
| 121 | 32D4_21D6 | VH | QLQLVESGGGVVQPGRSLRLSCAASGFTFSSYGMHWVRQAPGKGLE |
| | | | WVAVIWYDGSSEYYADSVKGRFTISRDNSKNTLYLQMNSLRAEDTA |
| | | | VYYCARENVYGSGWFFDYWGQGTLVTVSS |
| 101 | 32D4_21D6 | CDR-H1 | GFTFSSYGMH |
| 66 | 32D4_21D6 | CDR-H2 | VIWYDGSSEYYADSVKG |
| 67 | 32D4_21D6 | CDR-H3 | ENVYGSGWFFDY |
| 122 | 32D4_21D6 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYNNWPP |
| | | | WTFGQGTKVEIK |
| 69 | 32D4_21D6 | CDR-L1 | RASQSVGSNLA |
| 62 | 32D4_21D6 | CDR-L2 | GASTRAT |
| 70 | 32D4_21D6 | CDR-L3 | QQYNNWPPWT |
| 123 | 32F9_21A5 | VH | QLQLVESGGGVVQPGRSLRLSCAASGFTFSSHGMHWVRQAPGKGLE |
| | | | WVAVIWYDGSSEYYADSVKGRFTISRDNSKNTLYLQMNSLRAEDTA |
| | | | VYYCARENVYGSGWFFDYWGQGSLVTVSS |
| 26 | 32F9_21A5 | CDR-H1 | GFTFSSHGMH |
| 66 | 32F9_21A5 | CDR-H2 | VIWYDGSSEYYADSVKG |
| 67 | 32F9_21A5 | CDR-H3 | ENVYGSGWFFDY |
| 124 | 32F9_21A5 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYNNWPP |
| | | | WTFGQGTKLEIK |
| 69 | 32F9_21A5 | CDR-L1 | RASQSVGSNLA |
| 62 | 32F9_21A5 | CDR-L2 | GASTRAT |
| 70 | 32F9_21A5 | CDR-L3 | QQYNNWPPWT |
| 125 | 33E9_21A6 | VH | QLQLVESGGGVVQFGRSLRLSCAASGFTFSSHGMHWVRQAPGKGLE |
| | | | WVAVIWYDGSSEYYADSVKGRFTISRDNSKNTLYLQMNSLRAEDTA |
| | | | VYYCARENVYGSGWFFDYWGQGTLVTVSS |
| 26 | 33E9_21A6 | CDR-H1 | GFTFSSHGMH |
| 66 | 33E9_21A6 | CDR-H2 | VIWYDGSSEYYADSVKG |
| 67 | 33E9_21A6 | CDR-H3 | ENVYGSGWFFDY |
| 126 | 33E9_21A6 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYNNWPP |
| | | | WTFGQGTKLEIK |
| 69 | 33E9_21A6 | CDR-L1 | RASQSVGSNLA |
| 62 | 33E9_21A6 | CDR-L2 | GASTRAT |
| 70 | 33E9_21A6 | CDR-L3 | QQYNNWPPWT |
| 127 | 35D11_22A1 | VH | QLQLVESGGGVVQPGRSLRLSCAASGFTFSSYGMHWVRQAPGKGLE |
| | | | WVAVIWYDGSSEYYADSVKGRFTISRDNSKNTLFLQMNSLRADDTA |
| | | | VYYCARENVYGSGWFFDYWGQGTLVTVSS |
| 101 | 35D11_22A1 | CDR-H1 | GFTFSSYGMH |
| 66 | 35D11_22A1 | CDR-H2 | VIWYDGSSEYYADSVKG |
| 67 | 35D11_22A1 | CDR-H3 | ENVYGSGWFFDY |
| 128 | 35D11_22A1 | VL | EIVMTQSPATLSVSPGERASLSCRASQSVGSNLAWYQQKPGQAPRLLI |
| | | | FGASTRATGIPARFSGSRSGTEFTLTISSLQSEDFALYYCQQYDNWPP |
| | | | WTFGQGTKLEIK |
| 69 | 35D11_22A1 | CDR-L1 | RASQSVGSNLA |
| 62 | 35D11_22A1 | CDR-L2 | GASTRAT |
| 129 | 35D11_22A1 | CDR-L3 | QQYDNWPPWT |
| 130 | 39A9_28A4 | VH | QVQLVESGGGVVQPGRSLRLSCAASGFTLSGYGMHWVRQAPGKGLE |
| | | | WVAVIWYDGSNKYYADSVKGRFTISRDNSKNMLFLQMNSLRAEDTA |
| | | | VYYCVRENGFGSGWFFDYWGQGNLVTVSS |
| 131 | 39A9_28A4 | CDR-H1 | GFTLSGYGMH |
| 132 | 39A9_28A4 | CDR-H2 | VIWYDGSNKYYADSVKG |
| 133 | 39A9_28A4 | CDR-H3 | ENGFGSGWFFDY |
| 134 | 39A9_28A4 | VL | EIVMTQSPATLSVSPGERATLSCRTSQSVSRDLAWYQQKPGQAPRLLI |
| | | | YGASTRATGIPVRFSGSGSGTEFTLTISSLQSEDFAVYYCQQYNNWPP |
| | | | WTFGQGTKLEIK |
| 135 | 39A9_28A4 | CDR-L1 | RTSQSVSRDLA |
| 62 | 39A9_28A4 | CDR-L2 | GASTRAT |
| 70 | 39A9_28A4 | CDR-L3 | QQYNNWPPWT |
| 136 | 34D1_21B3 | VH | QGQLVESGGGVVQPGRSLRLSCAASGFTFSSYTLHWVRQAPGKGLE |
| | | | WVAVIWYDGSNKYYVDSVKGRFTISRDNSKNTLYLQMNSLRAEDTA |
| | | | VYYCAREYGSGSYYNVYYGMDVWGQGTTVTVSS |
| 49 | 34D1_21B3 | CDR-H1 | GFTFSSYTLH |
| 50 | 34D1_21B3 | CDR-H2 | VIWYDGSNKYYVDSVKG |
| 51 | 34D1_21B3 | CDR-H3 | EYGSGSYYNVYYGMDV |
| 137 | 34D1_21B3 | VL | DIVMTQSPLSLPVTPGEPASISCRSSQSLLHSNGYKYLDWYLQKAGQS |
| | | | PHLLIYLGSNRASGVPDRFSGSGSGTDFTLKISRVEAEDVGFYYCMQT |
| | | | LQIPLTFGGGTKLEIK |
| 138 | 34D1_21B3 | CDR-L1 | RSSQSLLHSNGYKYLD |
| 139 | 34D1_21B3 | CDR-L2 | LGSNRAS |
| 55 | 34D1_21B3 | CDR-L3 | MQTLQIPLT |
| 140 | 33H2_21B1 | VH | EVQVVESGGGLVQPGGSLRLSCATSGFTFRSYWMTWVRQAPGKGLE |
| | | | WVANIKQDGSERHYVDSVKGRFTISRDNAKTSLYLQMSSLRAEDTA |
| | | | MYYCAREGYYGSGYFDYWGQGTLVTVSS |
| 141 | 33H2_21B1 | CDR-H1 | GFTFRSYWMT |
| 42 | 33H2_21B1 | CDR-H2 | NIKQDGSERHYVDSVKG |
| 142 | 33H2_21B1 | CDR-H3 | EGYYGSGYFDY |
| 143 | 33H2_21B1 | VL | DIQMTQSPSTLSASVGDRVIITCRASQSISRGLAWYQQKPGKAPKLLIY |
| | | | KASNLESGVPSRFSGSGSGTEFTLTISSLQPDDFAAYYCQQYSYYSHTF |
| | | | GQGTKVEIK |
| 45 | 33H2_21B1 | CDR-L1 | RASQSISRGLA |
| 46 | 33H2_21B1 | CDR-L2 | KASNLES |
| 47 | 33H2_21B1 | CDR-L3 | QQYSYYSHT |
| 144 | 36A12_21C1 | VH | EVQLVESGGGLVQPGGSLRLSCAASGFTLSFYWMTWVRQAPGKGLE |
| | | | WVANIKQDGNEKNYVDSVKGRFTISKDNAKKSVFLQMNSLRAEDTA |
| | | | VYYCAREGYFGSGYFDYWGQGTLVTVSS |
| 145 | 36A12_21C1 | CDR-H1 | GFTLSFYWMT |
| 146 | 36A12_21C1 | CDR-H2 | NIKQDGNEKNYVDSVKG |
| 43 | 36A12_21C1 | CDR-H3 | EGYFGSGYFDY |
| 147 | 36A12_21C1 | VL | DIQMTQSPSTLSASVGDRVTITCRASQSINSWLAWYQQKPGKAPKLLI |
| | | | YKASTLESGVPSRFSGSGSGTEFTLTISSLQPDDFAPYYCQHYNSYPHT |
| | | | FGQGTKVEIK |
| 148 | 36A12_21C1 | CDR-L1 | RASQSINSWLA |
| 149 | 36A12_21C1 | CDR-L2 | KASTLES |
| 150 | 36A12_21C1 | CDR-L3 | QHYNSYPHT |
| 151 | 38G11_28A2 | VH | EVQLVESGGGLVQPGGSLRLSCAASGFTFSSYWMSWVRQAPGKGLE |
| | | | WVANIKQDGSEKHYVDSVKGRFTISRDNAKNSLYLQMNSLRAEDTA |
| | | | VYYCAREGYFGSGYFDYWGQGTLVTVSS |
| 152 | 38G11_28A2 | CDR-H1 | GFTFSSYWMS |
| 153 | 38G11_28A2 | CDR-H2 | NIKQDGSEKHYVDSVKG |
| 43 | 38G11_28A2 | CDR-H3 | EGYFGSGYFDY |
| 154 | 38G11_28A2 | VL | DIQLTQSPSFLSASVGDRVTITCRASQGISSYLAWYQQKPGKAPKLLIY |
| | | | AASTLQSGVPSRFSGSGSGTEFTLTISSLQPEDFATYYCQQYSFYSHTF |
| | | | GQGTKLEIK |
| 155 | 38G11_28A2 | CDR-L1 | RASQGISSYLA |
| 156 | 38G11_28A2 | CDR-L2 | AASTLQS |
| 157 | 38G11_28A2 | CDR-L3 | QQYSFYSHT |
| 158 | 40C3_22B6 | VH | EVQLVESGGGLVQPGGSLRLSCAASGFTLSFYWMTWVRQAPGKGLE |
| | | | WVANIKQDGNEKNFVDSVKGRFTISRDNAKKSVFLQMNSLRAEDTA |
| | | | VYYCAREGYFGSGYFDYWGQGTLVTVSS |
| 145 | 40C3_22B6 | CDR-H1 | GFTLSFYWMT |
| 159 | 40C3_22B6 | CDR-H2 | NIKQDGNEKNFVDSVKG |
| 43 | 40C3_22B6 | CDR-H3 | EGYFGSGYFDY |
| 160 | 40C3_22B6 | VL | DIQMTQSPSTLSASVGDRVTITCRASQSINSWLAWYQQKPGKAPKLLI |
| | | | YKASSLESGVPSRFSGSGSGTEFTLTISSLQPDDFATYYCQQYNSYPHT |
| | | | FGQGTKLEIK |
| 148 | 40C3_22B6 | CDR-L1 | RASQSINSWLA |
| 161 | 40C3_22B6 | CDR-L2 | KASSLES |
| 162 | 40C3_22B6 | CDR-L3 | QQYNSYPHT |
| 163 | 5D12_18A4 | VH | QVQLQESGPGLVKPLGTLSLTCAVSGGSISNSNWWSWVRQPPGKGLE |
| | | | WIGEIYHSGSTNYNPSLKSRVTIALDWSKNQFSLQLRSVTAADTAVY |
| | | | YCARYGSGPFGGDCWGQGTLVTVSS |
| 164 | 5D12_18A4 | CDR-H1 | GGSISNSNWWS |
| 165 | 5D12_18A4 | CDR-H2 | EIYHSGSTNYNPSLKS |
| 166 | 5D12_18A4 | CDR-H3 | YGSGPFGGDC |
| 167 | 5D12_18A4 | VL | DIQLTQSPSFLSASVGDRVTISCRASQGISSYLAWYQQKPGKAPKLLIY |
| | | | AASTLQGGVPSRFSGSGSGTTFTLTISSLQPEDFATYYCQQLNTYPFTF |
| | | | GPGTKLEIK |
| 155 | 5D12_18A4 | CDR-L1 | RASQGISSYLA |
| 168 | 5D12_18A4 | CDR-L2 | AASTLQG |
| 169 | 5D12_18A4 | CDR-L3 | QQLNTYPFT |
| 170 | 5D12_C108Y | VH | QVQLQESGPGLVKPLGTLSLTCAVSGGSISNSNWWSWVRQPPGKGLE |
| | | | WIGEIYHSGSTNYNPSLKSRVTIALDWSKNQFSLQLRSVTAADTAVY |
| | | | YCARYGSGPFGGDYWGQGTLVTVSS |
| 164 | 5D12_C108Y | CDR-H1 | GGSISNSNWWS |
| 165 | 5D12_C108Y | CDR-H2 | EIYHSGSTNYNPSLKS |
| 171 | 5D12_C108Y | CDR-H3 | YGSGPFGGDY |
| 172 | 5D12-C108Y | VL | DIQLTQSPSFLSASVGDRVTISCRASQGISSYLAWYQQKPGKAPKLLIY |
| | | | AASTLQGGVPSRFSGSGSGTTFTLTISSLQPEDFATYYCQQLNTYPFTF |
| | | | GPGTKLEIK |
| 155 | 5D12-C108Y | CDR-L1 | RASQGISSYLA |
| 168 | 5D12-C108Y | CDR-L2 | AASTLQG |
| 169 | 5D12-C108Y | CDR-L3 | QQLNTYPFT |
| 173 | 37D11_21C2 | VH | QVQIKESGPGLVKPSETLSLTCAVSGFSFSSGYYWGWIRQPPGKGLEW |
| | | | LGSFFHNGNTYYNPSLRSRVTISVDTSKNHFSLKLTSVTAADTAVYYC |
| | | | AGFGDLPHYHYYVMDVWGQGTTVTVSS |
| 174 | 37D11_21C2 | CDR-H1 | GFSFSSGYYWG |
| 175 | 37D11_21C2 | CDR-H2 | SFFHNGNTYYNPSLRS |
| 176 | 37D11_21C2 | CDR-H3 | FGDLPHYHYYVMDV |
| 177 | 37D11_21C2 | VL | EIVLTQSPATLSLSPGERATLSCRASQSVSSYLAWYQQKPGQAPRLLIY |
| | | | DASNRAIGIPARFSGSGSGTDFTLTISSLEPEDFAVYYCQQRSNWPPYT |
| | | | FGQGTKLEIK |
| 79 | 37D11_21C2 | CDR-L1 | RASQSVSSYLA |
| 178 | 37D11_21C2 | CDR-L2 | DASNRAI |
| 179 | 37D11_21C2 | CDR-L3 | QQRSNWPPYT |
| 180 | 42D10_28A5 | VH | QVQPKESGPGVVKPSETLSLTCAVSGFPISRGYYWGWIRQPPGKGLE |
| | | | WIGNIFHSGTTYYNPSLKSRVTISVDTSKNQISLKLTSVTAADTAVYY |
| | | | CVGFGDLPHYQYYVMDIWGQGTTVTVSS |
| 181 | 42D10_28A5 | CDR-H1 | GFPISRGYYWG |
| 182 | 42D10_28A5 | CDR-H2 | NIFHSGTTYYNPSLKS |
| 183 | 42D10_28A5 | CDR-H3 | FGDLPHYQYYVMDI |
| 184 | 42D10_28A5 | VL | EIVLTQSPATLSLSPGERATLSCRASQSVSSYLAWYQQKPGQAPRLLIY |
| | | | DASNRATGIPARFSGSGSGTDFTLTISSLEPEDFAVYYCQQRSNWPPYT |
| | | | FGQGTKLEIK |
| 79 | 42D10_28A5 | CDR-L1 | RASQSVSSYLA |
| 80 | 42D10_28A5 | CDR-L2 | DASNRAT |
| 179 | 42D10_28A5 | CDR-L3 | QQRSNWPPYT |
| 185 | 34H8_21B4 | VH | QVQLQESGPGLVKPSETLSLTCAVSGYSASGLYYWAWIRQPPGKGLE |
| | | | WIGNIYHSGRTYYNPSLESRVSISLDTSKHQVSLKLKSVTYADTAVYF |
| | | | CARDRFDGFDIWGQGTMVTVSS |
| 186 | 34H8_21B4 | CDR-H1 | GYSASGLYYWA |
| 187 | 34H8_21B4 | CDR-H2 | NIYHSGRTYYNPSLES |
| 188 | 34H8_21B4 | CDR-H3 | DRFDGFDI |
| 189 | 34H8_21B4 | VL | EIVMTQSPATLSVSPGERATLSCRASQTINNILAWYQQKPGQAPRLLIY |
| | | | GASTRATGIPARFSGSGSGTEFTLTISSLQSEDFAVYYCQQYNNWPLTF |
| | | | GGGTKLEIK |
| 190 | 34H8_21B4 | CDR-L1 | RASQTINNILA |
| 62 | 34H8_21B4 | CDR-L2 | GASTRAT |
| 63 | 34H8_21B4 | CDR-L3 | QQYNNWPLT |

Complete nucleic acid sequences encoding the heavy and light chains from these 36 antibodies are set forth in Table 6 of U.S. Pat. No. 11,248,054, as SEQ ID NOs:191-259 in U.S. Pat. No. 11,248,054, which is incorporated herein by reference in its entirety for all purposes.

The affinity of the thirty six antibodies was calculated using huIL1RAP ECD recombinant protein by Octet™. Values ranged from 0.2 nM to 127 nM (see Table 7, below).

TABLE 7
KD values for anti-IL1RAP Antibodies

| | KD | |
|---|---|---|
| Sample | (human) | |
| 5G8_18A1 | 0.4 | nM |
| 10C8_15A1 | 30 | nM |
| 10C8_C43A | 13 | nM |
| 10C8_15A4 | 1 | nM |
| 12F3_17C2 | 12 | nM |
| 16H2_17D2 | 0.2 | nM |
| 32C12_21A4 | 1.4 | nM |
| 32C12-N26S | 19 | nM |
| 34C11_21B2 | 0.2 | nM |
| 36A10_21B6 | 0.4 | nM |
| 39G1_21C4 | 1.6 | nM |
| 37E10_15B5 | 0.3 | nM |
| 44E5_15C5 | 0.4 | nM |
| 38E10_21C3 | 2 | nM |
| 16H2_17D1 | 50 | nM |
| 24G3_17C5 | 37 | nM |
| 26C5_15B4 | 127 | nM |
| 17E9_15B1 | 21 | nM |
| 32A2_21A3 | 2 | nM |
| 41G4_15B6 | 5 | nM |
| 31F9_21A1 | 2 | nM |
| 32D4_21D6 | 3 | nM |
| 32F9_21A5 | 2 | nM |
| 33E9_21A6 | 2 | nM |
| 35D11_22A1 | 3 | nM |
| 39A9_28A4 | 3 | nM |
| 34D1_21B3 | 3 | nM |
| 33H2_21B1 | 7 | nM |
| 36A12_21C1 | 24 | nM |
| 38G11_28A2 | 29 | nM |
| 40C3_22B6 | 23 | nM |
| 5D12_18A4 | 19 | nM |
| 5D12-C108Y | 13 | nM |
| 37D11_21C2 | 13 | nM |
| 42D10_28A5 | 7 | nM |
| 34H8_21B4 | 5 | nM |

### Results

Fully human antibodies against IL1RAP were generated by hybridoma procedures. Briefly, transgenic mice were immunized with either recombinant human IL1RAP-ECD or 293T cells over-expressing IL1RAP and boosted with rabbit splenocytes expressing full length human IL1RAP or with ECD of IL1RAP recombinant protein. Splenocytes were fused with the mouse myeloma cell line X63-Ag8.653. Clones from the transgenic mice were identified by immunofluorescence (IF) based high content screening (HCS) on CHO cells overexpressing hIL1RAP, and parental CHO cells not expressing IL1RAP.

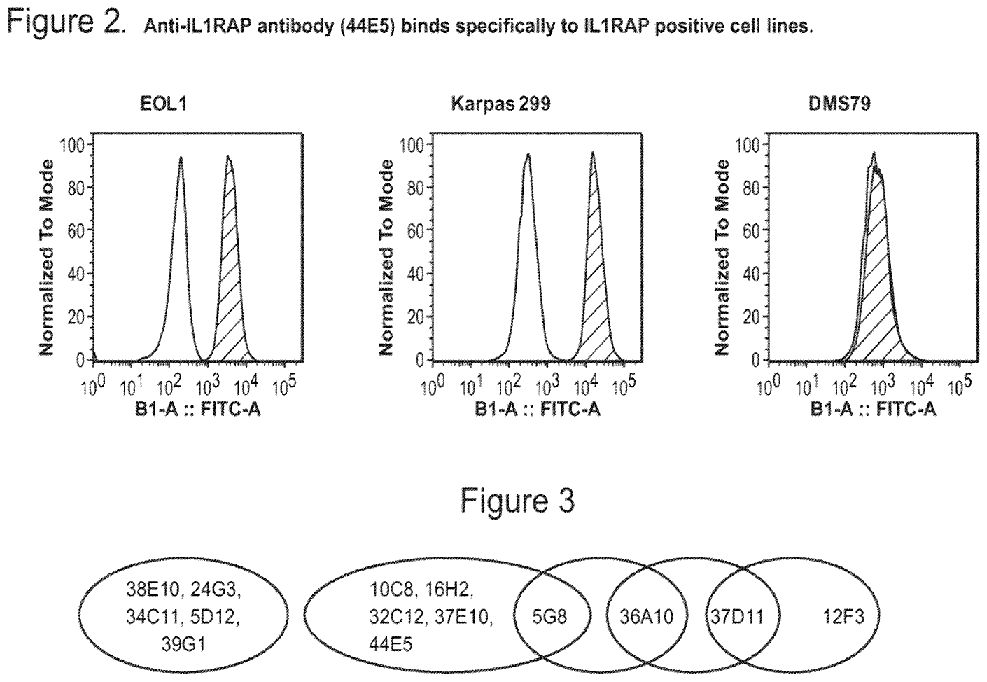

Over 1,000 hits were identified that bind strongly to CHO-human IL1RAP, but not parental CHO cells. 68 clones were selected for molecular cloning. Based on unique CDR3 sequences from the heavy chain variable domain, 35 antibodies from 13 families were confirmed by binding in IL1RAP positive cell lines EOL1 and Karpas 299 (see Table 8) by flow cytometry (MACSQuant® www.miltenybiotec.com). Binding properties of representative antibody clone 44E5_15C5 is shown in FIG. 2.

TABLE 8
Anti-IL1RAP antibodies binding to endogenous cell lines by flow cytometry.

| Ab families | Clone ID | EOL-1 | Karpas 299 | MOLT3 | DMS79 | H69 |
|---|---|---|---|---|---|---|
| 1 | 38E10_21C3 | (+++) | (++/+++) | (+) | | |
| 2 | 10C8_15A4 | (+++) | (+++) | | | (−) |
| | 16H2_17D1 | (+++) | (+++) | | | (−) |
| | 26C5_15B4 | (+++) | (+++) | | | (−) |
| | 24G3_17C5 | (++) | (++) | | | ND |
| 3 | 16H2_17D2 | (+++) | (++++) | | | (+/−) |
| 4 | 17E9_15B1 | (+++) | (+++) | | | (+/−) |
| | 12F3_17C2 | (+++) | (+++) | | | ND |
| 5 | 10C8_15A1 | (+++) | (++++) | (−) | | ND |
| 6 | 32A2_21A3 | (+++) | (++++) | (+) | | |
| | 41G4_15B6 | (+++) | ND | | | ND |
| | 32C12_21A4 | (+++) | (++++) | (+) | | |
| 7 | 31F9_21A1 | (+++) | (++++) | (+) | | |
| | 32F9_21A5 | (+++) | (++++) | (+) | | |
| | 33E9_21A6 | (+++) | (++++) | (+) | | |
| | 32D4_21D6 | (+++) | (++++) | (+) | | |
| | 35D11_22A1 | (+++) | (++++) | (+) | | |
| | 37E10_15B5 | (+++) | (++++) | | | (−) |
| | 44E5_15C5 | (+++) | (++++) | | | (−) |
| | 39A9_28A4 | (+++) | (++++) | | (−) | |
| 8 | 34D1_21B3 | (+++) | (++) | (+) | | |
| | 36A10_21B6 | (+++) | (++) | (+) | | |
| 9 | 36A12_21C1 | (+++) | (+++) | (+) | | |
| | 40C3_22B6 | +++) | (+++) | | | |
| | 33H2_21B1 | (+++) | (+++) | (+) | | |
| | 34C11_21B2 | (+++) | (+++) | (+) | | |
| | 38G11_28A2 | (++) | (++) | | (−) | |
| 10 | 5D12_18A4 | (+++) | (++) | | | ND |
| 11 | 5G8_18A1 | (+++) | (+/++) | | | (−) |
| | 9G4_22B3 | (++/+++) | (+) | | | |
| 12 | 37D11_21C2 | (++/+++) | (++) | (+/−) | | |
| | 42D10_28A5 | (++) | (++) | | (−) | |
| 13 | 34H8_21B4 | (+++) | (++++) | (+) | | |
| | 39G1_21C4 | (+++) | (++++) | (+) | | |

(−) No binding
(+) Weak binding
(++) Moderate binding
(+++) Strong binding
ND Not determined

Selected antibodies were evaluated in pairs for their ability to simultaneously bind recombinant human IL1RAP. The analysis was performed by Octet™ loaded with Protein A Biosensors. A total of five different bins were determined as follows. Some of the bins overlapped with each other such that a given antibody may be in more than one bin.
(1) The following five antibodies competed with each other for binding: 5D12_18A4, 24G3_17C5, 34C11_21B2, 38E10_21C3, 39G1_21C4.
(2) The following six antibodies competed with each other: 10C8_15A1, 16H2_17D2, 32C12_21A4, 37E10_15B5, 44E5_15C5, 5G8_18A1.
(3) Two antibodies competed with each other in a unique arrangement—5G8_18A1, 36A10_21B6.
(4) Two antibodies competed with each other in a unique arrangement—36A10_21B6, 37D11_21C2.
(5) Two antibodies competed with each other in a unique arrangement—37D11_21C2, 12F3_17C2.
A diagram representing the arrangement of these competing bins shown in FIG. 3.

### Example 3 Binding of Anti-IL1RAP Monoclonal Antibodies to IL1RAP Orthologs

Experiments were performed to determine the binding of anti-IL1RAP human monoclonal antibodies to IL1RAP in different species. The following methods were used.

### Methods

### Tissue Culture and Cell Lines

293T cell line was purchased from American Type Culture Collection (ATCC). 293T cells expressing human, Macaca fascicularis, rat, and mouse IL1RAP were maintained in DMEM medium (Invitrogen) with 10% fetal bovine serum (FBS) (Sigma) in the presence of 2 ug/ml puromycin (Invitrogen).

### Flow Cytometry

Staining for flow cytometry was performed in 1× cold PBS with 0.5% BSA. Primary antibodies (1 ug/ml) were incubated with live cells on ice for 30 minutes, after a brief wash, cells were incubated with Alexa Fluro® 488-conjugated anti-human IgG secondary antibody @1:1000 (709-546-149, Jackson ImmunoResearch). Acquisition of the data was performed on a MACSQuant® Flow Cytometers (Miltenyi Biotec) and analyzed with FlowJo software.

### Results

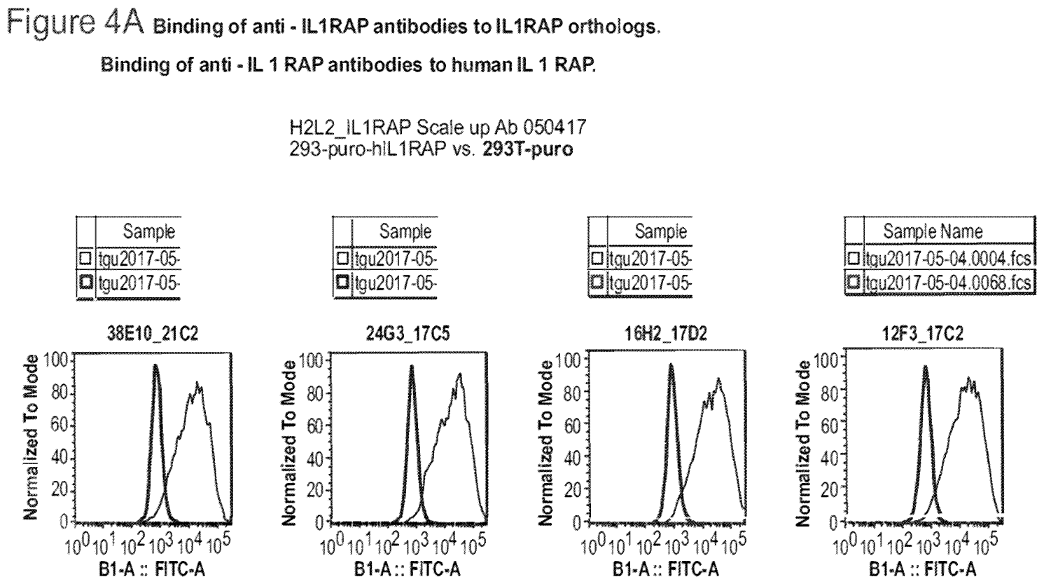

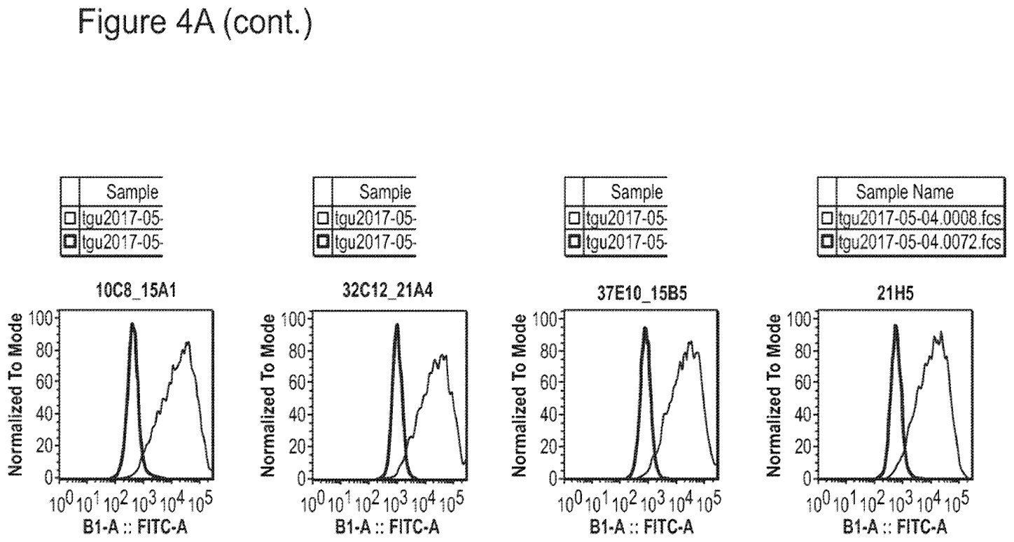

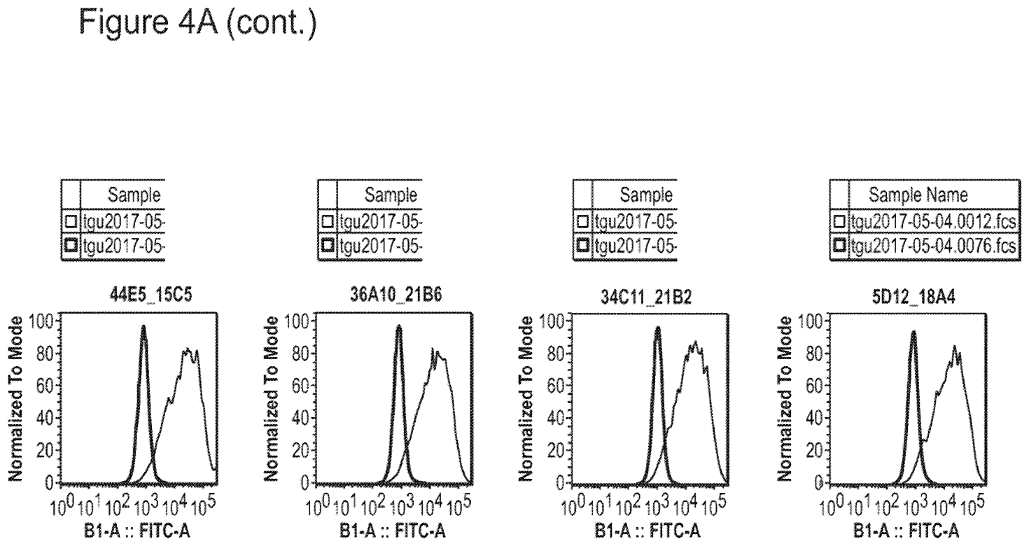

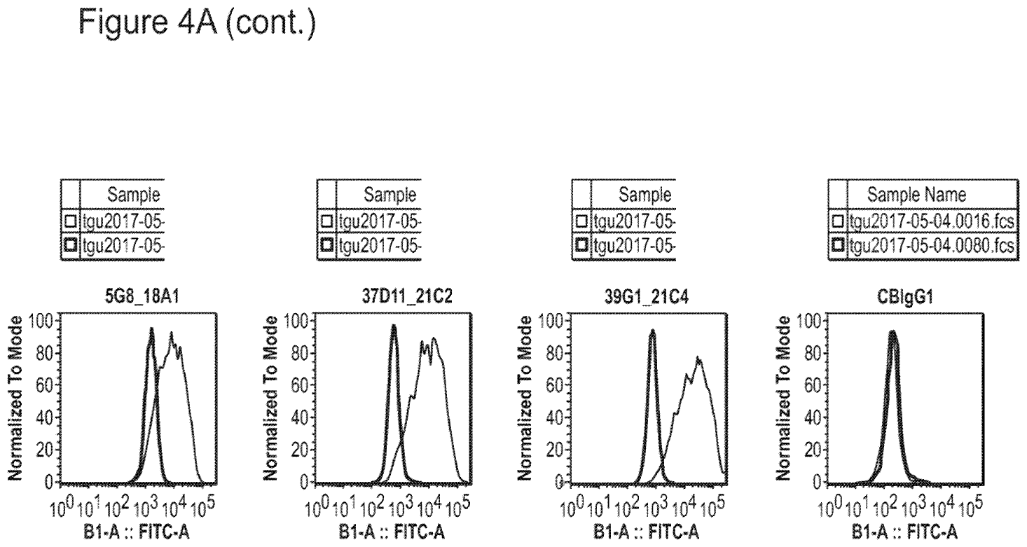

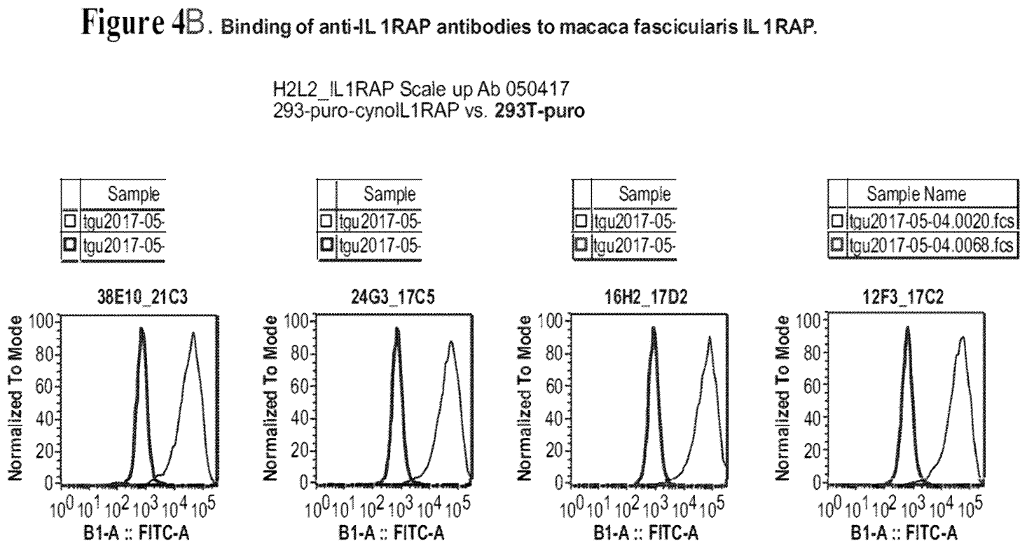

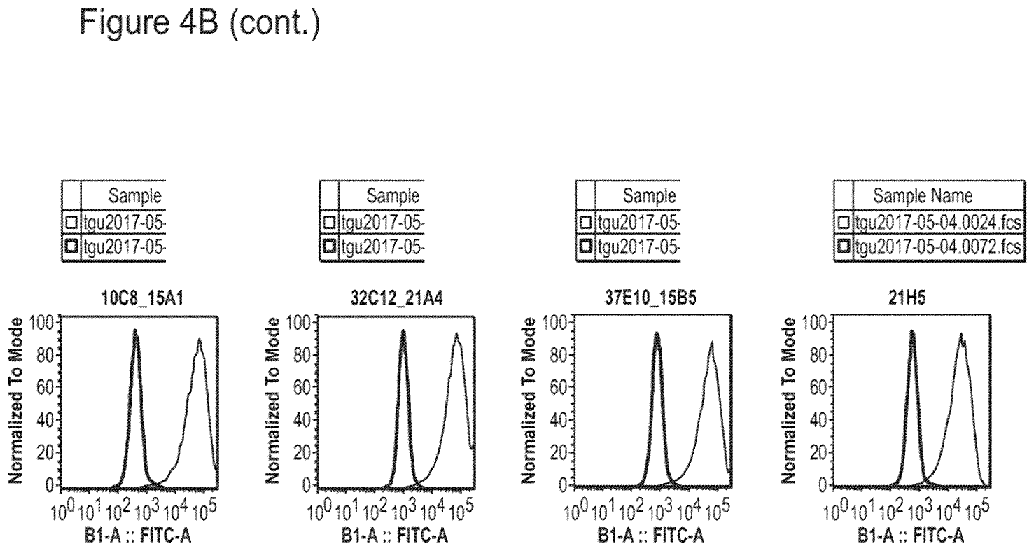

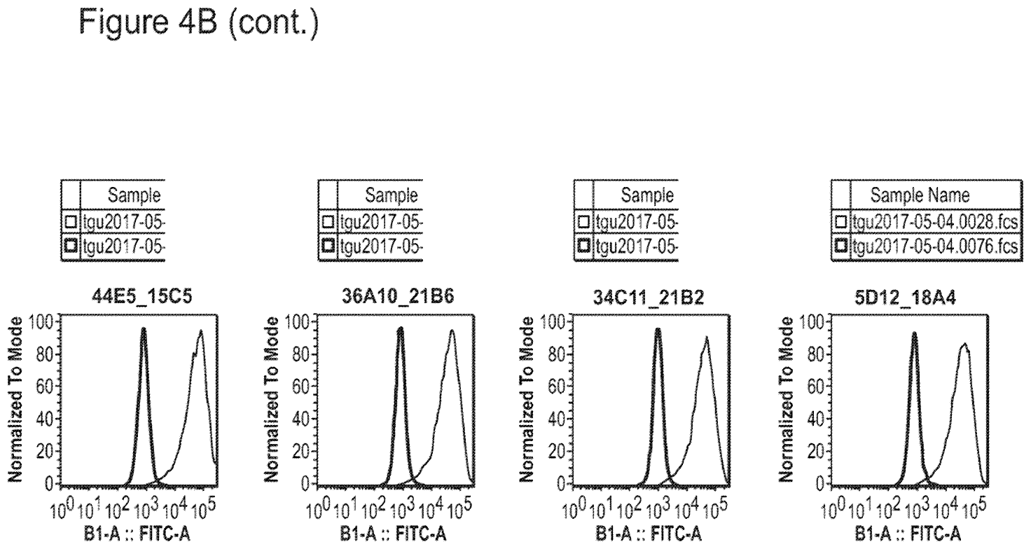

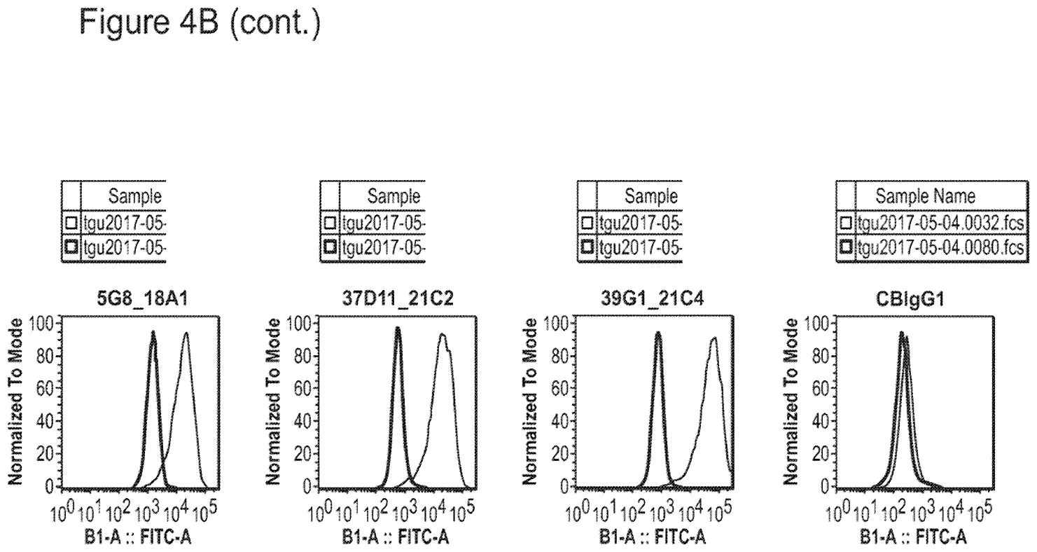

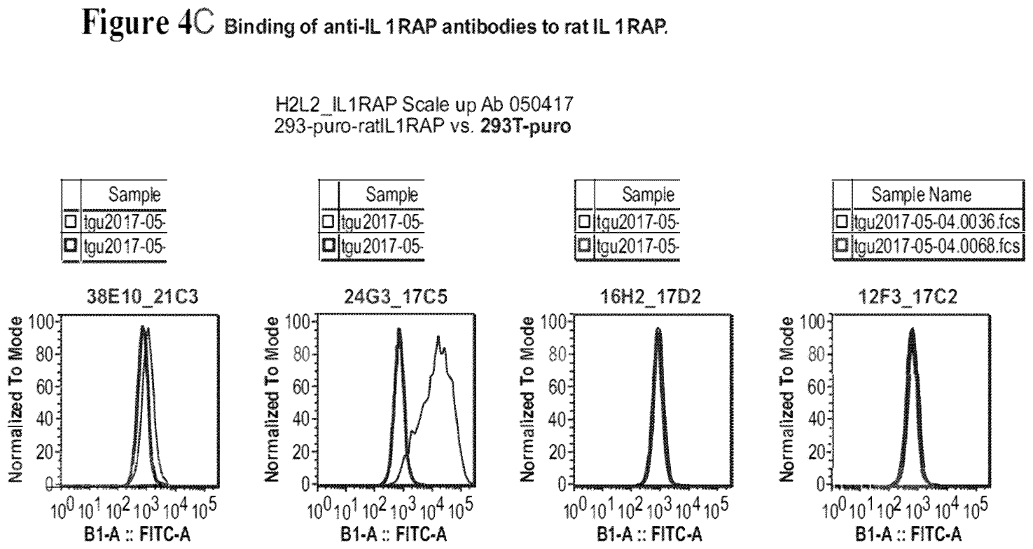

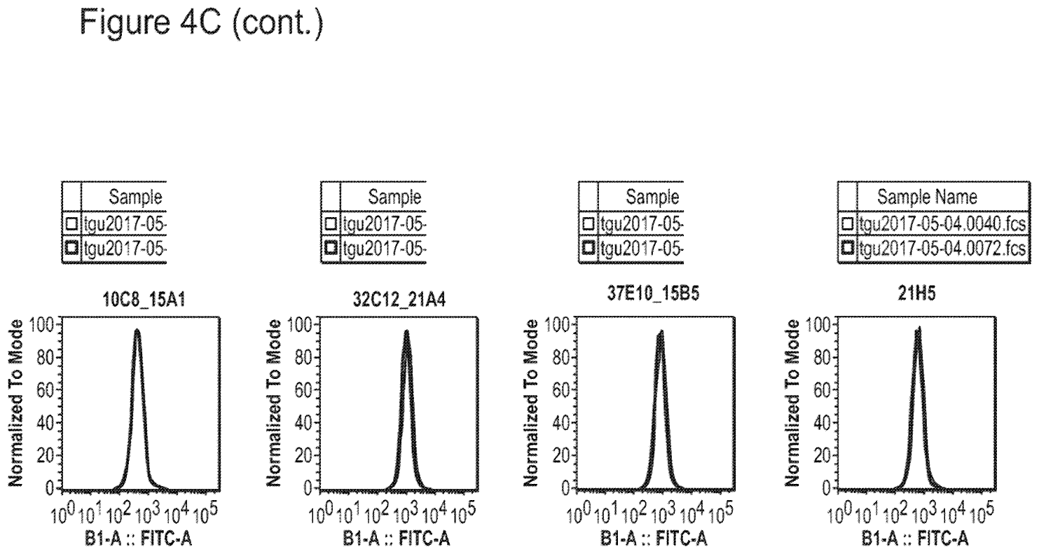

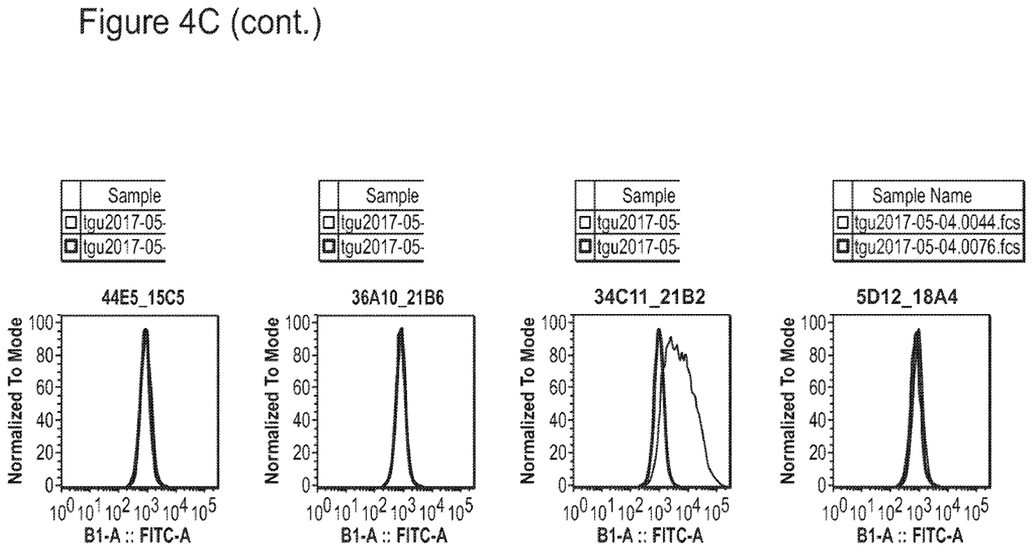

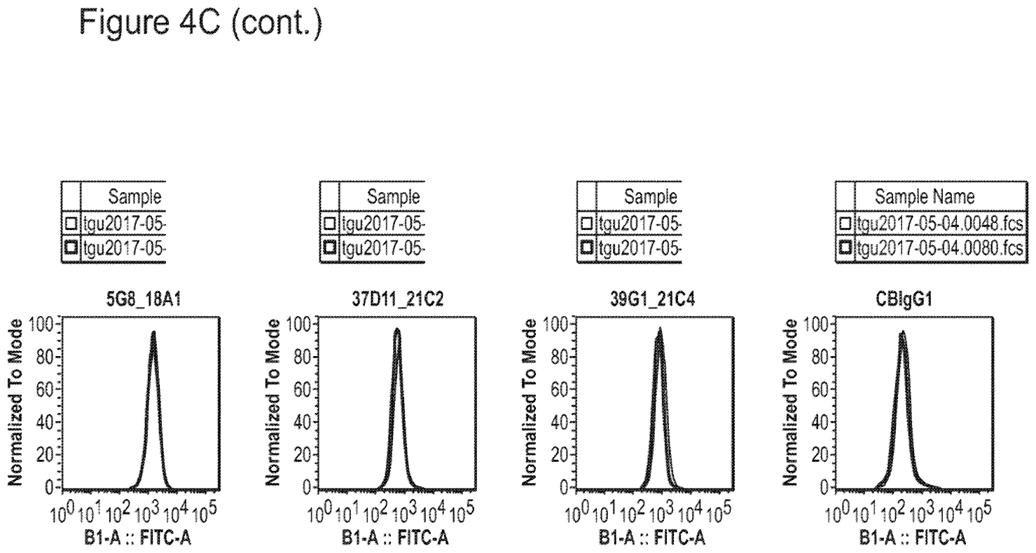

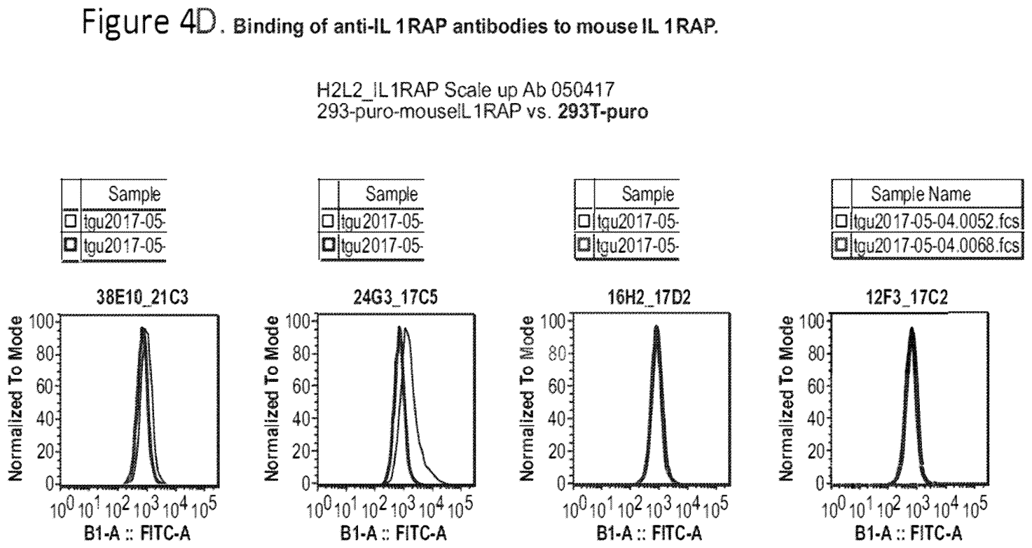

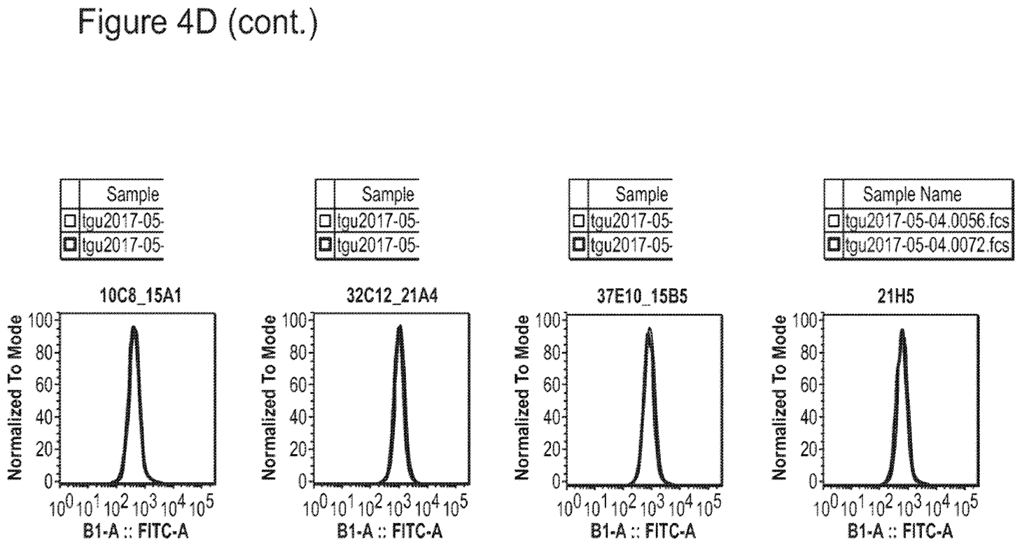

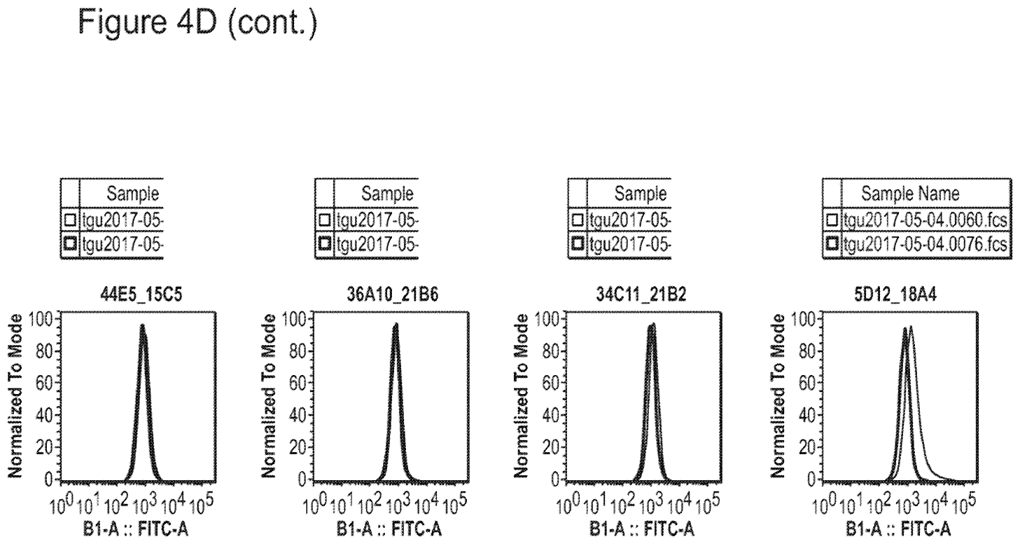

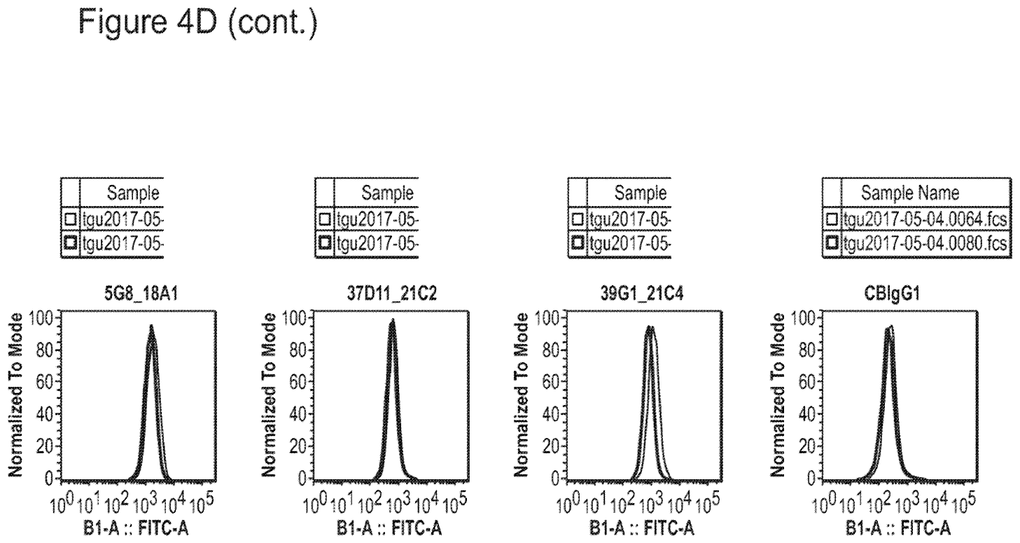

To evaluate the binding of anti-IL1RAP human monoclonal antibodies to IL1RAP in different species, 293 cells overexpressing human, Macaca fascicularis, rat, and mouse IL1RAP were generated. While all antibodies bind human and Macaca fascicularis IL1RAP (FIGS. 4A-4B), 24G3_17C5 and 34C11_21B2 bind 293 cells expressing rat IL1RAP and 24G3_17C5, 5D12_18A4, and 39G1_21C4 bind weakly to 293 cells expressing mouse IL1RAP (FIGS. 4C-4D).

### Example 4. Internalization of Anti-IL1RAP Antibody in AML Cell Line

Experiments were performed to characterize anti-IL1RAP antibody internalization in EOL-1 cells. The following methods were used.

### Methods

### Tissue Culture and Cell Lines

Human leukemia cell lines EOL1 was obtained from DSMZ. They were maintained in RPMI-1640 medium (Invitrogen) with 10% fetal bovine serum (FBS) (Sigma).

### Internalization Assay

Live EOL1 cells were incubated with 44E5_15C5 antibody for 30 minutes at 37° C. After cytospin, cells were fixed with 4% PFA and permeabalized with 100% methanol, and stained with LAMP1 antibody (#9091, Cell Signaling Technology, Inc.).

### Results

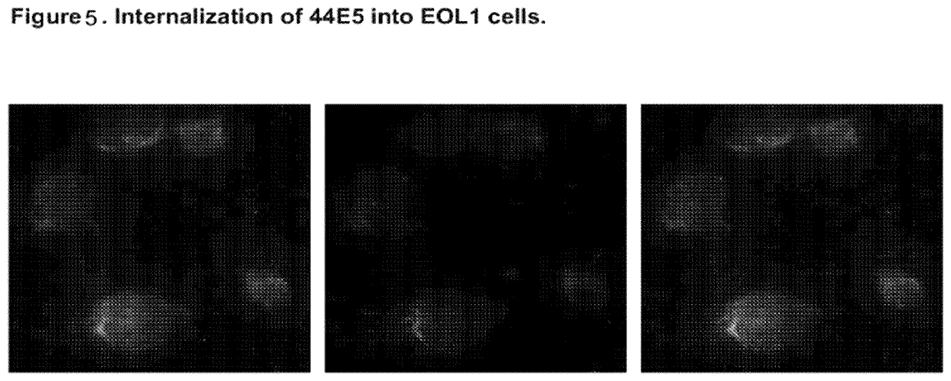

Live EOL1 cells were incubated with 44E5_15C5 for 0.5 hours at 37° C. Cells were then fixed, permeabalized, and co-stained with LAMP1 antibody. 44E5_15C5 is co-localized to lysozyme, marked by LAMP1 antibody (see FIG. 5).

### Example 5. Blockage of IL1 Signaling by Anti-IL1RAP Antibody

The following experiments were performed to characterize the ability of anti-IL1RAP antibody to block IL1 signaling.

### Methods

### IL-1 Signaling Reporter Cell Assay

HEK-Blue IL-1β cells (Invivogen, CA) were harvested and plated in technical duplicates at a density of 50,000 cells per well in a 96-well plate. IL1RAP antibodies, or a corresponding human IgG1 control antibody was added to the wells in a concentration range of 1-10 pg/ml. After incubating cells with antibodies for 30 minutes, IL-1β was added to a final concentration of 0.5 ng/ml, and the plate was incubated overnight. To examine whether any of the antibodies were able to induce IL-1R activation in the absence of IL-1β, samples were incubated with 10 μg/mL antibody without addition of the ligand. The following day, substrate was added to the supernatants, and samples were analyzed for absorbance at 620 nm.

### Results

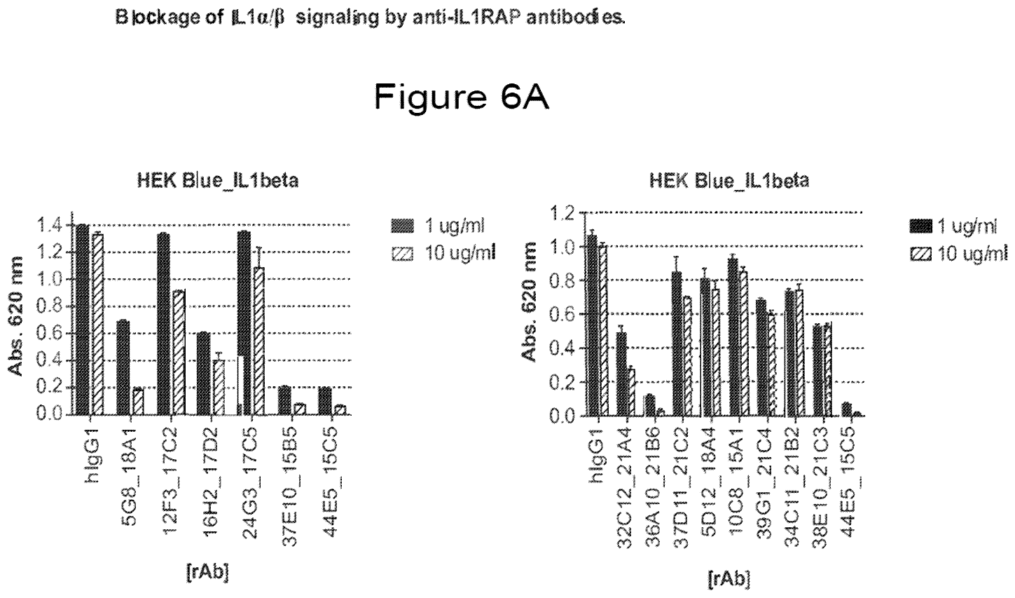

Since IL1RAP is essential for IL1 signaling, the ability of the IL1RAP antibodies described herein to inhibit IL-1 signaling was investigated. Whereas antibodies showed various degrees of inhibitory effect in an IL-1 reporter assay, IL1RAP antibodies 37E10_15B5, 44E5_15C5, 16H2_17D2, 5G8_18A1 and 36A10_21B6 displayed potent inhibition of IL1R1 signaling in a dose dependent manner (FIG. 6A).

None of the IL1RAP antibodies tested affected IL1R1 signaling in the absence of IL-1, thereby excluding agonistic functions on IL1 induced signaling.

In addition, serial dilution of IL1RAP antibodies was performed to determine EC₅₀ against IL-1β and IL-1α (FIGS. 6B and 6C). Many of these antibodies, including 37E10_15B5 and 44E5_15C5 can block IL-1β and IL-1α signaling with subnanomolar EC₅₀. On the other hand, 37D11_21C2 and 39G1_21C4 do not inhibit IL-1 signaling.

### Example 6. Blockage of IL-33 Signaling by Anti-IL1RAP Ab

Experiments were performed to characterize the ability of anti-IL1RAP antibody to block IL-33 signaling. The following methods were used.

### Methods

### IL-33 Signaling Reporter Cell Assay

HEK-Blue IL-33 cells (Invivogen, CA) were harvested and plated in technical duplicates at a density of 50,000 cells per well in a 96-well plate. antibodies, or a corresponding human IgG1 control antibody was added to the wells in a concentration range of 1-10 pg/ml. After incubating cells with antibodies for 30 minutes, IL-33 was added to a final concentration of 0.5 ng/ml, and the plate was incubated overnight. To examine whether any of the antibodies were able to induce ST2 activation in the absence of IL-33, samples were incubated with 10 μg/mL antibody without addition of the ligand. The following day, substrate was added to the supernatants, and samples were analyzed for absorbance at 620 nm.

### Results

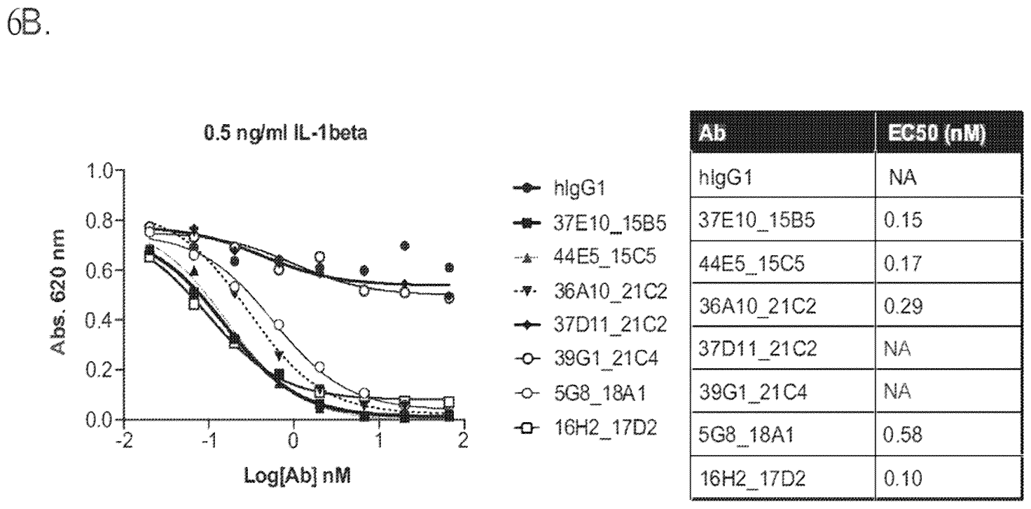

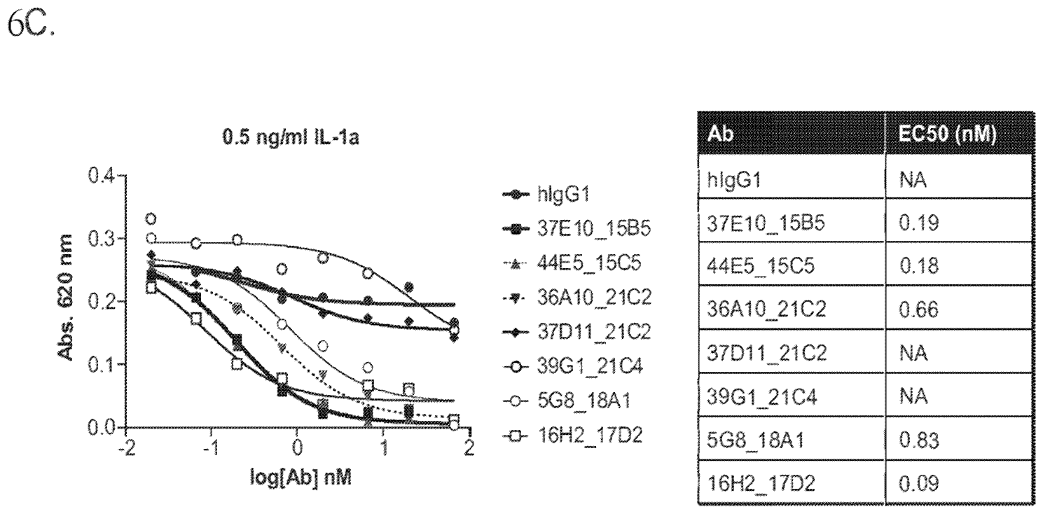

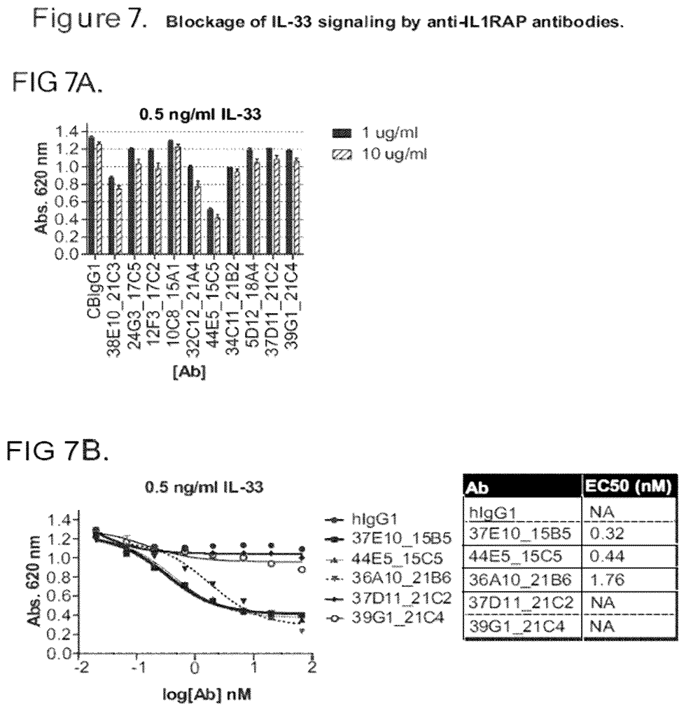

Since IL1RAP plays an important role in IL-33 signaling, the ability of the developed antibodies to inhibit IL-33 signaling was investigated. Whereas antibodies showed various degree of inhibitory effect in an IL-33 reporter assay, 37E10_15B5, 44E5_15C5, and 36A10_21B6 can partially inhibit IL-33 signaling in a dose dependent manner (see FIGS. 7A and 7B). Neither of the antibodies affected IL-33 signaling in the absence of IL-33, thereby excluding agonistic functions on IL-33 induced signaling (data not shown). On the other hand, 37D11_21C2 and 39G1_21C4 do not inhibit IL-33 signaling.

### Example 7. Efficacy of Anti-IL1RAP Ab in Mouse Model of Atopic Dermatitis

Experiments were performed to characterize the efficacy of anti-IL1RAP antibody in a mouse model of atopic dermatitis. The following methods were used.

### Methods

### Oxazolone/HDM Mouse Model of Atopic Dermatitis

Eight-week-old Balb/c mice were used. The mice either received no model induction (n=3; Group 1), injection of olive oil:acetone vehicle (1:4; n=3; Group 2), or 0.8% oxazolone in olive oil:acetone vehicle+house dust mites (HDM) (n=10 each; Groups 3 and 4) to induce atopic dermatitis. For induction of atopic dermatitis, oxazolone+HDM was administered on Day 0 (DO), D5, D8, D12, D15, and D17.

Mice in the oxazolone+HDM groups received either an anti-IL1RAP antibody of the present invention (Group 4; n=10) or an isotype control antibody (Group 3; n=10) via intraperitoneal injection at a dose of 20 mg/kg on D3, D6, D10, D13, and D17. Mice in the no-model and vehicle-only groups (Groups 1 and 2) received intraperitoneal injections of saline. The anti-IL1RAP antibody 19D2 used in this example is a surrogate antibody used for murine studies corresponding to a mouse IgG1 (analogous to human IgG4) directed to mouse IL-1RAP.

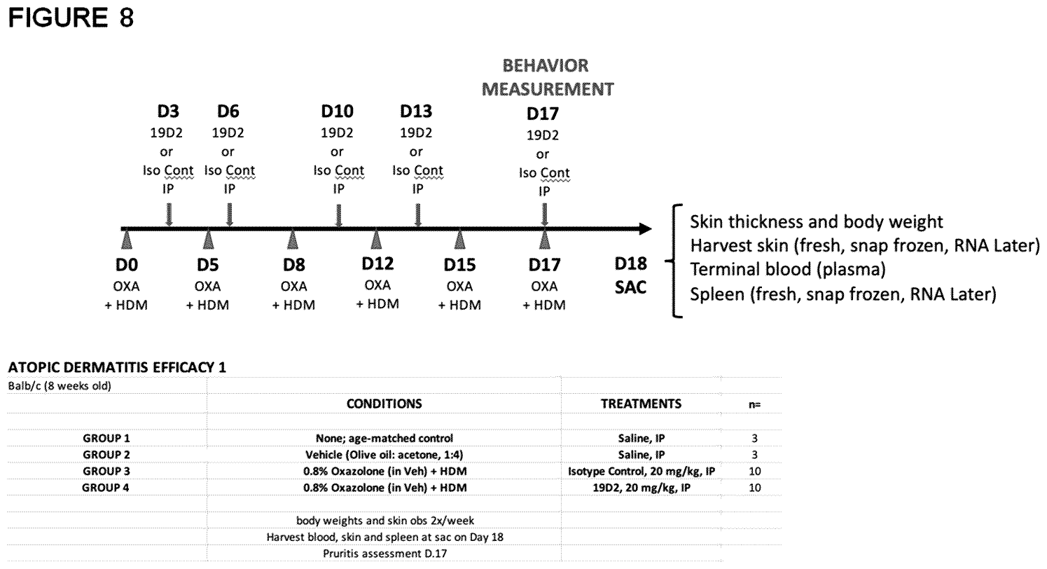

The body weight and skin condition of the mice were observed twice a week. Pruritis was assessed at D17 according to scratching at the disease induction site, quantified as the number of scratching events in 30 minutes. The animals were sacrificed at D18, and blood, skin, and spleen were harvested. To analyze levels of soluble inflammatory/atopic mediators, the skin samples were homogenized in the presence of protease inhibitors and then subjected to ELISA for the mediators of interest. To analyze levels of inflammatory cells, the skin samples were minced, digested with collagenase D, and subjected to cell sorting to obtain live CD45+ cells, which were stained with fluorophore-conjugated antibodies. The experimental design is shown in FIG. 8.

### Results

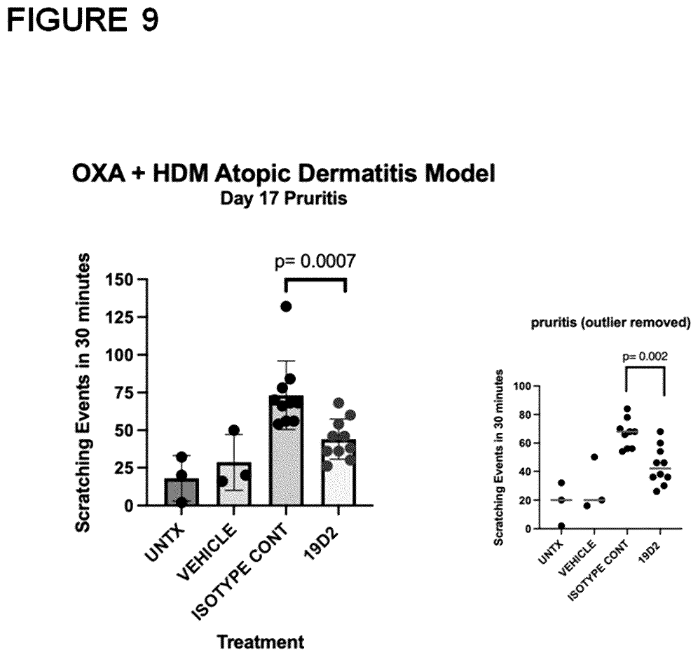

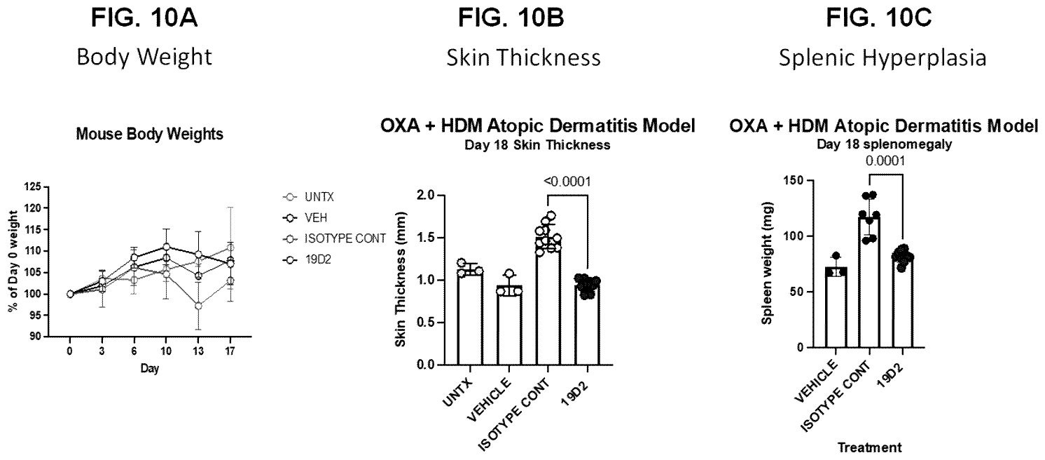

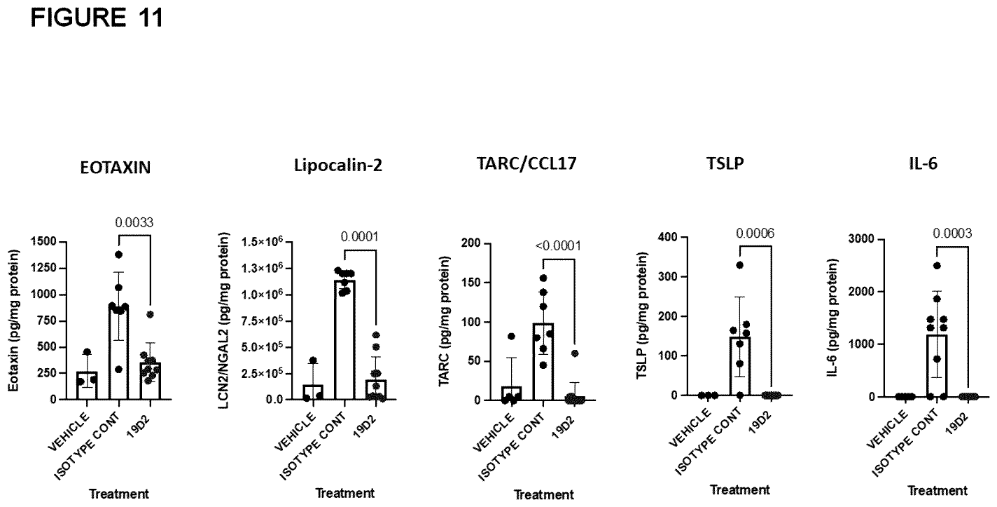

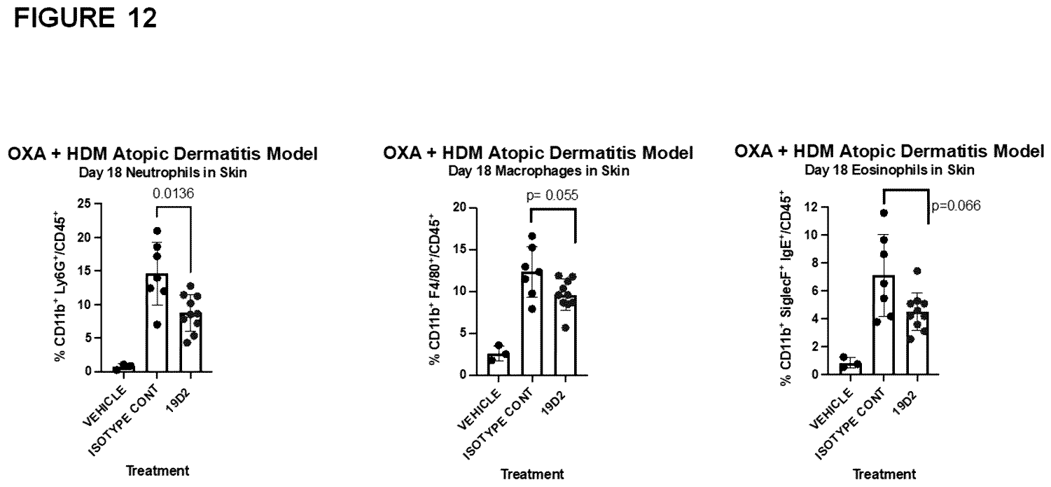

By D17, treatment with the anti-IL1RAP antibody significantly decreased pruritis, a major symptom of human atopic dermatitis, compared to treatment with the isotype control antibody, as shown in FIG. 9. The oxazolone+HDM model results in increased skin thickness (another important symptom of human atopic dermatitis), increased spleen size (splenomegaly), and inhibition of weight gain in mice. Here, by D18, treatment with the anti-IL1RAP antibody prevented or reduced skin thickening and spleen enlargement, such that the skin and spleen in treated mice were thinner and smaller, respectively, than those in mice receiving the isotype control. Additionally, treatment with the anti-IL1RAP antibody significantly reduced the inhibition of weight loss, such that treated mice put on more weight than mice receiving the isotype control. These findings are shown in FIGS. 10A, 10B, and 10C. Thus, treatment with the anti-IL1RAP antibody significantly reduced symptoms and pathology associated with human atopic dermatitis. Furthermore, treatment with the anti-IL1RAP antibody significantly reduced the levels of the inflammatory/atopic mediators eotaxin, lipocalin-2, TARC/CCL17, TSLP, and IL-6 in the skin at D18 compared to treatment with the isotype control, as determined by ELISA and shown in FIG. 11. Still furthermore, treatment with the anti-IL1RAP antibody significantly reduced the levels of neutrophils, macrophages, and eosinophils, i.e., immune cells mediating the inflammatory/atopic response, compared to the isotype control in the skin at D18, as shown in FIG. 12. These findings provide molecular and cellular evidence to support the efficacy of anti-IL1RAP antibodies of the present invention in atopic dermatitis.

### BFB759 In Vitro Studies

Multiple in vitro studies were conducted to determine if BFB759 combines favorably with various biologic and small molecule therapeutics that are approved and efficacious in inflammatory diseases such as atopic dermatitis and asthma. These studies confirm that therapeutics directed against IL-4Ra, IL-13, TNF alpha, and Janus kinases provide additive or synergistic effects when combined with BFB759. In addition, dexamethasone, a widely used corticosteroid also provides additive or synergistic effects when combined with BFB759. These studies demonstrate the utility of combination therapy with multiple anti-inflammatory agents when targeting IL-1R3 in inflammatory, autoimmune and autoinflammatory diseases.

### Example 8. BFB759 Inhibition of TARC Production in HDM Stimulated Human PBMCs

Experiments were performed to characterize inhibition of TARC production in HDM stimulated human PBMCs through the use of BFB759. The following methods were used.

### Methods

Materials included Human peripheral blood mononuclear cells (PBMCs) that were isolated from healthy donor blood and either frozen in liquid nitrogen and thawed prior to analysis or used fresh. Materials also included HDM (house dust mite extract) that was from Greer lab and purchased through Fisher Scientific (NC9756554, Lot #390844). Additionally, Human IL-4 (204-IL-010) was purchased from R&D Systems; Human IL-13 (200-13) was purchased from Peprotech; JAK inhibitors (JAKi) Abrocitinib (HY-107429) and Upadacitinib (HY-19569) were purchased from MedChemExpress; Dexamethasone (D4902) was purchased from Sigma-Aldrich; Dupilumab (HY-P9926) was purchased from MedChemExpress; Tralokinumab was produced by Bluefin Biomedicine according to the published protein sequence; and TARC ELISA kits (DuoSet Cat #DY364, Quantikine Cat #SDN00) were purchased from R&D Systems.

Blood was drawn in heparin tubes from healthy volunteers. PBMCs were isolated by centrifugation of the blood over Ficoll-Paque Plus density gradients using LeucoSep tubes.

0.2 million PBMCs in 100 ul medium (RPMI1640, 10% heat-inactivated FBS) were added to each well of 96 well cell culture plate. BFB759 at multiple concentrations in 50 ul medium was added to the wells. The cells were incubated in the incubator for 45 min. Then, 50 ul HDM solution (final concentration 10 ug/ml) was added to the wells. After 7 days incubation, the supernatant was transferred to a new 96 well plate, and used in TARC ELISA right away, or frozen in −80 C freezer for ELISA later. For the combination studies of BFB759 with other drugs, the single agent or combination of drugs were added to the PBMCs, incubated for 45 min, then HDM solution, or HDM and IL-4 or IL-13 solution were added to the wells. The assay was incubated for 7 days, and the supernatant was harvested for TARC ELISA.

### Results

TARC (thymus and activation-regulated chemokine) has an important role in allergic diseases such as atopic dermatitis and asthma. High serum concentrations of TARC are observed in patients with atopic dermatitis, and its concentration is closely related to disease activity. The measurement of serum TARC concentrations has already been clinically applied as a useful marker reflecting the disease activity of atopic dermatitis. In this study, PBMCs from different donors were stimulated with HDM, and treated with BFB759 at a wide range from 0.1 ug to 150 ug/ml. BFB759 can inhibit TARC level from 30%-70% depend on the donors. On the other hand, Dupilumab does not inhibit TARC level significantly in the same assay.

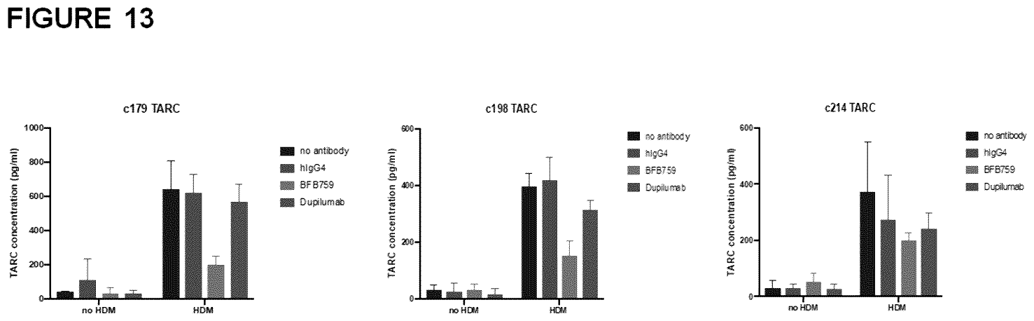

FIG. 13 shows that FB759 inhibits HDM induced TARC level in PBMCs, and that Dupilumab does not or minimally inhibits TARC in the same assay. In the data shown in FIG. 13, hIgG4 (human IgG4 isotype Ab), BFB759, and Dupilumab were used at 30 ug/ml, and HDM was used at 10 ug/ml. Data shown are mean±s.d.

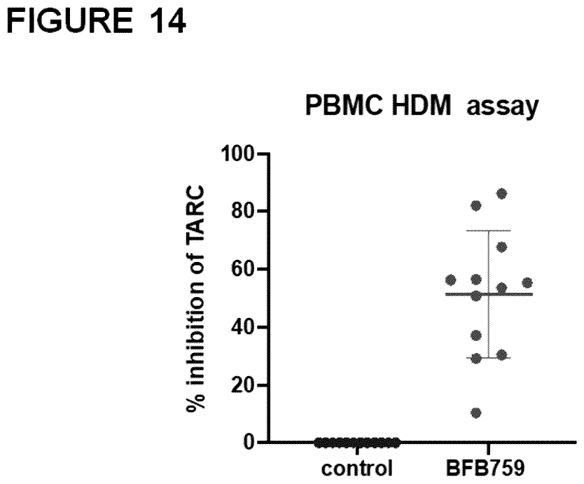

FIG. 14 shows a summary of inhibition of HDM induced TARC level in PBMCs from different donors by BFB759. In the data shown in FIG. 14, HDM was used at 10 ug/ml, and BFB759 from 1-30 ug/ml, PBMCs were from 12 different donors. Data shown are mean±s.d.

### Example 9. BFB759 and Dupilumab Inhibition of TARC in PBMCs Treated by HDM and hIL-4

Experiments were performed to characterize inhibition of TARC production in PBMCs treated by HDM and hIL-4 through the use of BFB759 and Dupilumab. The following methods were used.

### Methods and Results

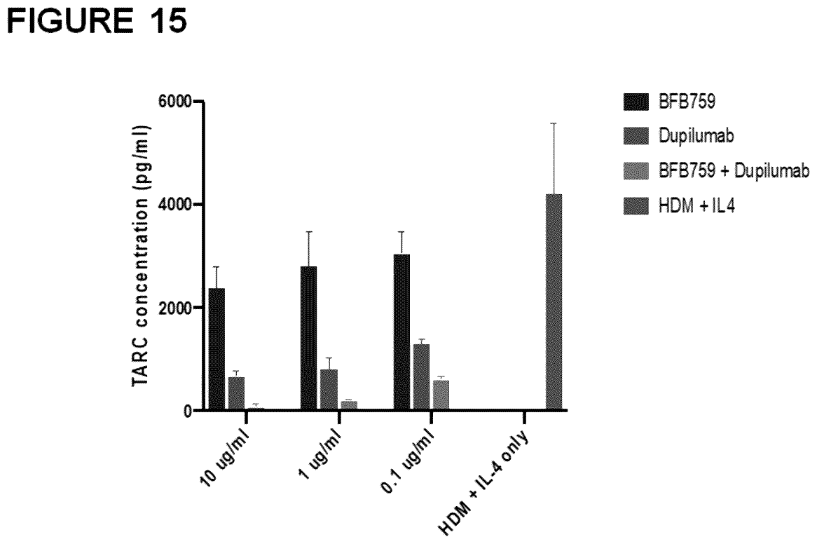

Dupilumab is approved for treatment of AD (Atopic Dermatitis). It inhibits IL-4/13 signaling by blocking their binding to IL-4Ra. When PBMCs were stimulated with HDM and IL-4, TARC expression was significantly increased. Treatment of cells with either BFB759 or Dupilumab alone partially inhibits TARC expression in the HDM and IL-4 model. These results also demonstrate that the combined effect of BFB759 and Dupilumab is greater than the effect of each drug individually, regardless of concentration tested. Almost complete inhibition of TARC expression was achieved when combining these antibodies at 10 ug/ml per antibody. These results were unexpected as reductions in TARC by inhibition of TH1 cytokines with a therapeutic antibody like BFB759 has not been reported. Furthermore, it was also surprising to see that minimal inhibition of TARC expression is seen when dosing with 1 or 0.1 ug/ml of BFB759 but when combined with dupilumab this combination is even more effective than dosing at 10 ug/ml dupilumab alone (i.e., an up to 100-fold reduction in dupilumab). This suggests that combination therapy with BFB759 would allow for a significant reduction in dupilumab dose in the clinic while providing for improved efficacy and a broader therapeutic window. (FIG. 15)

FIG. 15 shows BFB759 and Dupilumab can inhibit TARC level in PBMC treated by HDM and IL-4. In the data shown in FIG. 15, HDM was used at 10 ug/ml, IL-4 was used at 750 ug/ml, antibodies were at three different concentrations as shown in the graph. Data shown are mean±s.d.

### Example 10

### BFB759 and Anti-IL-13 Inhibition of TARC Level in PBMCs Treated by HDM and IL-13

Experiments were performed to characterize inhibition of TARC production in PBMCs treated by HDM and IL-13 through the use of BFB759 and anti-IL-13. The following methods were used.

### Methods and Results

The IL-13 neutralizing antibody tralokinumab was approved for atopic dermatitis (AD) in 2021. When PBMCs were stimulated with HDM and IL-13, TARC level was significantly increased.

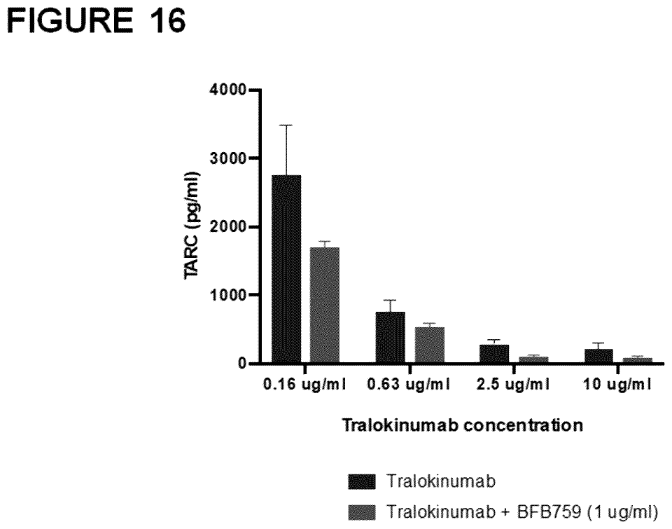

The treatment of HDM and IL-13 stimulated cells with tralokinumab showed dose dependent inhibition of TARC. The treatment with tralokinumab in combination of 1 ug/ml BFB759 further decreased the level of TARC, demonstrating that the combined effect of BFB759 and tralokinumab is greater than the effect of each drug individually, regardless of concentration tested. These results were unexpected as reductions in TARC by inhibition of TH1 cytokines with a therapeutic antibody like BFB759 has not been reported. (FIG. 16). Furthermore, these results are surprising as reduced TARC expression by combination therapy with an anti-IL-13 antibody and an anti-IL-1R3 antibody has not been described.

FIG. 16 shows BFB759 and Tralokinumab can inhibit TARC level in PBMC treated by HDM and IL-13. In the data shown in FIG. 16, HDM was used at 10 ug/ml, IL-13 was used at 0.5 nM, BFB759 was used at 1 ug/ml, tralokinumab was titrated from 10 ug/ml to 0.16 ug/ml (4 fold dilution, 4 series). Data shown are mean±s.d.

### Example 11

BFB759 and JAK Inhibitors or Dexamethasone Inhibition of TARC in PBMCs Treated with HDM

Experiments were performed to characterize inhibition of TARC production in PBMCs treated by HDM through the use of BFB759 and JAK inhibitors or Dexamethasone. The following methods were used.

### Methods and Results

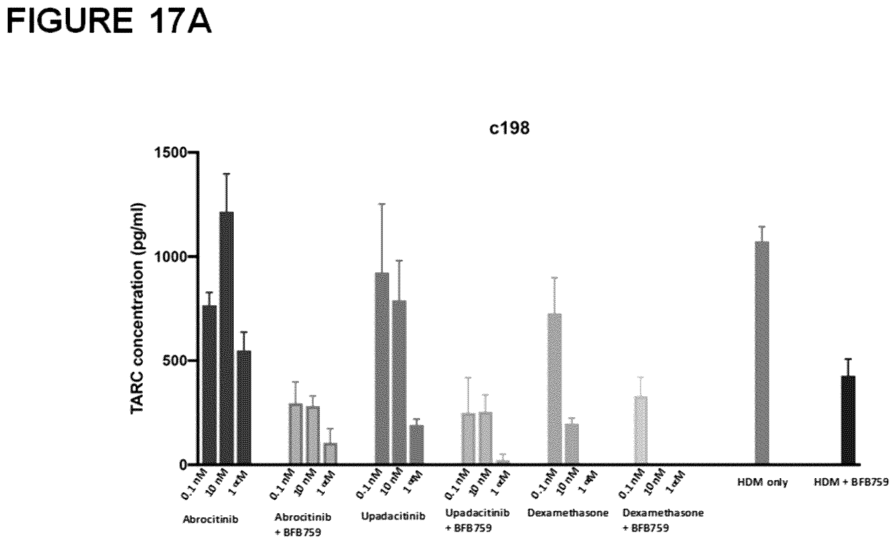

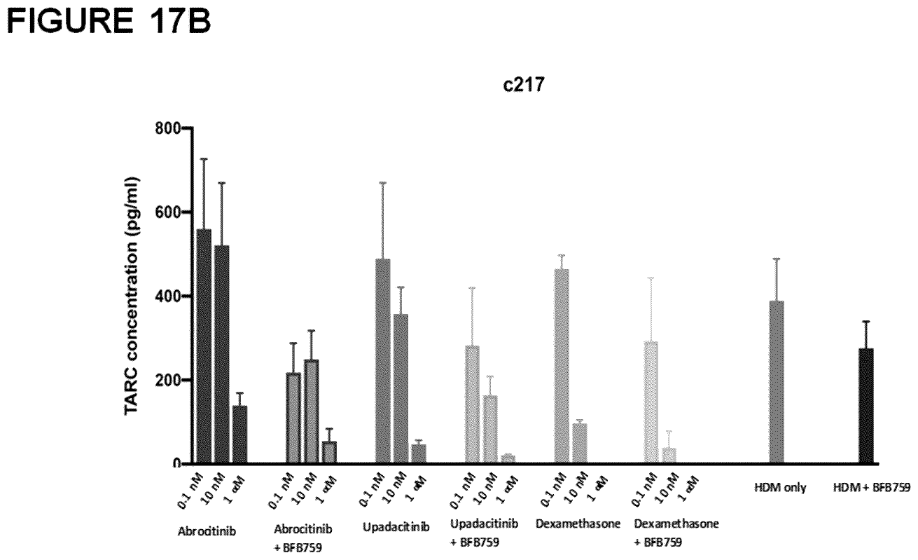

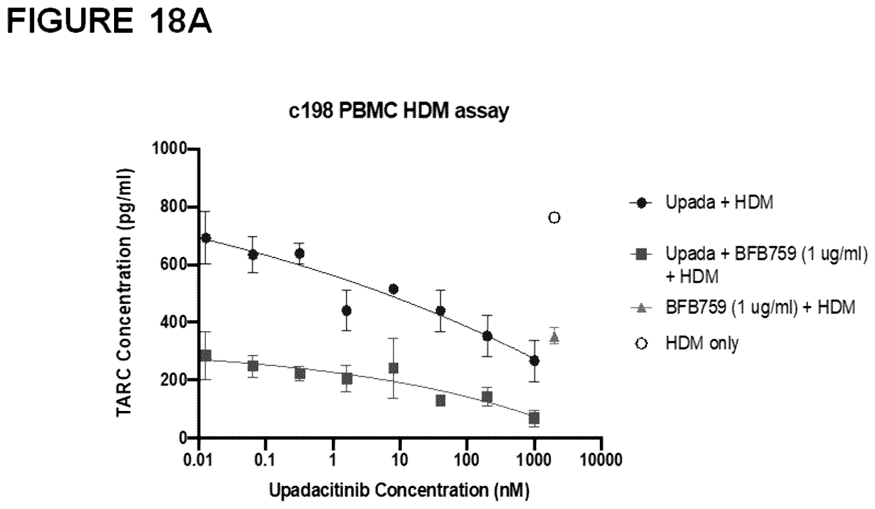

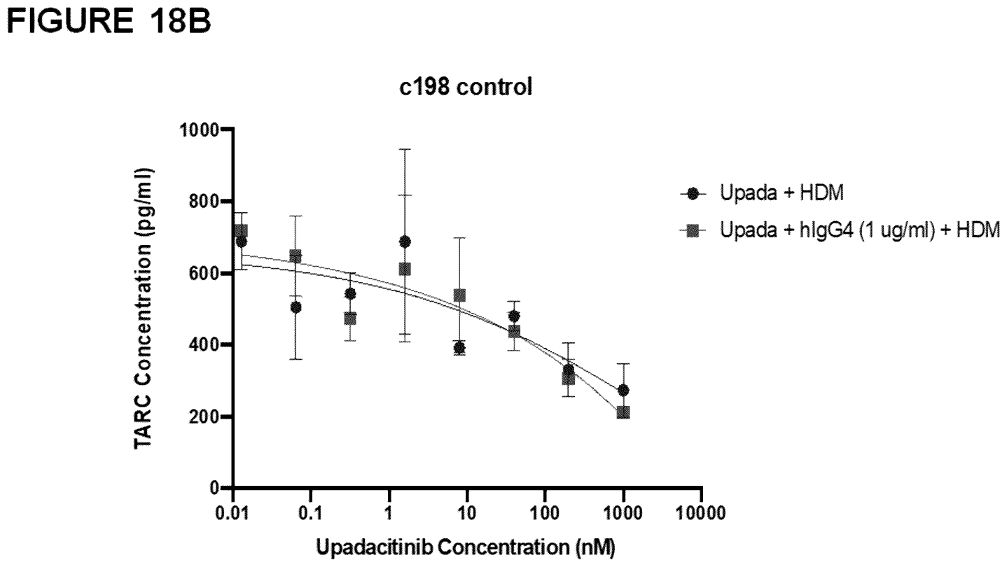

JAK inhibitors (Abrocitinib and Upadacitinib) and dexamethasone are used to treat AD patients. The treatment of HDM stimulated cells with JAKi or dexamethasone showed dose dependent inhibition of TARC. The treatment with JAKi or dexamethasone in combination of 1 ug/ml BFB759 further decreased the level of TARC, demonstrating that the combined effect of BFB759 and these agents is greater than the effect of each drug individually, regardless of concentration tested. These results were unexpected as reductions in TARC by inhibition of TH1 cytokines with a therapeutic antibody like BFB759 has not been reported. (FIGS. 17A and 17B). Furthermore, these results are surprising as reduced TARC expression by combination therapy with JAKi and dexamethasone and an anti-IL-1R3 antibody has not been described (FIGS. 17A, 17B, 18A, and 18B).

These results suggest that combination therapy with BFB759 would allow for a significant reduction in JAKi or dexamethasone dose in the clinic while providing for improved efficacy and a broader therapeutic window.

FIGS. 17A and 17B show that a combination of BFB759 and JAK inhibitors or Dexamethasone can inhibit TARC in PBMC treated by HDM. In the data shown in FIGS. 17A and 17B, HDM was used at 10 ug/ml, BFB759 was used at 1 ug/ml, JAKi and Dexamethasone were tested at three different concentrations (0.1 nM, 10 nM, and 1 uM) as shown in the graph. Data shown are mean±s.d.

FIGS. 18A and 18B show that a combination of BFB759 and Upadacitinib inhibit TARC in PBMC treated by HDM. In the data shown in FIGS. 18A and 18B, M was used at 10 ug/ml, BFB759 and hIgG4 (human IgG4 isotype Ab) were used at 1 ug/ml, JAKi Upadacitinib was tested at 5 fold serial titration from 1 uM to 0.01 nM. Data shown are mean±s.d.

### Example 12

Combination of BFB759 with Adalimumab Inhibition of IL-6 Production in Human Whole Blood Cultures Stimulated with Heat-Killed Candida albicans

Experiments were performed to characterize inhibition of IL-6 production in human whole blood cultures stimulated with heat-killed Candida albicans through the use of a combination of BFB759 with Adalimumab. The following methods were used.

### Methods

Materials included Human peripheral blood drawn from 5 healthy donors. Materials also included Anti-human TNF alpha (Adalimumab Biosimilar), BioXCell cat #SIM0001, lot #8024221F1; Heat-killed Candida albicans (HKCA), InvivoGen cat #tlrl-hkca, lot #6292-44-02; RPMI 1640 with L-glutamine and HEPES, Corning cat #10-041-CM; and Human IL-6 DuoSet ELISA, R and D Systems cat #DY206-05.

Blood was drawn into heparin tubes from healthy volunteers. Whole blood cultures comprised of whole blood diluted with plain RPMI 1640 medium were set up in sterile 5.0 ml polypropylene snap cap culture tubes. One of the following treatments were added for 1 hour prior to either being left unstimulated or receiving stimulation with 1.5×10\hat{ }6 cells heat-killed Candida albicans: medium alone (cells alone), 1 μg/ml IgG1 isotype control antibody alone, 1 μg/ml BFB759 alone, 1 μg/ml adalimumab alone, or one of two combination treatments of 1 μg/ml IgG1 isotype control antibody+0.5 μg/ml adalimumab or 1 μg/ml BFB759+0.5 μg/ml adalimumab. The final volume of each culture was 1.0 ml. Cultures were incubated for 24 hours at 37° C./5% CO₂. Supernatants harvested from each culture in duplicate were frozen at −80° C. until assayed in duplicate for IL-6 levels by ELISA.

### Results

BFB759 and adalimumab inhibit IL-6 secretion on human whole blood cultures challenged with HKCA, and this effect is enhanced with the combination of both treatments. High levels of IL-6 were induced in blood cultures following challenge with HKCA. Both adalimumab and BFB759 were able to reduce IL-6 secretion, with an enhanced effect by combining the treatments.

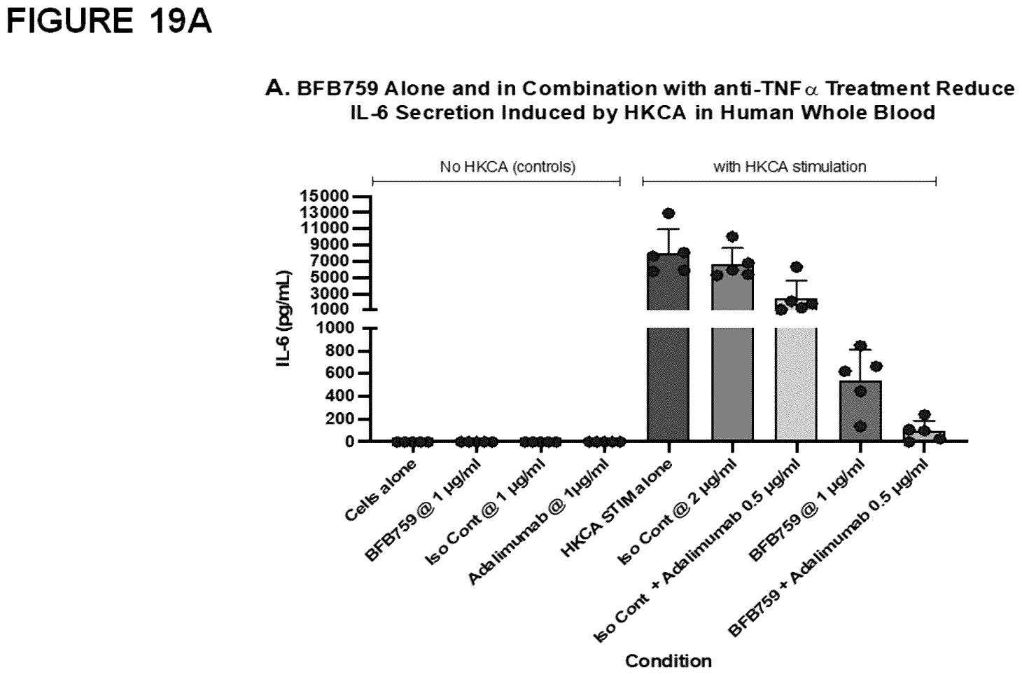

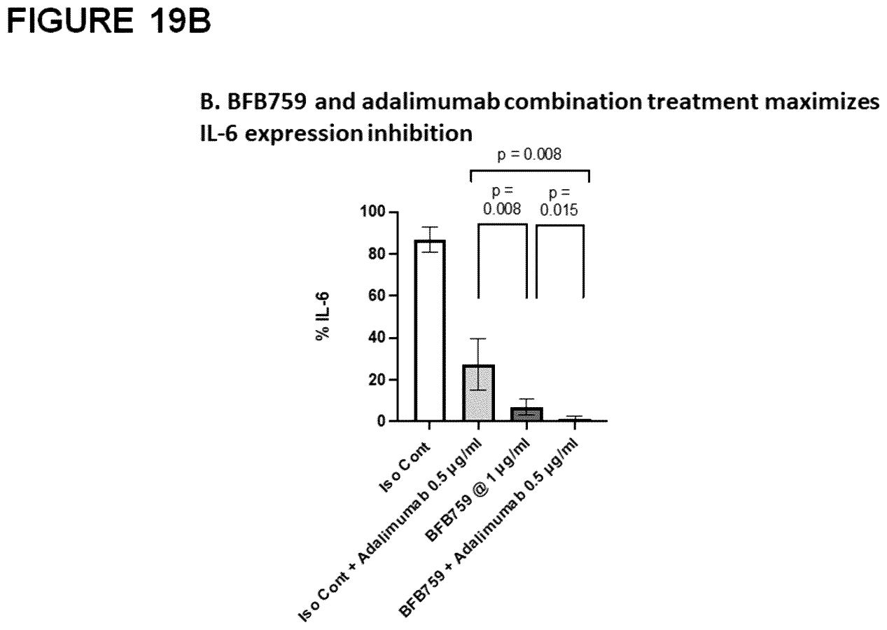

FIGS. 19A and 19B show that a combination of BFB759 and Adalimumab inhibit IL-6 secretion in human whole blood cultures stimulated with HKCA. In the data shown in FIG. 19A, IL-6 Levels from whole blood cultures is measured by ELISA. In the data shown in FIG. 19B, the percentage of IL-6 measured in cultures treated as indicated compared to the levels detected with HKCA and no intervening treatment.

### Example 13

Combination of BFB759 with Secukinumab Inhibition of IL-6 and IL-8 Production in Normal Human Dermal Fibroblasts (NHDF) Stimulated with Cytokine Combo

Experiments were performed to characterize inhibition of IL-6 and IL-8 production in normal human dermal fibroblasts stimulated with cytokine combo through the use of a combination of BFB759 with Secukinumab. The following methods were used.

### Materials:

| Test Material/Reagent | Catalog # | Manufacturer |
|---|---|---|
| BFB759 | IL1X_n37E10_547B3 | Bluefin |
| | | Biomedicine |
| FGM-2 Fibroblast Growth | CC-3132 | Lonza |
| Medium-2 BulletKit | | |
| Human dermal fibroblast, | CC-2511 | Lonza |
| adult (NHDF) | | |
| Human IL-1α | 200-LA-002 | R&D Systems |
| Human IL-1β | 201-LB-005 | R&D Systems |
| Human IL-17A | 7955-IL | R&D Systems |
| Human IL-36α | 6995-IL-010 | R&D Systems |
| Human IL-36β | 6834-ILB-025 | R&D Systems |
| Human IL-36γ | 6835-IL-010 | R&D Systems |
| Human IL-6 DuoSet ELISA | DY206 | R&D Systems |
| Human IL-8/CXCL8 | DY208 | R&D Systems |
| DuoSet ELISA | | |
| Penicillin/Streptomycin | 30-002-CI | Corning |
| ReagentPack Subculture | CC-5034 | Lonza |
| Reagents, 100 mL | | |
| Secukinumab | HY-P9927 | MedChem |
| | | Express |

### Methods

Human NHDF cells were plated on 96-well, flat-bottom plates at 4,000 cells/well. BFB759, Secukinumab (anti-IL-17A antibody), and Isotype control antibody at increasing concentrations (0-20 ug/ml, 5-fold dilution) were incubated with cells in duplicate for 45 minutes, prior to the addition of cytokine combo [IL-1α (10 μg/ml), IL-1β (5 pg/ml)□, IL-36α (25 ng/ml), IL-36β, (3 ng/ml), IL-36γ (7 ng/ml), and IL-17A (1 ng/ml)] at a concentration previously determined to be roughly equivalent to EC₅₀ for the cells (not shown). For combination therapy, 0.5 ug/ml BFB759 was mixed with Secukinumab or Isotype control antibody at increasing concentrations (0-20 ug/ml, 5-fold dilution) and incubated with cells in duplicate for 45 minutes, prior to the addition of cytokine combo. The plates were then incubated overnight at 37° C., 5% CO₂. 24 hours after treatment, supernatants were transferred and assayed for IL-6 and IL-8, as per manufacture's protocol (R&D Systems; Human IL-6 DuoSet ELISA, Human IL-8/CXCL8 DuoSet ELISA). The raw assay data were analyzed using GraphPad Prism 9 software to perform a non-linear regression.

### Results

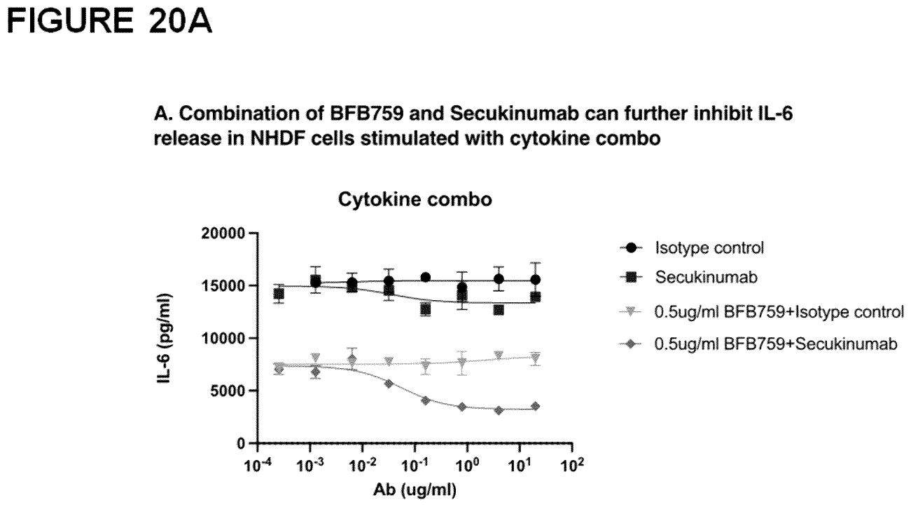

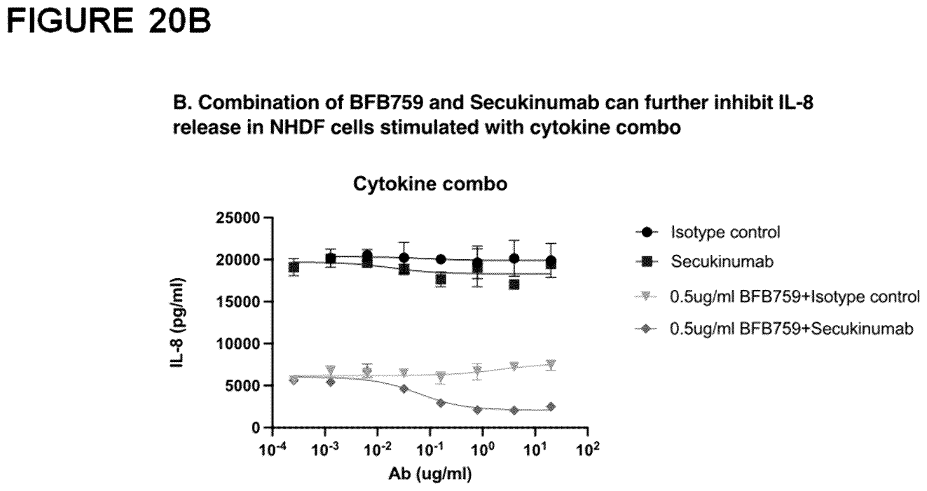

NHDF cells are known to respond to IL-1, IL-36, and IL-17A stimulation. To understand whether combination of BFB759 and secukinumab can further inhibit IL-6 and IL-8 production, NHDF cells were first incubated with BFB759, secukinumab, and a combination of BFB759 and secukinumab. Then, stimulated with cytokine combo composed of IL-1α/β, IL-36α/β/γ, and IL-17A. Treatment of NHDF with BFB759 resulted in a dose-dependent decrease in IL-6 and IL-8 cytokine production (FIG. 20). Secukinumab alone showed limited inhibition of IL-6 and IL-8 release in this assay. Interestingly, combination of BFB759 (0.5 ug/ml) with increasing concentration of secukinumab showed greater inhibition of IL-6 and IL-8 production than either antibody alone, demonstrate the utility of combination therapy in autoimmune and inflammatory diseases.

FIGS. 20A and 20B show that a combination of BFB759 with Secukinumab inhibit IL-6 and IL-8 release in NHDF cells stimulated with cytokine combo. Data shown are mean±s.d.

### Example 14

Combination of BFB759 with Adalimumab Inhibition of IL-6 and IL-8 Production in Normal Human Dermal Fibroblasts (NHDF) Stimulated with Cytokine Combo

Experiments were performed to characterize inhibition of IL-6 and IL-8 production in normal human dermal fibroblasts stimulated with cytokine combo through the use of a combination of BFB759 with Adalimumab. The following methods were used.

### Materials:

| Test Material/Reagent | Catalog # | Manufacturer |
|---|---|---|
| Adalimumab | SIM0001 | BioXcell |
| BFB759 | IL1X_n37E10_547B3 | Bluefin |
| | | Biomedicine |
| FGM-2 Fibroblast Growth | CC-3132 | Lonza |
| Medium-2 BulletKit | | |
| Human dermal fibroblast, | CC-2511 | Lonza |
| adult (NHDF) | | |
| Human IL-1α | 200-LA-002 | R&D Systems |
| Human IL-1β | 201-LB-005 | R&D Systems |
| Human TNF-α | 16769S | Cell Signaling |
| | | Technology, |
| | | Inc. |
| Human IL-36α | 6995-IL-010 | R&D Systems |
| Human IL-36β | 6834-ILB-025 | R&D Systems |
| Human IL-36γ | 6835-IL-010 | R&D Systems |
| Human IL-6 DuoSet ELISA | DY206 | R&D Systems |
| Human IL-8/CXCL8 | DY208 | R&D Systems |
| DuoSet ELISA | | |
| Penicillin/Streptomycin | 30-002-CI | Corning |
| ReagentPack Subculture | CC-5034 | Lonza |
| Reagents, 100 mL | | |

### Methods

Human NHDF cells were plated on 96-well, flat-bottom plates at 4,000 cells/well. BFB759, adalimumab (anti-TNF-α antibody), and isotype control antibody at increasing concentrations (0-20 ug/ml, 5-fold dilution) were incubated with cells in duplicate for 45 minutes, prior to the addition of cytokine combo [IL-1α (10 pg/ml), IL-1β (5 pg/ml), IL-36α (25 ng/ml), IL-36β, (3 ng/ml), IL-36γ (7 ng/ml), and TNF-α (0.05 ng/ml)] at a concentration previously determined to be roughly equivalent to EC₅₀ for the cells (not shown). For combination therapy, 0.5 ug/ml BFB759 was mixed with adalimumab or isotype control antibody at increasing concentrations (0-20 ug/ml, 5-fold dilution) and incubated with cells in duplicate for 45 minutes, prior to the addition of cytokine combo. The plates were then incubated overnight at 37° C., 5% CO₂. 24 hours after treatment, supernatants were transferred and assayed for IL-6 and IL-8, as per manufacture's protocol (R&D Systems; Human IL-6 DuoSet ELISA, Human IL-8/CXCL8 DuoSet ELISA). The raw assay data were analyzed using GraphPad Prism 9 software to perform a non-linear regression.

### Results

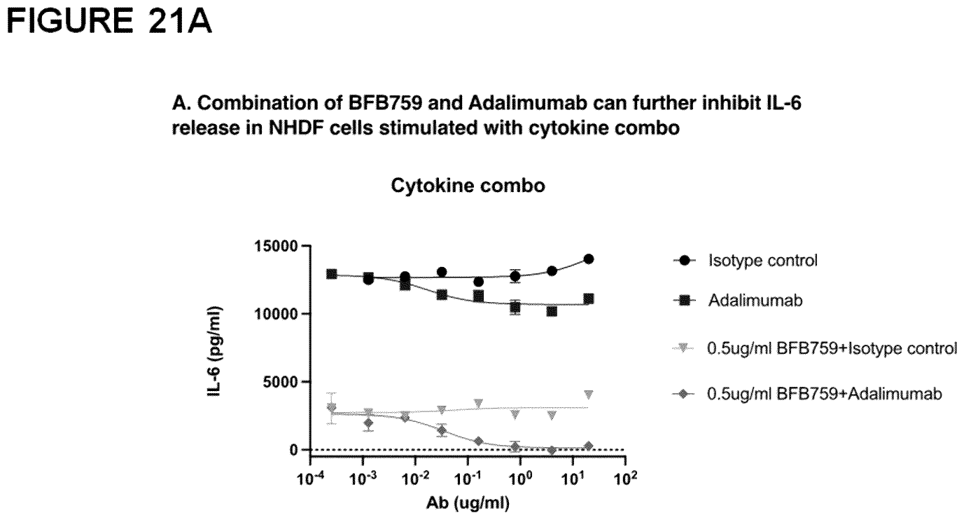

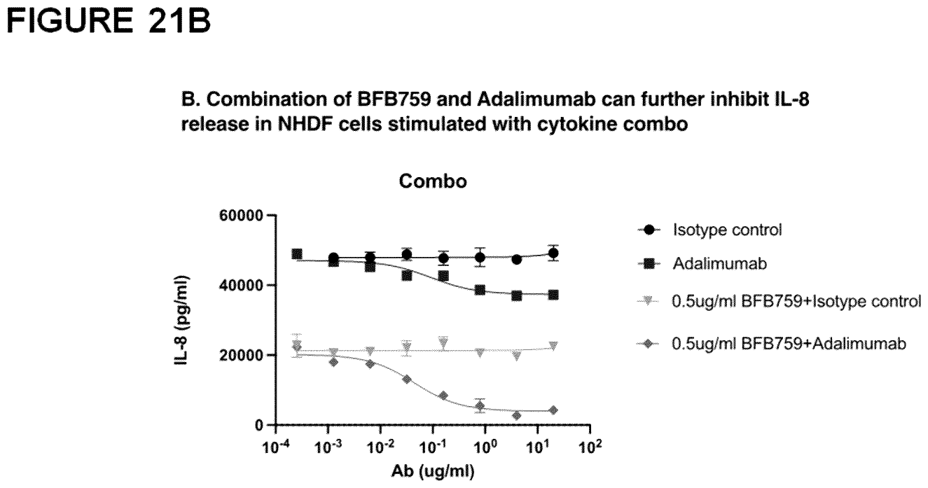

NHDF cells are known to respond to IL-1, IL-36, and TNF-α stimulation. To understand whether combination of BFB759 and adalimumab can further inhibit IL-6 and IL-8 production, NHDF cells were first incubated with BFB759, adalimumab, and a combination of BFB759 and adalimumab. Then, stimulated with cytokine combo composed of IL-1α/β, IL-36α/β/γ, and TNF-α. Treatment of NHDF with BFB759 resulted in a dose-dependent decrease in IL-6 and IL-8 cytokine production (FIG. 21). Adalimumab alone showed limited inhibition of IL-6 and IL-8 release in this assay. Interestingly, combination of BFB759 (0.5 ug/ml) with increasing concentration of adalimumab showed greater inhibition of IL-6 and IL-8 production than either antibody alone, demonstrate the utility of combination therapy in autoimmune and inflammatory diseases.

FIGS. 21A and 21B show that a combination of BFB759 with adalimumab inhibit IL-6 and IL-8 release in NHDF cells stimulated with cytokine combo. Data shown are mean s.d.

### INCORPORATION BY REFERENCE

The contents of all references, patents, pending patent applications and published patents, Sequence Listing, and Accession Numbers, cited throughout this application are hereby expressly incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

Sequence Summary

| SEQ ID | | |
|---|---|---|
| NO: | Clone Name | Protein Domain |
| 1 | 5G8_18A1 | VH amino acid sequence |
| 2 | 5G8_18A1 | CDR-H1 amino acid sequence |
| 3 | 5G8_18A1 | CDR-H2 amino acid sequence |
| 4 | 5G8_18A1 | CDR-H3 amino acid sequence |
| 5 | 5G8_18A1 | VL amino acid sequence |
| 6 | 5G8_18A1 | CDR-L1 amino acid sequence |
| 7 | 5G8_18A1 | CDR-L2 amino acid sequence |
| 8 | 5G8_18A1 | CDR-L3 amino acid sequence |
| 9 | 10C8_15A1, | VH amino acid sequence |
| | 10C8_C43A | |
| 10 | 10C8_15A1, | CDR-H1 amino acid sequence |
| | 10C8_C43A | |
| 11 | 10C8_15A1, | CDR-H2 amino acid sequence |
| | 10C8_C43A | |
| 12 | 10C8_15A1, | CDR-H3 amino acid sequence |
| | 10C8_C43A | |
| 13 | 10C8_15A1 | VL amino acid sequence |
| 14 | 10C8_15A1, | CDR-L1 amino acid sequence |
| | 10C8_C43A | |
| 15 | 10C8_15A1, | CDR-L2 amino acid sequence |
| | 10C8_C43A | |
| 16 | 10C8_15A1, | CDR-L3 amino acid sequence |
| | 10C8_C43A | |
| 17 | 12F3_17C2 | VH amino acid sequence |
| 18 | 12F3_17C2 | CDR-H1 amino acid sequence |
| 19 | 12F3_17C2 | CDR-H2 amino acid sequence |
| 20 | 12F3_17C2, | CDR-H3 amino acid sequence |
| | 17E9_15B1 | |
| 21 | 12F3_17C2 | VL amino acid sequence |
| 22 | 12F3_17C2 | CDR-L1 amino acid sequence |
| 23 | 12F3_17C2, | CDR-L2 amino acid sequence |
| | 16H2_17D2 | |
| 24 | 12F3_17C2, | CDR-L3 amino acid sequence |
| | 17E9_15B1 | |
| 25 | 16H2_17D2 | VH amino acid sequence |
| 26 | 16H2_17D2, | CDR-H1 amino acid sequence |
| | 31F9_21A1, | |
| | 32F9_21A5, | |
| | 33E9_21A6 | |
| 27 | 16H2_17D2 | CDR-H2 amino acid sequence |
| 28 | 16H2_17D2 | CDR-H3 amino acid sequence |
| 29 | 16H2_17D2 | VL amino acid sequence |
| 30 | 16H2_17D2 | CDR-L1 amino acid sequence |
| 31 | 16H2_17D2 | CDR-L3 amino acid sequence |
| 32 | 32C12_21A4, | VH amino acid |
| | 32C12-N26S | sequence |
| 33 | 32C12_21A4, | CDR-H1 amino |
| | 32C12-N26S | acid sequence |
| 34 | 32C12_21A4, | CDR-H2 amino |
| | 32C12-N26S, | acid sequence |
| | 41G4_15B6 | |
| 35 | 32C12_21A4, | CDR-H3 amino |
| | 32C12-N26S, | acid sequence |
| | 41G4_15B6 | |
| 36 | 32C12_21A4, | VL amino acid |
| | 41G4_15B6 | sequence |
| 37 | 32C12_21A4, | CDR-L1 amino |
| | 32A2_21A3, | acid sequence |
| | 41G4_15B6 | |
| 38 | 32C12_21A4, | CDR-L2 amino |
| | 32A2_21A3, | acid sequence |
| | 32C12-N26S, | |
| | 41G4_15B6 | |
| 39 | 32C12_21A4, | CDR-L3 amino |
| | 32A2_21A3, | acid sequence |
| | 32C12-N26S, | |
| | 41G4_15B6 | |
| 40 | 34C11_21B2 | VH amino acid sequence |
| 41 | 34C11_21B2 | CDR-H1 amino acid sequence |
| 42 | 34C11_21B2, | CDR-H2 amino |
| | 33H2_21B1 | acid sequence |
| 43 | 34C11_21B2, | CDR-H3 amino |
| | 36A12_21C1, | acid sequence |
| | 38G11_28A2, | |
| | 40C3_22B6 | |
| 44 | 34C11_21B2 | VL amino acid sequence |
| 45 | 34C11_21B2, | CDR-L1 amino |
| | 33H2_21B1 | acid sequence |
| 46 | 34C11_21B2, | CDR-L2 amino |
| | 33H2_21B1 | acid sequence |
| 47 | 34C11_21B2, | CDR-L3 amino |
| | 33H2_21B1 | acid sequence |
| 48 | 36A10_21B6 | VH amino acid |
| | | sequence |
| 49 | 36A10_21B6, | CDR-H1 amino |
| | 34D1_21B3 | acid sequence |
| 50 | 36A10_21B6, | CDR-H2 amino |
| | 34D1_21B3 | acid sequence |
| 51 | 36A10_21B6, | CDR-H3 amino |
| | 34D1_21B3 | acid sequence |
| 52 | 36A10_21B6 | VL amino acid sequence |
| 53 | 36A10_21B6 | CDR-L1 amino acid sequence |
| 54 | 36A10_21B6 | CDR-L2 amino acid sequence |
| 55 | 36A10_21B6, | CDR-L3 amino acid sequence |
| | 34D1_21B3 | |
| 56 | 39G1_21C4 | VH amino acid sequence |
| 57 | 39G1_21C4 | CDR-H1 amino acid sequence |
| 58 | 39G1_21C4 | CDR-H2 amino acid sequence |
| 59 | 39G1_21C4 | CDR-H3 amino acid sequence |
| 60 | 39G1_21C4 | VL amino acid sequence |
| 61 | 39G1_21C4 | CDR-L1 amino acid sequence |
| 62 | 39G1_21C4, | CDR-L2 amino |
| | 37E10_15B5, | acid sequence |
| | 44E5_15C5, | |
| | 31F9_21A1, | |
| | 32D4_21D6, | |
| | 32F9_21A5, | |
| | 33E9_21A6, | |
| | 35D11_22A1, | |
| | 39A9_28A4, | |
| | 34H8_21B4 | |
| 63 | 39G1_21C4, | CDR-L3 amino |
| | 34H8_21B4 | acid sequence |
| 64 | 37E10_15B5 | VH amino acid sequence |
| 65 | 37E10_15B5, | CDR-H1 amino |
| | 44E5_15C5 | acid sequence |
| 66 | 37E10_15B5, | CDR-H2 amino |
| | 32D4_21D6, | acid sequence |
| | 32F9_21A5, | |
| | 33E9_21A6, | |
| | 35D11_22A1 | |
| 67 | 37E10_15B5, | CDR-H3 amino |
| | 44E5_15C5, | acid sequence |
| | 31F9_21A1, | |
| | 32D4_21D6, | |
| | 32F9_21A5, | |
| | 33E9_21A6, | |
| | 35D11_22A1 | |
| 68 | 37E10_15B5 | VL amino acid sequence |
| 69 | 37E10_15B5, | CDR-L1 amino |
| | 44E5_15C5, | acid sequence |
| | 31F9_21A1, | |
| | 32D4_21D6, | |
| | 32F9_21A5, | |
| | 33E9_21A6, | |
| | 35D11_22A1 | |
| 70 | 37E10_15B5, | CDR-L3 amino |
| | 44E5_15C5, | acid sequence |
| | 31F9_21A1, | |
| | 32D4_21D6, | |
| | 32F9_21A5, | |
| | 33E9_21A6, | |
| | 39A9_28A4 | |
| 71 | 44E5_15C5 | VH amino acid sequence |
| 72 | 44E5_15C5 | CDR-H2 amino acid sequence |
| 73 | 44E5_15C5 | VL amino acid sequence |
| 74 | 38E10_21C3, | VH amino acid sequence |
| 75 | 38E10_21C3 | CDR-H1 amino acid sequence |
| 76 | 38E10_21C3 | CDR-H2 amino acid sequence |
| 77 | 38E10_21C3 | CDR-H3 amino acid sequence |
| 78 | 38E10_21C3 | VL amino acid sequence |
| 79 | 38E10_21C3, | CDR-L1 amino |
| | 26C5_15B4, | acid sequence |
| | 37D11_21C2, | |
| | 42D10_28A5 | |
| 80 | 38E10_21C3, | CDR-L2 amino |
| | 16H2_17D1, | acid sequence |
| | 24G3_17C5, | |
| | 26C5_15B4, | |
| | 10C8-15A4, , | |
| | 42D10_28A5 | |
| 81 | 38E10_21C3, | CDR-L3 amino |
| | 42D10_28A5 | acid sequence |
| 82 | 10C8_C43A | VL amino acid |
| | | sequence |
| 83 | 16H2_17D1 | VH amino acid sequence |
| 84 | 16H2_17D1, | CDR-H1 amino |
| | 24G3_17C5, | acid sequence |
| | 26C5_15B4, | |
| | 10C8-15A4 | |
| 85 | 16H2_17D1 | CDR-H2 amino acid sequence |
| 86 | 16H2_17D1, | CDR-H3 amino acid sequence |
| | 10C8-15A4 | |
| 87 | 16H2_17D1 | VL amino acid sequence |
| 88 | 16H2_17D1, | CDR-L1 amino |
| | 10C8-15A4 | acid sequence |
| 89 | 16H2_17D1, | CDR-L3 amino |
| | 10C8-15A4 | acid sequence |
| 90 | 24G3_17C5 | VH amino acid sequence |
| 91 | 24G3_17C5 | CDR-H2 amino acid sequence |
| 92 | 24G3_17C5, | CDR-H3 amino |
| | 26C5_15B4 | acid sequence |
| 93 | 24G3_17C5 | VL amino acid sequence |
| 94 | 24G3_17C5 | CDR-L1 amino acid sequence |
| 95 | 24G3_17C5 | CDR-L3 amino acid sequence |
| 96 | 26C5_15B4 | VH amino acid sequence |
| 97 | 26C5_15B4 | CDR-H2 amino acid sequence |
| 98 | 26C5_15B4 | VL amino acid sequence |
| 99 | 26C5_15B4 | CDR-L3 amino acid sequence |
| 100 | 17E9_15B1 | VH amino acid sequence |
| 101 | 17E9_15B1, | CDR-H1 amino |
| | 32D4_21D6, | acid sequence |
| | 35D11_22A1 | |
| 102 | 17E9_15B1 | CDR-H2 amino acid sequence |
| 103 | 17E9_15B1 | VL amino acid sequence |
| 104 | 17E9_15B1 | CDR-L1 amino acid sequence |
| 105 | 17E9_15B1 | CDR-L2 amino acid sequence |
| 106 | 10C8-15A4 | VH amino acid |
| | | sequence |
| 107 | 10C8-15A4 | CDR-H2 amino acid sequence |
| 108 | 10C8-15A4 | VL amino acid sequence |
| 109 | 32A2_21A3 | VH amino acid sequence |
| 110 | 32A2_21A3 | CDR-H1 amino acid sequence |
| 111 | 32A2_21A3 | CDR-H2 amino acid sequence |
| 112 | 32A2_21A3 | CDR-H3 amino acid sequence |
| 113 | 32A2_21A3 | VL amino acid sequence |
| 114 | 32C12-N26S | VL amino acid sequence |
| 115 | 32C12-N26S | CDR-L1 amino acid sequence |
| 116 | 41G4_15B6 | VH amino acid sequence |
| 117 | 41G4_15B6 | CDR-H1 amino acid sequence |
| 118 | 31F9_21A1 | VH amino acid sequence |
| 119 | 31F9_21A1 | CDR-H2 amino acid sequence |
| 120 | 31F9_21A1 | VL amino acid sequence |
| 121 | 32D4_21D6 | VH amino acid sequence |
| 122 | 32D4_21D6 | VL amino acid sequence |
| 123 | 32F9_21A5 | VH amino acid sequence |
| 124 | 32F9_21A5 | VL amino acid sequence |
| 125 | 33E9_21A6 | VH amino acid sequence |
| 126 | 33E9_21A6 | VL amino acid sequence |
| 127 | 35D11_22A1 | VH amino acid sequence |
| 128 | 35D11_22A1 | VL amino acid sequence |
| 129 | 35D11_22A1 | CDR-L3 amino acid sequence |
| 130 | 39A9_28A4 | VH amino acid sequence |
| 131 | 39A9_28A4 | CDR-H1 amino acid sequence |
| 132 | 39A9_28A4 | CDR-H2 amino acid sequence |
| 133 | 39A9_28A4 | CDR-H3 amino acid sequence |
| 134 | 39A9_28A4 | VL amino acid sequence |
| 135 | 39A9_28A4 | CDR-L1 amino acid sequence |
| 136 | 34D1_21B3 | VH amino acid sequence |
| 137 | 34D1_21B3 | VL amino acid sequence |
| 138 | 34D1_21B3 | CDR-L1 amino acid sequence |
| 139 | 34D1_21B3 | CDR-L2 amino acid sequence |
| 140 | 33H2_21B1 | VH amino acid sequence |
| 141 | 33H2_21B1 | CDR-H1 amino acid sequence |
| 142 | 33H2_21B1 | CDR-H3 amino acid sequence |
| 143 | 33H2_21B1 | VL amino acid sequence |
| 144 | 36A12_21C1 | VH amino acid sequence |
| 145 | 36A12_21C1, | CDR-H1 amino acid sequence |
| | 40C3_22B6 | |
| 146 | 36A12_21C1 | CDR-H2 amino acid sequence |
| 147 | 36A12_21C1 | VL amino acid sequence |
| 148 | 36A12_21C1, | CDR-L1 amino |
| | 40C3_22B6 | acid sequence |
| 149 | 36A12_21C1 | CDR-L2 amino acid sequence |
| 150 | 36A12_21C1 | CDR-L3 amino acid sequence |
| 151 | 38G11_28A2 | VH amino acid sequence |
| 152 | 38G11_28A2 | CDR-H1 amino acid sequence |
| 153 | 38G11_28A2 | CDR-H2 amino acid sequence |
| 154 | 38G11_28A2 | VL amino acid sequence |
| 155 | 38G11_28A2, | CDR-L1 amino acid sequence |
| | 5D12_18A4, | |
| | 5D12-C108Y | |
| 156 | 38G11_28A2 | CDR-L2 amino acid sequence |
| 157 | 38G11_28A2 | CDR-L3 amino acid sequence |
| 158 | 40C3_22B6 | VH amino acid sequence |
| 159 | 40C3_22B6 | CDR-H2 amino acid sequence |
| 160 | 40C3_22B6 | VL amino acid sequence |
| 161 | 40C3_22B6 | CDR-L2 amino acid sequence |
| 162 | 40C3_22B6 | CDR-L3 amino acid sequence |
| 163 | 5D12_18A4 | VH amino acid sequence |
| 164 | 5D12_18A4, | CDR-H1 amino acid sequence |
| | 5D12_C108Y | |
| 165 | 5D12_18A4, | CDR-H2 amino acid sequence |
| | 5D12_C108Y | |
| 166 | 5D12_18A4 | CDR-H3 amino acid sequence |
| 167 | 5D12_18A4 | VL amino acid sequence |
| 168 | 5D12_18A4, | CDR-L2 amino acid sequence |
| | 5D12-C108Y | |
| 169 | 5D12_18A4, | CDR-L3 amino acid sequence |
| | 5D12-C108Y | |
| 170 | 5D12_C108Y | VH amino acid sequence |
| 171 | 5D12_C108Y | CDR-H3 amino acid sequence |
| 172 | 5D12-C108Y | VL amino acid sequence |
| 173 | 37D11_21C2 | VH amino acid sequence |
| 174 | 37D11_21C2 | CDR-H1 amino acid sequence |
| 175 | 37D11_21C2 | CDR-H2 amino acid sequence |
| 176 | 37D11_21C2 | CDR-H3 amino acid sequence |
| 177 | 37D11_21C2 | VL amino acid sequence |
| 178 | 37D11_21C2 | CDR-L2 amino acid sequence |
| 179 | 37D11_21C2, | CDR-L3 amino acid sequence |
| | 42D10_28A5 | |
| 180 | 42D10_28A5 | VH amino acid sequence |
| 181 | 42D10_28A5 | CDR-H1 amino acid sequence |
| 182 | 42D10_28A5 | CDR-H2 amino acid sequence |
| 183 | 42D10_28A5 | CDR-H3 amino acid sequence |
| 184 | 42D10_28A5 | VL amino acid sequence |
| 185 | 34H8_21B4 | VH amino acid sequence |
| 186 | 34H8_21B4 | CDR-H1 amino acid sequence |
| 187 | 34H8_21B4 | CDR-H2 amino acid sequence |
| 188 | 34H8_21B4 | CDR-H3 amino acid sequence |
| 189 | 34H8_21B4 | VL amino acid sequence |
| 190 | 34H8_21B4 | CDR-L1 amino acid sequence |
| 191 | 5G8_18A1 | VH nucleotide sequence |
| 192 | 5G8_18A1 | VL nucleotide sequence |
| 193 | 10C8_15A1, | VH nucleotide sequence |
| | 10C8_C43A | |
| 194 | 10C8_15A1 | VL nucleotide sequence |
| 195 | 12F3_17C2 | VH nucleotide sequence |
| 196 | 12F3_17C2 | VL nucleotide sequence |
| 197 | 16H2_17D2 | VH nucleotide sequence |
| 198 | 16H2_17D2 | VL nucleotide sequence |
| 199 | 32C12_21A4, | VH nucleotide sequence |
| | 32C12_N26S | |
| 200 | 32C12_21A4 | VL nucleotide sequence |
| 201 | 34C11_21B2 | VH nucleotide sequence |
| 202 | 34C11_21B2 | VL nucleotide sequence |
| 203 | 36A10_21B6 | VH nucleotide sequence |
| 204 | 36A10_21B6 | VL nucleotide sequence |
| 205 | 39G1_21C4 | VH nucleotide sequence |
| 206 | 39G1_21C4 | VL nucleotide sequence |
| 207 | 37E10_15B5 | VH nucleotide sequence |
| 208 | 37E10_15B5 | VL nucleotide sequence |
| 209 | 44E5_15C5 | VH nucleotide sequence |
| 210 | 44E5_15C5 | VL nucleotide sequence |
| 211 | 38E10_21C3 | VH nucleotide sequence |
| 212 | 38E10_21C3 | VL nucleotide sequence |
| 213 | 10C8_C43A | VL nucleotide sequence |
| 214 | 16H2_17D1 | VH nucleotide sequence |
| 215 | 16H2_17D1 | VL nucleotide sequence |
| 216 | 24G3_17C5 | VH nucleotide sequence |
| 217 | 24G3_17C5 | VL nucleotide sequence |
| 218 | 26C5_15B4 | VH nucleotide sequence |
| 219 | 26C5_15B4 | VL nucleotide sequence |
| 220 | 17E9_15B1 | VH nucleotide sequence |
| 221 | 17E9_15B1 | VL nucleotide sequence |
| 222 | 10C8_15A4 | VH nucleotide sequence |
| 223 | 10C8-15A4 | VL nucleotide sequence |
| 224 | 32A2_21A3 | VH nucleotide sequence |
| 225 | 32A2_21A3 | VL nucleotide sequence |
| 226 | 32C12_N26S | VL nucleotide sequence |
| 227 | 41G4_15B6 | VH nucleotide sequence |
| 228 | 41G4_15B6 | VL nucleotide sequence |
| 229 | 31F9_21A1 | VH nucleotide sequence |
| 230 | 31F9_21A1 | VL nucleotide sequence |
| 231 | 32D4_21D6 | VH nucleotide sequence |
| 232 | 32D4_21D6 | VL nucleotide sequence |
| 233 | 32F9_21A5 | VH nucleotide sequence |
| 234 | 32F9_21A5 | VL nucleotide sequence |
| 235 | 33E9_21A6 | VH nucleotide sequence |
| 236 | 33E9_21A6 | VL nucleotide sequence |
| 237 | 35D11_22A1 | VH nucleotide sequence |
| 238 | 35D11_22A1 | VL nucleotide sequence |
| 239 | 39A9_28A4 | VH nucleotide sequence |
| 240 | 39A9_28A4 | VL nucleotide sequence |
| 241 | 34D1_21B3 | VH nucleotide sequence |
| 242 | 34D1_21B3 | VL nucleotide sequence |
| 243 | 33H2_21B1 | VH nucleotide sequence |
| 244 | 33H2_21B1 | VL nucleotide sequence |
| 245 | 36A12_21C1 | VH nucleotide sequence |
| 246 | 36A12_21C1 | VL nucleotide sequence |
| 247 | 38G11_28A2 | VH nucleotide sequence |
| 248 | 38G11_28A2 | VL nucleotide sequence |
| 249 | 40C3_22B6 | VH nucleotide sequence |
| 250 | 40C3_22B6 | VL nucleotide sequence |
| 251 | 5D12_18A4 | VH nucleotide sequence |
| 252 | 5D12_18A4, | VL nucleotide sequence |
| | 5D12-C108Y | |
| 253 | 5D12_C108Y | VH nucleotide sequence |
| 254 | 37D11_21C2 | VH nucleotide sequence |
| 255 | 37D11_21C2 | VL nucleotide sequence |
| 256 | 42D10_28A5 | VH nucleotide sequence |
| 257 | 42D10_28A5 | VL nucleotide sequence |
| 258 | 34H8_21B4 | VH nucleotide sequence |
| 259 | 34H8_21B4 | VL nucleotide sequence |

| | |
|---|---|
| 260 | Human IL1RAP protein |

1. A method of treating an inflammatory or autoimmune condition in a mammal in need thereof, comprising administering a therapeutically effective amount of an antibody against IL1RAP.

2. The method of claim 1, wherein the inflammatory or autoimmune condition is an atopic condition, and wherein the antibody decreases levels of TARC/CCL17, PARC, periostin, IL-22, eotaxin-1, eotaxin-3, or combinations thereof at the site of atopy or in serum.

3. The method of claim 1, wherein the inflammatory or autoimmune condition is characterized or caused by increased neutrophil or eosinophil cell counts; neutrophil or eosinophil dysfunction; or increased TARC/CCL17 levels.

4. The method of claim 3, wherein the inflammatory or autoimmune condition is characterized or caused by increased neutrophil cell counts or neutrophil dysfunction.

5. The method of claim 4, wherein the inflammatory or autoimmune condition characterized or caused by increased neutrophil cell counts or neutrophil dysfunction is selected from the group consisting of: hidradenitis suppurativa, generalized pustular psoriasis (GPP), COPD, idiopathic fibrosis, neutrophilic asthma, Neutrophil dermatose, Pyoderma Gangrenosum Schnitzler syndrome, Behçet's disease, Sweet's syndrome, rheumatoid arthritis, systemic lupus erythematosus (SLE), inflammatory bowel disease (Crohn's disease, ulcerative colitis), psoriasis, vasculitis, Alzheimer Disease, COVID-19, and Gout.

6. The method of claim 3, wherein the inflammatory or autoimmune condition is further characterized by increased eosinophil cell counts or eosinophil dysfunction.

7. The method of claim 6, wherein the inflammatory or autoimmune condition characterized or caused by increased eosinophil cell counts or eosinophil dysfunction is selected from the group consisting of: Atopic dermatitis (eczema), Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, and Eosinophilic Myocarditis.

8. The method of claim 3, wherein the inflammatory or autoimmune condition is further characterized by increased TARC/CCL17 levels.

9. The method of claim 8, wherein the inflammatory or autoimmune condition characterized by increased TARC/CCL17 levels is selected from the group consisting of: Atopic Dermatitis, COPD, bronchial asthma, allergic rhinitis, eosinophilic pneumonia, Hypersensitivity Pneumonitis, Lichen Planus, Sarcoidosis, Urticaria, Mastocytosis, eosinophilic-associated disorders.

10. The method of claim 1, wherein the inflammatory or autoimmune condition is selected from the group consisting of sepsis, acute respiratory distress syndrome, COVID-19, myocardial infarction, cystic fibrosis, irritable bowel disease, ulcerative colitis, Crohn's disease, atopic dermatitis, psoriasis, multiple sclerosis, asthma, neutrophilic asthma, Alzheimer's disease, stroke, diabetic kidney disease, diabetes, diabetic retinopathy, Chronic obstructive pulmonary disease (COPD), idiopathic pulmonary fibrosis, non-alcoholic fatty liver disease, rheumatoid arthritis, psoriatic arthritis, ankylosing spondylitis, axial spondyloarthritis, systemic lupus erythematosus (SLE), vasculitis, Gout. Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria, and Mastocytosis.

11. The method of claim 1, further comprising a step of identifying the mammal in need thereof according to abnormal serum levels of IL-1α, IL-1β, IL-33, IL-36α, IL-36β, IL-36γ, or combinations thereof.

12. The method of claim 1, further comprising a step of identifying the mammal in need thereof according to abnormal serum levels of CXCL1, CXCL8, LCN-2, TARC, or combinations thereof.

13. The method of claim 11, wherein the serum levels of IL-1α, IL-1β, IL-33, IL-36α, IL-36β, IL-36γ, or combinations thereof are elevated.

14. The method of claim 12, wherein the serum levels of CXCL1, CXCL8, LCN-2, TARC, or combinations thereof are elevated.

15. The method of claim 1, wherein the antibody is characterized by its inhibition of IL-8 release by intestinal epithelial cells, intestinal myofibroblasts, or dermal fibroblasts stimulated with IL-1α, IL-1β, IL-36α, IL-36β, and/or IL-36γ, or combinations thereof.

16. The method of claim 2, wherein the atopic condition is selected from the group consisting of atopic dermatitis, asthma, COPD, allergic rhinitis, allergic conjunctivitis, food allergy, drug allergy, and angioedema.

17. The method of claim 2, wherein the atopic condition is atopic dermatitis and the antibody decreases levels of TARC/CCL17, PARC, periostin, IL-22, eotaxin-1, eotaxin-3, or combinations thereof in the skin or serum.

18. The method of claim 2, wherein the atopic condition is asthma.

19. The method of claim 2, wherein the atopic condition is COPD.

20. The method of claim 1, wherein the antibody is administered in combination with at least one other therapeutic agent.

21. The method of claim 20, wherein the at least one other therapeutic agent is a therapeutic antibody, corticosteroid, small molecule, siRNA, mRNA, or combination thereof.

22. The method of claim 20, wherein the at least one other therapeutic agent inhibits IL-4 signaling.

23. The method of claim 22, wherein the at least one other therapeutic agent is an IL-4Ra inhibitor or antagonist, a Pan-JAK inhibitor or antagonist, or combinations thereof.

24. The method of claim 22, wherein the at least one other therapeutic agent is dupilumab, CBP-201, AK120, cerdulatinib, CEE321, jaktinib, delgocitinib, filgotinib, tofacitinib, dexamethasone, triamcinolone, prednisone, or combinations thereof.

25. The method of claim 22, wherein the inflammatory or autoimmune condition is rheumatoid arthritis, psoriasis, psoriatic arthritis, ankylosing spondylitis, axial spondyloarthritis, Polyarticular Course Juvenile Idiopathic Arthritis, Crohn's disease, ulcerative colitis, atopic dermatitis, COPD, or asthma.

26. The method of claim 20, wherein the at least one other therapeutic agent inhibits IL-13 signaling.

27. The method of claim 26, wherein the at least one other therapeutic agent is an IL-13 inhibitor or antagonist, a JAK1 inhibitor or antagonist, TYK2 inhibitor or antagonist, or combinations thereof.

28. The method of claim 26, wherein the at least one other therapeutic agent is upadacitinib, abrocitinib, tralokinumab, lebrikizumab, eblasakimab, baricitinib, ruxolitinib, filgotinib, PF-06651600, dexamethasone, triamcinolone, prednisone, or combinations thereof.

29. The method of claim 26, wherein the inflammatory or autoimmune condition is rheumatoid arthritis, Systemic lupus erythematosus, vitiligo, psoriasis, psoriatic arthritis, ankylosing spondylitis, axial spondyloarthritis, Crohn's disease, ulcerative colitis, atopic dermatitis, COPD or asthma.

30. The method of claim 20, wherein the at least one other therapeutic agent inhibits IL-22 signaling.

31. The method of claim 30, wherein the at least one other therapeutic agent is an IL-22 inhibitor or antagonist, an IL-22R1 inhibitor or antagonist, a JAK1 inhibitor or antagonist, a JAK2 inhibitor or antagonist, a TYK2 inhibitor or antagonist, or combinations thereof.

32. The method of claim 30, wherein the at least one other therapeutic agent is fezakinumab, LEO 138559, brepocitinib, ATI-1777, deucravacitinib, TAK-279, upadacitinib and abrocitinib, or combinations thereof.

33. The method of claim 30, wherein the inflammatory or autoimmune condition is rheumatoid arthritis, Systemic lupus erythematosus, vitiligo, psoriasis, psoriatic arthritis, ankylosing spondylitis, axial spondyloarthritis, Crohn's disease, ulcerative colitis, atopic dermatitis, COPD, or asthma.

34. The method of claim 20, wherein the at least one other therapeutic agent inhibits IL-33 signaling.

35. The method of claim 34, wherein the at least one other therapeutic agent is an IL-33 or IL-33R inhibitor or antagonist.

36. The method of claim 34, wherein the at least one other therapeutic agent is etokimab, itepekimab, astegolimab, PF-06817024, tozorakimab, CNTO 7160, or combinations thereof.

37. The method of claim 34, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, chronic obstructive pulmonary disease (COPD), asthma, allergic contact dermatitis, irritant contact dermatitis, rosacea, psoriasis vulgaris, pustular psoriasis, mastocytosis, systemic lupus erythematosus, systemic sclerosis, chronic spontaneous urticaria, autoimmune blistering diseases, Behcet's disease, or vitiligo.

38. The method of claim 20, wherein the at least one other therapeutic agent inhibits IL-17 signaling.

39. The method of claim 38, wherein the at least one other therapeutic agent is an IL-17A, IL-17C, IL-17F, IL17A/F, or an IL-17RA inhibitor or antagonist; or combinations thereof.

40. The method of claim 38, wherein the at least one other therapeutic agent is secukinumab, ixekizumab, brodalumab, bimekizumab, izokibep, sonelokimab, or combinations thereof.

41. The method of claim 38, wherein the inflammatory or autoimmune condition is rheumatoid arthritis, psoriasis, psoriatic arthritis, ankylosing spondylitis, axial spondyloarthritis, enthesitis-related arthritis, Crohn's disease, ulcerative colitis, atopic dermatitis, hidradenitis suppurativa, COPD, or asthma.

42. The method of claim 20, wherein the at least one other therapeutic agent inhibits IL-36 signaling.

43. The method of claim 42, wherein the at least one other therapeutic agent is an IL-36 inhibitor or antagonist or an IL-36R inhibitor or antagonist.

44. The method of claim 42, wherein the at least one other therapeutic agent is spesolimab, imsidolimab, REGN6490, or combinations thereof.

45. The method of claim 42, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, asthma, neutrophilic asthma, neutrophilic lung inflammation, COVID-19, Chronic obstructive pulmonary disease (COPD), idiopathic pulmonary fibrosis, non-alcoholic fatty liver disease, neutrophilic dermatoses, hidradenitis suppurativa, generalized pustular psoriasis, palmoplantar pustular psoriasis, deficiency of IL-36 receptor antagonist (DITRA), psoriasis vulgaris, CARD14-mediated psoriasis, acute generalized exanthematous pustulosis, pyoderma gangrenosum, Sweet's syndrome, systemic lupus erythematosus, systemic sclerosis, autoimmune blistering diseases, acne, allergic contact dermatitis, or folliculitis and eosinophilic pustular folliculitis.

46. The method of claim 20, wherein the at least one other therapeutic agent inhibits IL-18 signaling.

47. The method of claim 46, wherein the at least one other therapeutic agent is an IL-18 inhibitor or antagonist.

48. The method of claim 46, wherein the at least one other therapeutic agent is tadekinig alfa.

49. The method of claim 46, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, asthma, adult-onset Still disease, cutaneous lupus erythematosus, chronic spontaneous urticaria, contact dermatitis, alopecia areata, cutaneous drug eruptions, graft-versus-host disease, cryopyrin-associated periodic syndromes, granulomatosis with polyangiitis, systemic sclerosis, hidradenitis suppurativa, pyogenic arthritis, pyoderma gangrenosum and acne (PAPA), familial Mediterranean fever, rosacea, synovitis, acne, pustulosis, hyperostosis, osteitis (SAPHO), bullous pemphigoid, pemphigus vulgaris, Behcet's disease, or Schnitzler syndrome.

50. The method of claim 20, wherein the at least one other therapeutic agent inhibits IL-23 signaling.

51. The method of claim 50, wherein the at least one other therapeutic agent is an IL-23 inhibitor or antagonist, an IL-12/23 p40 subunit inhibitor or antagonist, an IL-23 p19 subunit inhibitor or antagonist, or combinations thereof.

52. The method of claim 50, wherein the at least one other therapeutic agent is risankizumab, ustekinumab, guselkumab, tildrakizumab, mirikizumab, brazikumab, or combinations thereof.

53. The method of claim 50, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.

54. The method of claim 20, wherein the at least one other therapeutic agent inhibits OX40 signaling.

55. The method of claim 54 wherein the at least one other therapeutic agent is an OX40 or OX40L inhibitor or antagonist.

56. The method of claim 54, wherein the at least one other therapeutic agent is rocatinlimab, GBR 830, amlitelimab, or combinations thereof.

57. The method of claim 54, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.

58. The method of claim 20, wherein the at least one other therapeutic agent inhibits IL-5 signaling.

59. The method of claim 58 wherein the at least one other therapeutic agent is an IL-5Rα inhibitor or antagonist, a JAK1 inhibitor or antagonist, or combinations thereof.

60. The method of claim 58, wherein the at least one other therapeutic agent is benralizumab, upadacitinib, abrocitinib, SHR0302, filgotinib, PF-06651600, dexamethasone, triamcinolone, prednisone, or combinations thereof.

61. The method of claim 58, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.

62. The method of claim 20, wherein the at least one other therapeutic agent inhibits T cell migration.

63. The method of claim 62 wherein the at least one other therapeutic agent is an S1PR1 inhibitor or antagonist, an S1PR4 inhibitor or antagonist, an S1PR5 inhibitor or antagonist, a CCR4 inhibitor or antagonist, or combinations thereof.

64. The method of claim 62, wherein the at least one other therapeutic agent is etrasimod, ozanimod, SCD-044, LC51-0255, BMS-986166, RPT193, or combinations thereof.

65. The method of claim 62, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.

66. The method of claim 20, wherein the at least one other therapeutic agent inhibits pruritis.

67. The method of claim 66 wherein the at least one other therapeutic agent is an IL-1α inhibitor or antagonist, an OSMRβ inhibitor or antagonist, an NK1R inhibitor or antagonist, a P2X3 inhibitor or antagonist, an IL-31 inhibitor or antagonist, or combinations thereof.

68. The method of claim 66, wherein the at least one other therapeutic agent is bermekimab, vixarelimab, serlopitant, tradipitant, BLU-5937, nemolizumab, or combinations thereof.

69. The method of claim 66, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, or asthma.

70. The method of claim 20, wherein the at least one other therapeutic agent inhibits TH1-associated immune response.

71. The method of claim 70 wherein the at least one other therapeutic agent is an IL-1α inhibitor or antagonist, IL-1β inhibitor or antagonist, IL-1R1 inhibitor or antagonist, IL-36R inhibitor or antagonist, a TNFα inhibitor or antagonist, or combinations thereof.

72. The method of claim 70, wherein the at least one other therapeutic agent is bermekimab, anakinra, canakinumab, gevokizumab, rilonacept, MEDI8968, spesolimab, imsidolimab, REGN6490, adalimumab, infliximab, etanercept, certolizumab, golimumab, or combinations thereof.

73. The method of claim 70, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, or monogenic autoinflammatory skin disorders.

74. The method of claim 20, wherein the at least one other therapeutic agent inhibits TH2-associated immune responses.

75. The method of claim 74 wherein the at least one other therapeutic agent is an IL-4 inhibitor or antagonist, Type I IL-4 receptor inhibitor or antagonist, Type II IL-4 receptor inhibitor or antagonist, IL-13 inhibitor or antagonist, Type I IL-13 receptor inhibitor or antagonist, Type II IL-13 receptor inhibitor or antagonist, and IL-5 inhibitor or antagonist, Type I IL-5 receptor inhibitor or antagonist, Type II IL-5 receptor inhibitor or antagonist, IL-9 inhibitor or antagonist, Type I IL-9 receptor inhibitor or antagonist, IL-10 inhibitor or antagonist, homodimeric IL-10 receptor inhibitor or antagonist, heterodimeric IL-10 receptor inhibitor or antagonist or combinations thereof.

76. The method of claim 74, wherein the at least one other therapeutic agent is dupilumab, omalizumab, mepolizumab, benralizumab, tralokinumab, lebrikizumab, or combinations thereof.

77. The method of claim 74, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, or monogenic autoinflammatory skin disorders Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria.

78. The method of claim 20, wherein the at least one other therapeutic agent inhibits TH17-associated immune responses.

79. The method of claim 78 wherein the at least one other therapeutic agent is an IL-17 inhibitor or antagonist, Type I IL-17 receptor inhibitor or antagonist, Type II IL-17 receptor inhibitor or antagonist, IL-23 inhibitor or antagonist, IL-23 receptor inhibitor or antagonist, or combinations thereof.

80. The method of claim 78, wherein the at least one other therapeutic agent is secukinumab, ixekizumab, brodalumab, bimekizumab, izokibep, sonelokimab, risankizumab, ustekinumab, guselkumab, tildrakizumab, mirikizumab, brazikumab or combinations thereof.

81. The method of claim 78, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, COPD, asthma, adult-onset Still disease, Behcet's disease, hidradenitis suppurativa, pyoderma gangrenosum, SAPHO, acne vulgaris, psoriasis vulgaris, Schnitzler syndrome, urticarial vasculitis, familial Mediterranean fever (FMF), pyogenic arthritis, pyoderma gangrenosum, acne (PAPA), cryopyrin-associated periodic syndromes (CAPS), hyper-IgD syndrome (HIDS), also known as mevalonate kinase deficiency (MKD), TNF receptor-associated periodic syndrome (TRAPS), deficiency of IL-1 receptor antagonist (DIRA), sweet syndrome, PASH, PFAPA, generalized pustular psoriasis (GPP), palmoplantar pustular psoriasis (PPP), dermatomyositis, panniculitis, Erdheim-Chester syndrome, deficiency of adenosine deaminase (DADA2), Majeed syndrome, deficiency of IL-36 receptor antagonist (DITRA), haploinsufficiency of A20 (HA20), PAPASH, rosacea, acute generalized exanthematous pustulosis (AGEP), allergic contact dermatitis, irritant contact dermatitis, mastocytosis, systemic sclerosis, chronic spontaneous urticaria, autoimmune blistering diseases, vitiligo, CARD-14 mediated pustular psoriasis (CAMPS), familiar keratosis lichenoides chronica (FKLC), multiple self-healing palmoplantar carcinoma (MSPC), pyrin-associated autoinflammation with neutrophilic dermatosis (PAAND), NLRC4-related macrophage activation syndrome (NLRC4-MAS), neutrophilic dermatoses, Allergic rhinitis, Allergic Conjunctivitis, Eosinophilic Esophagitis (EoE), Eosinophilic Asthma, Hypereosinophilic Syndrome (HES), Eosinophilic Granulomatosis, Eosinophilic Fasciitis, Eosinophilic Gastrointestinal Disorders, Eosinophilic Pneumonia, Eosinophilic Myocarditis, Hypersensitivity Pneumonitis, Lichen Planus, Sarcoidosis, Urticaria, or monogenic autoinflammatory skin disorders.

82. The method of claim 20, wherein the at least one other therapeutic agent inhibits IRAK4 signaling.

83. The method of claim 82 wherein the at least one other therapeutic agent is an IRAK4 inhibitor, degrader, or antagonist.

84. The method of claim 82, wherein the at least one other therapeutic agent is PF-06650833, CA-4948, KT-474, or combinations thereof.

85. The method of claim 82, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, hidradenitis suppurativa, COPD, or asthma.

86. The method of claim 20, wherein the at least one other therapeutic agent inhibits complement signaling.

87. The method of claim 86 wherein the at least one other therapeutic agent is a C5a inhibitor or antagonist, a C5aR inhibitor or antagonist, a TSLP inhibitor or antagonist, a CD80/CD86 inhibitor or antagonist, an IL-6 inhibitor or antagonist, a CD20 inhibitor or antagonist, an integrin oc4 inhibitor or antagonist, an AhR agonist, or combinations thereof.

88. The method of claim 86, wherein the at least one other therapeutic agent is vilobelimab, FX002, INF904, tezepelumab, abatacept, tocilizumab, sarilumab, rituximab, vedolizumab, tapinarof, tadekinig alfa, or combinations thereof.

89. The method of claim 86, wherein the inflammatory or autoimmune condition is rheumatoid arthritis, psoriasis, psoriatic arthritis, ankylosing spondylitis, axial spondyloarthritis, Crohn's disease, ulcerative colitis, atopic dermatitis, hidradenitis suppurativa, COVID-19, COPD, or asthma.

90. The method of claim 20, wherein the at least one other therapeutic agent inhibits PDE4 signaling.

91. The method of claim 90, wherein the at least one other therapeutic agent is a PDE4 inhibitor or antagonist.

92. The method of claim 90, wherein the at least one other therapeutic agent is apremilast, crisaborole, difamilast, roflumilast, or combinations thereof.

93. The method of claim 90, wherein the inflammatory or autoimmune condition is rheumatoid arthritis, psoriasis, psoriatic arthritis, ankylosing spondylitis, ankylosing spondylitis, axial spondyloarthritis, Crohn's disease, ulcerative colitis, atopic dermatitis, hidradenitis suppurativa, COPD or asthma Crohn's disease, ulcerative colitis, atopic dermatitis, or asthma.

94. The method of claim 20, wherein the at least one other therapeutic agent is administered before, simultaneously with, or after the at least one other therapeutic agent.

95. The method of claim 1, wherein the antibody is administered subcutaneously or intravenously at a dose selected from: 1-80 mg/kg, 1-60 mg/kg, 1-50 mg/kg, 1-40 mg/kg, 1-30 mg/kg, 1-25 mg/kg, or 1-20 mg/kg.

96. The method of claim 17, wherein the antibody is administered subcutaneously or intravenously at a dose selected from: 1-80 mg/kg, 1-60 mg/kg, 1-50 mg/kg, 1-40 mg/kg, 1-30 mg/kg, 1-25 mg/kg, or 1-20 mg/kg.

97. The method of claim 20, wherein the antibody is administered subcutaneously or intravenously at a dose selected from: 1-80 mg/kg, 1-60 mg/kg, 1-50 mg/kg, 1-40 mg/kg, 1-30 mg/kg, 1-25 mg/kg, or 1-20 mg/kg.

98. The method of claim 1, wherein the antibody is administered as a subcutaneous injection or as a bolus intravenously, less frequently than once per week, twice per week, more than twice per week, or in a continuous infusion.

99. The method of claim 17, wherein the antibody is administered as a subcutaneous injection or as a bolus intravenously, less frequently than once per week, twice per week, more than twice per week, or in a continuous infusion.

100. The method of claim 20, wherein the antibody is administered as a subcutaneous injection or as a bolus intravenously, less frequently than once per week, twice per week, more than twice per week, or in a continuous infusion.

101. The method of claim 1, 17 or 20, wherein the antibody comprises:
BFB759 (i.e., 37E10_15B5) corresponding to a heavy chain variable region comprising a CDR3 domain comprising the amino acid sequence of SEQ ID NO: 67, a CDR2 domain comprising the amino acid sequence of SEQ ID NO: 66, and a CDR1 domain comprising the amino acid sequence of SEQ ID NO: 65; and a light chain variable region comprising a CDR3 domain comprising the amino acid sequence of SEQ ID NO: 70, a CDR2 domain comprising the amino acid sequence of SEQ ID NO: 62, and a CDR1 domain comprising the amino acid sequence of SEQ ID NO: 69;
a heavy chain variable region comprising a CDR3 domain comprising the amino acid sequence of SEQ ID NO: 12, a CDR2 domain comprising the amino acid sequence of SEQ ID NO: 11, and a CDR1 domain comprising the amino acid sequence of SEQ ID NO: 10; and a light chain variable region comprising a CDR3 domain comprising the amino acid sequence of SEQ ID NO: 16, a CDR2 domain comprising the amino acid sequence of SEQ ID NO: 15, and a CDR1 domain comprising the amino acid sequence of SEQ ID NO: 14;
a heavy chain variable region comprising a CDR3 domain comprising the amino acid sequence of SEQ ID NO: 51, a CDR2 domain comprising the amino acid sequence of SEQ ID NO: 50, and a CDR1 domain comprising the amino acid sequence of SEQ ID NO: 49; and a light chain variable region comprising a CDR3 domain comprising the amino acid sequence of SEQ ID NO: 55, a CDR2 domain comprising the amino acid sequence of SEQ ID NO: 54, and a CDR1 domain comprising the amino acid sequence of SEQ ID NO: 53;
a heavy chain variable region comprising a CDR3 domain comprising the amino acid sequence of SEQ ID NO: 59, a CDR2 domain comprising the amino acid sequence of SEQ ID NO: 58, and a CDR1 domain comprising the amino acid sequence of SEQ ID NO: 57; and a light chain variable region comprising a CDR3 domain comprising the amino acid sequence of SEQ ID NO: 63, a CDR2 domain comprising the amino acid sequence of SEQ ID NO: 62, and a CDR1 domain comprising the amino acid sequence of SEQ ID NO: 61; or
a heavy chain variable region comprising a CDR3 domain comprising the amino acid sequence of SEQ ID NO: 176, a CDR2 domain comprising the amino acid sequence of SEQ ID NO: 175, and a CDR1 domain comprising the amino acid sequence of SEQ ID NO: 174; and a light chain variable region comprising a CDR3 domain comprising the amino acid sequence of SEQ ID NO: 179, a CDR2 domain comprising the amino acid sequence of SEQ ID NO: 178, and a CDR1 domain comprising the amino acid sequence of SEQ ID NO: 79.

102. The method of claim 1, 17 or 20, wherein the antibody comprises:
BFB759 (i.e., 37E10_15B5) corresponding to a heavy chain variable region comprising the amino acid sequence of SEQ ID NO: 64 and a light chain variable region comprising the amino acid sequence of SEQ ID NO: 68;
a heavy chain variable region comprising the amino acid sequence of SEQ ID NO: 9 and a light chain variable region comprising the amino acid sequence of SEQ ID NO: 82;
a heavy chain variable region comprising the amino acid sequence of SEQ ID NO: 48 and a light chain variable region comprising the amino acid sequence of SEQ ID NO: 52;
a heavy chain variable region comprising the amino acid sequence of SEQ ID NO: 56 and a light chain variable region comprising the amino acid sequence of SEQ ID NO: 60; or
a heavy chain variable region comprising the amino acid sequence of SEQ ID NO: 173 and a light chain variable region comprising the amino acid sequence of SEQ ID NO: 177.


SEQUENCE LISTING (260 sequences)

SEQ ID NO: 1 - protein, 114 aa, synthetic construct
  REGION 1..114: note = Description of Artificial Sequence: Synthetic polypeptide
QVELQESGPGLVKPSDTLSLTCTVTGGSISTYYWSWIRQPPGKGLEWIGYIFYTGTTNYNPSLKSRVTISVDASKNQFSLKLNSVTAADTAVYYCARDGSLDYWGQGALVTVSS

SEQ ID NO: 2 - protein, 10 aa, synthetic construct
  REGION 1..10: note = Description of Artificial Sequence: Synthetic peptide
GGSISTYYWS

SEQ ID NO: 3 - protein, 16 aa, synthetic construct
  REGION 1..16: note = Description of Artificial Sequence: Synthetic peptide
YIFYTGTTNYNPSLKS

SEQ ID NO: 4 - protein, 6 aa, synthetic construct
  REGION 1..6: note = Description of Artificial Sequence: Synthetic peptide
DGSLDY

SEQ ID NO: 5 - protein, 107 aa, synthetic construct
  REGION 1..107: note = Description of Artificial Sequence: Synthetic polypeptide
DIQMTQSPSSLSASVGDRVTITCRASQSISNYLNWYQQKPGKAPKLLIFVASLLQSGVPSRFSGSGSGTDFTLTISSLQPEDFATYYCQQSYSTPFTFGPGTKLEIK

SEQ ID NO: 6 - protein, 11 aa, synthetic construct
  REGION 1..11: note = Description of Artificial Sequence: Synthetic peptide
RASQSISNYLN

SEQ ID NO: 7 - protein, 7 aa, synthetic construct
  REGION 1..7: note = Description of Artificial Sequence: Synthetic peptide
VASLLQS

SEQ ID NO: 8 - protein, 9 aa, synthetic construct
  REGION 1..9: note = Description of Artificial Sequence: Synthetic peptide
QQSYSTPFT

SEQ ID NO: 9 - protein, 124 aa, synthetic construct
  REGION 1..124: note = Description of Artificial Sequence: Synthetic polypeptide
QVQLVESGGGVVQPGRSLRISCAASGFTFRSYGMHWVRQAPGKGLEWVAIIWHDESYKYYVDSVKGRFTISRDNSKNTLYLQMNSLRAEDTAVYYCARGDYYGSGSYYDAFDIWGQGTMVTVSS

SEQ ID NO: 10 - protein, 10 aa, synthetic construct
  REGION 1..10: note = Description of Artificial Sequence: Synthetic peptide
GFTFRSYGMH

SEQ ID NO: 11 - protein, 17 aa, synthetic construct
  REGION 1..17: note = Description of Artificial Sequence: Synthetic peptide
IIWHDESYKYYVDSVKG

SEQ ID NO: 12 - protein, 15 aa, synthetic construct
  REGION 1..15: note = Description of Artificial Sequence: Synthetic peptide
GDYYGSGSYYDAFDI

SEQ ID NO: 13 - protein, 107 aa, synthetic construct
  REGION 1..107: note = Description of Artificial Sequence: Synthetic polypeptide
DIQMTQSPSSLSASVGDRVIITCRASQGISNYLAWFQQKPGKCPNLLIYAASTLRSGVPSRFSGSGSGTDFTLTISSLQPEDVATYYCQKYNSAPYTFGQGTKVEIK

SEQ ID NO: 14 - protein, 11 aa, synthetic construct
  REGION 1..11: note = Description of Artificial Sequence: Synthetic peptide
RASQGISNYLA

SEQ ID NO: 15 - protein, 7 aa, synthetic construct
  REGION 1..7: note = Description of Artificial Sequence: Synthetic peptide
AASTLRS

SEQ ID NO: 16 - protein, 9 aa, synthetic construct
  REGION 1..9: note = Description of Artificial Sequence: Synthetic peptide
QKYNSAPYT

SEQ ID NO: 17 - protein, 121 aa, synthetic construct
  REGION 1..121: note = Description of Artificial Sequence: Synthetic polypeptide
QVQLVESGGGVVQPGRSLRLSCAASGFTFSHYGMHWVRQAPGKGLEWVAVIWYDGSKKYYVDSVKGRFTISRDNSKNTLYLQMNSLRGEDTAVYYCARDGGAVADNWIDSWGQGTLVTVSS

SEQ ID NO: 18 - protein, 10 aa, synthetic construct
  REGION 1..10: note = Description of Artificial Sequence: Synthetic peptide
GFTFSHYGMH

SEQ ID NO: 19 - protein, 16 aa, synthetic construct
  REGION 1..16: note = Description of Artificial Sequence: Synthetic peptide
IWYDGSKKYYVDSVKG

SEQ ID NO: 20 - protein, 12 aa, synthetic construct
  REGION 1..12: note = Description of Artificial Sequence: Synthetic peptide
DGGAVADNWIDS

SEQ ID NO: 21 - protein, 107 aa, synthetic construct
  REGION 1..107: note = Description of Artificial Sequence: Synthetic polypeptide
DIQMTQSPSSLSASVGDRVTITCRASQSIRSYLNWYQQKPGKAPKLLIFAASSLQSGVPSRFSGSGSGTDFTLTVSSLQPEDFATYSCQQSYSTPYTFGQGTKVEIK

SEQ ID NO: 22 - protein, 11 aa, synthetic construct
  REGION 1..11: note = Description of Artificial Sequence: Synthetic peptide
RASQSIRSYLN

SEQ ID NO: 23 - protein, 7 aa, synthetic construct
  REGION 1..7: note = Description of Artificial Sequence: Synthetic peptide
AASSLQS

SEQ ID NO: 24 - protein, 9 aa, synthetic construct
  REGION 1..9: note = Description of Artificial Sequence: Synthetic peptide
QQSYSTPYT

SEQ ID NO: 25 - protein, 120 aa, synthetic construct
  REGION 1..120: note = Description of Artificial Sequence: Synthetic polypeptide
QVQLVESGGGVVQPGGSLRLSCAASGFTFSSHGMHWVRQAPGKGLEWVAVIWYDGSNKFYTDSVQGRFTISRDNSKNTLNLQMNSLRAEDTAVYYCAREGLRAGYYFDFWGQGTLVTVSS

SEQ ID NO: 26 - protein, 10 aa, synthetic construct
  REGION 1..10: note = Description of Artificial Sequence: Synthetic peptide
GFTFSSHGMH

SEQ ID NO: 27 - protein, 17 aa, synthetic construct
  REGION 1..17: note = Description of Artificial Sequence: Synthetic peptide
VIWYDGSNKFYTDSVQG

SEQ ID NO: 28 - protein, 11 aa, synthetic construct
  REGION 1..11: note = Description of Artificial Sequence: Synthetic peptide
EGLRAGYYFDF

SEQ ID NO: 29 - protein, 107 aa, synthetic construct
  REGION 1..107: note = Description of Artificial Sequence: Synthetic polypeptide
DIQMTQSPSSLSASVGDRVTITCRASQRISFYSNWYQQKPGKAPKLLIYAASSLQSGVPSRFSGSGSGTDFTLTISSLQPEDFATYFCQQSHSTPLTFGGGTKLEIK

SEQ ID NO: 30 - protein, 11 aa, synthetic construct
  REGION 1..11: note = Description of Artificial Sequence: Synthetic peptide
RASQRISFYSN

SEQ ID NO: 31 - protein, 9 aa, synthetic construct
  REGION 1..9: note = Description of Artificial Sequence: Synthetic peptide
QQSHSTPLT

SEQ ID NO: 32 - protein, 120 aa, synthetic construct
  REGION 1..120: note = Description of Artificial Sequence: Synthetic polypeptide
QVQLVESGGGVVQPGRSLRLSCAASGFTFRNYGIHWVRQAPGKGLEWVAVIWYDGSNKFYADSVKGRFTISRDNSKNTLYLQMNSLRVEDTAVYYCAREEGIAVAPFDYWGQGTLVTVSS

SEQ ID NO: 33 - protein, 10 aa, synthetic construct
  REGION 1..10: note = Description of Artificial Sequence: Synthetic peptide
GFTFRNYGIH

SEQ ID NO: 34 - protein, 17 aa, synthetic construct
  REGION 1..17: note = Description of Artificial Sequence: Synthetic peptide
VIWYDGSNKFYADSVKG

SEQ ID NO: 35 - protein, 11 aa, synthetic construct
  REGION 1..11: note = Description of Artificial Sequence: Synthetic peptide
EEGIAVAPFDY

SEQ ID NO: 36 - protein, 107 aa, synthetic construct
  REGION 1..107: note = Description of Artificial Sequence: Synthetic polypeptide
DIQMTQSPSSLSASVGDRVAITCRANQSIASYLNWYQQKPGKAPKLLIYGASSLQNGVPSRFSGSGSRTDFTLTISSLQPEDFATYYCQQSYSTEITFGQGTRLEIK

SEQ ID NO: 37 - protein, 11 aa, synthetic construct
  REGION 1..11: note = Description of Artificial Sequence: Synthetic peptide
RANQSIASYLN

SEQ ID NO: 38 - protein, 7 aa, synthetic construct
  REGION 1..7: note = Description of Artificial Sequence: Synthetic peptide
GASSLQN

SEQ ID NO: 39 - protein, 9 aa, synthetic construct
  REGION 1..9: note = Description of Artificial Sequence: Synthetic peptide
QQSYSTEIT

SEQ ID NO: 40 - protein, 120 aa, synthetic construct
  REGION 1..120: note = Description of Artificial Sequence: Synthetic polypeptide
EVQVVESGGGLVQPGGSLRLSCAASGFTFRSYWMSWVRQAPGKGLEWVANIKQDGSERHYVDSVKGRFTISRDNAKTSLYLQMSSLRAEDTAVYYCAREGYFGSGYFDYWGQGTLVTVSS

SEQ ID NO: 41 - protein, 10 aa, synthetic construct
  REGION 1..10: note = Description of Artificial Sequence: Synthetic peptide
GFTFRSYWMS

SEQ ID NO: 42 - protein, 17 aa, synthetic construct
  REGION 1..17: note = Description of Artificial Sequence: Synthetic peptide
NIKQDGSERHYVDSVKG

SEQ ID NO: 43 - protein, 11 aa, synthetic construct
  REGION 1..11: note = Description of Artificial Sequence: Synthetic peptide
EGYFGSGYFDY

SEQ ID NO: 44 - protein, 107 aa, synthetic construct
  REGION 1..107: note = Description of Artificial Sequence: Synthetic polypeptide
DIQMTQSPSTLSASVGDRVIITCRASQSISRGLAWYQQKPGKAPKLLIYKASNLESGVPSRFSGSGSGTEFTLTISSLQPDDFAAYYCQQYSYYSHTFGQGTKLEIK

SEQ ID NO: 45 - protein, 11 aa, synthetic construct
  REGION 1..11: note = Description of Artificial Sequence: Synthetic peptide
RASQSISRGLA

SEQ ID NO: 46 - protein, 7 aa, synthetic construct
  REGION 1..7: note = Description of Artificial Sequence: Synthetic peptide
KASNLES

SEQ ID NO: 47 - protein, 9 aa, synthetic construct
  REGION 1..9: note = Description of Artificial Sequence: Synthetic peptide
QQYSYYSHT

SEQ ID NO: 48 - protein, 125 aa, synthetic construct
  REGION 1..125: note = Description of Artificial Sequence: Synthetic polypeptide
QGQVVESGGGVVQPGRSLRLSCAASGFTFSSYTLHWVRQAPGKGLEWVAVIWYDGSNKYYVDSVKGRFTISRDNSKNTLYLQMNSLRAEDTAVYYCAREYGSGSYYNVYYGMDVWGQGTTVTLSS

SEQ ID NO: 49 - protein, 10 aa, synthetic construct
  REGION 1..10: note = Description of Artificial Sequence: Synthetic peptide
GFTFSSYTLH

SEQ ID NO: 50 - protein, 17 aa, synthetic construct
  REGION 1..17: note = Description of Artificial Sequence: Synthetic peptide
VIWYDGSNKYYVDSVKG

SEQ ID NO: 51 - protein, 16 aa, synthetic construct
  REGION 1..16: note = Description of Artificial Sequence: Synthetic peptide
EYGSGSYYNVYYGMDV

SEQ ID NO: 52 - protein, 112 aa, synthetic construct
  REGION 1..112: note = Description of Artificial Sequence: Synthetic polypeptide
DIVMTQSPLSLPVTPGEPASISCRSSQSLLHSNGYNYLDWYLQKPGQSPHLLIYLASNRASGVPDRFSGSGSGTDFTLKISRVEAEDVGFYYCMQTLQIPLTFGGGTKVEIK

SEQ ID NO: 53 - protein, 16 aa, synthetic construct
  REGION 1..16: note = Description of Artificial Sequence: Synthetic peptide
RSSQSLLHSNGYNYLD

SEQ ID NO: 54 - protein, 7 aa, synthetic construct
  REGION 1..7: note = Description of Artificial Sequence: Synthetic peptide
LASNRAS

SEQ ID NO: 55 - protein, 9 aa, synthetic construct
  REGION 1..9: note = Description of Artificial Sequence: Synthetic peptide
MQTLQIPLT

SEQ ID NO: 56 - protein, 117 aa, synthetic construct
  REGION 1..117: note = Description of Artificial Sequence: Synthetic polypeptide
QVQLQESGPGLVKPSETLSLTCAVSGYSASGVYYWGWIRQPPGKGLEWIGNIYHSGSTYYNPSLERRVSISLDTSKNHFSLRLNFVTAADTAVYYCARDRFDAFDIWGQGTMVTVSS

SEQ ID NO: 57 - protein, 11 aa, synthetic construct
  REGION 1..11: note = Description of Artificial Sequence: Synthetic peptide
GYSASGVYYWG

SEQ ID NO: 58 - protein, 16 aa, synthetic construct
  REGION 1..16: note = Description of Artificial Sequence: Synthetic peptide
NIYHSGSTYYNPSLER

SEQ ID NO: 59 - protein, 8 aa, synthetic construct
  REGION 1..8: note = Description of Artificial Sequence: Synthetic peptide
DRFDAFDI

SEQ ID NO: 60 - protein, 107 aa, synthetic construct
  REGION 1..107: note = Description of Artificial Sequence: Synthetic polypeptide
EIVMTQSPATLSVSPGERATLSCRASQSINNILAWYQQKPGQAPRLLIYGASTRATGIPARFSGSGSGTEFTLTISSLQSEDFAVYYCQQYNNWPLTFGGGTKVEIK

SEQ ID NO: 61 - protein, 11 aa, synthetic construct
  REGION 1..11: note = Description of Artificial Sequence: Synthetic peptide
RASQSINNILA

SEQ ID NO: 62 - protein, 7 aa, synthetic construct
  REGION 1..7: note = Description of Artificial Sequence: Synthetic peptide
GASTRAT

SEQ ID NO: 63 - protein, 9 aa, synthetic construct
  REGION 1..9: note = Description of Artificial Sequence: Synthetic peptide
QQYNNWPLT

SEQ ID NO: 64 - protein, 121 aa, synthetic construct
  REGION 1..121: note = Description of Artificial Sequence: Synthetic polypeptide
QVQLVESGGGVVQPGRSLRLSCAASGFTFRSHGMHWVRQAPGKGLEWVAVIWYDGSSEYYADSVKGRFTISRDNSKNTLYLQMNSLRAEDTAVYYCARENVYGSGWFFDYWGQGTLVTVSS

SEQ ID NO: 65 - protein, 10 aa, synthetic construct
  REGION 1..10: note = Description of Artificial Sequence: Synthetic peptide
GFTFRSHGMH

SEQ ID NO: 66 - protein, 17 aa, synthetic construct
  REGION 1..17: note = Description of Artificial Sequence: Synthetic peptide
VIWYDGSSEYYADSVKG

SEQ ID NO: 67 - protein, 12 aa, synthetic construct
  REGION 1..12: note = Description of Artificial Sequence: Synthetic peptide
ENVYGSGWFFDY

SEQ ID NO: 68 - protein, 108 aa, synthetic construct
  REGION 1..108: note = Description of Artificial Sequence: Synthetic polypeptide
EIVMTQSPATLSVSPGERASLSCRASQSVGSNLAWYQQRPGQAPRLLIFGASTRATGIPARFSGSRSGTEFTLTISSLQSEDFALYYCQQYNNWPPWTFGQGTKLEIK

SEQ ID NO: 69 - protein, 11 aa, synthetic construct
  REGION 1..11: note = Description of Artificial Sequence: Synthetic peptide
RASQSVGSNLA

SEQ ID NO: 70 - protein, 10 aa, synthetic construct [sequence omitted]

SEQ ID NO: 71 - protein, 121 aa, synthetic construct
  REGION 1..121: note = Description of Artificial Sequence: Synthetic polypeptide
QVQLVESGGGVVQPGRSLRLSCAASGFTFRSHGMHWVRQAPGKGLEWVAVIWYDGSSDYYADSVKGRFTISRDNSKNTLFLQMNSLSAEDTAVYYCARENVYGSGWFFDYWGQGTLVTVSS

SEQ ID NO: 72 - protein, 17 aa, synthetic construct
  REGION 1..17: note = Description of Artificial Sequence: Synthetic peptide
VIWYDGSSDYYADSVKG

SEQ ID NO: 73 - protein, 108 aa, synthetic construct
  REGION 1..108: note = Description of Artificial Sequence: Synthetic polypeptide
EIVMTQSPATLSVSPGERASLSCRASQSVGSNLAWYQQKPGQAPRLLIFGASTRATGIPARFSGSRSGTEFTLTISSLQSEDFALYYCQQYNNWPPWTFGQGTKLEIK

SEQ ID NO: 74 - protein, 117 aa, synthetic construct
  REGION 1..117: note = Description of Artificial Sequence: Synthetic polypeptide
EVQLLESGGGLVQPGGSLRLSCAASGFTFSNYAMTWVRQAPGKGLEWVSSISISRAGTYYADSVKGRFTISRDNSKNTLNLQMNSLRAEDTAEYYCAREYYYGMDVWGQGTTVTVSS

SEQ ID NO: 75 - protein, 10 aa, synthetic construct
  REGION 1..10: note = Description of Artificial Sequence: Synthetic peptide
GFTFSNYAMT

SEQ ID NO: 76 - protein, 17 aa, synthetic construct
  REGION 1..17: note = Description of Artificial Sequence: Synthetic peptide
SISISRAGTYYADSVKG

SEQ ID NO: 77 - protein, 8 aa, synthetic construct
  REGION 1..8: note = Description of Artificial Sequence: Synthetic peptide
EYYYGMDV

SEQ ID NO: 78 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQTPRLLIYDASNRATGIPARFSGSGSGTDFTLTISSLETEDFAVYYCQQLINWPLTFGGGTKLEIK

SEQ ID NO: 79 - protein, 11 aa, synthetic construct
  REGION 1..11: note = Description of Artificial Sequence: Synthetic peptide
RASQSVSSYLA

SEQ ID NO: 80 - protein, 7 aa, synthetic construct
  REGION 1..7: note = Description of Artificial Sequence: Synthetic peptide
DASNRAT

SEQ ID NO: 81 - protein, 9 aa, synthetic construct
  REGION 1..9: note = Description of Artificial Sequence: Synthetic peptide
QQLINWPLT

SEQ ID NO: 82 - protein, 107 aa, synthetic construct
  REGION 1..107: note = Description of Artificial Sequence: Synthetic polypeptide
DIQMTQSPSSLSASVGDRVIITCRASQGISNYLAWFQQKPGKAPNLLIYAASTLRSGVPSRFSGSGSGTDFTLTISSLQPEDVATYYCQKYNSAPYTFGQGTKVEIK

SEQ ID NO: 83 - protein, 117 aa, synthetic construct
  REGION 1..117: note = Description of Artificial Sequence: Synthetic polypeptide
EVQLLESGADLVQPGGSLRLSCAASGFTFSSYAMSWVRQAPGKGLEWVSTIRISGGTTYYADSVKGRFTISRDNSKNTLYLQMNSLRAEDTAVYYCVRDYYNGMDVWGQGTTVTVSS

SEQ ID NO: 84 - protein, 10 aa, synthetic construct
  REGION 1..10: note = Description of Artificial Sequence: Synthetic peptide
GFTFSSYAMS

SEQ ID NO: 85 - protein, 17 aa, synthetic construct
  REGION 1..17: note = Description of Artificial Sequence: Synthetic peptide
TIRISGGTTYYADSVKG

SEQ ID NO: 86 - protein, 8 aa, synthetic construct
  REGION 1..8: note = Description of Artificial Sequence: Synthetic peptide
DYYNGMDV

SEQ ID NO: 87 - protein, 107 aa, synthetic construct
  REGION 1..107: note = Description of Artificial Sequence: Synthetic polypeptide
EIVLTQSPATLSLSPGERATLSCRASQRVSSYLAWYQQKPGQAPRLLIYDASNRATGIPARFSGSGSGTDFTLTISSLEPEDFAVYYCQQRSYWPPAFGQGTKLEIK

SEQ ID NO: 88 - protein, 11 aa, synthetic construct
  REGION 1..11: note = Description of Artificial Sequence: Synthetic peptide
RASQRVSSYLA

SEQ ID NO: 89 - protein, 9 aa, synthetic construct
  REGION 1..9: note = Description of Artificial Sequence: Synthetic peptide
QQRSYWPPA

SEQ ID NO: 90 - protein, 117 aa, synthetic construct
  REGION 1..117: note = Description of Artificial Sequence: Synthetic polypeptide
EVQLLESGGGLVQRGGSLRLSCAASGFTFSSYAMSWVRQAPGKGLEWVSSISGSGDSTNYADSVKGRFTISRDNSKNTLHLQMNSLRAEDTAVYYCVRDYYYGMDVWGHGTTVTVSS

SEQ ID NO: 91 - protein, 17 aa, synthetic construct
  REGION 1..17: note = Description of Artificial Sequence: Synthetic peptide
SISGSGDSTNYADSVKG

SEQ ID NO: 92 - protein, 8 aa, synthetic construct
  REGION 1..8: note = Description of Artificial Sequence: Synthetic peptide
DYYYGMDV

SEQ ID NO: 93 - protein, 107 aa, synthetic construct
  REGION 1..107: note = Description of Artificial Sequence: Synthetic polypeptide
EIVLTQSPATLSLSPGERATLSCRASQSVNSYLAWYQQKPGQAPRLLIYDASNRATGIPARFSGSGSGTDFTLTISSLEPEDFAVYYCQQRSYWPITFGQGTRLEIK

SEQ ID NO: 94 - protein, 11 aa, synthetic construct
  REGION 1..11: note = Description of Artificial Sequence: Synthetic peptide
RASQSVNSYLA

SEQ ID NO: 95 - protein, 9 aa, synthetic construct
  REGION 1..9: note = Description of Artificial Sequence: Synthetic peptide
QQRSYWPIT

SEQ ID NO: 96 - protein, 117 aa, synthetic construct
  REGION 1..117: note = Description of Artificial Sequence: Synthetic polypeptide
EAQLLESGGGLVQPGGSLRLSCAASGFTFSSYAMSWVRQAPGKGLEWVSTISGSGGSTHYADSVKGRFTISRDNSKNTLYLQMNSLRAEDTAVYYCVRDYYYGMDVWGQGTTVTVSS

SEQ ID NO: 97 - protein, 17 aa, synthetic construct
  REGION 1..17: note = Description of Artificial Sequence: Synthetic peptide
TISGSGGSTHYADSVKG

SEQ ID NO: 98 - protein, 107 aa, synthetic construct
  REGION 1..107: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSYWPPTFGQGTKLEIK

SEQ ID NO: 99 - protein, 9 aa, synthetic construct
  REGION 1..9: note = Description of Artificial Sequence: Synthetic peptide
QQRSYWPPT

SEQ ID NO: 100 - protein, 121 aa, synthetic construct
  REGION 1..121: note = Description of Artificial Sequence: Synthetic polypeptide
QVQLVESGGGVVQPGRSLRLSCAASGFTFSSYGMHWVRQAPGKGLEWVAVIWYDGSKKYYVDSVQGRFTISRDNSKNTLYLQMNSLRGEDTAVYYCARDGGAVADNWIDSWGQGTLVTVSS

SEQ ID NO: 101 - protein, 10 aa, synthetic construct
  REGION 1..10: note = Description of Artificial Sequence: Synthetic peptide
GFTFSSYGMH

SEQ ID NO: 102 - protein, 17 aa, synthetic construct
  REGION 1..17: note = Description of Artificial Sequence: Synthetic peptide
VIWYDGSKKYYVDSVQG

SEQ ID NO: 103 - protein, 107 aa, synthetic construct
  REGION 1..107: note = Description of Artificial Sequence: Synthetic polypeptide
DIQMTQSPSSLSASVGDRVTLTCRASQNIRSYLNWYQQKPGKAPKLLIFAASSLPSGVPSRFSGSGSVTDFTLTVSSLQPEDFATYSCQQSYSTPYTFGQGTKLEIK

SEQ ID NO: 104 - protein, 11 aa, synthetic construct
  REGION 1..11: note = Description of Artificial Sequence: Synthetic peptide
RASQNIRSYLN

SEQ ID NO: 105 - protein, 7 aa, synthetic construct
  REGION 1..7: note = Description of Artificial Sequence: Synthetic peptide
AASSLPS

SEQ ID NO: 106 - protein, 117 aa, synthetic construct
  REGION 1..117: note = Description of Artificial Sequence: Synthetic polypeptide
EEQLLESGADLAQPGGSLRLSCAASGFTFSSYAMSWVRQAPGKGLEWVSTIRISGDTTYYADSVKGRFTISRDNSKNTLFLQMNSLRAEDTAVYYCVRDYYNGMDVWGHGTTVTVSS

SEQ ID NO: 107 - protein, 17 aa, synthetic construct
  REGION 1..17: note = Description of Artificial Sequence: Synthetic peptide
TIRISGDTTYYADSVKG

SEQ ID NO: 108 - protein, 107 aa, synthetic construct; identical to SEQ ID NO: 87
  REGION 1..107: note = Description of Artificial Sequence: Synthetic polypeptide

SEQ ID NO: 109 - protein, 121 aa, synthetic construct
  REGION 1..121: note = Description of Artificial Sequence: Synthetic polypeptide
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCAREAVAGTSDAFDIWGQGTMVTVSS

SEQ ID NO: 110 - protein, 10 aa, synthetic construct
  REGION 1..10: note = Description of Artificial Sequence: Synthetic peptide
GYTFTGYYMH

SEQ ID NO: 111 - protein, 17 aa, synthetic construct
  REGION 1..17: note = Description of Artificial Sequence: Synthetic peptide
WINPNSGGTNYAQKFQG

SEQ ID NO: 112 - protein, 12 aa, synthetic construct
  REGION 1..12: note = Description of Artificial Sequence: Synthetic peptide
EAVAGTSDAFDI

SEQ ID NO: 113 - protein, 107 aa, synthetic construct
  REGION 1..107: note = Description of Artificial Sequence: Synthetic polypeptide
DIQMTQSPSSLSASVGDRVTITCRANQSIASYLNWYQQKPGKVPKLLIYGASSLQNGVPSRFSGSGSRTDFTLTISSLQPEDFATYYCQQSYSTEITFGQGTRLEIK

SEQ ID NO: 114 - protein, 107 aa, synthetic construct
  REGION 1..107: note = Description of Artificial Sequence: Synthetic polypeptide
DIQMTQSPSSLSASVGDRVAITCRASQSIASYLNWYQQKPGKAPKLLIYGASSLQNGVPSRFSGSGSRTDFTLTISSLQPEDFATYYCQQSYSTEITFGQGTRLEIK

SEQ ID NO: 115 - protein, 11 aa, synthetic construct
  REGION 1..11: note = Description of Artificial Sequence: Synthetic peptide
RASQSIASYLN

SEQ ID NO: 116 - protein, 120 aa, synthetic construct
  REGION 1..120: note = Description of Artificial Sequence: Synthetic polypeptide
QVQLVESGGGVVQPGRSLRLSCAASGFTFRNYGMHWVRQAPGKGLEWVAVIWYDGSNKFYADSVKGRFTISRDNSKNTLYLQMNSLRVEDTAVYYCAREEGIAVAPFDYWGQGTLVTVSS

SEQ ID NO: 117 - protein, 10 aa, synthetic construct
  REGION 1..10: note = Description of Artificial Sequence: Synthetic peptide
GFTFRNYGMH

SEQ ID NO: 118 - protein, 121 aa, synthetic construct
  REGION 1..121: note = Description of Artificial Sequence: Synthetic polypeptide
QVQLVESGGGMVQPGRSLRLSCTASGFTFSSHGMHWVRQAPGKGLEWVAVIWFDGSNEYYVDSVKGRFTISRDNSKNTLYLQMNSLRAEDTAVYYCARENVYGSGWFFDYWGQGTLVTVSS

SEQ ID NO: 119 - protein, 17 aa, synthetic construct
  REGION 1..17: note = Description of Artificial Sequence: Synthetic peptide
VIWFDGSNEYYVDSVKG

SEQ ID NO: 120 - protein, 108 aa, synthetic construct
  REGION 1..108: note = Description of Artificial Sequence: Synthetic polypeptide
EIVMTQSPATLSVSPGERASLSCRASQSVGSNLAWYQQKPGQAPRLLIFGASTRATGIPARFSGSRSGTEFTLTISSLQSEDFALYYCQQYNNWPPWTFGQGTKVEIK

SEQ ID NO: 121 - protein, 121 aa, synthetic construct
  REGION 1..121: note = Description of Artificial Sequence: Synthetic polypeptide
QLQLVESGGGVVQPGRSLRLSCAASGFTFSSYGMHWVRQAPGKGLEWVAVIWYDGSSEYYADSVKGRFTISRDNSKNTLYLQMNSLRAEDTAVYYCARENVYGSGWFFDYWGQGTLVTVSS

SEQ ID NO: 122 - protein, 108 aa, synthetic construct; identical to SEQ ID NO: 120
  REGION 1..108: note = Description of Artificial Sequence: Synthetic polypeptide

SEQ ID NO: 123 - protein, 121 aa, synthetic construct
  REGION 1..121: note = Description of Artificial Sequence: Synthetic polypeptide
QLQLVESGGGVVQPGRSLRLSCAASGFTFSSHGMHWVRQAPGKGLEWVAVIWYDGSSEYYADSVKGRFTISRDNSKNTLYLQMNSLRAEDTAVYYCARENVYGSGWFFDYWGQGSLVTVSS

SEQ ID NO: 124 - protein, 108 aa, synthetic construct; identical to SEQ ID NO: 73
  REGION 1..108: note = Description of Artificial Sequence: Synthetic polypeptide

SEQ ID NO: 125 - protein, 121 aa, synthetic construct
  REGION 1..121: note = Description of Artificial Sequence: Synthetic polypeptide
QLQLVESGGGVVQFGRSLRLSCAASGFTFSSHGMHWVRQAPGKGLEWVAVIWYDGSSEYYADSVKGRFTISRDNSKNTLYLQMNSLRAEDTAVYYCARENVYGSGWFFDYWGQGTLVTVSS

SEQ ID NO: 126 - protein, 108 aa, synthetic construct; identical to SEQ ID NO: 73
  REGION 1..108: note = Description of Artificial Sequence: Synthetic polypeptide

SEQ ID NO: 127 - protein, 121 aa, synthetic construct
  REGION 1..121: note = Description of Artificial Sequence: Synthetic polypeptide
QLQLVESGGGVVQPGRSLRLSCAASGFTFSSYGMHWVRQAPGKGLEWVAVIWYDGSSEYYADSVKGRFTISRDNSKNTLFLQMNSLRADDTAVYYCARENVYGSGWFFDYWGQGTLVTVSS

SEQ ID NO: 128 - protein, 108 aa, synthetic construct
  REGION 1..108: note = Description of Artificial Sequence: Synthetic polypeptide
EIVMTQSPATLSVSPGERASLSCRASQSVGSNLAWYQQKPGQAPRLLIFGASTRATGIPARFSGSRSGTEFTLTISSLQSEDFALYYCQQYDNWPPWTFGQGTKLEIK

SEQ ID NO: 129 - protein, 10 aa, synthetic construct
  REGION 1..10: note = Description of Artificial Sequence: Synthetic peptide
QQYDNWPPWT

SEQ ID NO: 130 - protein, 121 aa, synthetic construct
  REGION 1..121: note = Description of Artificial Sequence: Synthetic polypeptide
QVQLVESGGGVVQPGRSLRLSCAASGFTLSGYGMHWVRQAPGKGLEWVAVIWYDGSNKYYADSVKGRFTISRDNSKNMLFLQMNSLRAEDTAVYYCVRENGFGSGWFFDYWGQGNLVTVSS

SEQ ID NO: 131 - protein, 10 aa, synthetic construct
  REGION 1..10: note = Description of Artificial Sequence: Synthetic peptide
GFTLSGYGMH

SEQ ID NO: 132 - protein, 17 aa, synthetic construct
  REGION 1..17: note = Description of Artificial Sequence: Synthetic peptide
VIWYDGSNKYYADSVKG

SEQ ID NO: 133 - protein, 12 aa, synthetic construct
  REGION 1..12: note = Description of Artificial Sequence: Synthetic peptide
ENGFGSGWFFDY

SEQ ID NO: 134 - protein, 108 aa, synthetic construct
  REGION 1..108: note = Description of Artificial Sequence: Synthetic polypeptide
EIVMTQSPATLSVSPGERATLSCRTSQSVSRDLAWYQQKPGQAPRLLIYGASTRATGIPVRFSGSGSGTEFTLTISSLQSEDFAVYYCQQYNNWPPWTFGQGTKLEIK

SEQ ID NO: 135 - protein, 11 aa, synthetic construct
  REGION 1..11: note = Description of Artificial Sequence: Synthetic peptide
RTSQSVSRDLA

SEQ ID NO: 136 - protein, 125 aa, synthetic construct
  REGION 1..125: note = Description of Artificial Sequence: Synthetic polypeptide
QGQLVESGGGVVQPGRSLRLSCAASGFTFSSYTLHWVRQAPGKGLEWVAVIWYDGSNKYYVDSVKGRFTISRDNSKNTLYLQMNSLRAEDTAVYYCAREYGSGSYYNVYYGMDVWGQGTTVTVSS

SEQ ID NO: 137 - protein, 112 aa, synthetic construct
  REGION 1..112: note = Description of Artificial Sequence: Synthetic polypeptide
DIVMTQSPLSLPVTPGEPASISCRSSQSLLHSNGYKYLDWYLQKAGQSPHLLIYLGSNRASGVPDRFSGSGSGTDFTLKISRVEAEDVGFYYCMQTLQIPLTFGGGTKLEIK

SEQ ID NO: 138 - protein, 16 aa, synthetic construct
  REGION 1..16: note = Description of Artificial Sequence: Synthetic peptide
RSSQSLLHSNGYKYLD

SEQ ID NO: 139 - protein, 7 aa, synthetic construct
  REGION 1..7: note = Description of Artificial Sequence: Synthetic peptide
LGSNRAS

SEQ ID NO: 140 - protein, 120 aa, synthetic construct
  REGION 1..120: note = Description of Artificial Sequence: Synthetic polypeptide
EVQVVESGGGLVQPGGSLRLSCATSGFTFRSYWMTWVRQAPGKGLEWVANIKQDGSERHYVDSVKGRFTISRDNAKTSLYLQMSSLRAEDTAMYYCAREGYYGSGYFDYWGQGTLVTVSS

SEQ ID NO: 141 - protein, 10 aa, synthetic construct
  REGION 1..10: note = Description of Artificial Sequence: Synthetic peptide
GFTFRSYWMT

SEQ ID NO: 142 - protein, 11 aa, synthetic construct
  REGION 1..11: note = Description of Artificial Sequence: Synthetic peptide
EGYYGSGYFDY

SEQ ID NO: 143 - protein, 107 aa, synthetic construct
  REGION 1..107: note = Description of Artificial Sequence: Synthetic polypeptide
DIQMTQSPSTLSASVGDRVIITCRASQSISRGLAWYQQKPGKAPKLLIYKASNLESGVPSRFSGSGSGTEFTLTISSLQPDDFAAYYCQQYSYYSHTFGQGTKVEIK

SEQ ID NO: 144 - protein, 120 aa, synthetic construct
  REGION 1..120: note = Description of Artificial Sequence: Synthetic polypeptide
EVQLVESGGGLVQPGGSLRLSCAASGFTLSFYWMTWVRQAPGKGLEWVANIKQDGNEKNYVDSVKGRFTISKDNAKKSVFLQMNSLRAEDTAVYYCAREGYFGSGYFDYWGQGTLVTVSS

SEQ ID NO: 145 - protein, 10 aa, synthetic construct
  REGION 1..10: note = Description of Artificial Sequence: Synthetic peptide
GFTLSFYWMT

SEQ ID NO: 146 - protein, 17 aa, synthetic construct
  REGION 1..17: note = Description of Artificial Sequence: Synthetic peptide
NIKQDGNEKNYVDSVKG

SEQ ID NO: 147 - protein, 107 aa, synthetic construct
  REGION 1..107: note = Description of Artificial Sequence: Synthetic polypeptide
DIQMTQSPSTLSASVGDRVTITCRASQSINSWLAWYQQKPGKAPKLLIYKASTLESGVPSRFSGSGSGTEFTLTISSLQPDDFAPYYCQHYNSYPHTFGQGTKVEIK

SEQ ID NO: 148 - protein, 11 aa, synthetic construct
  REGION 1..11: note = Description of Artificial Sequence: Synthetic peptide
RASQSINSWLA

SEQ ID NO: 149 - protein, 7 aa, synthetic construct
  REGION 1..7: note = Description of Artificial Sequence: Synthetic peptide
KASTLES

SEQ ID NO: 150 - protein, 9 aa, synthetic construct
  REGION 1..9: note = Description of Artificial Sequence: Synthetic peptide
QHYNSYPHT

SEQ ID NO: 151 - protein, 120 aa, synthetic construct
  REGION 1..120: note = Description of Artificial Sequence: Synthetic polypeptide
EVQLVESGGGLVQPGGSLRLSCAASGFTFSSYWMSWVRQAPGKGLEWVANIKQDGSEKHYVDSVKGRFTISRDNAKNSLYLQMNSLRAEDTAVYYCAREGYFGSGYFDYWGQGTLVTVSS

SEQ ID NO: 152 - protein, 10 aa, synthetic construct
  REGION 1..10: note = Description of Artificial Sequence: Synthetic peptide
GFTFSSYWMS

SEQ ID NO: 153 - protein, 17 aa, synthetic construct
  REGION 1..17: note = Description of Artificial Sequence: Synthetic peptide
NIKQDGSEKHYVDSVKG

SEQ ID NO: 154 - protein, 107 aa, synthetic construct
  REGION 1..107: note = Description of Artificial Sequence: Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQYSFYSHTFGQGTKLEIK

SEQ ID NO: 155 - protein, 11 aa, synthetic construct
  REGION 1..11: note = Description of Artificial Sequence: Synthetic peptide
RASQGISSYLA

SEQ ID NO: 156 - protein, 7 aa, synthetic construct
  REGION 1..7: note = Description of Artificial Sequence: Synthetic peptide
AASTLQS

SEQ ID NO: 157 - protein, 9 aa, synthetic construct
  REGION 1..9: note = Description of Artificial Sequence: Synthetic peptide
QQYSFYSHT

SEQ ID NO: 158 - protein, 120 aa, synthetic construct
  REGION 1..120: note = Description of Artificial Sequence: Synthetic polypeptide
EVQLVESGGGLVQPGGSLRLSCAASGFTLSFYWMTWVRQAPGKGLEWVANIKQDGNEKNFVDSVKGRFTISRDNAKKSVFLQMNSLRAEDTAVYYCAREGYFGSGYFDYWGQGTLVTVSS

SEQ ID NO: 159 - protein, 17 aa, synthetic construct
  REGION 1..17: note = Description of Artificial Sequence: Synthetic peptide
NIKQDGNEKNFVDSVKG

SEQ ID NO: 160 - protein, 107 aa, synthetic construct
  REGION 1..107: note = Description of Artificial Sequence: Synthetic polypeptide
DIQMTQSPSTLSASVGDRVTITCRASQSINSWLAWYQQKPGKAPKLLIYKASSLESGVPSRFSGSGSGTEFTLTISSLQPDDFATYYCQQYNSYPHTFGQGTKLEIK

SEQ ID NO: 161 - protein, 7 aa, synthetic construct
  REGION 1..7: note = Description of Artificial Sequence: Synthetic peptide
KASSLES

SEQ ID NO: 162 - protein, 9 aa, synthetic construct
  REGION 1..9: note = Description of Artificial Sequence: Synthetic peptide
QQYNSYPHT

SEQ ID NO: 163 - protein, 119 aa, synthetic construct
  REGION 1..119: note = Description of Artificial Sequence: Synthetic polypeptide
QVQLQESGPGLVKPLGTLSLTCAVSGGSISNSNWWSWVRQPPGKGLEWIGEIYHSGSTNYNPSLKSRVTIALDWSKNQFSLQLRSVTAADTAVYYCARYGSGPFGGDCWGQGTLVTVSS

SEQ ID NO: 164 - protein, 11 aa, synthetic construct
  REGION 1..11: note = Description of Artificial Sequence: Synthetic peptide
GGSISNSNWWS

SEQ ID NO: 165 - protein, 16 aa, synthetic construct
  REGION 1..16: note = Description of Artificial Sequence: Synthetic peptide
EIYHSGSTNYNPSLKS

SEQ ID NO: 166 - protein, 10 aa, synthetic construct
  REGION 1..10: note = Description of Artificial Sequence: Synthetic peptide
YGSGPFGGDC

SEQ ID NO: 167 - protein, 107 aa, synthetic construct
  REGION 1..107: note = Description of Artificial Sequence: Synthetic polypeptide
DIQLTQSPSFLSASVGDRVTISCRASQGISSYLAWYQQKPGKAPKLLIYAASTLQGGVPSRFSGSGSGTTFTLTISSLQPEDFATYYCQQLNTYPFTFGPGTKLEIK

SEQ ID NO: 168 - protein, 7 aa, synthetic construct
  REGION 1..7: note = Description of Artificial Sequence: Synthetic peptide
AASTLQG

SEQ ID NO: 169 - protein, 9 aa, synthetic construct
  REGION 1..9: note = Description of Artificial Sequence: Synthetic peptide
QQLNTYPFT

SEQ ID NO: 170 - protein, 119 aa, synthetic construct
  REGION 1..119: note = Description of Artificial Sequence: Synthetic polypeptide
QVQLQESGPGLVKPLGTLSLTCAVSGGSISNSNWWSWVRQPPGKGLEWIGEIYHSGSTNYNPSLKSRVTIALDWSKNQFSLQLRSVTAADTAVYYCARYGSGPFGGDYWGQGTLVTVSS

SEQ ID NO: 171 - protein, 10 aa, synthetic construct
  REGION 1..10: note = Description of Artificial Sequence: Synthetic peptide
YGSGPFGGDY

SEQ ID NO: 172 - protein, 107 aa, synthetic construct; identical to SEQ ID NO: 167
  REGION 1..107: note = Description of Artificial Sequence: Synthetic polypeptide

SEQ ID NO: 173 - protein, 123 aa, synthetic construct
  REGION 1..123: note = Description of Artificial Sequence: Synthetic polypeptide
QVQIKESGPGLVKPSETLSLTCAVSGFSFSSGYYWGWIRQPPGKGLEWLGSFFHNGNTYYNPSLRSRVTISVDTSKNHFSLKLTSVTAADTAVYYCAGFGDLPHYHYYVMDVWGQGTTVTVSS

SEQ ID NO: 174 - protein, 11 aa, synthetic construct
  REGION 1..11: note = Description of Artificial Sequence: Synthetic peptide
GFSFSSGYYWG

SEQ ID NO: 175 - protein, 16 aa, synthetic construct
  REGION 1..16: note = Description of Artificial Sequence: Synthetic peptide
SFFHNGNTYYNPSLRS

SEQ ID NO: 176 - protein, 14 aa, synthetic construct
  REGION 1..14: note = Description of Artificial Sequence: Synthetic peptide
FGDLPHYHYYVMDV

SEQ ID NO: 177 - protein, 108 aa, synthetic construct
  REGION 1..108: note = Description of Artificial Sequence: Synthetic polypeptide
EIVLTQSPATLSLSPGERATLSCRASQSVSSYLAWYQQKPGQAPRLLIYDASNRAIGIPARFSGSGSGTDFTLTISSLEPEDFAVYYCQQRSNWPPYTFGQGTKLEIK

SEQ ID NO: 178 - protein, 7 aa, synthetic construct
  REGION 1..7: note = Description of Artificial Sequence: Synthetic peptide
DASNRAI

SEQ ID NO: 179 - protein, 10 aa, synthetic construct
  REGION 1..10: note = Description of Artificial Sequence: Synthetic peptide
QQRSNWPPYT

SEQ ID NO: 180 - protein, 123 aa, synthetic construct
  REGION 1..123: note = Description of Artificial Sequence: Synthetic polypeptide
QVQPKESGPGVVKPSETLSLTCAVSGFPISRGYYWGWIRQPPGKGLEWIGNIFHSGTTYYNPSLKSRVTISVDTSKNQISLKLTSVTAADTAVYYCVGFGDLPHYQYYVMDIWGQGTTVTVSS

SEQ ID NO: 181 - protein, 11 aa, synthetic construct
  REGION 1..11: note = Description of Artificial Sequence: Synthetic peptide
GFPISRGYYWG

SEQ ID NO: 182 - protein, 16 aa, synthetic construct
  REGION 1..16: note = Description of Artificial Sequence: Synthetic peptide
NIFHSGTTYYNPSLKS

SEQ ID NO: 183 - protein, 14 aa, synthetic construct
  REGION 1..14: note = Description of Artificial Sequence: Synthetic peptide
FGDLPHYQYYVMDI

SEQ ID NO: 184 - protein, 108 aa, synthetic construct
  REGION 1..108: note = Description of Artificial Sequence: Synthetic polypeptide
EIVLTQSPATLSLSPGERATLSCRASQSVSSYLAWYQQKPGQAPRLLIYDASNRATGIPARFSGSGSGTDFTLTISSLEPEDFAVYYCQQRSNWPPYTFGQGTKLEIK

SEQ ID NO: 185 - protein, 117 aa, synthetic construct
  REGION 1..117: note = Description of Artificial Sequence: Synthetic polypeptide
QVQLQESGPGLVKPSETLSLTCAVSGYSASGLYYWAWIRQPPGKGLEWIGNIYHSGRTYYNPSLESRVSISLDTSKHQVSLKLKSVTYADTAVYFCARDRFDGFDIWGQGTMVTVSS

SEQ ID NO: 186 - protein, 11 aa, synthetic construct
  REGION 1..11: note = Description of Artificial Sequence: Synthetic peptide
GYSASGLYYWA

SEQ ID NO: 187 - protein, 16 aa, synthetic construct
  REGION 1..16: note = Description of Artificial Sequence: Synthetic peptide
NIYHSGRTYYNPSLES

SEQ ID NO: 188 - protein, 8 aa, synthetic construct
  REGION 1..8: note = Description of Artificial Sequence: Synthetic peptide
DRFDGFDI

SEQ ID NO: 189 - protein, 107 aa, synthetic construct
  REGION 1..107: note = Description of Artificial Sequence: Synthetic polypeptide
EIVMTQSPATLSVSPGERATLSCRASQTINNILAWYQQKPGQAPRLLIYGASTRATGIPARFSGSGSGTEFTLTISSLQSEDFAVYYCQQYNNWPLTFGGGTKLEIK

SEQ ID NO: 190 - protein, 11 aa, synthetic construct
  REGION 1..11: note = Description of Artificial Sequence: Synthetic peptide
RASQTINNILA

SEQ ID NO: 191 - DNA, 342 bp, synthetic construct
  misc_feature 1..342: note = Description of Artificial Sequence: Synthetic polynucleotide
  source 1..342: mol_type = other DNA
caggtggagctgcaggagtcgggcccaggactggtgaagccttcggacaccctgtccctcacctgcactgtcactggtggctccatcagtacttactactggagctggatccggcagcccccagggaagggactggagtggattgggtatatcttttacactgggaccaccaactacaacccctccctcaagagtcgagtcaccatatcagtagacgcgtccaagaaccagttctccctgaagttgaactctgtgaccgctgcggacacggccgtgtattactgtgcgagagatgggagcctggactactggggccagggagccctggtcaccgtctcctca

SEQ ID NO: 192 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcaccatcacttgccgggcaagtcagagcattagcaactatttaaattggtatcagcagaaaccagggaaagcccctaagctcctgatctttgttgcatcccttttgcaaagtggggtcccatcaaggttcagtggcagtggatctgggacagatttcactctcaccatcagcagtctgcaacctgaagattttgcaacttactactgtcaacagagttacagtaccccattcactttcggccctgggaccaagctggagatcaaa

SEQ ID NO: 193 - DNA, 372 bp, synthetic construct
  misc_feature 1..372: note = Description of Artificial Sequence: Synthetic polynucleotide
  source 1..372: mol_type = other DNA
caggtgcagctggtggagtctgggggaggcgtggtccagcctgggaggtccctgagaatctcctgtgcagcgtctggattcaccttcaggagctatggcatgcactgggtccgccaggctccaggcaagggactggagtgggtggcaattatctggcatgatgaaagttataaatattatgtagactccgtgaagggccgattcaccatctccagagacaattccaagaacacgctgtatctgcaaatgaacagcctgagagccgaggacacggctgtgtattattgtgcgagaggcgattactatggttcggggagttattatgatgcttttgatatctggggccaagggacaatggtcaccgtctcctca

SEQ ID NO: 194 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcatcatcacttgccgggcgagtcaaggcattagcaattatttagcctggtttcagcagaaaccagggaaatgtcctaacctcctgatctatgctgcatccactttgcgatcaggggtcccatctcggttcagtggcagtggatctgggacagatttcactctcaccatcagcagcctgcagcctgaagatgttgcaacttattactgtcaaaagtataacagtgccccgtacacttttggccaggggaccaaggtggagatcaaa

SEQ ID NO: 195 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtccctgagactctcctgtgcagcgtctggattcaccttcagtcactatggcatgcactgggtccgccaggctccaggcaaggggctggagtgggtggcagttatatggtatgatggaagtaaaaaatactatgtagactccgtgaagggccgattcaccatctccagagacaattccaagaacacgctgtatctgcaaatgaacagcctgagaggcgaggacacggctgtgtattactgtgcgagagatgggggagcagtggctgacaactggatcgactcctggggccagggaaccctggtcaccgtctcctca

SEQ ID NO: 196 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcactatcacttgccgggcaagtcagagcattagaagctatttaaattggtatcagcagaaacccgggaaagcccctaagctcctgatctttgctgcatccagtttgcaaagtggggtcccatccaggttcagtggcagtggatctgggacagatttcactctcaccgtcagcagtctgcaacctgaagattttgcaacttactcctgtcaacaaagttacagtaccccgtacacttttggccaggggaccaaggtggagatcaaa

SEQ ID NO: 197 - DNA, 360 bp, synthetic construct
  misc_feature 1..360: note = Description of Artificial Sequence: Synthetic polynucleotide
  source 1..360: mol_type = other DNA
caggtgcagttggtggagtctgggggaggcgtggtccagcctggggggtccctgagactctcctgtgcagcgtctggattcaccttcagtagccatggcatgcactgggtccgccaggctccaggcaaggggctggagtgggtggcagttatatggtatgatggaagtaataaattctatacagactccgtgcagggccgattcaccatttccagagacaattccaagaacacgctgaatctgcaaatgaacagtctgagagccgaggacacggctgtatattactgtgcgagagagggtttgagggccgggtactactttgacttctggggccagggaaccctggtcaccgtctcctca

SEQ ID NO: 198 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcaccatcacttgccgggcaagtcagaggattagcttctattcaaattggtatcagcagaaaccagggaaagcccctaagctcctgatctatgctgcatccagtttgcaaagtggggtcccatcaaggttcagtggcagtggctctgggacagatttcactctcaccatcagcagtctgcaacctgaagattttgcaacttacttctgtcaacagagtcacagtaccccgctcactttcggcggagggaccaagctggagatcaaa

SEQ ID NO: 199 - DNA, 360 bp, synthetic construct
  misc_feature 1..360: note = Description of Artificial Sequence: Synthetic polynucleotide
  source 1..360: mol_type = other DNA
caggtgcagctggtggagtctgggggaggcgtggtccagcctgggaggtccctgcgactctcctgtgcagcgtctggattcaccttcaggaactatggcattcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagtcgaggacacggctgtatattactgtgcgagagaggaggggatagcagtggccccctttgactactggggccagggaaccctggtcaccgtctcctca

SEQ ID NO: 200 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcgccatcacttgccgggcaaatcagagtattgccagttatttaaattggtatcagcagaaaccagggaaagcccctaaactcctgatctatggtgcatccagtttgcaaaatggggtcccatcaaggttcagtggcagtggatctaggacagatttcactctcaccatcagcagtctgcaacctgaagattttgcaacttactactgtcaacagagttacagcaccgagatcaccttcggccaagggacacgactggagattaaa

SEQ ID NO: 201 - DNA, 360 bp, synthetic construct
  misc_feature 1..360: note = Description of Artificial Sequence: Synthetic polynucleotide
  source 1..360: mol_type = other DNA
gaggtgcaggtggtggagtctgggggaggcttggtccagcctggggggtccctgagactctcctgtgcagcctctggattcacctttagaagctattggatgagctgggtccgccaggctccagggaaggggctggagtgggtggccaacataaagcaggatggaagtgagagacactatgtggactctgtgaagggccgattcaccatctccagagacaacgccaagacatcactgtatctgcaaatgagcagcctgagagccgaggacacggctgtgtattactgtgcgagagaggggtactttggttcgggctactttgactactggggccagggaaccctggtcaccgtctcctca

SEQ ID NO: 202 - DNA, 321 bp, synthetic construct
  misc_feature 1..321: note = Description of Artificial Sequence: Synthetic polynucleotide
  source 1..321: mol_type = other DNA
gacatccagatgacccagtctccctccaccctgtctgcatctgtaggagacagagtcatcatcacttgccgggccagtcagagtattagtagggggttggcctggtatcagcagaaaccagggaaagcccctaaactcttgatctataaggcgtctaatttagaaagtggggtcccatcaaggttcagcggcagtggatctgggacagaattcactctcaccatcagcagcctgcagcctgatgattttgcagcttattactgccaacagtatagttattattctcacacttttggccaggggaccaagctggagatcaaa

SEQ ID NO: 203 - DNA, 375 bp, synthetic construct
  misc_feature 1..375: note = Description of Artificial Sequence: Synthetic polynucleotide
  source 1..375: mol_type = other DNA
caggggcaggtggtggagtctgggggaggcgtggtccagcctgggaggtccctgagactctcctgtgcagcgtctggattcaccttcagtagttataccttgcactgggtccgccaggctccaggcaaggggctggagtgggtggcagtcatatggtatgatggaagtaataagtattatgtagactccgtgaagggccgattcaccatctccagagacaattccaagaacacgctgtatctgcaaatgaacagcctgagagccgaggacacggctgtgtattactgtgcgagagagtatggttcggggagttattataacgtctactacggtatggacgtctggggccaggggaccacggtcaccctctcctca

SEQ ID NO: 204 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctccttcatagtaatggatacaactatttggattggtatctgcagaagccaggccagtctccacacctcttgatctatttggcttctaatcgggcctccggggtccctgacaggttcagtggcagtggatcaggcacagattttacactgaaaatcagcagagtggaggctgaggatgttgggttttattactgcatgcaaactctacaaattccgctcactttcggcggagggaccaaggtggagatcaaa

SEQ ID NO: 205 - DNA, 351 bp, synthetic construct
  misc_feature 1..351: note = Description of Artificial Sequence: Synthetic polynucleotide
  source 1..351: mol_type = other DNA
caggtgcagctgcaggagtcgggcccaggactggtgaagccttcggagaccctgtccctcacctgcgctgtctctggttactccgccagcggtgtttactactggggctggatccggcagcccccagggaaggggctggagtggattggaaatatctatcatagtgggagcacctattacaacccgtccctcgagaggcgagttagtatatcactagacacgtccaagaaccacttctccctgaggctgaattttgtgaccgccgcagacacggccgtgtattactgtgcgagagataggtttgatgcttttgatatctggggccaagggacaatggtcaccgtctcctca

SEQ ID NO: 206 - DNA, 321 bp, synthetic construct
  misc_feature 1..321: note = Description of Artificial Sequence: Synthetic polynucleotide
  source 1..321: mol_type = other DNA
gaaatagtgatgacgcagtctccagccaccctgtctgtgtctccaggggaaagagccaccctctcctgcagggccagtcagagtattaacaacatcttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaaggtggagatcaaa

SEQ ID NO: 207 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtccctgagactctcctgtgcagcgtctggattcacctttaggagtcatggcatgcactgggtccgccaggctccaggcaagggactggagtgggtggcagttatatggtatgatggaagtagtgagtactatgcagactccgtgaagggccgattcaccatctccagagacaattccaagaacacgctgtatctgcaaatgaacagcctgagagccgaggacacggctgtgtattattgtgcgagagaaaacgtgtatggcagtggctggttttttgactactggggccagggaaccctggtcaccgtctcctca

SEQ ID NO: 208 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagcctccctctcctgcagggccagtcagagtgttggcagcaacttagcctggtaccagcagagacctggccaggctcccaggctcctcatctttggtgcatccaccagggccactggtatcccagccaggttcagtggcagtaggtctgggacagagttcactctcaccatcagcagcctgcagtctgaagattttgcactttattactgtcagcagtataataactggcctccgtggacgttcggccaagggaccaagctggagatcaaa

SEQ ID NO: 209 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtccctgagactctcctgtgcagcgtctggattcaccttcaggagtcatggcatgcactgggtccgccaggcaccaggcaagggactggagtgggtggcagttatatggtatgatggaagtagtgactactatgcagactccgtgaagggccgattcaccatctccagagacaattccaagaacacactatttctgcaaatgaacagcctgagcgccgaggacacggctgtgtattattgtgcgagagaaaacgtgtatggcagtggctggttttttgactactggggccagggaaccctggtcaccgtctcctca

SEQ ID NO: 210 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagcctccctctcctgcagggccagtcagagtgttggcagcaacttagcctggtaccagcagaaacctggccaggctcccaggctcctcatctttggtgcatccaccagggccactggtatcccagccaggttcagtggcagtaggtctgggacagagttcactctcaccatcagcagcctgcagtctgaagattttgcactttattactgtcagcagtataataactggcctccgtggacgttcggccaagggaccaagctggagatcaaa

SEQ ID NO: 211 - DNA, 351 bp, synthetic construct
  misc_feature 1..351: note = Description of Artificial Sequence: Synthetic polynucleotide
  source 1..351: mol_type = other DNA
gaggtgcagctgttggagtctgggggaggcttggtacagcctggggggtccctgagactctcctgtgcagcctctggattcacctttagcaactatgccatgacctgggtccgccaggctccagggaaggggctggagtgggtctcaagtattagtattagtcgtgctggcacatactacgcagactccgtgaagggccggttcaccatctccagagacaattccaagaacacgctgaatctgcaaatgaacagcctgagagccgaggacacggccgaatattactgtgcgagagagtactactacggcatggacgtctggggccaagggaccacggtcaccgtctcctca

SEQ ID NO: 212 - DNA, 321 bp, synthetic construct
  misc_feature 1..321: note = Description of Artificial Sequence: Synthetic polynucleotide
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccagactcccaggctcctcatctatgatgcatccaacagggccactggcatcccagccaggttcagtggcagtgggtctgggacagacttcacgctcaccatcagcagcctagagactgaagattttgcagtttattactgtcagcagcttatcaactggccgctcactttcggcggagggaccaagctggagatcaaa

SEQ ID NO: 213 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcatcatcacttgccgggcgagtcaaggcattagcaattatttagcctggtttcagcagaaaccagggaaagctcctaacctcctgatctatgctgcatccactttgcgatcaggggtcccatctcggttcagtggcagtggatctgggacagatttcactctcaccatcagcagcctgcagcctgaagatgttgcaacttattactgtcaaaagtataacagtgccccgtacacttttggccaggggaccaaggtggagatcaaa

SEQ ID NO: 214 - DNA, 351 bp, synthetic construct
  misc_feature 1..351: note = Description of Artificial Sequence: Synthetic polynucleotide
  source 1..351: mol_type = other DNA
gaggtgcagttgttggaatctggggcagacttggtacagcctggggggtccctgagactctcctgtgcagcctctggattcacctttagcagctatgccatgagttgggtccgccaggctccaggaaaggggctggagtgggtctcaactattcgtattagtggtggcaccacatactacgcagactccgtgaagggccggttcaccatctccagagacaattccaagaacacgctgtatctgcaaatgaacagcctgagagccgaagacacggccgtatattactgcgtaagggactactataacggtatggacgtctggggccaagggaccacggtcaccgtctcctca

SEQ ID NO: 215 - DNA, 321 bp, synthetic construct
  misc_feature 1..321: note = Description of Artificial Sequence: Synthetic polynucleotide
  source 1..321: mol_type = other DNA
gaaattgtgttgacacagtctccagccaccctgtctttgtctccaggggaaagagccaccctctcctgcagggccagtcagcgtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagctactggcctcccgcttttggccaggggaccaagctggagatcaaa

SEQ ID NO: 216 - DNA, 261 bp, synthetic construct
  misc_feature 1..261: note = Description of Artificial Sequence: Synthetic polynucleotide
  source 1..261: mol_type = other DNA
agctatgccatgagctgggtccgccaggctccagggaaggggctggagtgggtctcaagtattagtggtagtggtgatagcacaaactacgcagactccgtgaagggccggttcaccatctccagagacaattccaagaacacgctgcatctgcaaatgaacagcctgagagccgaggacacggccgtatattactgcgtaagggactactactacggtatggacgtctggggccacgggaccacggtcaccgtctcctca

SEQ ID NO: 217 - DNA, 321 bp, synthetic construct
  misc_feature 1..321: note = Description of Artificial Sequence: Synthetic polynucleotide
  source 1..321: mol_type = other DNA
gaaattgtgttgacacagtctccagccaccctgtctttgtctccaggggaaagagccaccctctcctgcagggccagtcagagtgttaacagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagctactggccgatcaccttcggccaagggacacgactggagattaaa

SEQ ID NO: 218 - DNA, 351 bp, synthetic construct
  misc_feature 1..351: note = Description of Artificial Sequence: Synthetic polynucleotide
  source 1..351: mol_type = other DNA
gaggcgcagctgttggagtctgggggaggcttggtacagcctggggggtccctgagactctcctgtgcagcctctggattcacctttagcagctatgccatgagctgggtccgccaggctccagggaaggggctggagtgggtctcaactattagtggtagtggtggtagtacacactacgcagactccgtgaagggccggttcaccatctccagagacaattccaagaatacgttgtatctgcaaatgaacagcctgagagccgaggacacggccgtatattactgcgtaagggactactactacggtatggacgtctggggccaagggaccacggtcaccgtctcctca

SEQ ID NO: 219 - DNA, 321 bp, synthetic construct
  misc_feature 1..321: note = Description of Artificial Sequence: Synthetic polynucleotide
  source 1..321: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagctactggcctcccacttttggccaggggaccaagctggagatcaaa

SEQ ID NO: 220 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtccctgagactctcctgtgcagcgtctggattcaccttcagtagctatggaatgcactgggtccgccaggctccaggcaaggggctggagtgggtggcagttatatggtatgatggaagtaaaaaatactatgtagactccgtgcagggccgattcaccatctccagagacaattccaagaatacgctgtatctgcaaatgaacagcctgagaggcgaggacacggctgtgtattactgtgcgagagatgggggagcagtggctgacaattggatcgactcctggggccagggaaccctggtcaccgtctcctca

SEQ ID NO: 221 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcgtctgtaggagacagagtcactctcacttgccgggcaagtcagaacattagaagctatttaaattggtatcagcagaaaccagggaaagcccctaagctcctgatctttgctgcatccagtttgccaagtggggtcccatcaaggttcagtggcagtggatctgtgacagatttcactctcaccgtcagcagtctgcaacctgaagattttgcaacttactcctgtcaacagagttacagtaccccgtacacttttggccaggggaccaagctggagatcaaa

SEQ ID NO: 222 - DNA, 351 bp, synthetic construct
  misc_feature 1..351: note = Description of Artificial Sequence: Synthetic polynucleotide
  source 1..351: mol_type = other DNA
gaggaacagctgttggaatctggggcagacttggcacagccgggggggtccctgagactctcctgtgcagcctctggattcacctttagcagctatgccatgagttgggtccgccaggctccaggaaagggactggagtgggtctcaactattcgtattagtggtgataccacttactacgcagactccgtgaagggccggttcaccatctccagagacaattccaagaacacgctgtttctgcaaatgaacagcctgagagccgaagacacggccgtgtattactgcgtaagggactactataacggtatggacgtctggggccatgggaccacggtcaccgtctcctca

SEQ ID NO: 223 - DNA, 321 bp, synthetic construct
  misc_feature 1..321: note = Description of Artificial Sequence: Synthetic polynucleotide
  source 1..321: mol_type = other DNA
gaaattgtgttgacacagtctccagccaccctgtctttgtctccaggggaaagagccaccctctcctgcagggccagtcagcgtgttagcagctatttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagctactggcctcccgcttttggccaggggaccaagctggagatcaaa

SEQ ID NO: 224 - DNA, 363 bp, synthetic construct
  misc_feature 1..363: note = Description of Artificial Sequence: Synthetic polynucleotide
  source 1..363: mol_type = other DNA
caggtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggatggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagaagcagtggctggtacctctgatgcttttgatatctggggccaagggacaatggtcaccgtctcctca

SEQ ID NO: 225 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgttggagacagagtcaccatcacttgccgggcaaatcagagtattgccagttatttaaattggtatcagcagaaaccaggaaaagtccctaaactcctgatctatggtgcatccagtttgcaaaatggggtcccatcaaggttcagtggcagtggatctaggacagatttcactctcaccatcagcagtctgcaacctgaagattttgcaacttactactgtcaacaaagttacagcaccgaaatcaccttcggccaagggacacgactggagattaaa

SEQ ID NO: 226 - DNA, 321 bp, synthetic construct
  misc_feature 1..321: note = Description of Artificial Sequence: Synthetic polynucleotide
  source 1..321: mol_type = other DNA
gacatccagatgacccagtctccatcctccctgtctgcatctgtaggagacagagtcgccatcacttgccgggcaagtcagagtattgccagttatttaaattggtatcagcagaaaccagggaaagcccctaaactcctgatctatggtgcatccagtttgcaaaatggggtcccatcaaggttcagtggcagtggatctaggacagatttcactctcaccatcagcagtctgcaacctgaagattttgcaacttactactgtcaacagagttacagcaccgagatcaccttcggccaagggacacgactggagattaaa

SEQ ID NO: 227 - DNA, 360 bp, synthetic construct
  misc_feature 1..360: note = Description of Artificial Sequence: Synthetic polynucleotide
  source 1..360: mol_type = other DNA
caggtgcagctggtggagtctgggggaggcgtggtccagcctgggaggtccctgcgactctcctgtgcagcgtctggattcacctttaggaactatggcatgcactgggtccgccaggctccaggcaaggggctggagtgggtggcagttatatggtatgatggaagtaataaattctatgcagactccgtgaagggccgattcaccatctccagagacaattccaagaacacgctgtatctgcaaatgaacagcctgagagtcgaggacacggctgtatattactgtgcgagagaggaggggatagcagtggccccctttgactactggggccagggaaccctggtcaccgtctcctca

SEQ ID NO: 228 - DNA, 321 bp, synthetic construct; identical to SEQ ID NO: 200
  misc_feature 1..321: note = Description of Artificial Sequence: Synthetic polynucleotide
  source 1..321: mol_type = other DNA

SEQ ID NO: 229 - DNA, 363 bp, synthetic construct
  misc_feature 1..363: note = Description of Artificial Sequence: Synthetic polynucleotide
  source 1..363: mol_type = other DNA
caggtgcagctggtggaatctgggggaggcatggtccagcctgggaggtccctgagactctcctgtacagcgtctggattcaccttcagtagtcatggcatgcactgggtccgccaggctccaggcaagggactggagtgggtggcagttatttggtttgatggaagtaatgaatattatgtagactccgtgaagggccgattcaccatctccagagacaattccaagaacacactgtatctgcaaatgaacagcctgagagccgaggacacggctgtgtattattgtgcgagagaaaacgtttatggcagtggctggttttttgactactggggccagggaaccctggtcaccgtctcctca

SEQ ID NO: 230 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagcctccctctcctgcagggccagtcagagtgttggcagcaacttagcctggtaccagcagaaacctggccaggctcccaggctcctcatctttggtgcatccaccagggccactggtatcccagccaggttcagtggcagtaggtctgggacagagttcactctcaccatcagcagcctgcagtctgaagattttgcactttattactgtcagcagtataataactggcctccgtggacgttcggccaagggaccaaggtggagatcaaa

SEQ ID NO: 231 - DNA, 363 bp, synthetic construct
  misc_feature 1..363: note = Description of Artificial Sequence: Synthetic polynucleotide
  source 1..363: mol_type = other DNA
cagttgcagctggtggagtctgggggaggcgtggtccagcctggcaggtccctgagactctcctgtgcagcgtctggattcaccttcagtagttatggcatgcactgggtccgccaggctccaggcaagggactggagtgggtggcagttatatggtatgatggaagtagtgagtactatgcagactccgtgaagggccgattcaccatctccagagacaattccaagaacacgctgtatctgcaaatgaacagcctgagagccgaggacacggctgtgtattattgtgcgagagaaaacgtgtatggcagtggctggttttttgactactggggccagggaaccctggtcaccgtctcctca

SEQ ID NO: 232 - DNA, 324 bp, synthetic construct; identical to SEQ ID NO: 230
  misc_feature 1..324: note = Description of Artificial Sequence: Synthetic polynucleotide
  source 1..324: mol_type = other DNA

SEQ ID NO: 233 - DNA, 272 bp, synthetic construct
  misc_feature 1..272: note = Description of Artificial Sequence: Synthetic polynucleotide
  source 1..272: mol_type = other DNA
gtcatggcatgcactgggtccgccaggctccaggcaagggactggagtgggtggcagttatatggtatgatggaagtagtgagtactatgcagactccgtgaagggccgattcaccatctccagagacaattccaagaacacgctgtatctgcaaatgaacagcctgagagccgaggacacggctgtgtattattgtgcgagagaaaacgtatatggcagtggctggttttttgactactggggccagggaagcctggtcaccgtctcctca

SEQ ID NO: 234 - DNA, 324 bp, synthetic construct; identical to SEQ ID NO: 210
  misc_feature 1..324: note = Description of Artificial Sequence: Synthetic polynucleotide
  source 1..324: mol_type = other DNA

SEQ ID NO: 235 - DNA, 363 bp, synthetic construct
  misc_feature 1..363: note = Description of Artificial Sequence: Synthetic polynucleotide
  source 1..363: mol_type = other DNA
cagttgcagttggtggagtctgggggaggcgtggtccagtttggcaggtccctgagactctcctgtgcagcgtctggattcaccttcagtagtcatggcatgcactgggtccgccaggctccaggcaagggactggagtgggtggcagttatatggtatgatggaagtagtgagtactatgcagactccgtgaagggccgattcaccatctccagagacaattccaagaacacgctgtatctgcaaatgaacagcctgagagccgaggacacggctgtgtattattgtgcgagagaaaacgtgtatggcagtggctggttttttgactactggggccagggaaccctggtcaccgtctcctca

SEQ ID NO: 236 - DNA, 324 bp, synthetic construct; identical to SEQ ID NO: 210
  misc_feature 1..324: note = Description of Artificial Sequence: Synthetic polynucleotide
  source 1..324: mol_type = other DNA

SEQ ID NO: 237 - DNA, 363 bp, synthetic construct
  misc_feature 1..363: note = Description of Artificial Sequence: Synthetic polynucleotide
  source 1..363: mol_type = other DNA
cagttgcagctggtggagtctgggggaggcgtggtccagcctggcaggtccctaagactctcctgtgcagcgtctggattcaccttcagtagttatggcatgcactgggtccgccaggctccaggcaagggactggagtgggtggcagttatatggtatgatggaagtagtgagtattatgcagactccgtgaagggccgattcaccatctccagagacaattccaagaacacgctgtttctgcaaatgaacagcctgagagccgatgacacggctgtgtattattgtgcgagagaaaacgtgtatggcagtggctggttttttgactactggggccagggaaccctggtcaccgtctcctca

SEQ ID NO: 238 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagcctccctctcctgcagggccagtcagagtgttggcagcaacttagcctggtaccagcagaaacctggccaggctcccaggctcctcatctttggtgcatccaccagggccactggtatcccagccaggttcagtggcagtaggtctgggacagagttcactctcaccatcagcagcctgcagtctgaagattttgcactttattactgtcagcagtatgataactggcctccgtggacgttcggccaagggaccaagctggagatcaaa

SEQ ID NO: 239 - DNA, 363 bp, synthetic construct
  misc_feature 1..363: note = Description of Artificial Sequence: Synthetic polynucleotide
  source 1..363: mol_type = other DNA
caggtgcagctggtggagtctgggggaggcgtggtccagcctgggaggtctctgagactctcctgtgcagcgtctggattcaccttaagtggctatggcatgcactgggtccgccaggctccaggcaagggactggagtgggtggcagttatatggtatgatggaagtaataaatactatgcagactccgtgaagggccgattcaccatctccagagacaattccaagaacatgctgtttctgcaaatgaacagcctgagagccgaggacacggctgtgtattactgtgtgagagaaaacgggtttggcagtggctggttttttgactactggggccagggaaacctggtcaccgtctcctca

SEQ ID NO: 240 - DNA, 324 bp, synthetic construct
  misc_feature 1..324: note = Description of Artificial Sequence: Synthetic polynucleotide
  source 1..324: mol_type = other DNA
gaaatagtgatgacgcagtctccagccaccctgtctgtgtctccaggggaaagagccaccctctcctgcaggaccagtcagagtgtaagtagagacttagcctggtaccagcagaaacctggccaggctcccaggctcctcatctatggtgcatccaccagggccactggtattccagtcaggttcagtggcagtgggtctgggacagagttcactctcaccatcagcagcctgcagtctgaagattttgcagtttattactgtcagcagtataataactggcctccgtggacgttcggccaagggaccaagctggagatcaaa

SEQ ID NO: 241 - DNA, 375 bp, synthetic construct
  misc_feature 1..375: note = Description of Artificial Sequence: Synthetic polynucleotide
  source 1..375: mol_type = other DNA
caggggcagctggtggagtctgggggaggcgtggtccagcctgggaggtccctgagactctcctgtgcagcgtctggattcaccttcagtagttataccttgcactgggtccgccaggctccaggcaaggggctggagtgggtggcagtcatatggtatgatggaagtaataagtattatgtagactccgtgaagggccgattcaccatctccagagacaattccaagaacacgctgtatctgcaaatgaacagcctgagagccgaggacacggctgtgtattactgtgcgagagagtatggttcggggagttattataacgtctactacggtatggacgtctggggccaggggaccacggtcaccgtctcctca

SEQ ID NO: 242 - DNA, 336 bp, synthetic construct
  misc_feature 1..336: note = Description of Artificial Sequence: Synthetic polynucleotide
  source 1..336: mol_type = other DNA
gatattgtgatgactcagtctccactctccctgcccgtcacccctggagagccggcctccatctcctgcaggtctagtcagagcctcctgcatagcaatggatacaagtatttggattggtatctgcagaaagcagggcagtctccacacctcttgatctatttgggttctaatcgggcctccggggtccctgacaggttcagtggcagtggatcaggcacagattttacactgaaaatcagcagagtggaggctgaggatgttggattttattactgcatgcaaactctacaaattccgctcactttcggcggagggaccaagctggagatcaaa

SEQ ID NO: 243 - DNA, 360 bp, synthetic construct
  misc_feature 1..360: note = Description of Artificial Sequence: Synthetic polynucleotide
  source 1..360: mol_type = other DNA
gaggtgcaggtggtggagtctgggggaggcttggtccagcctggggggtccctgagactctcctgtgcaacctctggattcacctttagaagctattggatgacctgggtccgccaggctccagggaaggggctggagtgggtggccaatataaagcaggatggaagtgagagacactatgtggactctgtgaagggccgattcaccatctccagagacaacgccaagacatcactgtatctgcaaatgagcagcctgagagccgaggacacggctatgtattactgtgcgagagaggggtactatggttcgggctactttgactactggggccagggaaccctggtcaccgtctcctca

SEQ ID NO: 244 - DNA, 321 bp, synthetic construct
  misc_feature 1..321: note = Description of Artificial Sequence: Synthetic polynucleotide
  source 1..321: mol_type = other DNA
gacatccagatgacccagtctccctccaccctgtctgcatctgtaggagacagagtcatcatcacttgccgggccagtcagagtattagtagggggttggcctggtatcagcagaaaccagggaaagcccctaaactcttgatctataaggcgtctaatttagaaagtggggtcccatcaaggttcagcggcagtggatctgggacagaattcactctcaccatcagcagcctgcagcctgatgattttgcagcttattactgccaacagtatagttattattctcacacttttggccaggggaccaaggtggagatcaaa

SEQ ID NO: 245 - DNA, 360 bp, synthetic construct
  misc_feature 1..360: note = Description of Artificial Sequence: Synthetic polynucleotide
  source 1..360: mol_type = other DNA
gaggtgcaactggtggagtctgggggaggcttggtccagcctggggggtccctgagactctcctgtgcagcctctggattcaccttaagtttctattggatgacctgggtccgccaggctccagggaaggggctggagtgggtggccaacataaagcaagatggaaatgagaaaaactatgtggactctgtgaagggccgattcaccatctccaaagacaacgccaagaaatcagtgtttctgcaaatgaacagcctgagagccgaggacacggctgtgtattactgtgcgagagaggggtactttggttcgggctactttgactactggggccagggaaccctggtcaccgtctcctca

SEQ ID NO: 246 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtgggagacagagtcaccatcacttgtcgggccagtcagagtattaatagctggttggcctggtatcagcagaaaccagggaaagcccctaagctcctgatctataaggcgtctactttagaaagtggggtcccatcaaggttcagcggcagtggatctgggacagaattcactctcaccatcagcagcctgcagcctgatgattttgcaccttattactgccagcattataatagttatcctcacacttttggccaggggaccaaggtggagatcaaa

SEQ ID NO: 247 - DNA, 360 bp, synthetic construct
  misc_feature 1..360: note = Description of Artificial Sequence: Synthetic polynucleotide
  source 1..360: mol_type = other DNA
gaggtgcagctggtggagtctgggggaggcttggtccagcctggggggtccctgagactctcctgtgcagcctctggattcacttttagtagttattggatgagctgggtccgccaggctccagggaaggggctggagtgggtggccaacataaagcaagatggaagtgagaaacactatgtggactctgtgaagggccgattcaccatctccagagacaacgccaagaactcactgtatctgcaaatgaacagcctgagagccgaggacacggctgtgtattactgtgcgagagaggggtactttggttcgggctactttgactactggggccagggaaccctggtcaccgtctcctca

SEQ ID NO: 248 - DNA, 321 bp, synthetic construct
  misc_feature 1..321: note = Description of Artificial Sequence: Synthetic polynucleotide
  source 1..321: mol_type = other DNA
gacatccagttgacccagtctccatccttcctgtctgcatctgtaggagacagagtcaccatcacttgccgggccagtcagggcattagcagttatttagcctggtatcagcaaaaaccagggaaagcccctaagctcctgatctatgctgcatccactttgcaaagtggggtcccatcaaggttcagcggcagtggatctgggacagaattcactctcacaatcagcagcctgcagcctgaagattttgcaacttattactgccaacagtatagtttttattctcacacttttggccaggggaccaagctggagatcaaa

SEQ ID NO: 249 - DNA, 360 bp, synthetic construct
  misc_feature 1..360: note = Description of Artificial Sequence: Synthetic polynucleotide
  source 1..360: mol_type = other DNA
gaggtgcaactggtggagtctgggggaggcttggtccagcctggggggtccctgagactctcctgtgcagcctctggattcaccttaagtttctattggatgacctgggtccgccaggctccagggaaggggctggagtgggtggccaatataaagcaagatggaaatgagaaaaactttgtggactctgtgaagggccgattcaccatctccagagacaacgccaagaaatcagtgtttctgcaaatgaacagcctgagagccgaggacacggctgtgtattactgtgcgagagaggggtactttggttcgggctactttgactactggggccagggaaccctggtcaccgtctcctca

SEQ ID NO: 250 - DNA, 321 bp, synthetic construct
  misc_feature 1..321: note = Description of Artificial Sequence: Synthetic polynucleotide
  source 1..321: mol_type = other DNA
gacatccagatgacccagtctccttccaccctgtctgcatctgtaggagacagagtcaccatcacttgccgggccagtcagagtattaatagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccagcagtataatagttatcctcacacttttggccaggggaccaagctggagatcaaa

SEQ ID NO: 251 - DNA, 321 bp, synthetic construct
  misc_feature 1..321: note = Description of Artificial Sequence: Synthetic polynucleotide
  source 1..321: mol_type = other DNA
gacatccagttgacccagtctccatccttcctgtctgcatctgtaggagacagagtcaccatctcttgccgggccagtcagggcattagcagttatttggcctggtatcagcaaaaaccagggaaagcccctaagctcctgatctatgctgcatccactttgcaaggtggggtcccatcaaggttcagcggcagtggatctgggacaacattcactctcacaatcagcagcctgcagcctgaagattttgcaacttattactgtcaacagcttaatacttacccattcactttcggccctgggaccaagctggagatcaaa

SEQ ID NO: 252 - DNA, 321 bp, synthetic construct; identical to SEQ ID NO: 251
  misc_feature 1..321: note = Description of Artificial Sequence: Synthetic polynucleotide
  source 1..321: mol_type = other DNA

SEQ ID NO: 253 - DNA, 357 bp, synthetic construct
  misc_feature 1..357: note = Description of Artificial Sequence: Synthetic polynucleotide
  source 1..357: mol_type = other DNA
caggtgcagctgcaggagtcgggcccaggactggtgaagcctttggggaccctgtccctcacctgcgctgtctctggtggctccatcagcaatagtaactggtggagttgggtccgccagcccccaggaaaggggctggagtggattggagaaatctatcatagtgggagcaccaactacaacccgtccctcaagagtcgagtcaccatagcactagactggtccaagaaccagttctccctgcagctgaggtctgtgaccgccgcggacacggccgtgtattactgtgcgcggtatggttcgggtccttttggcggtgactactggggccagggaaccctggtcaccgtctcctca

SEQ ID NO: 254 - DNA, 369 bp, synthetic construct
  misc_feature 1..369: note = Description of Artificial Sequence: Synthetic polynucleotide
  source 1..369: mol_type = other DNA
caggtgcagataaaggagtcgggcccaggactggtgaagccttcggagaccctgtccctcacctgcgctgtctctggtttctccttcagcagtggttattactggggctggatccggcagcccccagggaagggtctggagtggcttgggagtttctttcataatgggaatacctactacaacccgtccctcaggagtcgagtcaccatctcagtagacacgtccaagaaccacttctccctgaagctgacctctgtgaccgccgcagacacggccgtgtattactgtgcgggattcggggacttaccccattatcattattacgttatggacgtctggggccaagggaccacggtcaccgtctcctca

SEQ ID NO: 255 - DNA, 324 bp, synthetic construct
  misc_feature 1..324: note = Description of Artificial Sequence: Synthetic polynucleotide
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccattggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaa

SEQ ID NO: 256 - DNA, 369 bp, synthetic construct
  misc_feature 1..369: note = Description of Artificial Sequence: Synthetic polynucleotide
  source 1..369: mol_type = other DNA
caggtgcagccaaaggagtcgggcccaggagtggtgaagccttcggagaccctgtccctcacctgcgctgtctctggtttccccatcagccgtggttattactggggctggatccggcagcccccagggaagggtctggagtggattgggaatatctttcatagtgggaccacctactacaatccgtccctcaagagtcgagtcaccatctcagtagacacgtccaagaaccagatctccctgaagctgacctctgtgaccgccgcagacacggccgtatattattgtgtgggattcggggacttgccccactaccaatattacgttatggacatctggggccaagggaccacggtcaccgtctcctca

SEQ ID NO: 257 - DNA, 324 bp, synthetic construct
  misc_feature 1..324: note = Description of Artificial Sequence: Synthetic polynucleotide
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgtacacttttggccaggggaccaagctggagatcaaa

SEQ ID NO: 258 - DNA, 351 bp, synthetic construct
  misc_feature 1..351: note = Description of Artificial Sequence: Synthetic polynucleotide
  source 1..351: mol_type = other DNA
caggtgcagctgcaggagtcgggcccaggactggtgaagccttcggagaccctgtccctcacctgcgctgtctctggttactccgccagcggtctttactactgggcctggatccggcagcccccagggaagggactggagtggattggaaatatctatcatagtgggagaacctactacaatccgtccctcgagagtcgagtcagcatatcactagacacgtccaagcaccaggtctccctgaaactgaaatctgtgacctacgcagacacggccgtgtatttctgtgcgagagataggtttgatggttttgatatttggggccaagggacaatggtcaccgtctcctca

SEQ ID NO: 259 - DNA, 321 bp, synthetic construct
  misc_feature 1..321: note = Description of Artificial Sequence: Synthetic polynucleotide
  source 1..321: mol_type = other DNA
gaaatagtgatgacgcagtctccagccaccctgtctgtgtctccaggggaaagagccaccctctcctgcagggccagtcagactattaacaacatcttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctctcactttcggcggagggaccaagctggagatcaaa

SEQ ID NO: 260 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV